(19) United States
(12) Patent Application Publication
(10) Pub. No.: US 2007/0054271 A1
(43) Pub. Date: Mar. 8, 2007

(54) Gene expression in breast cancer

(75) Inventors: Kornelia Polyak, Brookline, MA (US); Dale Porter, Boston, MA (US); Minna Allinen, Espoo (FI)

Correspondence Address: FISH & RICHARDSON PC, P.O. BOX 1022, MINNEAPOLIS, MN, 55440-1022, US

(73) Assignee: DANA-FARBER CANCER INSTITUTE, INC., Boston, MA (US)

(21) Appl. No.: 10/550,162

(22) PCT Filed: Mar. 22, 2004
(86) PCT No.: PCT/US04/08866

§ 371 Date: Aug. 29, 2006

Related U.S. Application Data
(60) Provisional application No. 60/456,735, filed on Mar. 20, 2003.

(51) Int. Cl. C12Q 1/68

(52) U.S. Cl. 435/6

(57) ABSTRACT

The invention features nucleic acids encoding proteins that are expressed at a higher or a lower level in breast cancer cells than in normal breast cells or in a cell of one grade or stage of breast cancer than in a cell of another grade or stage of breast cancer. The invention also includes proteins encoded by the nucleic acids, vectors containing the nucleic acids, and cells containing the vectors. In another aspect, the invention features methods of diagnosing and treating breast cancers of various grades and stages.

This application claims priority of U.S. Provisional Application No. 60/456,735, filed Mar. 20, 2003, the disclosure of which is incorporated herein by reference in its entirety.

### STATEMENT REGARDING FEDERALLY SPONSORED RESEARCH OR DEVELOPMENT

The research, described in this application was supported in part by a grant (No. P50 CA89393-01) and a National Research Service Award (No. 5F32 CA94788-02) from the National Cancer Institute of the National Institutes of Health and a grant (No. DAMD 17 01 1 0221) from the Department of Defense. Thus the government has certain rights in the invention.

### TECHNICAL FIELD

This invention relates to breast cancer, and more particularly to genes expressed in breast cancer cells.

### BACKGROUND

Ductal carcinoma in situ (DCIS) of the breast includes a heterogeneous group of pre-invasive breast tumors with a wide range of invasive potential. In order to initiate early aggressive treatment where needed but to avoid such treatment, and its frequent harsh side effects, where not needed, it is important that methods to distinguish between DCIS and invasive breast cancer and between different types of DCIS be developed.

### SUMMARY

The invention is based on the inventors' discovery of differing patterns of gene expression in breast cancer cells versus normal cells, in DCIS cells versus invasive and/or metastatic breast cancer cells, and between different grades of DCIS. The invention thus includes “methods of diagnosis, methods of treatment, nucleic acids corresponding to newly identified genes, polypeptides encoded by such genes, and methods of screening for gene expression.

More specifically, the invention features a method of diagnosis. The method includes the steps of; (a) providing a test sample of breast tissue; (b) determining the level of expression in the test sample of a gene selected from those listed in Table 1; and (c) if the gene is expressed in the test sample at a lower level than in a control normal breast tissue sample, diagnosing the test sample as containing cancer cells.

The invention also provides a method of determining the grade of a ductal carcinoma in situ (DCIS). The method-includes the steps of: (a) providing a test sample of DCIS tissue; (b) deriving a test expression profile for the test sample by determining the level of expression in the test sample of ten or more genes selected from those listed in Tables 2-16; (c) comparing the test expression profile to control expression profiles of the ten or more genes in control samples of high grade, intermediate grade, and low grade DCIS; (d) selecting the control expression profile that most closely resembles the test expression profile; and (e) assigning to the test sample a grade that matches the grade of the control expression profile selected in step (d). The ten or more genes can be: 25 or more genes; 50 or more genes; 100 or more genes; 200 or more genes; 500 or more genes.

Another aspect of the invention is a method of determining the likelihood of a breast cancer being DCIS or invasive breast cancer. The method includes the steps of: (a) providing a test sample of breast tissue; (b) determining the level of expression in the test sample of a gene selected from the group consisting of a gene encoding CD74, a gene encoding MGC2328, a gene encoding S100A7, a gene encoding KRT19, a gene encoding trefoil factor 3 (TFF3), a gene encoding osteonectin, and a gene identified by a SAGE tag consisting of the nucleotide sequence CTGGGCGCCC; and (c) determining whether the level of expression of the selected gene in the test sample more closely resembles the level of expression of the selected gene in control cells of (i) DCIS or (ii) invasive breast cancer; and (d) classifying the test sample as: (i) likely to be DCIS if the level of expression of the gene in the test sample more closely resembles the level of expression of the gene in DCIS cells; or (ii) likely to be invasive breast cancer if the level of expression of the gene in the test sample more closely resembles the level of expression of the gene in invasive breast cancer cells.

Also embraced by the invention is a method of predicting the prognosis of a breast cancer patient. The method includes the steps of: (a) providing a sample of primary invasive breast cancer tissue from a test patient; and (b) determining the level of expression in the sample of a gene encoding S100A7 or a gene encoding fatty acid synthase (FASN). A level of expression higher than in a control sample of primary invasive breast carcinoma from a patient with a good prognosis is an indication that the prognosis of the test patient is poor.

Another method of diagnosis includes the steps of: (a) providing a test sample of breast tissue comprising a test stromal cell; and (b) determining the level of expression in the stromal cell of a gene selected from those listed in Tables 7, 8 and 10, 15, and 16, the gene being one that is, expressed in a cell of the same type as the test stromal cell at a substantially higher level when present in breast cancer tissue than when present in normal breast tissue; and (c) classifying the test sample as: (i) normal breast tissue if the level of expression of the gene in the test stromal cell is not substantially higher than a control level of expression for a cell of the same type as the test stromal cell in normal breast tissue; (ii) breast cancer tissue if the level of expression of the gene in the test stromal cell is substantially higher than a control level of expression for a cell of the same type as the test stromal cell in normal breast tissue. The stromal cells in the test sample and the standard samples can be leukocytes and the genes selected from those listed in Tables 7 and 15, e.g., genes encoding, for example, interleukin-1β (IL1β) or macrophage inhibitory protein 1α (MIP1α). The stromal cells in the test sample and the standard samples can also be myoepithelial cells or myofibroblasts and the genes selected from those listed in Tables 8, 15, and 16, e.g., genes encoding cathepsins F, K, and L, MMP2, PRSS11, thrombospondin 2, SERPING1, cytostatin C, TIMP3, platelet-derived growth factor receptor β-like (PDGFRBL), a collagen, collagen triple helix repeat containing 1 (CTHRC1), CXCL12, or CXCL14. The stromal cells in the test sample and the standard samples can be endothelial cells and the genes selected from those listed in Tables 10 and 15. Moreover, the stromal cells in the test sample and the standard samples can be fibroblasts and the genes selected from those listed in Table 15.

Another feature of the invention is method of diagnosis that involves: (a) providing a test sample of breast tissue comprising a test stromal cell; and (b) determining the level of expression in the stromal cell of a gene selected from those listed in Tables 7, 8, 10, and 15, the gene being one that is expressed in a cell of the same type as the test stromal cell at a substantially higher level when present in normal breast tissue than when present in breast cancer tissue; and (c) classifying the test sample as: (i) normal breast tissue if the level of expression of the gene in the test stromal cell is not substantially lower than a control level of expression for a cell of the same type as the test stromal cell in normal breast tissue; (ii) breast cancer tissue if the level of expression of the gene in the test stromal cell is substantially lower than a control level of expression for a cell of the same type as the test stromal cell in normal breast tissue. The stromal cells in the test sample and the standard samples can be leukocytes and the genes selected from those listed in Tables 7 and 15. Alternatively, the stromal cells in the test sample and the standard samples can be myoepithelial cells or myofibroblasts and the genes selected from those listed in Tables 8 and 15. Furthermore, the stromal cells in the test sample and the standard samples can be endothelial cells and the genes can be selected from those listed in Tables 10 and 15. In addition, the stromal cells in the test sample and the standard samples can be fibroblasts, and the genes selected from those listed in Table 15.

In another aspect, the invention provides a method of diagnosis that involves: (a) providing a test sample of breast tissue comprising a test epithelial cell of the luminal epithelial type; (b) determining the level of expression in the test epithelial cell of a gene selected from those listed in Tables 9 and 15, the gene being one that is expressed in cancerous epithelial cells of the luminal epithelial cell type at a substantially higher level than those in normal breast tissue; and (c) classifying the test sample as: (i) normal breast tissue if the level of expression of the gene in the test epithelial cell is not substantially higher than a control level of expression for an epithelial cell of luminal epithelial cell type in normal breast tissue; (ii) breast cancer tissue if the level of expression of the gene in the test epithelial cell is substantially higher than a control level of expression for an epithelial cell of the luminal epithelial type in normal breast tissue.

Also featured by the invention is a method of diagnosis that includes: (a) providing a test sample of breast tissue comprising a test epithelial cell of the luminal, epithelial type; and (b) determining the level of expression in the test epithelial cell of a gene selected from those listed in Table 9, the gene being one that is expressed in epithelial cells of the luminal epithelial cell type at a substantially lower level when present in breast cancer tissue than when present in normal breast tissue; and (c) classifying the test sample as: (i) normal breast tissue if the level of expression of the gene in the test epithelial cell is not substantially lower than a control level of expression for an epithelial cell of luminal epithelial cell type in normal breast tissue; (ii) breast cancer tissue if the level of expression of the gene in the test epithelial cell is substantially lower than a control level of expression for an epithelial cell of the luminal epithelial type in normal breast tissue.

In all the above methods of the invention the level of expression of the gene can determined as a function of the level of protein encoded by the gene or as a function of the level of mRNA transcribed from the gene.

Another embodiment of the invention is a method of inhibiting proliferation or survival of a breast cancer cell. The method involves contacting a breast cancer cell with a polypeptide that is encoded by a gene selected from those listed in. Tables 1, 7-10, and 15, the gene being one that is expressed in the cancer cell, or a stromal cell in a tumor comprising the cancer cell, at a level substantially lower than in a normal cell of the same type. In the method, the cancer cell can be in vitro. Alternatively, it can be in a mammal, e.g., a human; The contacting can include administering the polypeptide to the mammal or administering a polynucleotide encoding the polypeptide to the mammal. The method can also involve: (a) providing a recombinant cell that is the progeny of a cell obtained from the mammal and has been transfected or transformed ex vivo with a nucleic acid encoding the polypeptide; and (b) administering the recombinant cell to the mammal, so that the recombinant cell expresses the polypeptide in the mammal.

Another feature of the invention is a method of inhibiting pathogenesis of a breast cancer cell or stromal cell in a tumor of a mammal. The method includes: (a) identifying a mammal with a breast cancer tumor; and (b) administering to the mammal an agent that inhibits binding of a polypeptide encoded by a gene selected from those listed in Tables 2-10, 15, and 16 to its receptor or ligand, the gene being one that is expressed in a breast cancer cell in the tumor, or in a stromal cell in the tumor, at a level substantially higher than in a corresponding cell in a non-cancerous breast. The polypeptide is a secreted polypeptide or a cell-surface polypeptide. The agent can be a non-agonist antibody that binds to the polypeptide, a soluble form of the receptor, or a non-agonist antibody that binds to the receptor or ligand. The polypeptide can be, for example, CXCL12 or CXCL14 and the receptor can be, for example, CXCR4 or a receptor for CXCL14.

Another aspect of the invention is a method of inhibiting expression of a gene in a cell. The method includes introducing into a target cell selected from the group consisting of (a) a breast cancer cell and (b) stromal cell in a tumor comprising a breast cancer cell, an agent that inhibits expression of a gene selected from those listed in Tables 2-10, 15, and 16, the gene being one that is expressed in the target cell at a level substantially higher than in a corresponding cell in normal breast tissue. The agent can be an antisense oligonucleotide that hybridizes to an mRNA transcribed from the gene. The introducing step can involve administration of the antisense oligonucleotide to the target cell. The introducing step comprises administering to the target cell a nucleic acid comprising a transcriptional regulatory element (TRE) operably linked to a nucleotide sequence complementary to, the antisense oligonucleotide, wherein transcription of the nucleotide sequence inside the target cell produces the antisense oligonucleotide. The agent can also be an RNAi molecule, one strand of the RNAi molecule having the ability to hybridize to a mRNA transcribed from the gene. The agent can also be a small molecule that inhibits expression of the gene. The gene can be one that encodes, for example, can be, for example, CXCL12, CXCL14, CXCR4, or a receptor for CXCL14.

Also provided by the invention is an isolated DNA that includes: (a) the nucleotide sequence of a tag selected from those listed in FIG. 7; or (b) the complement of the nucleotide sequence. Also embraced by the invention is a vector containing the DNA. In the vector, the DNA can optionally be operatively linked to a transcriptional regulatory element (TRE). A cell comprising any of the vectors of the invention is also an aspect of the invention. Also included in the invention is an isolated polypeptide encoded by the DNA of the invention.

In another aspect, the invention embraces a single stranded nucleic acid probe that includes: (a) the nucleotide sequence of a tag selected from those listed in Tables 1-5, 7-10, 15, and 16; or (b) the complement of the nucleotide sequence.

Also embodied by the invention is an array that includes a substrate having at least 10 addresses, each address having disposed on it a capture probe that includes a nucleic acid sequence consisting of a tag nucleotide sequence selected from those listed in Tables 1-5, 7-10, 15, and 16. The tag nucleotide sequence can be one that corresponds to a gene encoding a protein selected from the group consisting of fatty acid synthase (FASN), trefoil factor 3 (TFF3), X-box binding protein 1 (XBP1), interferon alpha inducible protein 6-16 (IFI-6-16), cysteine-rich protein 1 (CRIP1), interferon-stimulated protein 15 kDa (ISG15), interferon alpha inducible protein 27 (IFI27), brain expressed X linked 1 (BEX1), helicase/primase protein (LOC150678), anaphase promoting complex subunit 11 (ANAPC11), Fer-1-like 4 (FER1L4), psoriasin, connective tissue growth factor (CTGF), regulator of G-protein signaling 5 (RGS5), paternally expressed 10 (PEG10), osteonectin (SPARC), LOC51235, CD74, MGC23280, Invasive Breast Cancer 1 (IBC-1), Apolipoprotein D (APOD), carboxypeptidase B1 (CPB1), retinal binding protein 1 (RBP1), FLJ30428, calmodulin-like skin protein (CLSP), nudix (NUDT8), MGC14480, interleukin-1β (ILβ), macrophage inhibitory protein 1α (MIP1α), cathepsins F, K, and L, MMP2, PRSS11, thrombospondin 2, SERPING1, cytostatin C, TIMP3, platelet-derived growth factor receptor β-like (PDGFRBL), a collagen, collagen triple helix repeat containing 1 (CTHRC1), CXCL12, CXCL14, and a protein encoded by a gene identified by a SAGE tag consisting of the nucleotide sequence CTGGGCGCCC. The array can contain at least 25 addresses; at least 50 addresses; at least 100 addresses; at least 200 addresses; or at least 500 addresses.

The invention also features a kit comprising at least 10 probes, each probe including a nucleic acid sequence that includes a tag nucleotide sequence selected from those listed in Tables 1-5, 7-10, 15, and 16. The kit can contain at least 25 probes; at least 50 probes; at least 100 probes; at least 200 probes; at least 500 probes.

Another kit provided by the invention is one that contains at least 10 antibodies each of which is specific for a different protein encoded by a gene identified by a tag selected from the group consisting of the tags listed in Tables 1-5, 7-10, 15, and 16. The antibodies can, for example, be specific for a protein selected from the group consisting of fatty acid synthase (FASN), trefoil factor 3 (TFF3), X-box binding protein 1 (XBP1), interferon alpha inducible protein 6-16 (IF1-6-16), cysteine-rich protein 1 (CRIP1), interferon-stimulated protein15 kDa (ISG15), interferon alpha inducible protein 27 (IFI27), brain expressed X linked 1 (BEX1), helicase/primase protein (LOC150678), anaphase promoting complex subunit 11 (ANAPC11), Fer-1-like 4 (FER1 L4), psoriasin, connective tissue growth factor (CTGF), regulator of G-protein signaling 5 (RGS5), paternally expressed 10 (PEG110), osteonectin (SPARC), LOC51235, CD74, MGC23280, Invasive Breast Cancer 1 (IBC-1), Apolipoprotein D (APOD), carboxypeptidase B1 (CPB1), retinal binding protein 1 (RBP1), FLJ30428, calmodulin-like skin protein (CLSP), nudix (NUDT8), MGC14480, interleukin-1β (ILβ), macrophage inhibitory protein 1α (MIP1α), cathepsins F, K, and L, MMP2, PRSS11, thrombospondin 2, SERPING1, cytostatin C, TIMP3, platelet-derived growth factor receptor β-like (PDGFRBL), a collagen, collagen triple helix repeat containing 1 (CTHRC1), CXCL12, CXCL14, and a protein encoded by a gene identified by a SAGE tag consisting of the nucleotide sequence CTGGGCGCCC. The kit can contain at least 25 antibodies; at least 50 antibodies; at least 100 antibodies; at least 200 antibodies; or at least 500 antibodies.

In addition the invention provides a method of identifying the grade of a DCIS. The method involves: (a) providing a test sample of DCIS tissue, (b) using the above-described array to determine a test expression profile of the sample; (c) providing a plurality of reference profiles, each derived from a DCIS of a defined grade, the test expression profile and each reference profile having a plurality of values, each value representing the expression level of a gene corresponding to a tag selected from those listed in Tables 1-5, 7-10, 15, and 16; and (d) selecting the reference profile most similar to the test expression profile, to thereby identify the grade of the test DCIS.

In another embodiment, the invention provides a method of determining whether a breast cancer is a DCIS or an invasive breast cancer. The method involves: (a) providing a test sample of breast cancer tissue; (b) determining the level of expression of CXCL14 in myofibroblasts in the test sample; (c) determining whether the level of expression of CXCL14 in the myofibroblasts in the test sample more closely resembles the level of expression of CXCL14 in control myofibroblasts of (i) DCIS or (ii) invasive breast cancer; and (d) classifying the test sample as: (i) DCIS if the level of expression of CXCL14 in myofibroblasts in the test sample more closely resembles the level of expression of CXCL14 in control myofibroblasts of DCIS; (ii) invasive breast cancer if the level of expression of CXCL14 in myofibroblasts in the test sample more closely resembles the level of expression of CXCL14 in control myofibroblasts of invasive breast cancer.

Polypeptide” and “protein” are used interchangeably and mean any peptide-linked chain of amino acids, regardless of length or post-translational modification.

The term “isolated” polypeptide or peptide fragment as used herein refers to a polypeptide or a peptide fragment which either has no naturally-occurring counterpart or has been separated or purified from components which naturally accompany it, e.g., in tissues such as pancreas, liver, spleen, ovary, testis, muscle, joint tissue, neural tissue, gastrointestinal tissue, or breast tissue or tumor tissue (e.g., breast cancer tissue), or body fluids such as blood, serum, or urine. Typically, the polypeptide or peptide fragment is considered “isolated” when it is at least 70%, by dry weight, free from the proteins and other naturally-occurring organic molecules with which it is naturally associated. Preferably, a preparation of a polypeptide (or peptide fragment thereof) of the invention is at least 80%, more preferably at least 90%, and most preferably at least 99%, by dry weight, the polypeptide (or the peptide fragment thereof), respectively, of the invention. Since a polypeptide that is chemically synthesized is, by its nature, separated from the components that naturally accompany it, the synthetic polypeptide is “isolated.”

An isolated polypeptide (or peptide fragment) of the invention can be obtained, for example, by extraction from a natural source (e.g., from tissues or bodily fluids); by expression of a recombinant nucleic acid encoding the polypeptide; or by chemical synthesis. A polypeptide that is produced in a cellular system different from the source from which it naturally originates is “isolated,” because it will necessarily be free of components which naturally accompany it. The degree of isolation or purity can be measured by any appropriate method, e.g., column chromatography, polyacrylamide gel electrophoresis, or HPLC analysis.

An “isolated DNA” is either (1) a DNA that contains sequence not identical to that of any naturally occurring sequence, or (2), in the context of a DNA with a naturally-occurring sequence (e.g., a cDNA or genomic DNA), a DNA free of at least one of the genes that flank the gene containing the DNA of interest in the genome of the organism in which the gene containing the DNA of interest naturally occurs. The term therefore includes a recombinant DNA incorporated into a vector; into an autonomously replicating plasmid or virus, or into the genomic DNA of a prokaryote or eukaryote. The term also includes a separate molecule such as: a cDNA where the corresponding genomic DNA has introns and therefore a different sequence; a genomic fragment that lacks at least one of the flanking genes; a fragment of cDNA or genomic DNA produced by polymerase chain reaction (PCR) and that lacks at least one of the flanking genes; a restriction fragment that lacks at least one of the flanking genes; a DNA encoding a non-naturally occurring protein such as a fusion protein, mutein, or fragment of a given protein; and a nucleic acid which is a degenerate variant of a cDNA or a naturally occurring nucleic acid. In addition, it includes a recombinant nucleotide sequence that is part of a hybrid gene, i.e., a gene encoding a non-naturally occurring fusion protein. It will be apparent from the foregoing that isolated DNA does not mean a DNA present among hundreds to millions of other DNA molecules within, for example, cDNA or genomic DNA libraries or genomic DNA restriction digests in, for example, a restriction digest reaction mixture or an electrophoretic gel slice.

As used herein, a “functional fragment” of a polypeptide is a fragment of the polypeptide that is shorter than the full-length; mature polypeptide and has at least 5% (e.g., at least: 5%; 10%; 20%; 30%; 40%; 50%; 60%; 70%; 80%; 90%; 95%; 98%; 99%; 100%; or more) of the activity (e.g., ability to inhibit proliferation of breast cancer cells) of the full-length, mature polypeptide. Fragments of interest can be made either by recombinant, synthetic, or proteolytic digestive methods. Such fragments can then be isolated and tested for their ability, for example, to inhibit the proliferation of cancer cells as measured by [³H]-thymidine incorporation or cell counting.

As used herein, “operably linked” means incorporated into a genetic construct so that expression control sequences effectively control expression of a coding sequence of interest.

As used herein, the term “antibody” refers not only to whole antibody-molecules, but also to antigen-binding fragments, e.g., Fab, F(ab′)₂, Fv, and single chain Fv (ScFv) fragments. Also included are chimeric antibodies.

As used-herein, the term “pathogenesis” of a cell (e.g., a cancer cell or stromal cell within a tumor containing a cancer cell) means proliferation of a cell, survival of a cell, invasiveness of a cell, migratory potential of a cell, metastatic potential of cell, ability of a cell to evade immune effector mechanisms, ability of a cell to induce or enhance angiogenesis, or ability of a cell to induce or enhance lymphangenesis.

As used herein, a gene that is expressed at a “substantially higher level” in a first cell (or first issue) than in a second cell (or second tissue) is a gene that is expressed in the first cell (or tissue) at a level at least 2 (e.g., at least: 2; 3; 4; 5; 6; 7; 8; 9; 10; 15; 20; 30; 40; 50; 75; 100; 200; 500; 1,000; 2000; 5,000; or 10,000) times higher than in the second cell (or second tissue).

As used herein, a gene that is expressed at a “substantially lower level” in a first cell (or first issue) than in a second cell (or second tissue) is a gene that is expressed in the first cell (or tissue) at a level at least 2 (e.g., at least: 2; 3; 4; 5; 6; 7; 8; 9; 10; 15; 20; 30; 40; 50; 75; 100; 200; 500; 1,000; 2000; 5,000; or 10,000) times lower than in the second cell (or second tissue).

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention, e.g., diagnosing breast cancer, will be apparent from the following description, from the drawings and from the claims.

### DESCRIPTION OF DRAWINGS

FIG. 1 is diagrammatic representation of the antibody-based procedure used to purify epithelial and stromal cells from DCIS and normal breast tissue for the analysis described in Example 6.

FIG. 2 is a series of photographs of ethidium bromide-stained electrophoretic gels of the products of RT-PCRs. The RT-PCR analysis was carried out on mRNA isolated from: (a) luminal epithelial cells (“epithelium”), myoepthelial cells (“myoepithelium”), leukocytes, and endothelial cells (“endothelium”) purified from two DCIS tumor sample (“DCIS6” and “DCIS7”); and (6) leukocytes and endothelial cells (“endothelium”) from normal breast tissue (“Normal”). The PCR phases of the RT-PCRs were carried out with oligonucleotide primers specific for two constitutively expressed genes (β-actin (“BAC”) and L19) and for HER2 (expressed by some breast cancers), CALLA (a myoepithelial cell marker), CD45 (a pan-leukocyte marker), and a cell surface protein specifically expressed by endothelial cells (“CDH5”). The numbers at the bottom of each column of photographs (“25”, “30”, and “35”) indicate numbers of PCR cycles.

FIG. 3A is a dendrogram showing the relatedness of SAGE libraries generated from normal mammary luminal epithelial cells (N1 and N2), DCIS cells (D1-D7 and T18), primary invasive breast cancer cells (11-16), breast cancer cells in lymph node metastases (LN1 and LN2), and breast cancer cells in a distant lung metastasis (M1) and analyzed by hierarchical clustering.

FIG. 3B is a dendrogram showing similarities among intermediate and high grade DCIS tumor SAGE libraries analyzed by hierarchical clustering using 582 genes.

FIG. 3C is a dendrogram showing similarities among intermediate and high grade DCIS tumor SAGE libraries analyzed by hierarchical clustering using 26 genes selected from the 582 genes used for the analysis depicted in FIG. 1B.

FIG. 4A is a series of photomicrographs showing the hybridization of riboprobes corresponding to genes encoding IFI-6-16, S100A7, CTGF, and RGS5 to frozen sections of DCIS tumors (T18, 96-331, 6164) and normal breast tissue (N24). Strong expression (indicated by dark staining) of IFI-6-16 and S100A7 is detected in tumor cells of a subset of DCIS tumors but not in normal breast tissue epithelial cells. Expression of CTGF and RGS5 is seen mostly in DCIS stromal fibroblasts and myoepithelial cells, respectively, but not in the corresponding cells in normal breast tissue.

FIG. 4B is dendrogram showing the relatedness of five normal breast tissues, and 18 DCIS and invasive tumors-analyzed for expression of 14 genes (SCGB3A1, TM4SF1, CTGF, XBP1, IFI27, ISG15, RGS5, RGS5, LOC150678, BEX1, PEG10, IFI-6-16, TFF3, CRIP1, S100A7, and CTGF) by mRNA in situ hybridization. Numbers are specimen identifiers. “N” denotes normal breast tissue, “D” denotes DCIS tissue, and “I” denotes invasive breast cancer tissue.

FIG. 4C is series of photomicrographs showing immunohistochemical staining of sections of a representative DCIS tumor in a tissue microarray. The tissue sections were stained with monoclonal antibodies specific for the indicated proteins. Dark staining indicates the presence of the protein. The data thus indicate the presence of S100A7, TFF3, SPARC, and CTGF but absence of IBC-1 in the DCIS tumor.

FIG. 5 is diagrammatic representation of the antibody-based procedure used to purify epithelial and stromal cells from DCIS and normal breast tissue for the analysis described in Example 7.

FIG. 6A is a line graph depicting the results of a Scatchard analysis of alkaline phosphate (AP) conjugated CXCL14 (AP-CXCL14) binding to MDA-MB-231 breast cancer cells.

FIG. 6B is a series of line graphs showing the effect of AP-CXCL14 (left and right panels) and CXCL12 (center panel) on the growth of MDA-MB-231 breast cancer cells (left and center panels) and MCF10A immortalized normal breast epithelial cells (right panel).

FIG. 6C is a pair of bar graphs showing the ability of CXCL14 N-terminally conjugated with AP (AP-CXCL14), or C-terminally conjugated with AP (CXCL14-AP), to enhance migration (left panel) and invasion (right panel) of MDA-MB-231 breast cancer cells. The cultures containing the CXCL14 conjugates (and corresponding control cultures) were in serum-free medium. Data from control-cultures carried out in medium containing 10% FBS and no CXCL14 conjugate are shown (“10% FBS”).

FIG. 7 is a depiction of the nucleotide sequences of SAGE tags that are listed in Tables 1-4, 7, 8, 10, and 15 and that correspond to no cDNA or mRNA nucleotide sequences present in the publicly available databases searched by the inventors.

### DETAILED DESCRIPTION

Various aspects of the invention are described below.

Nucleic Acid Molecules

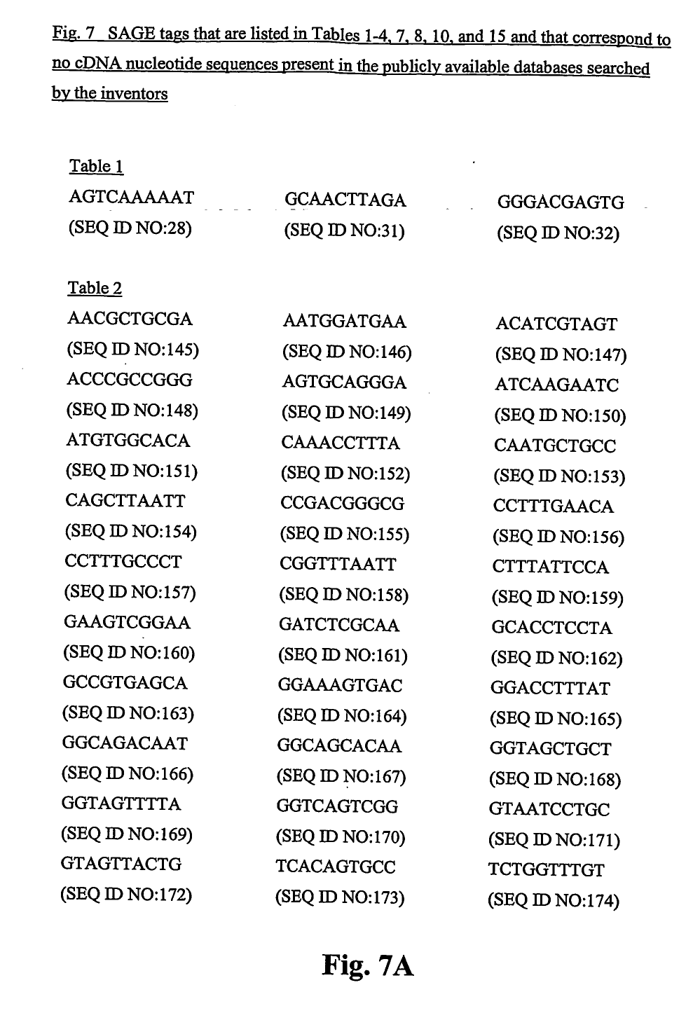

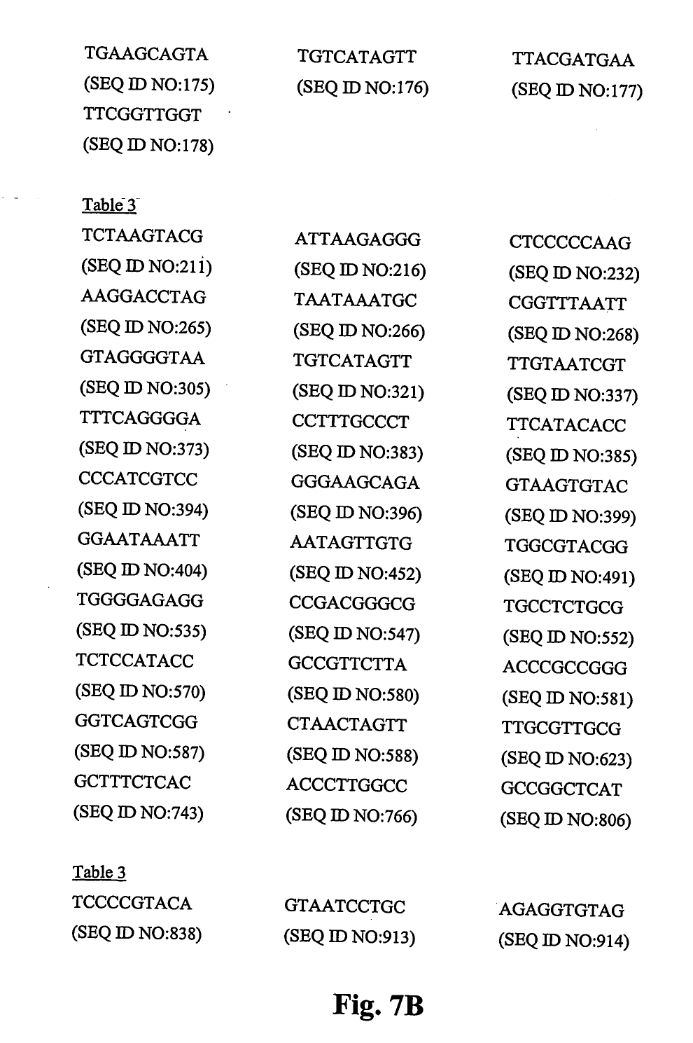

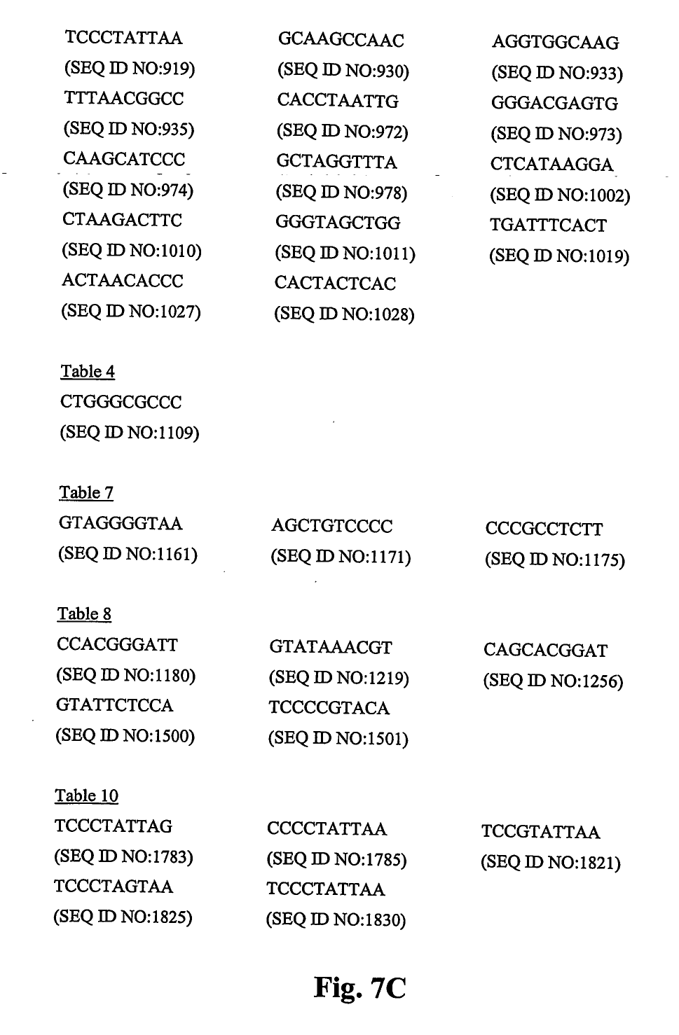

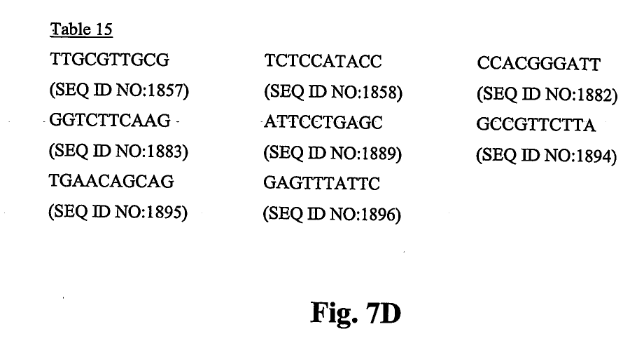

The nucleic acid molecules of the invention include those containing or consisting of the nucleotide sequences (or the complements thereof) of the SAGE (serial analysis of gene expression) tags listed in FIG. 7. The nucleic acid molecules of the invention can be cDNA, genomic DNA, synthetic DNA, or RNA, and can be double-stranded or single-stranded (i.e., either a sense or an antisense strand). Segments of these molecules are also considered within the scope of the invention, and can be produced by, for example, the polymerase chain reaction (PCR) or generated by treatment with one or more restriction endonucleases. A ribonucleic acid (RNA) molecule can be produced by in vitro transcription. Preferably, the nucleic acid molecules encode polypeptides that, regardless of length, are soluble under normal physiological conditions.

The nucleic acid molecules of the invention can contain naturally occurring sequences, or sequences that differ from those that occur naturally, but, due to the degeneracy of the genetic code, encode the same polypeptide. In addition, these nucleic acid molecules are not limited to coding sequences, e.g., they can include some or all of the non-coding sequences that lie upstream or downstream from a coding sequence. They can also contain irrelevant sequences at their 5′ and/or 3′ ends (e.g., sequences derived from a vector).

The nucleic acid molecules of the invention can be synthesized (for example, by phosphoramidite-based synthesis) or obtained from a biological cell, such as the cell of a mammal. The nucleic acids can be those of a human, non-human primate (e.g., monkey), mouse, rat, guinea pig, cow, sheep, horse, pig, rabbit, dog, or cat. Combinations or modifications of the nucleotides within these types of nucleic acids are also encompassed.

In addition, the isolated nucleic acid molecules of the invention encompass segments that are not found as such in the natural state. Thus, the invention encompasses recombinant nucleic acid molecules incorporated into a vector (for example, a plasmid or viral vector) or into the genome of a heterologous cell (or the genome of a homologous cell, at a position other than the natural chromosomal location). Recombinant nucleic acid molecules and uses therefor are discussed further below.

Techniques associated with detection or regulation of genes are well known to skilled artisans. Such techniques can be used to diagnose and/or treat disorders (e.g., DCIS or invasive cancer) associated with aberrant expression of the genes corresponding to the SAGE tags listed in FIG. 7.

Family members of the genes or proteins or proteins of the invention can be identified based on their similarity to the relevant gene or protein, respectively. For example, the identification can be based on sequence identity. The invention features isolated nucleic acid molecules which are at least 50% (or at least: 55%; 65%; 75%; 85%; 95%; 98%; 99%; 99.5%; or even 100%) identical to: (a) nucleic acid molecules that encode polypeptides encoded by genes corresponding to the SAGE tags listed in FIG. 7; (b) the nucleotide sequences of the coding regions of genes corresponding to the SAGE tags listed in FIG. 7; (c) nucleic acid molecules that include a segments of at least 30 (e.g., at least: 40; 50; 60; 80; 100; 125; 150; 175; 200; 250; 300; 500; 700; 1,000; 2,000; 3000; 5,000, 10,000; or more) nucleotides of the coding regions of genes corresponding to the SAGE tags listed in FIG. 7; and (d) nucleic acid molecules that include the genomic sequences of genes corresponding to the SAGE tags listed in FIG. 7; (e) nucleic acid molecules that include a segments of at least 30 (e.g., at least: 40; 50; 60; 80; 100; 125; 150; 175; 200; 250; 300; 500; 700; 1,000; 2,000; 3000; 5,000, 10,000; or more) nucleotides of the genomic sequences of genes listed corresponding to the SAGE tags listed in FIG. 7; (f) nucleic acid molecules containing or consisting of the SAGE tags listed in FIG. 7.

The determination of percent identity between two sequences is accomplished using the mathematical algorithm of Karlin and Altschul [(1990) Proc. Natl. Acad. Sci. USA 87:2264-2268] modified as in Karlin and Altschul [(1993) Proc. Natl. Acad. Sci. USA 90: 5873-5877]. Such an algorithm is incorporated into the BLASTN and BLASTP programs of Altschul et al. [(1990) J. Mol. Biol. 215: 403-410]. BLAST nucleotide searches are performed with the BLASTN program, score=100, wordlength=12, to obtain nucleotide sequences homologous to any of the nucleic acid molecules described herein. BLAST protein searches are performed with the BLASTP program; score=50, wordlength=3; to obtain amino acid sequences homologous to the polypeptides by encoded by any of the nucleic acid molecules described herein. To obtain gapped alignments for comparative purposes, Gapped BLAST is utilized as described in Altschul et al. [(1997) Nucleic Acids Res. 25:3389-3402]. When utilizing BLAST and Gapped BLAST programs, the default parameters of the respective programs (e.g., XBLAST and NBLAST) are used.

Hybridization cane also be used as a measure of homology between two nucleic acid sequences. A nucleic acid sequence, or a portion thereof, can be used as a hybridization probe according to standard hybridization techniques. The hybridization of a nucleic acid probe specific for a target DNA or RNA of interest to DNA or RNA from a test source (e.g., a mammalian cell) is an indication of the presence of the target DNA or RNA in the test source. Hybridization conditions are known to those skilled in the art and can be found in Current Protocols in Molecular Biology, John Wiley & Sons, N.Y., 6.3.1-6.3.6, 1991. Moderate hybridization conditions are defined as equivalent to hybridization in 2× sodium chloride/sodium citrate (SSC) at 30° C., followed by a wash in 1×SSC, 0.1% SDS at 50° C. Highly stringent conditions are defined as equivalent to hybridization in 6× sodium chloride/sodium citrate (SSC) at 45° C., followed by awash in 0.2×SSC, 0.1% SDS at 65° C.

The invention also encompasses: (a) vectors (see below) that contain any of the foregoing coding sequences and/or their complements (that is, “antisense” sequences); (b) expression vectors that contain any of the foregoing coding sequences operably linked to any transcriptional/translational regulatory elements (examples of which are given below) necessary to direct expression of the coding sequences; (c) expression vectors encoding, in addition to a polypeptide encoded by any of the foregoing sequences, a sequence unrelated to the polypeptide, such as a reporter, a marker, or a signal peptide fused to the polypeptide; and (d) genetically engineered host cells (see below) that contain any of the foregoing expression vectors and thereby express the nucleic acid molecules of the invention.

Recombinant nucleic acid molecules can contain a sequence encoding a polypeptide of the invention having a heterologous signal sequence. The full length polypeptide of the invention, or a fragment thereof, may be fused to such heterologous signal sequences or to additional polypeptides, as described below. Similarly, the nucleic acid molecules of the invention can encode the mature forms of the polypeptides of the invention or forms that include an exogenous polypeptide that facilitates secretion.

The transcriptional/translational regulatory elements referred to above include but are not limited to inducible and non-inducible promoters, enhancers, operators and other elements that are known to those skilled in the art and that drive or otherwise regulate gene expression. Such regulatory elements include but are not limited, to the cytomegalovirus hCMV immediate early gene, the early or late promoters of SV40 adenovirus, the lac system, the trp system, the TAC system, the TRC system, the major operator and promoter regions of phage A, the control regions of fd coat protein, the promoter for 3-phosphoglycerate kinase, the promoters of acid phosphatase, and the promoters of the yeast α-mating factors.

Similarly, the nucleic acid can form part of a hybrid gene encoding additional polypeptide sequences, for example, a sequence that functions as a marker or reporter. Examples of marker and reporter genes include β-lactamase, chloramphenicol acetyltransferase (CAT), adenosine deaminase (ADA), aminoglycoside phosphotransferase (neo^{r}, G418^{r}), dihydrofolate reductase (DBFR), hygromycin-B-phosphotransferase (HPH), thymidine kinase (TK), lacZ (encoding β-galactosidase), and xanthine guanine phosphoribosyltransferase (XGPRT). As with many of the standard procedures associated with the practice of the invention, skilled artisans will be aware of additional useful reagents, for example, additional sequences that can serve the function of a marker or reporter. Generally, the hybrid polypeptide will include a first portion and a second portion; the first portion being one of the proteins encoded by genes corresponding to the SAGE tags listed in FIG. 7 (or a functional fragment of such a protein) and the second portion being, for example, one of the reporters described above or an Ig constant region or part of an Ig constant region, e.g., the CH2 and CH3 domains of IgG2a heavy chain. Other hybrids could include an antigenic tag or His tag to facilitate purification.

The expression systems that may be used for purposes of the invention include but are not limited to microorganisms such as bacteria (for example, E. coli and B. subtilis) transformed with recombinant bacteriophage DNA, plasmid DNA, or cosmid DNA expression vectors containing the nucleic acid molecules of the invention; yeast (for example, Saccharomyces and Pichia) transformed with recombinant yeast expression vectors containing the nucleic acid molecule of the invention; insect cell systems infected with recombinant virus expression vectors (for example, baculovirus) containing the nucleic acid molecule of the invention; plant cell systems infected with recombinant virus expression vectors (for example, cauliflower mosaic virus (CaMV) or tobacco mosaic virus (TMV)) or transformed with recombinant plasmid expression vectors (for example, Ti plasmid) containing any of the nucleotide sequences recited above; or mammalian cell systems (for example, COS, CHO, BHK, 293, VERO, HeLa, MDCK, WI38, and NIH 3T3 cells) harboring recombinant expression constructs containing promoters derived from the genome of mammalian cells (for example, the metallothionein promoter) or from mammalian viruses (for example, the adenovirus late promoter and the vaccinia virus 7.5K promoter). Also useful as host cells are primary or secondary cells obtained directly from a mammal and transfected with a plasmid vector or infected with a viral vector.

Polypeptides and Polypeptide Fragments

The polypeptides of the invention include all those encoded by the nucleic acids described above and functional fragments of these polypeptides. The polypeptides embraced by the invention also include fusion proteins that contain either a full-length polypeptide, or a functional fragment thereof, fused to unrelated amino acid sequence. The unrelated sequences can be additional functional domains or signal peptides. The polypeptides can be any of those described-above but with not more than 50 (e.g., not more than: 50; 40; 30; 25; 20; 15; 12, 10; nine; eight; seven; six; five; four; three; two; or one) conservative substitution(s). Conservative substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. All that is required of a polypeptide with one or more conservative substitutions is that it have at least 5% (e.g., at least: 5%; 10%; 20%; 30%; 40%; 50%; 60%; 70%; 80%; 90%; 95%; 98%; 99%; 100%; or more) of the activity (e.g., ability to inhibit proliferation of breast cancer cells) of the relevant wild-type, mature polypeptide.

Polypeptides of the invention and those useful for the invention can be purified from natural sources (e.g., blood, serum, plasma, tissues or cells such as normal breast or cancerous breast epithelial cells (of the luminal type), myoepithelial cells, leukocytes, or endothelial cells). Smaller peptides (less than 50 amino acids long) can also be conveniently synthesized by standard chemical means. In addition, both polypeptides and peptides can be produced by standard in vitro recombinant DNA techniques and in vivo transgenesis, using nucleotide sequences encoding the appropriate polypeptides or peptides. Methods well-known to those skilled in the art can be used to construct expression vectors containing relevant coding sequences and appropriate transcriptional/translational control signals. See, for example; the techniques described in Sambrook et al., Molecular Cloning: A Laboratory Manual (2nd Ed.) [Cold Spring Harbor Laboratory, N.Y., 1989], and Ausubel et al., Current Protocols in Molecular Biology [Green Publishing Associates and Wiley Interscience, N.Y., 1989].

Polypeptides and fragments of the invention, and those useful for the invention, also include those described above, but modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. This can be done either chemically during the synthesis of the peptide or by recombinant DNA technology by methods familiar to artisans of average skill.

Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Likewise, the peptides can be covalently or noncovalently coupled to pharmaceutically acceptable “carrier” proteins prior to administration.

Also of interest are peptidomimetic compounds that are designed based upon the amino acid sequences of the functional peptide fragments. Peptidomimetic compounds are synthetic compounds having a three-dimensional conformation (i.e., a “peptide motif”) that is substantially the same as the three-dimensional conformation of a selected-peptide. The peptide motif provides the peptidomimetic compound with the ability to inhibit the pathogenesis of breast cancer cells in a manner qualitatively identical to that of the functional fragment from which the peptidomimetic was derived. Peptidomimetic compounds can have additional characteristics that enhance their therapeutic utility, such as increased cell permeability and prolonged biological half-life.

The peptidomimetics typically have a backbone that is partially or completely non-peptide, but with side groups that are identical to the side groups of the amino acid residues that occur in the peptide on which the peptidomimetic is based. Several types of chemical bonds, e.g., ester, thioester, thioamide, retroamide, reduced carbon A, dimethylene and ketomethylene bonds, are known in the art to be generally useful substitutes for peptide bonds in the construction of protease-resistant peptidomimetics.

In the sections below, a “gene X” represents any of the genes listed in Tables 1-16; mRNA transcribed from gene X is referred to as “mRNA X”; protein encoded by gene X is referred to as “protein X”; and cDNA produced from mRNA X is referred to as “cDNA X”. It is understood that, unless otherwise stated, descriptions containing these terms are applicable to any of the genes listed in Tables 1-16, mRNAs transcribed from such genes, proteins encoded by such genes, or cDNAs produced from the mRNAs.

Diagnostic Assays

The invention features diagnostic assays. Such assays are based on the findings that: (a) certain genes are expressed at a higher level, or a lower level, in breast epithelial cancer cells (or non-epithelial cells within a relevant breast tumor) compared to normal cells of the same types; and (b) breast cancers of various grades and/or stages differ from each other in terms of the patterns of genes they express and in the levels at which they express them. These findings provide the bases for assays to diagnose breast cancer and to define the grade and/or stage of a breast cancer. Such assays can be used on their own or, preferably, in conjunction with other procedures to diagnose breast cancer and/or identify the grade and/or stage of progression Of a breast cancer.

The diagnostic assays of the invention generally involve testing for levels of expression of one or a plurality of the genes listed in Tables 1-16. By testing for levels of expression in a cell of a plurality of genes, one obtains an “expression profile” of the cell.

In the assays of the invention either: (1) the presence of protein X or mRNA X in cells is tested for or their levels in cells are measured; or (2) the level of protein X is measured in a liquid sample such as a body fluid (e.g., urine, saliva, semen, blood, or serum or plasma derived from blood); a lavage such as a breast duct lavage, lung lavage, a gastric lavage, a rectal or colonic lavage, or a vaginal lavage; an aspirate such as a nipple aspirate; or a fluid such as a supernatant from a cell culture. In order to test for the presence, or measure the level, of mRNA. X in cells, the cells can be lysed and total RNA can be purified or semi-purified from lysates by any of a variety of methods known in the art. Methods of detecting or measuring levels of particular mRNA transcripts are also familiar to those in the art. Such assays include, without limitation, hybridization assays using detectably labeled mRNA X-specific DNA or RNA probes and quantitative or semi-quantitative RT-PCR methodologies employing appropriate mRNA X and cDNA X-specific oligonucleotide primers. Additional methods for quantitating mRNA in cell lysates include RNA protection assays and serial analysis of gene expression (SAGE). Alternatively, qualitative, quantitative, or semi-quantitative in situ hybridization assays can be carried out using, for example, tissue sections or unlysed cell suspensions, and detectably (e.g., fluorescently or enzyme) labeled DNA or RNA probes.

Methods of detecting or measuring the levels of a protein of interest in cells are known in the art. Many such methods employ antibodies (e.g., polyclonal antibodies or monoclonal antibodies (mAbs)) that bind specifically to the protein. In such assays, the antibody itself or a secondary antibody that binds to it can be detectably labeled. Alternatively, the antibody can be conjugated with biotin, and detectably labeled avidin (a protein that binds to biotin) can be used to detect the presence of the biotinylated antibody. Combinations of these approaches (including “multi-layer” assays) familiar to those in the art can be used to enhance the sensitivity of assays. Some of these assays (e.g., immunohistological methods or fluorescence flow cytometry) can be applied to histological sections or unlysed cell suspensions. The methods described below for detecting protein X in a liquid sample can also be used to detect protein X in cell lysates.

Methods of detecting protein X in a liquid sample (see above) basically involve contacting a sample of interest with an antibody that binds to protein X and testing for binding of the antibody to a component of the sample. In such assays the antibody need not be detectably labeled and can be used without a second antibody that binds to protein X. For example, by exploiting the phenomenon of surface plasmon resonance, an antibody specific for protein X bound to an appropriate solid substrate is exposed to the sample. Binding of protein X to the antibody on the solid substrate results in a change in the intensity of surface plasmon resonance that can be detected qualitatively or quantitatively by an appropriate instrument, e.g., a Biacore apparatus (Biacore International AB, Rapsgatan, Sweden).

Moreover, assays for detection of protein X in a liquid sample can involve the use, for example, of: (a) a single protein X-specific antibody that is detectably labeled; (b) an unlabeled protein X-specific antibody and a detectably labeled secondary antibody, or (c) a biotinylated protein X-specific antibody and detectably labeled avidin. In addition, as described above for detection of proteins in cells, combinations of these approaches (including “multi-layer” assays) familiar to those in the art can be used to enhance the sensitivity of assays. In these assays, the sample or an (aliquot of the sample) suspected of containing protein X can be immobilized on a solid substrate such as a nylon or nitrocellulose membrane by, for example, “spotting” an aliquot of the liquid sample or by blotting of an electrophoretic gel on which the sample or an aliquot of the sample has been subjected to electrophoretic separation. The presence or amount of protein X on the solid substrate is then assayed using any of the above-described forms of the protein X-specific antibody and, where required, appropriate detectably labeled secondary-antibodies or avidin.

The invention also features “sandwich” assays. In these sandwich assays, instead of immobilizing samples on solid substrates by the methods described above, any protein X that may be present in a sample can be immobilized on the solid substrate by, prior to exposing the solid substrate to the sample, conjugating a second (“capture”) protein X-specific antibody (polyclonal or mAb) to the solid substrate by any of a variety of methods known in the art. In exposing the sample to the solid substrate with the second protein X-specific antibody bound to it, any protein X in the sample (or sample aliquot) will bind to the second protein X-specific is antibody on the solid substrate. The presence or amount of protein X bound to the conjugated second protein X-specific antibody is then assayed using a “detection” protein X-specific antibody by methods essentially the same as those described above using a single protein X-specific antibody. It is understood that in these sandwich assays, the capture antibody should not bind to the same epitope (or range, of epitopes in the case of a polyclonal antibody) as the detection antibody. Thus, if a mAb is used as a capture antibody, the detection antibody can be either: (a) another in Ab that binds to an epitope that is either completely physically separated from or only partially overlaps with the epitope to which the capture mAb binds; or (b) a polyclonal antibody that binds to epitopes other than or in addition to that to which the capture mAb binds. On the other hand, if a polyclonal antibody is used as a capture antibody, the detection antibody can be either (a) a mAb that binds to an epitope to that is either completely physically separated from or partially overlaps with any of the epitopes to which the capture polyclonal antibody binds; or (b) a polygonal antibody that binds to epitopes other than or in addition to that to which the capture polyclonal antibody binds. Assays which involve the used of a capture and detection antibody include sandwich ELISA assays, sandwich Western blotting assays, and sandwich immunomagnetic detection assays.

Suitable solid substrates to which the capture antibody can be bound include, without limitation, the plastic bottoms and sides of wells of microtiter plates, membranes such as nylon or nitrocellulose membranes, polymeric (e.g., without limitation, agarose, cellulose, or polyacrylamide) beads or particles. It is noted that protein X-specific antibodies bound to such beads or particles can also be used for immunoaffinity purification of protein X.

Methods of detecting or for quantifying a detectable label depend on the nature of the label and are known in the art. Appropriate labels include, without limitation, radionuclides (e.g., ¹²⁵I, ¹³¹I, ³⁵S, ³H, ³²P, ³³P, or ¹⁴C), fluorescent moieties (e.g., fluorescein, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). The products of reactions catalyzed by appropriate enzymes can be, without limitation, fluorescent, luminescent, or radioactive or they may absorb visible or ultraviolet light. Examples of detectors include, without limitation, x-ray film, radioactivity counters, scintillation counters, spectrophotometers, calorimeters, fluorometers, luminometers, and densitometers.

In assays, for example, to diagnose breast cancer, the level, of protein X in, for example, serum, (or a breast cell) from a patient suspected of having, or at risk of having, breast cancer is compared to the level of protein X in sera (or breast cells) from a control subject (e.g., a subject not having breast cancer) or the mean level of protein X in sera (or breast cells) from a control group of subjects (e.g., subjects not having breast cancer). A significantly higher level, or lower level (depending on whether the gene of interest is expressed at higher or lower level in breast cancer or associated stromal cells), of protein X in the serum (or breast cells) of the patient relative to the mean level in sera (or breast cells) of the control group would indicate that the patient has breast cancer. Alternatively, if a sample of the subject's serum (or breast cells) that was obtained at a prior date at which the patient clearly did not have breast cancer is available, the level of protein in the test serum (or breast cell) sample can be compared to the level in the prior obtained sample. A higher level, or lower level (depending on whether the gene of interest is expressed at higher or lower level in breast cancer or associated stromal cells) in the test serum (or breast cell) sample would be an indication that the patient has breast cancer.

Moreover, a test expression profile of a gene in a test cell (or tissue) can be compared to control expression profiles of control cells (or tissues) previously established to be of defined category (e.g., DCIS grade, breast cancer stage, or state of differentiation). The category of the test cell (or tissue) will be that of the control cell (or tissue) whose expression profile the test cell's (or tissue's) expression profile most closely resembles. These expression profile comparison assays can be used to compare any of the normal breast tissue with any stage and/or grade of breast cancer recited herein and/or to compare between breast cancer grades and stages. The genes analyzed can be any of those listed in Tables 1-16 and the number of genes analyzed can be any number, i.e. one or more. Generally, at least two (e.g., at least: two; three; four; five; six; seven; eight; nine; ten; 11; 12; 13; 14; 15; 17; 18; 20; 23; 25; 30; 35; 40; 45; 50; 60; 70; 80; 90; 100; 120; 150; 200; 250; 300; 350; 400; 450; 500; or more) genes will be analyzed. It is understood that the genes analyzed will include at least one of those listed herein but can also include others not listed herein.

One of skill in the art will appreciate from this description how similar “test level” versus “control level” comparisons can be made between other test and control samples described herein.

It is noted that the patients and control subjects referred to above need not be human patients. They can be for example, non-human primates (e.g., monkeys), horses, sheep, cattle, goats, pigs, dogs, guinea pigs, hamsters, rats, rabbits or mice.

Methods of Inhibiting Expression of Genes

Also included in the invention are methods of inhibiting expression of the genes listed in Tables 2-10, 15, and 16 in cells, e.g., breast epithelial cancer cells and/or stromal cells (e.g., leukocytes, myoepithelial cells, myofibroblasts, endothelial cells, or fibroblasts) in a tumor containing the cancer cells; such methods are applicable where the expression of protein X in breast cancer cells, or stromal cells in a breast tumor, is higher than in corresponding normal cells. These methods can also be adapted to inhibit expression of a receptor for a ligand protein X. One such method involves introducing into a cell (a) an antisense oligonucleotide or (b) a nucleic acid comprising a transcriptional regulatory element (TRE) operably linked to a nucleic sequence that is transcribed in the cell into an antisense RNA. The antisense oligonucleotide and the antisense RNA hybridize to a mRNA X molecule (or mRNA molecule encoding a receptor for a ligand protein X) and have the effect in the cell of inhibiting expression of protein X (or receptor for protein X) in the cell. Inhibiting protein X/protein X receptor expression in the breast cancer cells or stromal cells can inhibit pathogenesis of breast cancer cells. The method can thus be useful in inhibiting pathogenesis of a breast cancer cell and can be applied to the therapy of breast cancer, e.g., DCIS, invasive breast cancer, or metastatic breast cancer.

Antisense compounds are generally used to interfere with protein-expression either by, for example, interfering directly with translation of a target mRNA molecule, by RNAse-H-mediated degradation of the target mRNA, by interference with 5′ capping of mRNA, by prevention of translation factor binding to the target mRNA by masking of the 5′ cap, or by inhibiting of mRNA polyadenylation. The interference with protein expression arises from the hybridization of the antisense compound with its target mRNA. A specific targeting site on a target mRNA of interest for interaction with an antisense compound is chosen. Thus, for example, for modulation of polyadenylation a preferred target site on an mRNA target is a polyadenylation signal or a polyadenylation site. For diminishing mRNA stability or degradation, destabilizing sequence are preferred target sites. Once one or more target sites have been identified, oligonucleotides are chosen which are sufficiently complementary to the target site (i.e., hybridize sufficiently well under physiological conditions and with sufficient specificity) to give the desired effect.

With respect to this invention, the term “oligonucleotide” refers to an oligomer or polymer of RNA, DNA, or a mimetic of either. The term includes oligonucleotides composed of naturally-occurring nucleobases, sugars, and covalent internucleoside (backbone) linkages. The normal linkage or backbone of RNA and DNA is a 3′ to 5′ phosphodiester bond. The term also refers however to oligonucleotides composed entirely of, or having portions containing, non-naturally occurring components which function in a similar manner to the oligonucleotides containing only naturally-occurring components. Such modified substituted oligonucleotides are often preferred over native forms because of desirable properties such as, for example, enhanced cellular uptake, enhanced affinity for target sequence, and increased stability in the presence of nucleases. In the mimetics, the core base (pyrimidine or purine) structure is generally preserved but (1) the sugars are either modified or replaced with other components and/or (2) the inter-nucleobase linkages are modified. One class of nucleic acid mimetic that has proven to be very useful is referred to as protein nucleic acid (PNA). In PNA molecules the sugar backbone is replaced with an amide-containing backbone, in particular an aminoethylglycine backbone. The bases are retained and are bound directly to the aza nitrogen atoms of the amide portion of the backbone. PNA and other mimetics useful in the instant invention are described in detail in U.S. Pat. No. 6,210,289, which is incorporated herein by reference in its entirety.

The antisense oligomers to be used in the methods of the invention generally comprise about 8 to about 100 (e.g., about 14 to about 80 or about 14 to about 35) nucleobases (or nucleosides where the nucleobases are naturally occurring).

The antisense oligonucleotides can themselves be introduced into a cell or an expression vector containing a nucleic sequence (operably linked to a TRE) encoding the antisense oligonucleotide can be introduced into the cell. In the latter case, the oligonucleotide produced by the expression vector is an RNA oligonucleotide and the RNA oligonucleotide will be composed entirely of naturally occurring components.

The methods of the invention can be in vitro or in vivo. In vitro applications of the methods can be useful, for example, in basic scientific studies on cancer cell pathogenesis, e.g., cancer cell proliferation and/or cell survival. In such in vitro methods, appropriate cells (see above), can be incubated for various lengths of time with (a) the antisense oligonucleotides or (b) expression vectors containing nucleic acid sequences encoding the antisense oligonucleotides at a variety of concentrations. Other incubation conditions known to those in art (e.g., temperature or cell concentration) can also be varied. Inhibition of protein X expression can be tested by methods known to those in the art. However, the methods of the invention will preferably be in vivo.

As used herein, “prophylaxis” can mean complete prevention of the symptoms of a disease (e.g., breast cancer such as DCIS), a delay in onset of the symptoms of a disease, or a lessening in the severity of subsequently developed disease symptoms. “Prevention” should mean that symptoms of the disease (e.g., breast cancer) are essentially absent. As used herein, “therapy” can mean a complete abolishment of the symptoms of a disease or a decrease in the severity of the symptoms of the disease. As used herein, a “protective” regimen is a regimen that is prophylactic and/or therapeutic.

The antisense methods are generally useful for cancer cells (e.g., a breast cancer cell) cancer cell pathogenesis-inhibiting therapy or prophylaxis. They can be administered to mammalian subjects (e.g., human breast cancer patients) alone or in conjunction with other drugs and/or radiotherapy.

Where antisense oligonucleotides per se are administered, they can be suspended in a pharmaceutically-acceptable carrier (e.g., physiological saline) and administered orally, intrarectally, intravaginally, intranasally, intragastrically, intratracheally, or intrapulmonarily, or injected subcutaneously, intramuscularly, intrathecally, intraperitoneally, intravenously. They can also be delivered directly to tumor cells, e.g., to a tumor or a tumor bed following surgical excision of the tumor, in order to kill any remaining tumor cells. The dosage required depends on the choice of the route of administration; the nature of the formulation; the nature of the patient's illness; the subject's size, weight, surface area, age, and sex; other drugs being administered; and the judgment of the attending physician. Suitable dosages are generally in the range of 0.01 mg/kg-100 mg/kg. Wide variations in the needed dosage are to be expected in view of the variety of compounds available and the differing efficiencies of various routes of administration. For example, oral administration would be expected to require higher dosages than administration by intravenous injection. Variations in these dosage levels can be adjusted using standard empirical routines for optimization as is well understood in the art. Administrations can be single or multiple (e.g., 2-, 3-, 4-, 6-, 8-, 10-, 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Where an expression vector containing a nucleic sequence (operably linked to a TRE) encoding the antisense oligonucleotide is administered to a subject, expression of the coding sequence can be directed to any cell in the body of the subject. However, expression will preferably be directed to cells in a tumor containing the cancer cells or cells in the immediate vicinity of the cancer cells whose pathogenesis it is desired to inhibit. Expression of the coding sequence can be directed to the tumor cells themselves. This can be achieved by, for example, the use of polymeric, biodegradable microparticle or microcapsule delivery devices known in the art.

Another way to achieve uptake of the nucleic acid is using liposomes, prepared by standard methods. The vectors can be incorporated alone into these delivery vehicles or co-incorporated with tissue-specific or tumor-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells [Cristiano et al; (1995), J. Mol. Med. 73:479]. Alternatively, tissue-specific targeting can be achieved by the use of tissue-specific transcriptional/translational regulatory elements (TRE), e.g., promoters and enhancers, which are known in the art. Delivery of “naked DNA” (i.e., without a delivery vehicle) to an intramuscular, intradermal, or subcutaneous site is another means to achieve in vivo expression.

Enhancers provide expression specificity in terms of time, location, and level. Unlike a promoter, an enhancer can function when located “at variable distances from the” transcription initiation site, provided a promoter is present. An enhancer can also be located downstream of the transcription initiation site. To bring a coding sequence under the control of a promoter, it is necessary to position the translation initiation site of the translational reading frame of the peptide or polypeptide between one and about fifty nucleotides downstream (3′) of the promoter. The coding sequence of the expression vector is operatively linked to a transcription terminating region.

The transcriptional/translational regulatory elements referred to above include, but are not limited to, inducible and non-inducible promoters, enhancers, operators and other elements that are known to those skilled in the art and that drive or otherwise regulate gene expression. Examples of such regulatory elements are provided above in the section on Nucleic Acids.

Suitable expression vectors include plasmids and viral vectors such as herpes viruses, retroviruses, vaccinia viruses, attenuated vaccinia viruses, canary pox viruses, adenoviruses and adeno-associated viruses, among others.

Polynucleotides can be administered in a pharmaceutically acceptable carrier. Pharmaceutically acceptable carriers are biologically compatible vehicles that are suitable for administration to a human, e.g., physiological saline or liposomes. A therapeutically effective amount is an amount of the polynucleotide that is capable of producing a medically desirable result (e.g., decreased proliferation and or survival of breast cancer cells) in a treated animal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 106 to approximately 1012 copies of the polynucleotide molecule. This dose can be repeatedly administered, as needed; Routes of administration can be any of those listed above.

Double-stranded interfering RNA (RNAi) homologous to mRNA X can also be used to reduce expression of protein X in a cell. See, e.g., Fire et al. (1998) Nature 391:806-811; Romano and Masino (1992) Mol. Microbiol. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017-1026.

The sense and anti-sense RNA strands of RNAi can be individually constructed using chemical synthesis and enzymatic ligation reactions using procedures known in the art. For example, each strand can be chemically synthesized using naturally occurring nucleotides or variously modified nucleotides designed to increase the biological stability of the molecule or to increase the physical stability of the duplex formed between the sense and anti-sense strands, e.g., phosphorothioate derivatives and acridine substituted nucleotides. The sense or anti-sense strand can also be produced biologically using an expression vector into which a target protein X sequence (full-length or a fragment) has been subcloned in a sense or anti-sense orientation. The sense and anti-sense RNA strands can be annealed in vitro before delivery of the dsRNA to any of cancer cells disclosed herein. Alternatively, annealing can occur in vivo after the sense and anti-sense strands are sequentially delivered to the cancer cells.

Double-stranded RNA interference can also be achieved by introducing into cancer cells a polynucleotide from which sense and anti-sense RNAs can be transcribed under the direction of separate promoters, or a single RNA molecule containing both sense and anti-sense sequences can be transcribed under the direction of a single promoter.

Also useful for inhibiting expression of gene X are “small molecule” inhibitors of gene expression. Such small-molecules are useful for inhibiting a function of protein X or a downstream activity initiated by or via protein X. For example, quinazoline compounds are useful in inhibiting tyrosine kinase activity that, for example, is stimulated by binding of a ligand to one of epidermal growth factor receptors (EGFR), e.g., erbB1 or erbB2. Small molecules of interest include, without limitation, small non-nucleic acid organic molecules, small inorganic molecules, peptides, peptides, peptidomimetics, non-naturally occurring nucleotides, and small nucleic acids (e.g., RNAi or antisense oligonucleotides). Generally, small molecules have molecular weights of less than 10 kd[a (e.g., less than: 10 kDa; 9 kDa; 8 kDa; 7 kDa; 6 kDa; 5 kDa; 4 kDa; 3 kDa; 2 kDa; or 1 kDa).

Other methods of interest include the recently described degrakine and intrakine techniques [Coffield et al. (2003) Nat. Biotech. 21:1321-1327; Chen et al. (1997) Nat. Med. 3:1110-1116], which result in inhibition of expression, on the surface of a target cell (e.g., a breast cancer cell), of a receptor for a ligand protein (e.g., a soluble ligand such as a cytokine, chemokine, or growth factor or a ligand on the surface of another cell). By inhibiting expression of the receptor on the target cell, responsiveness of the target cell to the ligand protein is inhibited or, optimally, prevented.

In the degrakine methodology, a fusion protein is used to inhibit cell surface expression of a receptor for a ligand protein X of interest (e.g., a receptor for CXCL14), the receptor being on the surface of a target cell of interest (e.g., a breast cancer cell). The fusion protein is a fusion between (a) a ligand protein X (or a fragment of the protein X ligand that retains the ability to bind to the receptor for the protein X ligand) and (b) the HIV-1 Vpu protein. The target cell of interest is contacted in vivo or in vitro with an expression vector (e.g., a viral vector such as any of those disclosed herein) expressing the fusion protein. After entry of the expression vector into the cell, the fusion protein is produced in the cytoplasm of the target cell. The fusion protein, due to the activity of the Vpu protein, then migrates to the endoplasmic reticulum (ER) of the target cell where it can bind to recently translated ligand protein X receptor molecules and inhibit or, optimally, prevent translocation of the receptor molecules to the surface of the target cell. Moreover, it is believed that the Vpu component of the fusion protein bound to newly made receptor molecules targets the receptor molecules for degradation by proteasomes within the target cell [Coffield et al. (2003)].

Intrakine methodologies are conceptually similar to the degrakine methodology. Instead of the Vpu protein, a signal sequence that serves to direct proteins containing it to the ER (e.g., the four amino acrid KDEL (SEQ ID NO:1956) sequence) is fused to the ligand protein X (or a fragment of the protein X ligand that retains the ability to bind to the receptor for the ligand protein X) [Coffield et al. (2003); Chen et al. (1997)].

The degrakine and intrakine methodologies can be modified as follows. The fusion protein itself can be contacted (in vivo or in vitro) with a target cell expressing a surface receptor for the ligand protein X. The fusion protein can then, e.g., by binding to such a receptor, enter the cytoplasm of the target cell. The fusion protein then, as in the vector-mediated method described above, migrates to the ER of the target cell and inhibits translocation of the receptor to the target cell surface.

One of skill in the art will appreciate that RNAi, small molecule, and degrakine/intrakine methods can be, as for the antisense methods described above, in vitro and in vivo. Moreover, methods and conditions of delivery for RNAi, small molecule, and degrakine/intrakine methods can be applied are the same as those for antisense oligonucleotides.

The antisense, RNAi, small molecule, and degrakine/intrakine methods of the invention can be applied to a wide range of species, e.g., humans, non-human primates, horses, cattle, pigs, sheep, goats, dogs, cats, rabbits, guinea pigs, hamsters, rats, and mice.

Passive Immunoprotection

The methods described in this section are applicable where the expression of protein X in breast cancer cells, or stromal cells in a breast tumor, is higher than in corresponding normal cells.

As used herein, “passive immunoprotection” means administration of one or more protein X-binding agents to a subject that has, is suspected of having, or is at risk of having a breast cancer, e.g., a DCIS, an invasive breast cancer, or a metastatic breast cancer. Thus, passive immunoprotection can be prophylactic and/or therapeutic. As used herein, “protein X-binding agents” are agents that bind to protein X and thereby inhibit the ability of protein X to enhance pathogenesis of breast cancer cells. It is understood that the term “inhibit” includes “completely inhibit” and “partially inhibit.” Protein X-binding agents can be, for example, a soluble (i.e., not cell-bound) full length form (or fragment such as a fragment lacking a transmembrane domain) of a receptor for protein X (where protein X is a ligand), a soluble, non-agonist form (or fragment of a ligand for protein X (where protein X is a receptor), or a non-agonist, antibody specific for protein X. Other useful agents include non-agonist molecules that bind to a receptor for a protein X (i.e., protein X receptor-binding agents). Such protein X receptor-binding agents include non-agonist antibodies specific for a protein X receptor and non-agonist fragments of a protein X that retain the ability to bind to the receptor for protein X. A protein X-binding agent (or a protein X receptor-binding agent) useful for the invention has the capacity to inhibit the ability of protein X to enhance the pathogenesis (e.g., proliferation and/or survival) of the breast cancer cells by at least 20% (e.g., at least: 20%; 30%; 40%; 50%; 60%; 70%; 80%; 90%; 95%; 98%; 99%; 99.5%, or even 100%).

Antibodies can be polyclonal or monoclonal antibodies; methods for producing both types of antibody are known in the art. The antibodies can be of any class (e.g., IgM, IgG, IgA, IgD, or IgE) and be generated in any of the species recited herein. They are preferably IgG antibodies. Recombinant antibodies, such as chimeric and humanized monoclonal antibodies comprising both human and non-human portions, can also be used in the methods of the invention. Such chimeric and humanized monoclonal antibodies can be produced by recombinant DNA techniques known in the art, for example, using methods described in Robinson et al., International Patent Publication PCT/US86/02269; Akira et al., European Patent Application 184,187; Taniguchi, European Patent Application 171,496; Morrison et al., European Patent Application 1-73,494; Neuberger et al., PCT Application WO 86/01533; Cabilly et al., U.S. Pat. No. 4,816,567; Cabilly et al., European Patent Application 125,023; Better et al. (1988) Science 240, 1041-43; Liu et al. (1987) J. Immunol. 139, 3521-26; Sun et al. (1987) PNAS 84, 214-18; Nishimura et al. (1981) Canc. Res. 47, 999-1005; Wood et al. (1985) Nature 314, 446-49; Shaw et al. (1988) J. Natl. Cancer Inst. 80, 1553-59; Morrison, (1985) Science 229, 1202-07; Oi et al. (1986) BioTechniques 4, 214; Winter, U.S. Pat. No. 5,225,539; Jones et al. (1986) Nature 321, 552-25; Veroeyan et al. (1988) Science 239, 1534; and Beidler et al. (1988) J. Immunol. 141, 4053-60.

Also useful for the invention are antibody fragments and derivatives that contain at least the functional portion of the antigen-binding domain of an antibody. Antibody fragments that contain the binding domain of the molecule can be generated by known techniques. Such fragments include, but are not limited to: F(ab′)₂ fragments that can be produced by pepsin digestion of antibody molecules; Fab fragments that can be generated by reducing the disulfide bridges of F(ab′)₂ fragments; and Fab fragments that can be generated by treating antibody molecules with papain and a reducing agent. See, e.g., National Institutes of Health, 1 Current Protocols In Immunology, Coligan et al., ed. 2.8, 2.10 (Wiley Interscience, 1991). Antibody fragments also include Fv fragments, i.e., antibody products in which there are few or no constant region amino acid residues. A single chain Fv fragment (scFv) is a single polypeptide chain that includes both the heavy and light chain variable regions of the antibody from which the scFv is derived. Such fragments can be produced, for example, as described in U.S. Pat. No. 4,642,334, which is incorporated herein by reference in its entirety. For a human subject, the antibody can be a “humanized” version of a monoclonal antibody originally generated in a different species.

The invention includes antibodies specific for the proteins encoded by genes corresponding to the SAGE tags listed in FIG. 7. The antibodies can be of any of the types and classed referred to herein.

Protein X-binding (or protein X receptor-binding) agents can be administered to any of the species listed herein. The binding agents will preferably, but not necessarily, be of the same species as the subject to which they are administered. A single polyclonal or monoclonal antibody can be administered, or two or more (e.g., two, three, four, five, six, seven, eight, nine, ten, 12, 14, 16, 18, or 20) polyclonal antibodies or monoclonal antibodies can be given. The binding agents can be administered to subjects prior to, subsequently to, or at the same time as the protein X-expression inhibitors (see above).

The dosage of protein X/protein X receptor-binding agents required depends on the route is of administration, the nature of the formulation, the nature of the patient's illness, the subject's size, weight, surface area, age, and sex, other drugs being administered, and the judgment of the attending physician. Suitable dosages are in the range of 0.01-100.0 mg/kg. The protein X/protein X receptor-binding agents can be administered by any of the routes disclosed herein, but will generally be administered intravenously, intramuscularly, or subcutaneously. Wide variations in the needed dosage are to be expected in view of the variety of protein X/protein X receptor-binding agents (e.g., protein X-specific antibodies) available and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold).

Methods to test whether a compound or antibody is therapeutic for, or prophylactic against, a particular disease are known in the art. Where a therapeutic effect is being tested, a test population displaying symptoms of the disease (e.g.; breast cancer such as DCIS) is treated with a protein X/protein X receptor expression inhibitor or protein X/protein X receptor-binding agent using any of the above-described strategies. A control population, also displaying symptoms of the disease, is treated, using the same methodology, with a placebo. Disappearance or a decrease of the disease symptoms in the test subjects would indicate that the compound or antibody was an effective therapeutic agent. By applying the same strategies to subjects at risk of having the disease, the compounds and antibodies can be tested for efficacy as prophylactic agents. In this situation, prevention of or delay in onset of disease symptoms is tested.

Methods of Inhibiting Pathogenesis of a Cancer Cell

Such methods are applicable where the expression of protein X in breast cancer cells, or stromal cells in a breast tumor, is lower than in corresponding normal cells (see Tables 1, 3-10, and 15). These methods involve contacting a breast cancer cell with a protein X, or a functional fragment thereof, in order to inhibit pathogenesis (e.g., proliferation or survival) of the cancer cell. Such polypeptides or functional fragments can have amino acid sequences identical to wild-type sequences or they can contain not more than 50 (e.g., not more than: 50; 40; 30; 25; 20; 15; 12; 10; nine; eight; seven; six; five; four; three; two; or one) conservative amino acid substitution(s). Alleles of the polypeptides encoded by listed in Tables 1, 3-10, and 15 are also useful for the invention.

The methods can be performed in vitro, in vivo, or ex vivo. In vitro application of protein X can be useful, for example, in basic scientific studies of tumor cell biology, e.g., studies on cancer cell proliferation, survival, invasion, metastasis, or escape from immunological effector mechanisms or studies on angiogenesis. In addition, protein X and the polynucleotides encoding protein X (DNA and/or RNA) can be used as “positive controls” in diagnostic assays (see below). However, the methods of the invention will preferably be in vivo or ex vivo (see below).

Protein X and variants thereof are generally useful as cancer cell (e.g., breast cancer cell) pathogenesis-inhibiting therapeutics. They can be administered to mammalian subjects (e.g., human breast cancer patients) alone or in conjunction with such drugs and/or radiotherapy.

These methods of the invention can be applied to a wide range of species, e.g., humans, non-human primates, horses, cattle, pigs, sheep, goats, dogs, cats, rabbits, guinea pigs, hamsters, rats, and mice.

In Vivo Approaches

In one in vivo approach, protein X (or a functional fragment thereof) itself is administered to the subject. Generally, the compounds of the invention will be suspended in a pharmaceutically-acceptable carrier (e.g., physiological saline) and administered orally or by intravenous infusion, or injected subcutaneously, intramuscularly, intrathecally, intraperitoneally, intrarectally, intravaginally, intranasally, intragastrically, intratracheally, or intrapulmonarily. They are preferably delivered directly to tumor cells, e.g., to a tumor or a tumor bed following surgical excision of the tumor, in order to kill any remaining tumor cells. The dosage required depends on the choice of the route of administration; the nature of the formulation; the nature of the patient's illness; the subject's size, weight, surface area, age, and sex; other drugs being administered; and the judgment of the attending physician. Suitable dosages are in the range of 0.01-100.0 μg/kg. Wide variations in the needed dosage are to be expected in view of the variety of polypeptides and fragments available and the differing efficiencies of various routes of administration. For example, oral administration would be expected to require higher dosages than administration by i.v. injection. Variations in these dosage levels can be adjusted using standard empirical routines for optimization as is well understood in the art. Administrations can be single or multiple (e.g., 2-, 3-, 4-, 6-, 8-, 10-; 20-, 50-, 100-, 150-, or more fold). Encapsulation of the polypeptide in a suitable delivery vehicle (e.g., polymeric microparticles or implantable devices) may increase the efficiency of delivery, particularly for oral delivery.

Alternatively, a polynucleotide containing a nucleic acid sequence encoding protein X or functional fragment thereof can be delivered to breast cancer cells in a mammal. Expression of the coding sequence will preferably be directed to lymphoid tissue of the subject by, for example, delivery of the polynucleotide to the lymphoid tissue. Expression of the coding sequence can be directed to any cell in the body of the subject. However, expression will preferably be directed to cells (e.g., stromal cells) in a tumor containing, or in the vicinity of, the cancer cells whose proliferation it is desired to inhibit. In certain embodiments, expression of the coding sequence can be directed to the tumor cells themselves. This can be achieved by, for example, the use of polymeric, biodegradable microparticle or microcapsule delivery devices known in the art.

Another way to achieve uptake of the nucleic acid is using liposomes (see section above on Methods of Inhibiting Expression of Genes).

In the relevant polynucleotides (e.g., expression vectors), the nucleic acid sequence encoding protein X or functional fragment of interest with an initiator methionine and optionally a targeting sequence is operatively linked to a promoter or enhancer-promoter combination.

Short amino acid sequences can act as signals to direct proteins to specific intracellular compartments. Such signal sequences are described in detail in U.S. Pat. No. 5,827,516, which is incorporated herein by reference in its entirety.

Appropriate enhancers, vectors, and methods of administration of polynucleotides are described above in the section on Methods of Inhibiting Gene Expression.

Ex Vivo Approaches

An ex vivo strategy can involve transfecting or transducing cells obtained from the subject with a polynucleotide encoding protein X or functional fragment-encoding nucleic acid sequences described above. The transfected or transduced cells are then returned to the subject. The cells can be any of a wide range of types including, without limitation, hemopoietic cells (including leukocytes) (e.g., bone marrow cells, macrophages, monocytes, dendritic cells, T cells, or B cells), fibroblasts, epithelial cells, endothelial cells, keratinocytes, or muscle cells. Such cells act as a source of the protein X or functional fragment for as long as they survive in the subject. Alternatively, tumor cells, preferably obtained from the subject but potentially from an individual other than the subject, can be transfected or transformed by a vector encoding a protein X or functional fragment thereof. The tumor cells, preferably treated with an agent (e.g., ionizing irradiation) that ablates their proliferative capacity, are then introduced into the patient, where they secrete exogenous protein Z.

The ex vivo methods include the steps of harvesting cells from a subject, culturing the cells, transducing them with an expression vector, and maintaining the cells under conditions suitable for expression of the protein polypeptide or functional fragment. These methods are known in the art of molecular biology. The transduction step is accomplished by any standard means used for ex vivo gene therapy, including calcium phosphate, lipofection, electroporation, viral infection, and biolistic gene transfer. Alternatively, liposomes or polymeric microparticles can be used. Cells that have been successfully transduced can then be selected, for example, for expression of the coding sequence or of a drug resistance gene. The cells may then be lethally irradiated (if desired) and injected or implanted into the patient.

Arrays and Uses Thereof

The invention features an array that includes a substrate having a plurality of addresses. At least one address of the plurality includes a capture probe that binds specifically to a nucleic acid X or a protein X. The array can have a density of at least, or less than, 10, 20 50, 100, 200, 500, 700, 1,000, 2,000, 5,000 or 10,000 or more addresses/cm², and ranges between. In a preferred embodiment, the plurality of addresses includes at least 10, 100, 500, 1,000, 5,000, 10,000, 50,000 addresses. In a preferred embodiment, the plurality of addresses includes equal to or less than 10, 100, 500, 1,000, 5,000, 10,000, or 50,000 addresses. The substrate can be a two-dimensional substrate such as a glass slide, a wafer (e.g., silica or plastic), a mass spectroscopy plate, or a three-dimensional substrate such as a gel pad. Addresses in addition to address of the plurality can be disposed on the array.

In one embodiment, at least one address of the plurality includes a nucleic acid capture probe that hybridizes specifically to a nucleic acid X, e.g., the sense or anti-sense strand. Nucleic acids of interest include, without limitation, all or part of any of the genes identified by the tags listed in Tables 1-16, all or part of mRNAs transcribed from such genes, or all or part of cDNA produced from such mRNA. Useful probes can, for example, be or contain the nucleotide sequences of the tags listed in Tables 1-5, 7-10, 15 and 16. Each address of the subset can include a capture probe that hybridizes to a different region of a nucleic acid. Each address of the subset is unique, overlapping, and complementary to a different variant of gene X (e.g., an allelic variant, or all possible hypothetical variants). The array can be used to sequence gene X, mRNA X, or cDNA X by hybridization (see, e.g., U.S. Pat. No. 5,695,940).

An array can be generated by any of a variety of methods. Appropriate methods include, e.g., photolithographic methods (see, e.g.; U.S. Pat. Nos. 5,143,854; 5,510,270; and 5,527,681), mechanical methods (e.g., directed-flow methods as described in U.S. Pat. No. 5,384,261), pin-based methods (e.g., as described in U.S. Pat. No. 5,288,514), and bead-based techniques (e.g., as described in PCT US/93/04145).

In another embodiment, at least one address of the plurality includes a polypeptide capture probe that binds specifically to protein X or fragment thereof. The polypeptide can be a naturally-occurring interaction partner of protein X, e.g., a ligand for protein X where protein X if a receptor or a receptor for protein X where protein X is ligand. Preferably, the polypeptide is an antibody, e.g., an antibody specific for protein X, such as a polyclonal antibody, a monoclonal antibody, or a single-chain antibody.

In another aspect, the invention features a method of analyzing the expression of gene X. The method includes providing an array as described above; contacting the array with a sample and detecting binding of a nucleic acid X or protein X to the array. In one embodiment, the array is a nucleic acid array. Optionally the method further includes amplifying nucleic acid from the sample prior or during contact with the array.

In another embodiment, the array can be used to assay gene expression in a tissue to ascertain tissue specificity of genes in the array, particularly the expression of gene X. If a sufficient number of diverse samples is analyzed, clustering (e.g., hierarchical clustering, k-means clustering, Bayesian clustering and the like) can be used to identify other genes which are co-regulated with gene X. For example, the array can be used for the quantitation of the expression of multiple genes. Thus, not only tissue specificity, but also the level of expression of a battery of genes in the tissue is ascertained. Quantitative data can be used to group (e.g., cluster) genes on the basis of their tissue expression per se and level of expression in that tissue.

For example, array analysis of gene expression can be used to assess the effect of cell-cell interactions on gene X expression. A first tissue can be perturbed and nucleic acid from a second tissue that interacts with the first tissue can be analyzed. In this context, the effect of one cell type on another cell type in response to a biological stimulus can be determined, e.g., to monitor the effect of cell-cell interaction at the level of gene expression.

Moreover, cells can be contacted with a therapeutic agent. The expression profile of the cells is determined using the array, and the expression profile is compared to the profile of like cells not contacted with the agent. For example, the assay can be used to determine or analyze the molecular basis of an undesirable effect of the therapeutic agent. If an agent is administered therapeutically to treat one cell type but has an undesirable effect on another cell type, the invention provides an assay to determine the molecular basis of the undesirable effect and thus provides the opportunity to co-administer a counteracting agent or otherwise treat the undesired effect. Similarly, even within a single cell type, undesirable biological effects can be determined at the molecular level. Thus, the effects of an agent on expression of other than the target gene can be ascertained and counteracted.

In another embodiment, the array can be used to monitor expression of one or more genes in the array with respect to time. For example, samples obtained from different time points can be probed with the array. Such analysis can identify and/or characterize the development of a gene X-associated disease or disorder (e.g., breast cancer such as invasive breast cancer); and processes, such as a cellular transformation associated with a gene X-associated disease or disorder. The method can also evaluate the treatment and/or progression of a gene X-associated disease or disorder.

The array is also useful for ascertaining differential expression patterns of one or more genes in normal and abnormal (e.g., malignant) cells. This provides a battery of genes (e.g., including gene X) that could serve as a molecular target for diagnosis or therapeutic intervention.

In another aspect, the invention features an array having a plurality of addresses. Each address of the plurality includes a unique polypeptide. At least one address of the plurality has disposed thereon a protein or fragment thereof. Methods of producing polypeptide arrays are described in the art [e.g., in De Wildt et al. (2000) Nature Biotech. 18:989-994; Lueking et al. (1999) Anal. Biochem. 270:103-111; Ge, H. (2000) Nucleic Acids Res. 28 e3:I-VII; MacBeath, G., and Schreiber, S. L. (2000) Science 289:1760-1763; and WO 99/51773A1]. In a preferred embodiment, each addresses of the plurality has disposed thereon a polypeptide at least 60, 70, 80, 85, 90, 95, or 99% identical to protein X or fragment thereof. For example, multiple variants of protein X (e.g., encoded by allelic variants, site-directed mutants, random mutants, or combinatorial mutants) can be disposed at individual addresses of the plurality. Addresses in addition to the address of the plurality can be disposed on the array.

The polypeptide array can be used to detect a protein X-binding compound, e.g., an antibody in a sample from a subject with specificity for protein X or the presence of a protein X-binding protein or ligand.

The array is also useful for ascertaining the effect of the expression of a gene on the expression of other genes in the same cell or in different cells (e.g., ascertaining the effect of gene X expression on the expression of other genes). This provides, for example, for a selection of alternate molecular targets for therapeutic intervention if the ultimate or downstream target cannot be regulated.

In another aspect, the invention features a method of analyzing a plurality of probes. The method is useful, e.g., for analyzing gene expression. The method includes: providing a first two dimensional array having a plurality of addresses, each address (of the plurality) being positionally distinguishable from each other address (of the plurality) having a unique capture probe, e.g., wherein the capture probes are from a cell or subject which express gene X or from a cell or subject in which a gene X-mediated response has been elicited, e.g., by contact of the cell with nucleic acid X or protein X, or administration to the cell or subject of a nucleic acid X or protein X; providing a second two dimensional array having a plurality of addresses, each address of the plurality being positionally distinguishable from each other address of the plurality, and each address of the plurality having a unique capture probe, e.g., wherein the capture probes are from a cell or subject which does not express gene X (or does not express as highly as in the case of the cell or subject described above for the first array) or from a cell or subject which in which a gene X-mediated response has not been elicited (or has been elicited to a lesser extent than in the first sample); contacting the first and second arrays with one or more inquiry probes (which are preferably other than a nucleic acid X, protein X, or antibody specific for protein X), and thereby evaluating the plurality of capture probes. Binding, e.g., in the case of a nucleic acid, hybridization with a capture probe at an address of the plurality, is detected, e.g., by signal generated from a label attached to the nucleic acid, polypeptide, or antibody.

The invention also features a method of analyzing a plurality of probes or a sample. The method is useful, e.g., for analyzing gene expression. The method includes: providing a first two dimensional array having a plurality of addresses, each address of the plurality being positionally distinguishable from each other address of the plurality having a unique capture probe, contacting the array with a first sample from a cell or subject which express or mis-express gene X or from a cell or subject in which a gene X-mediated response has been elicited, e.g., by contact of the cell with nucleic acid X or protein X, or administration to the cell or subject of nucleic acid X or protein X; providing a second two dimensional array having a plurality of addresses, each address of the plurality being positionally distinguishable from each other address of the plurality, and each address of the plurality having a unique capture probe, and contacting the array with a second sample from a cell or subject which does not express gene X (or does not express as highly as in the case of the as in the case of the cell or subject described for the first array) or from a cell or subject which in which a gene X-mediated response has not been elicited (or has been elicited to a lesser extent than in the first sample); and comparing the binding of the first sample with the binding of the second sample. Binding, e.g., in the case of a nucleic acid, hybridization with a capture probe at an address of the plurality, is detected, e.g., by a signal generated from a label attached to the nucleic acid, polypeptide, or antibody. The same array can be used for both samples or different arrays can be used. If different arrays are used the same plurality of addresses with capture probes should be present on both arrays.

In another aspect, the invention features a method of analyzing gene X, e.g., analyzing the structure, function, or relatedness to other nucleic acids or amino acid sequences. The method includes: providing a nucleic acid X or protein X amino acid sequence; comparing the nucleic acid or amino acid sequence with one or more sequences from a collection of sequences, e.g., a nucleic acid or protein sequence database; to thereby analyze gene X.

The following examples are meant to illustrate, not limit, the invention.

### EXAMPLES

### Example 1

### Methods and Materials

Tissue Samples and Tissue Microarrays (TMA)

All human tissue was collected following NIH guidelines and using protocols approved by the Institutional Review Boards of relevant institutions (see below).

Fresh tissue specimens obtained from the Brigham and Women's Hospital, Massachusetts General Hospital, and Faulkner Hospital (all Boston, Mass.), Duke University (Durham, N.C.), University Hospital Zagreb (Zagreb, Croatia), and the National Disease Research Interchange (Philadelphia, Pa.) were snap frozen on dry ice and stored at −80° C. until use. Tumors with significant DCIS components were identified based on pathology reports and confirmed by microscopic examination of hematoxylin-eosin stained frozen sections. Of the tumors used for SAGE analysis, D1, D3, D4, D5 and D6 were high-grade, comedo DCIS, and D2, D7 and T18 were intermediate-grade DCIS with no necrosis. Tumors used for mRNA in situ hybridization and immunohistochemistry included DCIS tumors of all three (low, intermediate, and high grade) histologic types. Most of the tumors used for in situ hybridization and immunohistochemistry were DCIS with concurrent invasive carcinoma and pure DCIS (i.e., without concurrent invasive carinoma), respectively. Tumors D3 and D6 used for SAGE were pure DCIS. The larger representation of frozen/fresh DCIS tumors with concurrent invasive disease was due to logistic issues; it is extremely difficult to obtain frozen or fresh pure DCIS specimens, especially ones with long term clinical follow up data. For in situ hybridization, 5 μm thick frozen sections were mounted on silylated slides (CEL Associates Inc, Pearland, Tex.), air dried, and stored at −80° C. until use.

Tissue microarrays (TMAs) were: (1) obtained from commercial sources (Imgenex, San Diego, Calif. (49 invasive breast tumors); Ambion, Austin, Tex. (92 primary invasive tumors and 41, distant metastases)); (2) provided by the Cooperative Breast Cancer Tissue Resource, Rockville, Md. (40 normal breast tissue samples, 10 pure DCIS tumors, 10 DCIS with concurrent invasive tumors, and 192 primary invasive breast tumors); (3) generated at Johns Hopkins University, Baltimore, Md. (299 invasive breast tumors and 10 distant metastases) and at Beth Israel Deaconess Medical Center (30 invasive breast tumors and 70 pure DCIS tumors of different histologic grades, all with matched normal breast tissue) following published protocols [Kononen et al. (1998) Nat. Med. 4:844-847]. With the exception of the Imgenex and the DCIS arrays (1 mm punches), all TMAs contained 0.6 mm punches, with at least 2 punches/tumor in order to control for tumor and immunohistochemical staining heterogeneity.

Cell Lines

Breast cancer cell lines were obtained from American Type Culture Collection (ATCC; Manassas, Va.) or were generously provided by Drs. Steve Ethier (University of Michigan) and Arthur Pardee (Dana-Farber Cancer Institute). Cells were grown in media recommended by the provider.

Generation and Analysis of SAGE Libraries from Normal and Malignant Breast Tissue

SAGE libraries were generated from DCIS tumors and normal breast tissue and analyzed essentially as previously described as part of the National Cancer Institute Cancer Gene Anatomy Project [Porter et al. (2001) Cancer Res. 61:5697-5702; Krop et al. (2001) Proc. Natl. Acad. Sci. U.S.A. 98:9796-9801; Lal et al. (1999) Cancer Res. 59:5403-5407; and Boon et al. (2002) Proc. Natl. Acad. Sci. U.S.A. 99:11287-11292]. Two of the DCIS tumors were pure DCIS (D3 and D6) and the others were obtained from patients with concurrent invasive breast carcinomas. Epithelial cells from normal breast tissue (N1 and N2) and some tumors (D2, D3, D6, and D7) were purified using epithelial cell-specific monoclonal antibody (BerEP4)-coated magnetic beads (Dynal, Oslo, Norway); other tumors were macroscopically dissected based on adjacent hematoxylin-eosin stained slides. Approximately 50,000 SAGE tags were obtained from each library. For further analyses libraries were normalized to the library with the highest tag number (89,541 total tags). Hierarchical clustering was applied to data using the Cluster program developed by Eisen et al. [Eisen et al. (1998) 95:14863-14868]. Differentially expressed genes were identified based on statistical analysis of comparisons of groups of normal (2 samples), DCIS: (8 samples), and invasive breast cancer (9 samples) SAGE libraries using the SAGE2000 software [Velculescu et al. (1995) Science 270:484-487]. Similarly for the identification of genes specifically expressed in DCIS or invasive breast cancer, the 8 DCIS samples were treated as a group and the 9 invasive or metastatic patients were treated as another group. First, the SAGE tag numbers highest in two normal libraries (N1 and N2) were used as the cut-off and tag numbers in the DCIS and invasive libraries above this “normal” value were calculated using a two-sided Fisher-exact test without multiple comparisons (see Table 4). In a second test, ROC (receiver operating characteristic) curve analysis was used to choose, the “best” cut-off for values (Table 4). A ROC area of 0.50 is no better than chance and a ROC area of 1.00 is the best possible.

mRNA In Situ Hybridization

To generate templates for in vitro transcription reactions, 300-500 base pair fragments derived from the 3′ untranslated region of the selected genes were PCR amplified and subcloned into the pZERO 1.0 expression vector (Invitrogen, Carlsbad, Calif.). pZERO 1.0 contains a multiple cloning site bounded by SP6 and T7 RNA polymerase promoters; therefore the same plasmid can be used for the generation of sense and anti-sense riboprobes for mRNA in situ hybridizations. Digitonin-labeled sense and anti-sense riboprobes were generated and mRNA in situ hybridization was performed as described [Qian et ale (2001) Genes Dev. 15:2533-2545; Porter et al. (2003a) Mol. Cancer Res. 1:362-375]. The hybridized sections were observed with a NIKON microscope, images were obtained using a SPOT CCD camera, and the images were processed with the Adobe (San Jose, Calif.) Photoshop program. Hybridizations were considered successful if the control sense probe gave no significant signal. The intensity and distribution of the hybridization signal were scored (0-3 for intensity and 0-3 for distribution using the scoring scheme described below for immunohistochemistry) independently by three investigators.

Immunohistochemistry

The expression of the indicated genes in primary breast tumors was determined by immunohistochemical analysis of eight tissue microarrays that contained evaluatable paraffin-embedded specimens derived from 80 DCIS, 675 primary invasive breast cancer, and 33 distant metastases. Antigen Retrieval Citra solution (Research Genetics, San Ramon, Calif.) and boiling in a microwave oven (5 minutes at high power) were used to enhance staining. Isotype control serum was used for negative control samples. A standard indirect immunoperoxidase protocol with 3,3′-diaminobenzidine as chromogen was used for the visualization of antibody binding (ABC-Elite; Vector Laboratories, Burlingame, Calif.).

Primary antibodies used were as follows: mouse monoclonal antibody specific for human psoriasin (“anti-psoriasin”) [Enerback et al. (2002) Cancer Res. 62:43-47]; affinity-purified rabbit polyclonal antibody specific for human Connective Tissue Growth Factor (CTGF) (“anti-CTGF”) (a generous gift of Dr. D. Brigstock, Childrens' Research Institute, Columbus, Ohio); affinity-purified rabbit polyclonal antibody specific for human Trefoil Factor 3 (TFF3) (“anti-TFF3”) (a kind gift of Prof. Hoffman, Universitaetsklinikum, Magdeburg, Germany); mouse monoclonal antibodies specific for human interleukin-8 (IL-8) (“anti-IL-8”), GRO-1 (“anti-GRO-1”), and GRO-2 (“anti-GRO-2”) (R&D Systems, Minneapolis, Minn.); monoclonal antibody specific for human osteonectin (SPARC) (“anti-SPARC”) (Hematologic Technologies, Essex Junction, Vt.); and monoclonal antibody specific for human fatty acid synthase (FASN) (“anti-FASN”) (Transduction Labs. San Diego, Calif.). Mouse monoclonal antibodies specific for interleukin-1β (IL1β) and CCL3 (chemokine (CC motif) ligand 3, also known as macrophage inhibitory protein 1α (MIP1α)) were purchased from R&D (Minneapolis, Minn.) while anti-CD45 mouse monoclonal antibody was obtained from DAKO (Carpinteria, Calif.). Antibodies were used at a 1:100 dilution in PBS (phosphate buffered saline) containing 10% heat-inactivated goat serum.

Antibody staining was subjectively scored by three investigators independently on a scale of 0-3 for intensity (0=no staining, 1=faint signal, 2=moderate and 3=intense staining) and 0-3 for extent (0=no, 1=≦30%, 2=30-70%, and 3=≧70% positive cells) of staining. Cumulative scores were obtained by adding the average intensity and extent scores assigned by the three independent observers. For statistical analyses a cumulative score at or above 3 was considered positive. Relationships between the expression of genes determined by mRNA in situ hybridization or immunohistochemistry were analyzed by Fishers exact test without correction for multiple comparisons.

Statistical Analyses of Clinical Correlates

The relationship of gene expression to clinico-pathologic parameters and the association between the expression of different genes determined by immunohistochemistry were analyzed by the following statistical methods.

The eight individual tissue microarray datasets and a combined dataset were analyzed for association of gene expression positivity and prognostic factors using a logistic regression model (with gene expression positivity as the outcome), and a forward, or step-up, selection procedure to determine the best fitting model. Clinico-pathologic factors analyzed were: expression of the estrogen and progesterone receptors and HER2 by immunohistochemistry, histologic grade, TNM (tumor, node metastasis) stage, tumor size, number of positive lymph nodes, patient age, and overall and distant metastasis-free survival. If all patients or no patients with a particular level of a covariate demonstrated gene expression positivity, then the logistic regression did not converge and a significance level was obtained using Fisher's exact test. If, however, there remained some patients with and without gene expression positivity after deleting patients with the particular level of the covariate, then a step-up logistic regression was performed on them. The significance of the variables in the logistic regression models was tested using likelihood ratio tests. The cut-off used for entry into the model was α=0.05. In addition to the analyses described above, Kaplan-Meier curves were generated and Cox models were run for two datasets that contained survival information. Calculated times to distant failure and times to survival were used and were based on the failure/death and accession dates.

Generation of SAGE Libraries from Epithelial and Non-Epithelial Cells of Normal Breast and DCIS Tissue

The procedure described in this section was used to obtain the data described in Example 6.

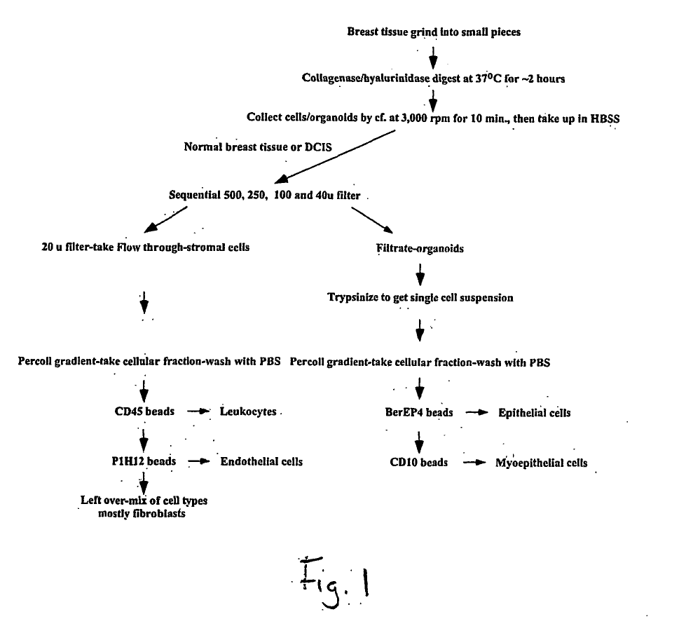

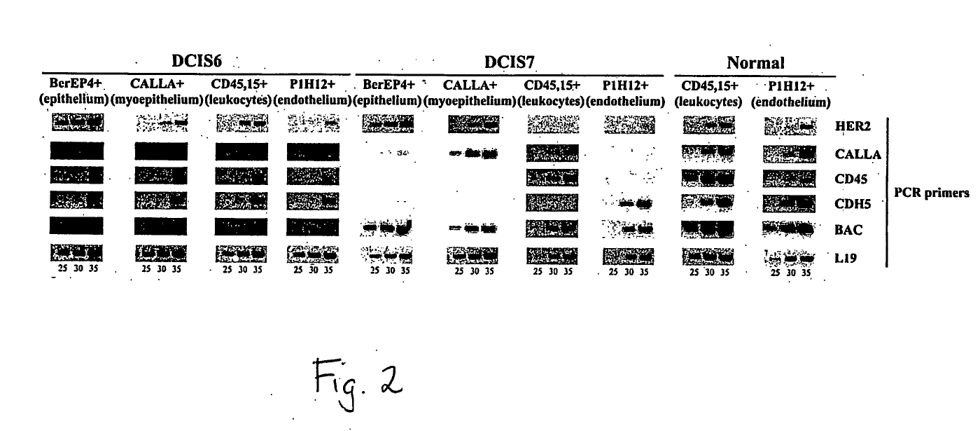

Some of the cell types present in normal and cancerous breast tissue comprise a minor fraction (a few percent) of all cells of the relevant tissue; thus, genes that are specifically expressed in such cell types may not be detected by analysis of the whole tissue. In order to analyze the comprehensive gene expression profiles of purified luminal epithelial cells, myoepithelial cells, endothelial cells, fibroblasts and leukocytes isolated from normal breast tissue and breast carcinomas using SAGE, a purification procedure that allows the isolation of pure cell populations was developed. A brief outline of the procedure is depicted in FIG. 1. In order to isolate specific cell types, antibodies specific for cell type-specific cell surface markers and magnetic beads were employed using well-established methods. Thus, luminal mammary epithelial cells were isolated using the BerEp4 monoclonal antibody, myoepithelial cells with a monoclonal antibody specific for CD10/Cella, infiltrating leukocytes with a monoclonal antibody specific for the CD45 panleukocyte marker, and endothelial cells with the P1H12 monoclonal antibody that binds to an endothelial-specific cell surface protein. Essentially all the cells separated, as luminal cells from breast cancer samples would be breast cancer cells. Thus, as used herein, breast “stromal cells” are breast cells other than epithelial cells. No antibody specific for a cell surface marker specific for fibroblasts was identified. Therefore, on the assumption that after removal of the above listed-cell types the “leftover” cells were enriched for fibroblasts, the leftover cells were considered to be a “fibroblast enriched” fraction. The success of the purification procedure and the purity of each cell fraction were confirmed by a RT-PCR (reverse transcription-polymerase chain reaction) analysis of RNA isolated from 1/10 of the cells using the cell type specific marker used for the isolation of the cells. In FIG. 2 is shown the results of such an RT-PCR analysis of RNA isolated from: (a) luminal epithelial cells (“epithelium”), myoepithelial cells (“myoepithelium”), leukocytes, and endothelial cells (“endothelium”) purified as described above from two DCIS tumors (DCIS6 and DCIS7); and (b) leukocytes and endothelial cells (“endothelium”) from normal breast tissue. The PCR phases of the RT-PCRs were carried out with oligonucleotide primers specific for β-actin (“BAC”) and L19 (both constitutively expressed by all cells), HER2 (expressed by some breast cancers), CALLA (a myoepithelial cell marker), CD45 (a pan-leukocyte marker) and an endothelial cell surface protein (“CDH5”, an endothelial cell marker). PCR were performed for 25, 30, and 35 cycles.

The cells not used for the RT-PCR analysis were used for the generation of micro-SAGE libraries. SAGE libraries were generated from luminal epithelial cells, myoepithelial cells, infiltrating lymphocytes, and endothelial cells from a normal breast reduction tissue (1 library/cell type) and from DCIS luminal and myoepithelial cells, infiltrating lymphocytes and endothelial cells (2 different tumors-2 libraries/cell type). Approximately 50,000 SAGE tags were obtained from, each library, thereby enabling the analysis of thousands of unique transcripts. Based on these SAGE data, genes that are differentially expressed in specific cell types of normal and DCIS breast tissue were identified.

Ligand Binding, Cell Growth, Migration and Invasion Assays

N-terminal or C-terminal alkaline phosphatase (AP) CXCL14 fusion proteins were generated using the AP-TAG-5 expression vector (GenHunter, Nashville, Tenn.). Mammalian cells were transfected with Fugene6 (Roche, Indianapolis, IN), Lipofectamine or Lipofectamine 2000 (LifeTechnologies, Rockville, Md.) reagents. In vivo and in vitro ligand binding assays were carried out on primary tissues and cell lines using AP-CXCL14 essentially as described (Flanagan et-al (1990) Cell 63:185-194; Porter et al. (2003b) Proc. Natl. Acad. Sci. USA 100:10931-10936]. Briefly, frozen sections of various human specimens were fixed, incubated with either AP-CXCL14 fusion protein or AP control conditioned medium, rinsed, and then incubated with AP substrate forming a blue/purple precipitate. For in vitro assays cells in suspension with conditioned media containing either AP alone or AP-CXCL14 fusion protein, rinsed, and then assayed for bound AP activity;

To determine the effect of CXCL14 on cell growth, MDA-MB-231 and MCF10A cells were plated (4,000 cells/well) in a 24 well tissue culture plate and grown in conditioned medium containing AP or AP-CXCL14. Conditioned medium was generated by transfecting 293 cells with pAP-tag5 or pAP-CXCL14 plasmids and growing them in McCoy's medium supplemented with 10% fetal bovine serum (FBS) (used for MDA-MB-231 cells) or in MCF10A media (ATCC; used for MCF10A cells). Cells were counted (3 wells/time point) on days 1, 2, 4, 6, and 8 after plating. 10 nM CXCL12 was used as a positive control in the experiment with MDA-MB-231 cells. The experiments were repeated three times.

In order to determine if CXCL14 binding to breast cancer cells has an effect on cell migration and invasion, the ability of conditioned medium containing AP-CXCL14 or pcDNA3.1 expressing HA (hemagglutinin)-tagged CXCL14 to induce the migration and invasion of MDA-MB-231 cells was tested using BIOCOAT Matrigel invasion chambers essentially as previously described [Muller (2001) Nature 410:50-56]. For invasion assays, cells were plated at a concentration of 2.5×10⁴ cells/well and assayed 24 hours later. For migration assays cells at a concentration of 1.25×10⁴ cells/well were used and cell numbers were determined 12 hours later. Conditioned media from cells transfected with pAP-Tag5 or pCDNA 3.1 empty vectors were used as negative controls.

### Example 2

### Normal and Cancerous Breast Transcriptomes Determined by SAGE

Genes differentially expressed between normal and cancerous breast tissues were identified using SAGE. Confirming previous studies of the inventors using a smaller number of SAGE libraries [Porter et al. (2001) Cancer Res. 61:5697-5702], the most dramatic difference in gene expression patterns was found to occur at the normal to in situ carcinoma transition and involves the uniform down-regulation of 32 genes (Table 1); while 34 tags and their corresponding genes are shown in Table 1, two genes (encoding interleukin-8 and GRO10 were each represented by two tags. Table 1 shows data from two normal breast tissue samples (N1 and N2), eight DCIS samples (D1-D7 and T18), six invasive breast cancer samples (11-16), two lymph node metastases (LN1 and LN2) from the same subjects that samples I1 and I2 were obtained from, and a lung metastasis (MET) from a breast cancer patient. In Table 1 and subsequent tables, Unigene identification numbers for relevant genes are shown in columns labeled “Unigene”. The contents (e.g., nucleic acid sequences and amino acid sequences) of database submissions identified by all the listed Unigene identification numbers are incorporated herein by reference in their entirety. Since many of the genes whose expression was found to be down-regulated after the normal to in situ transition encode secreted proteins and genes related to epithelial cell differentiation, loss of the differentiated epithelial phenotype and abnormal autocrine/paracrine interactions appear to play an essential role in the initiation of breast tumorigenesis.

The inventors also identified 144 genes up-regulated in a fraction of in situ, invasive and metastatic tumors (Table 2). The normal, DCIS, and lymph node samples studied in this analysis were the same as those shown in Table 1. Invasive breast cancer samples I1-I5 were the same as samples I1-I5 shown in Table 1 and T15 was an additional invasive breast cancer sample. Nearly ¼ of the relevant SAGE tags currently have no database match indicating that many transcripts specifically expressed in certain breast carcinomas remain to be identified.

TABLE 1
Genes universally down-regulated in breast cancer irrespective of pathologic stage

| SEQ | | | | | | | | | | | | | | | | | | | | | |
|---|---|---|---|---|---|---|---|---|---|---|---|---|---|---|---|---|---|---|---|---|---|
| ID | | | | | | | | | | | | | | | | | | | | | |
| NO: | Tag sequence | Unigene | Gene | N1 | N2 | D1 | D2 | D3 | D4 | D5 | D6 | D7 | T18 | 11 | 12 | 13 | 14 | 15 16 | LN1 | LN2 | MET |

Secreted proteins

| | | | | | | | | | | | | | | | | | | | | | | |
|---|---|---|---|---|---|---|---|---|---|---|---|---|---|---|---|---|---|---|---|---|---|---|
| 1 | AAATATCCAG | 624 | interleukin 8* | 15 | 5 | 0 | 0 | 0 | 0 | 0 | 1 | 0 | 0 | 0 | 0 | 0 | 0 | 0 | 0 | 0 | 0 | 0 |
| 2 | TGGAAGCACT | 624 | interleukin 8* | 368 | 352 | 8 | 39 | 12 | 1 | 0 | 94 | 15 | 0 | 2 | 0 | 1 | 0 | 0 | 0 | 0 | 0 | 0 |
| 3 | AAGCTCGCCG | 62492 | secretoglobin,family 3A, member 1 (HIN-1) | 125 | 44 | 0 | 0 | 0 | 3 | 0 | 9 | 0 | 0 | 0 | 0 | 0 | 0 | 0 | 0 | 0 | 0 | 4 |
| 4 | TTGAAACTTT | 789 | CXCL1 (GRO1)* | 394 | 453 | 11 | 12 | 14 | 1 | 0 | 61 | 1 | 4 | 0 | 0 | 1 | 0 | 1 | 0 | 0 | 0 | 2 |
| 5 | TTGCAGGCTC | 789 | CXCL1 (GRO1)* | 13 | 40 | 0 | 0 | 0 | 0 | 0 | 1 | 0 | 0 | 0 | 0 | 0 | 0 | 0 | 0 | 0 | 0 | 0 |
| 6 | ATAATAAAAG | 89690 | GRO3 | 24 | 205 | 4 | 0 | 6 | 4 | 4 | 2 | 0 | 5 | 7 | 5 | 3 | 8 | 4 | 8 | 6 | 7 | 11 |
| 7 | TTGGTTTTTG | 164021 | small inducible cytokine subfamily B (Cys-X-Cys), member 6 | 56 | 16 | 0 | 3 | 0 | 0 | 0 | 1 | 0 | 0 | 0 | 0 | 1 | 0 | 0 | 0 | 0 | 0 | 4 |
| 8 | GAGGGTTTAG | 75498 | small inducible cytokine subfamily A (Cys-Cys), member 20 | 44 | 30 | 2 | 0 | 0 | 0 | 0 | 2 | 2 | 0 | 0 | 0 | 1 | 0 | 0 | 0 | 0 | 0 | 0 |
| 9 | GTACTAGTGT | 303649 | small inducible cytokine A2 | 33 | 12 | 2 | 0 | 3 | 1 | 0 | 2 | 1 | 0 | 2 | 3 | 3 | 0 | 1 | 4 | 0 | 0 | 2 |
| 10 | GCCTTAACAA | 239138 | pre-B-cell colony-enhancing factor | 45 | 30 | 11 | 15 | 0 | 7 | 6 | 17 | 9 | 2 | 7 | 4 | 5 | 4 | 1 | 4 | 4 | 3 | 7 |
| 11 | GCCTTGGGTG | 2250 | leukemia inhibitory factor | 64 | 135 | 0 | 3 | 8 | 1 | 0 | 4 | 10 | 0 | 0 | 0 | 1 | 0 | 0 | 4 | 0 | 0 | 0 |

Cell surface proteins/receptors

| | | | | | | | | | | | | | | | | | | | | | | |
|---|---|---|---|---|---|---|---|---|---|---|---|---|---|---|---|---|---|---|---|---|---|---|
| 12 | ACCAAATTAA | 51233 | tumor necrosis factor receptor superfamily, member 10b | 31 | 35 | 11 | 0 | 0 | 1 | 2 | 6 | 13 | 2 | 4 | 8 | 1 | 3 | 7 | 12 | 6 | 7 | 7 |
| 13 | AGAAAGATGT | 78225 | annexin A1 | 83 | 77 | 11 | 3 | 15 | 12 | 10 | 9 | 4 | 23 | 4 | 16 | 19 | 3 | 7 | 16 | 6 | 0 | 20 |
| 14 | TGACTGGCAG | 278573 | CD59 antigen p18-20 | 49 | 33 | 15 | 9 | 11 | 0 | 4 | 6 | 9 | 4 | 4 | 1 | 14 | 11 | 1 | 0 | 0 | 3 | 5 |
| 15 | GTCCGAGTGC | 374348 | ESTs, Highly similar to A42926 L6 surface protein | 134 | 96 | 11 | 33 | 11 | 1 | 2 | 23 | 13 | 4 | 2 | 0 | 0 | 8 | 0 | 8 | 2 | 3 | 5 |

Cell growth and survival

| | | | | | | | | | | | | | | | | | | | | | | |
|---|---|---|---|---|---|---|---|---|---|---|---|---|---|---|---|---|---|---|---|---|---|---|
| 16 | GCTTGCAAAA | 372783 | superoxide dismutase 2, mitochondrial | 210 | 121 | 6 | 12 | 5 | 3 | 0 | 10 | 3 | 0 | 4 | 0 | 1 | 1 | 1 | 4 | 6 | 3 | 7 |
| 17 | ACCAGGCCAC | 101382 | tumor necrosis factor, alpha-induced protein 2 | 24 | 23 | 0 | 0 | 0 | 9 | 0 | 7 | 7 | 0 | 0 | 1 | 1 | 0 | 10 | 0 | 2 | 0 | 4 |
| 18 | TTTGAAATGA | 28491 | spermidine/spermine N1-acetyltransferase | 129 | 133 | 13 | 45 | 37 | 29 | 6 | 20 | 55 | 5 | 4 | 12 | 40 | 11 | 13 | 20 | 4 | 4 | 7 |
| 19 | CTTGCAAACC | 127799 | baculoviral IAP repeat-containing 3 | 16 | 26 | 0 | 6 | 2 | 1 | 0 | 1 | 2 | 0 | 2 | 1 | 1 | 0 | 1 | 4 | 0 | 1 | 4 |
| 20 | CCATTGAAAC | 75517 | laminin, beta 3 | 20 | 21 | 2 | 3 | 2 | 1 | 0 | 2 | 0 | 7 | 0 | 0 | 5 | 1 | 1 | 0 | 0 | 1 | 2 |
| 21 | CCCGAGGCAG | 155223 | stanniocalcin 2 | 62 | 23 | 4 | 6 | 0 | 0 | 2 | 4 | 4 | 2 | 0 | 4 | 6 | 3 | 4 | 0 | 0 | 1 | 2 |
| 22 | CTGGCCCTCG | 348024 | v-ral simian leukemia viral oncogene homolog B | 296 | 145 | 55 | 117 | 9 | 0 | 31 | 12 | 74 | 69 | 2 | 1 | 0 | 0 | 1 | 0 | 2 | 3 | 2 |
| 23 | GACACGAACA | 25829 | RAS, dexamethasone-induced 1 | 45 | 30 | 6 | 0 | 8 | 4 | 0 | 2 | 2 | 9 | 9 | 3 | 1 | 7 | 0 | 0 | 2 | 4 | 11 |
| 24 | GCTGCCCTTG | 272897 | tubulin, alpba 3 | 103 | 75 | 13 | 30 | 3 | 10 | 8 | 18 | 32 | 2 | 11 | 9 | 13 | 15 | 12 | 20 | 6 | 12 | 16 |

Differentiation

| | | | | | | | | | | | | | | | | | | | | | | |
|---|---|---|---|---|---|---|---|---|---|---|---|---|---|---|---|---|---|---|---|---|---|---|
| 25 | CGAATGTCCT | 335952 | keratin 6B | 53 | 49 | 0 | 0 | 17 | 0 | 0 | 4 | 0 | 0 | 0 | 0 | 0 | 1 | 0 | 0 | 0 | 0 | 2 |
| 26 | CTCACTTTTT | 76722 | CCAAT/enhancer binding protein (C/EBP), delta | 154 | 112 | 38 | 45 | 11 | 16 | 33 | 22 | 22 | 12 | 7 | 4 | 12 | 17 | 0 | 0 | 4 | 6 | 23 |

Unknown function

| | | | | | | | | | | | | | | | | | | | | | | |
|---|---|---|---|---|---|---|---|---|---|---|---|---|---|---|---|---|---|---|---|---|---|---|
| 27 | AGAATGTAGG | 105094 | ESTs | 13 | 26 | 2 | 0 | 0 | 0 | 0 | 0 | 0 | 2 | 0 | 1 | 3 | 0 | 1 | 0 | 2 | 0 | 0 |
| 28 | AGTCAAAAAT | NA | No reliable match | 13 | 14 | 0 | 0 | 0 | 0 | 0 | 1 | 4 | 0 | 0 | 0 | 0 | 0 | 1 | 0 | 0 | 0 | 0 |
| 29 | ATTAGTGTTG | 23740 | KIAAI598 protein | 15 | 7 | 0 | 0 | 0 | 0 | 0 | 1 | 1 | 0 | 0 | 0 | 1 | 0 | 0 | 0 | 4 | 0 | 0 |
| 30 | CTTTGGAAAT | 6820 | Homo sapien cDNA FLJ32718 fis | 16 | 54 | 4 | 0 | 3 | 1 | 0 | 4 | 5 | 0 | 0 | 0 | 0 | 0 | 0 | 8 | 2 | 0 | 9 |
| 31 | GCAACTTAGA | NA | No reliable match | 29 | 21 | 6 | 3 | 0 | 1 | 0 | 2 | 1 | 7 | 0 | 0 | 4 | 3 | 0 | 0 | 0 | 0 | 0 |
| 32 | GGGACGAGTG | NA | No reliable match | 250 | 460 | 48 | 493 | 34 | 29 | 53 | 89 | 51 | 49 | 25 | 9 | 8 | 117 | 3 | 32 | 16 | 19 | 88 |
| 33 | GGGTTTGTTT | 75969 | proline rich 2 | 38 | 44 | 4 | 0 | 3 | 4 | 4 | 20 | 8 | 0 | 2 | 1 | 6 | 11 | 1 | 8 | 2 | 1 | 14 |
| 34 | GTCTTAAAGT | 177781 | Homo sapiens, clone IMAGE:4711494, mRNA | 100 | 58 | 0 | 0 | 3 | 1 | 0 | 21 | 8 | 0 | 2 | 0 | 5 | 4 | 1 | 8 | 4 | 1 | 2 |

*From interleukin 8 and GRO1 two independent SAGE tags were derived and both were down-regulated in tumors.

TABLE 2
Genes up-regulated in breast cancer

| | | | Normal | | | In situ | | | | | | | | | Invasive | | | | | | | Metastatic | | | |
|---|---|---|---|---|---|---|---|---|---|---|---|---|---|---|---|---|---|---|---|---|---|---|---|---|---|
| Tag | Unigene | Gene | N1 | N2 | Ave | D1 | D2 | D3 | D4 | D5 | D6 | D7 | T18 | Ave | I1 | I2 | I3 | I4 | I5 | T15 | Ave | LN1 | LN2 | MET | Ave |

Secreted proteins and ECM related

| | | | | | | | | | | | | | | | | | | | | | | | | | |
|---|---|---|---|---|---|---|---|---|---|---|---|---|---|---|---|---|---|---|---|---|---|---|---|---|---|
| ATGTCTTTTC | 1516 | insulin-like growth factor binding protein 4 | 4 | 5 | 5 | 17 | 36 | 6 | 32 | 59 | 9 | 9 | 4 | 21 | 13 | 29 | 33 | 7 | 19 | 24 | 21 | 8 | 29 | 2 | 13 |
| CATATCATTA | 119206 | insulin-like growth factor binding protein 7 | 0 | 0 | 0 | 11 | 6 | 6 | 63 | 39 | 4 | 3 | 42 | 22 | 49 | 63 | 59 | 59 | 28 | 80 | 57 | 55 | 12 | 18 | 28 |
| CTCCACCCGA | 352107 | trefoil factor 3 (intestinal) | 34 | 7 | 21 | 511 | 854 | 17 | 26 | 451 | 31 | 38 | 261 | 274 | 369 | 124 | 15 | 0 | 94 | 16 | 103 | 285 | 244 | 2 | 177 |
| ACGTTAAAGA | 350570 | dermcidin (IBC-1) | 0 | 0 | 0 | 0 | 0 | 0 | 1 | 0 | 0 | 0 | 0 | 0 | 177 | 101 | 3 | 0 | 0 | 12 | 49 | 199 | 0 | 0 | 66 |
| ATTTTCTAAA | 91011 | anterior gradient 2 homolog | 4 | 7 | 5 | 13 | 75 | 2 | 39 | 2 | 7 | 5 | 0 | 18 | 13 | 17 | 3 | 0 | 12 | 0 | 7 | 2 | 54 | 0 | 19 |
| AGTGGTGGCT | 230 | fibromodulin | 0 | 0 | 0 | 17 | 0 | 2 | 22 | 0 | 0 | 2 | 34 | 9 | 34 | 36 | 3 | 1 | 70 | 12 | 26 | 22 | 6 | 25 | 18 |
| ATCTTGTTAC | 287820 | fibronectin 1 | 0 | 0 | 0 | 4 | 0 | 5 | 7 | 14 | 0 | 2 | 2 | 4 | 2 | 4 | 15 | 4 | 21 | 12 | 10 | 2 | 1 | 0 | 1 |
| TTATGTTTAA | 79914 | lumican | 0 | 0 | 0 | 2 | 3 | 2 | 28 | 4 | 1 | 1 | 11 | 6 | 0 | 20 | 21 | 1 | 25 | 20 | 14 | 16 | 6 | 11 | 11 |
| CTCATCTGCT | 82109 | syndecan 1 | 0 | 0 | 0 | 0 | 3 | 2 | 25 | 14 | 20 | 2 | 11 | 9 | 4 | 5 | 10 | 36 | 10 | 0 | 11 | 10 | 1 | 9 | 7 |
| ACATTCCAAG | 245188 | tissue inhibitor of metalloproteinase 3 | 0 | 2 | 1 | 13 | 24 | 0 | 12 | 12 | 2 | 7 | 9 | 10 | 7 | 3 | 9 | 1 | 15 | 4 | 6 | 6 | 9 | 7 | 7 |
| CCAGAGAGTG | 180884 | carboxypeptidase B1 (tissue) | 0 | 0 | 0 | 0 | 9 | 0 | 0 | 0 | 0 | 21 | 0 | 4 | 107 | 115 | 0 | 1 | 0 | 0 | 37 | 0 | 354 | 2 | 119 |
| TTTGGTTTTC | 179573 | collagen, type I, alpha 2 | 0 | 0 | 0 | 231 | 0 | 8 | 175 | 53 | 4 | 3 | 12 | 61 | 92 | 90 | 159 | 11 | 158 | 40 | 92 | 138 | 70 | 48 | 85 |
| ACCAAAAACC | 172928 | collagen, type I, alpha 1 | 2 | 5 | 3 | 282 | 3 | 8 | 108 | 41 | 22 | 8 | 85 | 70 | 92 | 71 | 83 | 3 | 185 | 189 | 104 | 153 | 34 | 57 | 81 |
| TGGAAATGAC | 172928 | collagen, type I, alpha 1 | 2 | 2 | 2 | 191 | 0 | 8 | 260 | 80 | 9 | 0 | 11 | 70 | 184 | 91 | 218 | 23 | 254 | 40 | 135 | 252 | 87 | 39 | 126 |
| TTTGTTTTTA | 3622 | procollagen-proline, 2-oxoglutarate | 0 | 0 | 0 | 0 | 3 | 2 | 3 | 2 | 1 | 4 | 2 | 2 | 7 | 7 | 27 | 4 | 21 | 4 | 11 | 2 | 18 | 0 | 7 |
| | | 4-dioxygenase | | | | | | | | | | | | | | | | | | | | | | | |
| TGGCCCCAGG | 268571 | apolipoprotein C-1 | 2 | 2 | 2 | 8 | 0 | 3 | 44 | 47 | 1 | 3 | 19 | 16 | 17 | 58 | 22 | 8 | 45 | 92 | 52 | 81 | 28 | 32 | 47 |
| CGACCCCACG | 169401 | apolipoprotein E | 5 | 2 | 4 | 13 | 0 | 15 | 16 | 33 | 4 | 2 | 65 | 18 | 29 | 37 | 14 | 3 | 54 | 173 | 52 | 31 | 28 | 32 | 31 |
| AACACAGCCT | 170250 | complement component 4A | 5 | 5 | 5 | 25 | 3 | 0 | 52 | 4 | 1 | 5 | 110 | 15 | 29 | 17 | 51 | 0 | 160 | 84 | 57 | 4 | 46 | 7 | 19 |
| GAATTTCCCA | 2353 | complement component 2 | 0 | 0 | 0 | 17 | 0 | 0 | 1 | 2 | 0 | 0 | 19 | 5 | 2 | 7 | 1 | 6 | 1 | 8 | 4 | 6 | 1 | 7 | 5 |
| CAAACTAACC | 153261 | immunoglobulin heavy constant mu | 0 | 0 | 0 | 11 | 0 | 2 | 50 | 0 | 1 | 0 | 28 | 11 | 172 | 70 | 40 | 1 | 0 | 0 | 47 | 320 | 13 | 193 | 176 |
| GAAATAAAGC | 300697 | immunoglobulin heavy constant gamma 3 | 0 | 0 | 0 | 55 | 0 | 129 | 459 | 10 | 1 | 0 | 247 | 113 | 721 | 665 | 53 | 43 | 0 | 2442 | 654 | 1445 | 109 | 770 | 775 |
| AAACCCCAAT | 181125 | immunoglobulin lambda joining 3 | 0 | 0 | 0 | 15 | 0 | 17 | 102 | 4 | 1 | 1 | 44 | 23 | 163 | 87 | 78 | 3 | 0 | 241 | 95 | 258 | 10 | 38 | 102 |

Cell surface proteins/receptors

| | | | | | | | | | | | | | | | | | | | | | | | | | |
|---|---|---|---|---|---|---|---|---|---|---|---|---|---|---|---|---|---|---|---|---|---|---|---|---|---|
| AAGCACAAAA | 9963 | TYRO protein tyrosine kinase binding protein | 0 | 0 | 0 | 2 | 0 | 0 | 13 | 12 | 0 | 0 | 0 | 3 | 20 | 12 | 8 | 3 | 16 | 12 | 12 | 14 | 7 | 23 | 15 |
| TGGTTTGCGT | 6459 | putative G-protein coupled receptor GPCR41 | 4 | 7 | 5 | 29 | 36 | 5 | 36 | 45 | 13 | 23 | 12 | 25 | 27 | 25 | 5 | 72 | 12 | 8 | 25 | 24 | 39 | 16 | 25 |
| TACAATAAAC | 9071 | progesterone receptor membrane component 2 | 0 | 0 | 0 | 4 | 9 | 0 | 17 | 18 | 1 | 5 | 0 | 7 | 9 | 5 | 14 | 6 | 18 | 8 | 10 | 20 | 16 | 9 | 15 |
| AGGAAGGAAC | 323910 | v-erb-b2 | 0 | 0 | 0 | 8 | 9 | 11 | 157 | 43 | 110 | 24 | 81 | 55 | 60 | 42 | 13 | 11 | 6 | 96 | 38 | 104 | 12 | 4 | 40 |
| ACATTCTTTT | 82226 | glycoprotein (transmembrane) nmb | 2 | 0 | 1 | 4 | 0 | 2 | 7 | 8 | 1 | 0 | 5 | 3 | 4 | 9 | 13 | 18 | 9 | 36 | 15 | 10 | 6 | 25 | 14 |
| CACCCTGTAC | 25450 | solute carrier family 29 | 0 | 0 | 0 | 0 | 0 | 2 | 3 | 8 | 0 | 0 | 44 | 7 | 4 | 1 | 5 | 157 | 9 | 20 | 33 | 2 | 9 | 4 | 5 |
| GTTCACATTA | 84298 | CD74 antigen | 7 | 33 | 20 | 29 | 6 | 25 | 188 | 70 | 6 | 13 | 28 | 46 | 159 | 208 | 226 | 32 | 428 | 474 | 154 | 203 | 72 | 72 | 115 |
| CAAGCAGGAC | 179516 | integral type I protein | 2 | 0 | 1 | 17 | 15 | 0 | 38 | 6 | 2 | 4 | 64 | 18 | 29 | 15 | 12 | 30 | 13 | 44 | 24 | 14 | 28 | 16 | 19 |
| TGCTGCCTGT | 118110 | bone marrow stromal cell antigen 2 | 4 | 9 | 6 | 13 | 57 | 2 | 38 | 14 | 12 | 85 | 57 | 35 | 22 | 41 | 22 | 10 | 21 | 153 | 45 | 6 | 78 | 41 | 42 |
| CCCATCATCC | 306122 | glycoprotein, synaptic 2 | 0 | 0 | 0 | 0 | 6 | 0 | 7 | 16 | 1 | 10 | 16 | 7 | 4 | 8 | 17 | 1 | 15 | 4 | 8 | 2 | 6 | 7 | 5 |
| GCAGTGGCCT | 184276 | solute carrier family 9 | 5 | 7 | 6 | 19 | 96 | 8 | 13 | 53 | 13 | 25 | 9 | 30 | 45 | 32 | 6 | 7 | 19 | 12 | 20 | 31 | 32 | 13 | 25 |

Cell cycle and apotosis

| | | | | | | | | | | | | | | | | | | | | | | | | | |
|---|---|---|---|---|---|---|---|---|---|---|---|---|---|---|---|---|---|---|---|---|---|---|---|---|---|
| AAAGTCTAGA | 82932 | cyclin D1 | 7 | 2 | 5 | 19 | 63 | 6 | 42 | 39 | 29 | 17 | 4 | 27 | 56 | 114 | 36 | 3 | 53 | 12 | 46 | 20 | 140 | 2 | 54 |
| CTGGCGCCGA | 183180 | APC11 anaphase promoting complex subunit 11 | 4 | 2 | 3 | 11 | 42 | 2 | 7 | 29 | 2 | 2 | 12 | 13 | 22 | 17 | 19 | 11 | 15 | 28 | 19 | 26 | 28 | 20 | 24 |

Protein synthesis, transport and degradation

| | | | | | | | | | | | | | | | | | | | | | | | | | |
|---|---|---|---|---|---|---|---|---|---|---|---|---|---|---|---|---|---|---|---|---|---|---|---|---|---|
| TTTCAGAGAG | 75975 | signal recognition particle 9kDa | 13 | 9 | 11 | 86 | 18 | 23 | 92 | 64 | 10 | 34 | 25 | 44 | 51 | 71 | 83 | 48 | 89 | 24 | 61 | 53 | 60 | 41 | 51 |
| TTCTTGCTTA | 189895 | ubiquitin-conjugating enzyme E2L 6 | 0 | 0 | 0 | 0 | 6 | 3 | 7 | 12 | 2 | 7 | 11 | 6 | 9 | 12 | 14 | 6 | 6 | 36 | 14 | 4 | 25 | 5 | 11 |
| GAGAGTGGGG | 252259 | ribosomal protein S3 | 0 | 0 | 0 | 6 | 0 | 0 | 0 | 0 | 0 | 0 | 14 | 3 | 18 | 4 | 0 | 0 | 0 | 12 | 6 | 10 | 25 | 0 | 12 |

Transcription, chromatin, other nuclear proteins

| | | | | | | | | | | | | | | | | | | | | | | | | | |
|---|---|---|---|---|---|---|---|---|---|---|---|---|---|---|---|---|---|---|---|---|---|---|---|---|---|
| TGAGCAAGCC | 27801 | zinc finger protein 278 | 0 | 0 | 0 | 6 | 0 | 2 | 1 | 2 | 1 | 0 | 7 | 2 | 18 | 11 | 3 | 0 | 9 | 4 | 7 | 14 | 16 | 2 | 11 |
| CCTGTACCCC | 32317 | high-nobility group 20B | 0 | 0 | 0 | 2 | 3 | 3 | 3 | 8 | 4 | 6 | 25 | 7 | 7 | 7 | 8 | 7 | 6 | 12 | 8 | 2 | 7 | 0 | 3 |
| CCTTTCACAC | 278589 | general transcription factor II, i | 4 | 2 | 3 | 13 | 15 | 5 | 22 | 59 | 1 | 13 | 14 | 18 | 27 | 24 | 31 | 47 | 37 | 8 | 29 | 16 | 35 | 9 | 20 |
| CACCAGCATT | 75847 | CREEBP/EP300 inhibitory protein 1 | 4 | 0 | 2 | 19 | 15 | 3 | 22 | 18 | 0 | 7 | 30 | 14 | 27 | 15 | 15 | 0 | 9 | 0 | 11 | 22 | 21 | 2 | 15 |
| TTTTGTAATT | 75890 | membrane-bound transcription factor protease | 0 | 0 | 0 | 0 | 3 | 3 | 4 | 0 | 1 | 3 | 14 | 4 | 4 | 9 | 8 | 0 | 7 | 4 | 5 | 2 | 16 | 9 | 9 |
| GTGCAGGGAG | 79414 | prostate-epithelium-specific Ets | 2 | 0 | 1 | 8 | 21 | 0 | 57 | 33 | 11 | 13 | 110 | 32 | 56 | 54 | 28 | 3 | 32 | 24 | 33 | 59 | 41 | 2 | 34 |
| | | transcription factor | | | | | | | | | | | | | | | | | | | | | | | |
| ATGACTCAAG | 239752 | nuclear receptor subfamily 2 | 0 | 0 | 0 | 15 | 9 | 3 | 19 | 39 | 7 | 16 | 5 | 14 | 27 | 21 | 24 | 29 | 23 | 8 | 22 | 18 | 48 | 11 | 26 |
| ATTGTTTATG | 181163 | high-nobility group nucleosomal binding | 2 | 9 | 6 | 13 | 18 | 3 | 55 | 55 | 4 | 21 | 14 | 23 | 60 | 53 | 60 | 43 | 47 | 20 | 47 | 51 | 34 | 9 | 31 |
| | | domain 2 | | | | | | | | | | | | | | | | | | | | | | | |
| AAGGATGCCA | 169946 | GATA binding protein 3 | 4 | 0 | 2 | 55 | 9 | 0 | 1 | 14 | 9 | 24 | 9 | 15 | 13 | 7 | 17 | 0 | 26 | 16 | 13 | 8 | 38 | 0 | 15 |
| CTTGTAATCC | 183253 | nucleolar RNA-associated protein | 9 | 2 | 6 | 4 | 72 | 78 | 22 | 55 | 7 | 80 | 4 | 40 | 27 | 21 | 14 | 19 | 7 | 104 | 32 | 4 | 62 | 7 | 24 |
| TAGTTTGTGG | 78934 | mut8 homolog 2 | 0 | 0 | 0 | 8 | 9 | 5 | 4 | 8 | 0 | 0 | 4 | 5 | 13 | 12 | 12 | 15 | 4 | 0 | 9 | 37 | 10 | 11 | 19 |

Signal transduction

| | | | | | | | | | | | | | | | | | | | | | | | | | |
|---|---|---|---|---|---|---|---|---|---|---|---|---|---|---|---|---|---|---|---|---|---|---|---|---|---|
| CGGTCTTATG | 75842 | dual-specificity phosphorylation regulated | 0 | 0 | 0 | 2 | 0 | 0 | 15 | 27 | 4 | 0 | 5 | 7 | 7 | 11 | 18 | 21 | 7 | 8 | 12 | 4 | 3 | 2 | 3 |
| | | kinase 1A | | | | | | | | | | | | | | | | | | | | | | | |
| TGAAAAGCTT | 2384 | tumor protein D52 | 2 | 2 | 2 | 19 | 21 | 5 | 26 | 47 | 5 | 15 | 2 | 17 | 49 | 44 | 22 | 69 | 19 | 28 | 38 | 18 | 109 | 25 | 50 |
| TTAAGAGGGA | 178137 | transducer of ERBB2, 1 | 0 | 0 | 0 | 11 | 3 | 8 | 13 | 16 | 0 | 1 | 2 | 7 | 18 | 19 | 28 | 47 | 12 | 4 | 21 | 29 | 12 | 2 | 14 |
| TATTTCACCG | 138860 | Rho GTPase activating protein 1 | 2 | 0 | 1 | 2 | 6 | 3 | 25 | 20 | 5 | 1 | 5 | 8 | 27 | 22 | 12 | 8 | 15 | 0 | 14 | 20 | 9 | 11 | 13 |
| GTCTTTCTTG | 151536 | RAB13, member RAS oncogene family | 2 | 2 | 2 | 13 | 0 | 2 | 12 | 20 | 0 | 6 | 4 | 7 | 11 | 19 | 32 | 37 | 25 | 8 | 22 | 22 | 9 | 13 | 14 |
| CCAGGGGAGA | 278613 | interferon, alpha-inducible protein 27 | 0 | 0 | 0 | 4 | 36 | 3 | 4 | 90 | 5 | 176 | 2 | 40 | 0 | 21 | 5 | 1 | 3 | 104 | 23 | 2 | 31 | 77 | 37 |
| GAGCAGCGCC | 112408 | S100 calcium binding protein A7 | 18 | 0 | 9 | 1018 | 3 | 3 | 373 | 16 | 1 | 2 | 890 | 288 | 0 | 0 | 0 | 1 | 0 | 20 | 4 | 0 | 0 | 0 | 0 |
| | | (psoriasin 1) | | | | | | | | | | | | | | | | | | | | | | | |
| GCTCTGCTTG | 112408 | S100 calcium binding protein A7 | 2 | 0 | 1 | 76 | 0 | 0 | 20 | 0 | 0 | 0 | 55 | 19 | 0 | 0 | 0 | 0 | 0 | 0 | 0 | 0 | 0 | 0 | 0 |
| | | (psoriasin 1) | | | | | | | | | | | | | | | | | | | | | | | |
| CGCCGACGAT | 265827 | interferon, alpha-inducible protein | 4 | 0 | 2 | 17 | 644 | 3 | 90 | 418 | 18 | 366 | 4 | 195 | 130 | 171 | 5 | 63 | 12 | 161 | 90 | 14 | 526 | 181 | 240 |
| | | (IFI-6-16) | | | | | | | | | | | | | | | | | | | | | | | |
| GTGTGTTTGT | 118787 | transforming growth factor, beta-induced, | 0 | 0 | 0 | 8 | 0 | 2 | 10 | 6 | 1 | 0 | 4 | 4 | 13 | 11 | 21 | 8 | 22 | 44 | 20 | 24 | 10 | 9 | 14 |
| | | 63kD | | | | | | | | | | | | | | | | | | | | | | | |
| CCAATAAAGT | 101850 | retinol binding protein 1, cellular | 2 | 0 | 1 | 0 | 3 | 0 | 0 | 2 | 6 | 11 | 7 | 4 | 49 | 28 | 6 | 8 | 0 | 0 | 15 | 102 | 32 | 21 | 52 |
| GTCTAGAATC | 92384 | vitamin A responsive; cytoskeleton related | 0 | 0 | 0 | 21 | 6 | 0 | 25 | 6 | 1 | 4 | 32 | 12 | 16 | 7 | 21 | 11 | 15 | 24 | 15 | 20 | 10 | 5 | 12 |
| ATCCGCGAGG | 180142 | canodulin-like skin protein | 0 | 0 | 0 | 0 | 0 | 3 | 22 | 0 | 20 | 0 | 0 | 6 | 47 | 25 | 0 | 52 | 19 | 0 | 24 | 20 | 0 | 0 | 7 |
| GATTTTGCAC | 274479 | nucleoside diphosphate kinase 7 | 0 | 0 | 0 | 19 | 6 | 0 | 7 | 0 | 6 | 1 | 16 | 7 | 9 | 1 | 4 | 1 | 6 | 0 | 4 | 2 | 18 | 2 | 7 |

*The above sequences are SEQ ID NOs:35-97, respectively

Metabolism

| | | | | | | | | | | | | | | | | | | | | | | | | | |
|---|---|---|---|---|---|---|---|---|---|---|---|---|---|---|---|---|---|---|---|---|---|---|---|---|---|
| ACCTTGTGCC | 878 | sorbitol dehydorgenase | 0 | 2 | 1 | 4 | 18 | 0 | 20 | 4 | 1 | 3 | 9 | 7 | 22 | 26 | 1 | 6 | 110 | 4 | 28 | 4 | 95 | 0 | 33 |
| TGCCGTTTTG | 2006 | glutathione S-transferase M3 (brain) | 0 | 2 | 1 | 0 | 48 | 0 | 1 | 20 | 7 | 25 | 2 | 13 | 9 | 12 | 3 | 4 | 19 | 8 | 9 | 4 | 13 | 7 | 8 |
| CCGTGCTCAT | 9857 | dicarbonyl/L-xylulose reductase | 11 | 7 | 9 | 2 | 51 | 8 | 20 | 18 | 4 | 5 | 67 | 22 | 99 | 56 | 21 | 7 | 12 | 56 | 41 | 77 | 34 | 7 | 39 |
| GTTTCTATCA | 12540 | lysophospholipase I | 0 | 2 | 1 | 6 | 15 | 0 | 25 | 49 | 1 | 7 | 0 | 13 | 25 | 12 | 26 | 45 | 19 | 8 | 22 | 12 | 38 | 2 | 17 |
| CAAATAAAAT | 71465 | squalese epoxidase | 2 | 2 | 2 | 0 | 24 | 2 | 19 | 55 | 4 | 0 | 5 | 14 | 9 | 8 | 3 | 40 | 13 | 12 | 14 | 4 | 6 | 39 | 16 |
| GGAACTTTTA | 43857 | similar to glucosamine-6-sulfatases | 0 | 2 | 1 | 17 | 36 | 3 | 7 | 6 | 4 | 14 | 25 | 14 | 9 | 8 | 26 | 0 | 60 | 0 | 17 | 10 | 10 | 5 | 8 |
| TTACCTTTTT | 79222 | galactosidase, beta 1 | 0 | 0 | 0 | 4 | 3 | 0 | 10 | 14 | 0 | 2 | 2 | 4 | 2 | 4 | 8 | 18 | 6 | 16 | 9 | 18 | 3 | 5 | 9 |
| TTGGGGAAAC | 81029 | biliverdin reductase A | 4 | 5 | 4 | 4 | 24 | 0 | 22 | 27 | 1 | 9 | 7 | 12 | 43 | 19 | 8 | 3 | 18 | 32 | 20 | 22 | 29 | 11 | 21 |
| TGATCTCCAA | 83190 | fatty acid synthase | 16 | 5 | 10 | 53 | 63 | 6 | 201 | 182 | 31 | 47 | 5 | 74 | 168 | 33 | 105 | 17 | 314 | 4 | 107 | 254 | 46 | 21 | 107 |
| TTTGGTGTTT | 83190 | fatty acid synthase | 5 | 0 | 3 | 8 | 24 | 2 | 57 | 27 | 5 | 28 | 21 | 21 | 36 | 41 | 62 | 14 | 57 | 12 | 37 | 28 | 10 | 4 | 14 |
| TTAACCCCTC | 78224 | ribonuclease, RNase A family, 1 (pancreatic) | 2 | 0 | 1 | 25 | 0 | 6 | 20 | 10 | 1 | 1 | 5 | 9 | 31 | 57 | 13 | 6 | 0 | 32 | 23 | 18 | 46 | 9 | 24 |
| GCTTTGATGA | 89649 | epoxide hydrolase 1, microsomal (xenobiotic) | 0 | 2 | 1 | 0 | 6 | 2 | 52 | 20 | 2 | 9 | 12 | 13 | 16 | 29 | 13 | 6 | 29 | 40 | 22 | 29 | 6 | 14 | 17 |
| TACAGTATGT | 170171 | glutamate-ammonia Ilgase | 0 | 5 | 2 | 13 | 12 | 3 | 36 | 82 | 4 | 24 | 228 | 50 | 4 | 19 | 87 | 26 | 56 | 56 | 41 | 4 | 16 | 0 | 7 |
| TGGGGTTCTT | 272499 | dehydorgenase/reductase (SDR family) | 2 | 2 | 2 | 0 | 0 | 2 | 0 | 113 | 0 | 84 | 0 | 25 | 7 | 13 | 10 | 0 | 0 | 0 | 5 | 0 | 32 | 0 | 11 |
| | | member 2 | | | | | | | | | | | | | | | | | | | | | | | |
| TTACTTCCCC | 184641 | fatty acid desaturase 2 | 2 | 0 | 1 | 2 | 0 | 0 | 138 | 29 | 9 | 2 | 0 | 22 | 29 | 19 | 10 | 32 | 43 | 4 | 23 | 53 | 4 | 4 | 20 |
| AAGAATCTGA | 183435 | NADH dehydrogenase | 0 | 0 | 0 | 15 | 0 | 3 | 31 | 31 | 1 | 3 | 0 | 10 | 34 | 20 | 14 | 17 | 35 | 0 | 20 | 71 | 46 | 2 | 39 |
| GTCCCTGCCT | 279837 | glutathione S-transferase M2 | 0 | 5 | 2 | 4 | 18 | 0 | 10 | 53 | 1 | 6 | 5 | 12 | 4 | 13 | 22 | 8 | 47 | 0 | 16 | 4 | 12 | 11 | 9 |
| AATATGTGGG | 351875 | cytochrome c oxidase subunit VIc | 11 | 5 | 8 | 38 | 707 | 6 | 19 | 219 | 2 | 112 | 23 | 141 | 325 | 337 | 77 | 30 | 185 | 24 | 163 | 28 | 1250 | 14 | 431 |
| GGAGCTCTGT | 227750 | NADH dehydrogenase I beta subcomplex, 4 | 4 | 5 | 4 | 11 | 39 | 5 | 17 | 27 | 5 | 21 | 14 | 17 | 18 | 11 | 30 | 22 | 29 | 16 | 21 | 16 | 31 | 9 | 19 |
| GAAGGAGATA | 171889 | choline phosphotransferase I | 0 | 0 | 0 | 4 | 3 | 0 | 0 | 10 | 0 | 1 | 0 | 2 | 9 | 15 | 14 | 34 | 4 | 4 | 13 | 2 | 23 | 2 | 9 |
| TCAGACTTTT | 334305 | diacylglycerol O-acyltransferase homolog 2 | 0 | 0 | 0 | 11 | 0 | 0 | 15 | 0 | 2 | 0 | 28 | 7 | 2 | 22 | 1 | 17 | 0 | 4 | 8 | 2 | 0 | 30 | 11 |
| TCTTGTAACT | 256549 | nucleotide binding protein 2 | 0 | 0 | 0 | 0 | 12 | 0 | 9 | 4 | 5 | 4 | 2 | | 11 | 13 | 4 | 1 | 4 | 48 | 14 | 22 | 12 | 2 | 12 |

ESTs

| | | | | | | | | | | | | | | | | | | | | | | | | | |
|---|---|---|---|---|---|---|---|---|---|---|---|---|---|---|---|---|---|---|---|---|---|---|---|---|---|
| TGATGAGTGT | 356209 | ESTs | 0 | 0 | 0 | 2 | 0 | 0 | 1 | 6 | 0 | 3 | 0 | 2 | 2 | 0 | 6 | 6 | 7 | 0 | 4 | 2 | 0 | 0 | 1 |
| CTGCAACCTA | 374393 | ESTs | 2 | 0 | 1 | 11 | 6 | 2 | 13 | 8 | 4 | 8 | 9 | 7 | 2 | 7 | 8 | 4 | 7 | 12 | 7 | 12 | 16 | 16 | 15 |
| TGAGTGGTTT | 29672 | ESTs | 0 | 0 | 0 | 4 | 0 | 0 | 3 | 14 | 0 | 0 | 2 | 3 | 4 | 3 | 10 | 12 | 6 | 8 | 7 | 2 | 6 | 5 | 4 |
| CACTGTGTTG | 350475 | EST clone IMAGE:4430514 | 4 | 0 | 2 | 2 | 3 | 0 | 4 | 2 | 1 | 3 | 18 | 4 | 9 | 7 | 12 | 12 | 7 | 12 | 10 | 6 | 21 | 5 | 11 |
| TTAAGAAGTT | 275360 | ESTs | 7 | 0 | 4 | 15 | 0 | 3 | 63 | 0 | 0 | 0 | 2 | 10 | 2 | 1 | 55 | 0 | 18 | 0 | 13 | 14 | 6 | 0 | 7 |
| GCGACAGTAA | 170853 | ESTs | 0 | 0 | 0 | 4 | 0 | 0 | 6 | 16 | 0 | 5 | 16 | 6 | 9 | 8 | 9 | 3 | 15 | 20 | 11 | 2 | 1 | 4 | 2 |
| TCAACTTGAA | 99244 | ESTs | 0 | 0 | 0 | 21 | 3 | 3 | 7 | 4 | 12 | 0 | 0 | 6 | 16 | 19 | 9 | 3 | 10 | 0 | 9 | 28 | 40 | 16 | 28 |
| TTTCTGGAGG | 129943 | KIAA0545 protein | 2 | 0 | 1 | 15 | 3 | 3 | 4 | 12 | 6 | 1 | 2 | 6 | 16 | 12 | 12 | 6 | 7 | 4 | 9 | 20 | 6 | 13 | 13 |
| GGGGCTGGAG | 301685 | KIAA0620 protein | 0 | 0 | 0 | 11 | 6 | 5 | 13 | 29 | 6 | 6 | 4 | 10 | 2 | 9 | 14 | 6 | 7 | 16 | 9 | 8 | 13 | 18 | 13 |
| GTCTCATTTC | 90419 | KIAA0882 protein | 4 | 0 | 2 | 8 | 3 | 2 | 4 | 23 | 1 | 33 | 0 | 9 | 0 | 13 | 14 | 3 | 21 | 0 | 8 | 0 | 29 | 0 | 10 |
| ACCGCCTGTG | 79625 | chromosome 20 open reading frame 149 | 2 | 5 | 3 | 4 | 36 | 2 | 1 | 80 | 4 | 121 | 19 | 33 | 4 | 7 | 13 | 19 | 21 | 12 | 13 | 6 | 6 | 9 | 7 |
| GAAGAACAGA | 29341 | chromosome 20 open reading frame 81 | 0 | 0 | 0 | 13 | 3 | 3 | 4 | 16 | 0 | 2 | 2 | 5 | 4 | 9 | 14 | 8 | 6 | 0 | 7 | 6 | 15 | 7 | 9 |
| TCGTAACGAG | 11197 | chromosome 20 open reading frame 92 | 4 | 2 | 3 | 11 | 0 | 0 | 15 | 8 | 4 | 3 | 23 | 8 | 25 | 8 | 18 | 19 | 4 | 12 | 14 | 22 | 10 | 16 | 16 |
| GTGATGGGGC | 62620 | chromosome 6 open reading frame 1 | 2 | 0 | 1 | 2 | 12 | 0 | 13 | 2 | 0 | 4 | 11 | 5 | 16 | 3 | 6 | 6 | 13 | 0 | 7 | 20 | 10 | 9 | 13 |
| GAGAGAAAAT | 181444 | hypothetical protein LOC51235 | 0 | 2 | 1 | 40 | 9 | 0 | 10 | 6 | 7 | 7 | 21 | 13 | 4 | 8 | 9 | 11 | 18 | 0 | 8 | 6 | 10 | 27 | 14 |
| GCCCACATCC | 84753 | hypothetical protein FLJ12442 | 4 | 0 | 2 | 0 | 0 | 3 | 4 | 0 | 4 | 1 | 26 | 5 | 63 | 26 | 1 | 12 | 6 | 48 | 26 | 49 | 1 | 11 | 20 |
| GTATTTAACT | 209065 | hypothetical protein FLJ14225 | 0 | 0 | 0 | 17 | 6 | 3 | 28 | 12 | 6 | 8 | 9 | 11 | 9 | 16 | 15 | 6 | 16 | 0 | 10 | 20 | 10 | 18 | 16 |
| GGCTGGTCTC | 324844 | hypothetical protein IMAGE3455200 | 2 | 2 | 2 | 6 | 6 | 5 | 6 | 12 | 2 | 3 | 11 | 6 | 18 | 7 | 10 | 18 | 12 | 16 | 13 | 6 | 18 | 20 | 14 |
| AACACTTCTC | 333526 | hypothetical protein MGC14832 | 4 | 0 | 2 | 2 | 6 | 0 | 25 | 8 | 1 | 2 | 4 | 6 | 27 | 19 | 4 | 0 | 9 | 4 | 10 | 18 | 6 | 4 | 9 |
| AATAAAGAGA | 28149 | hypothetical protein BCO10626 | 0 | 2 | 1 | 0 | 3 | 0 | 6 | 23 | 0 | 1 | 60 | 12 | 7 | 4 | 21 | 0 | 31 | 0 | 10 | 6 | 0 | 2 | 3 |
| GAGAAACATT | 267245 | hypothetical protein FLJ14803 | 0 | 2 | 1 | 17 | 0 | 0 | 4 | 8 | 1 | 2 | 2 | 4 | 7 | 5 | 14 | 12 | 13 | 4 | 9 | 14 | 12 | 5 | 10 |
| TTTGGTCTTT | 109773 | hypothetical protein FLJ20625 | 0 | 0 | 0 | 8 | 0 | 3 | 6 | 10 | 4 | 4 | 4 | 5 | 20 | 28 | 12 | 15 | 15 | 24 | 19 | 10 | 10 | 0 | 7 |
| TGTGGTGGTG | 83422 | MLN51 protein | 5 | 2 | 4 | 6 | 3 | 2 | 55 | 39 | 7 | 7 | 4 | 15 | 87 | 25 | 18 | 22 | 13 | 36 | 34 | 92 | 18 | 5 | 38 |
| GAAAGATGCT | 334370 | brain expressed, X-linked 1 | 2 | 0 | 1 | 6 | 48 | 0 | 1 | 0 | 1 | 1 | 0 | 7 | 29 | 37 | 1 | 1 | 1 | 0 | 12 | 0 | 162 | 2 | 54 |
| TAGCAGACCC | 349195 | myeloid/lymphoid or mixed-lineage leukemia | 0 | 0 | 0 | 0 | 3 | 3 | 1 | 4 | 2 | 7 | 12 | 4 | 13 | 13 | 12 | 7 | 4 | 20 | 12 | 18 | 1 | 0 | 6 |

*The above sequences are SEQ ID NOs:98-144, respectively

No database match

| | | | | | | | | | | | | | | | | | | | | | | | | | |
|---|---|---|---|---|---|---|---|---|---|---|---|---|---|---|---|---|---|---|---|---|---|---|---|---|---|
| AACGCTGCGA | NA | No reliable match | 7 | 5 | 6 | 36 | 24 | 0 | 4 | 35 | 1 | 10 | 0 | 14 | 31 | 60 | 23 | 1 | 19 | 0 | 22 | 29 | 101 | 23 | 51 |
| AATGGATGAA | NA | No reliable match | 0 | 0 | 0 | 38 | 0 | 0 | 3 | 2 | 1 | 0 | 44 | 11 | 2 | 0 | 0 | 0 | 0 | 60 | 10 | 4 | 1 | 0 | 2 |
| ACATCGTAGT | NA | No reliable match | 0 | 0 | 0 | 0 | 15 | 0 | 3 | 31 | 0 | 2 | 2 | 7 | 13 | 20 | 4 | 4 | 10 | 4 | 9 | 0 | 60 | 0 | 20 |
| ACCCGCCGGG | NA | No reliable match | 11 | 7 | 9 | 103 | 18 | 3 | 4 | 0 | 1 | 6 | 166 | 38 | 20 | 8 | 0 | 1 | 4 | 193 | 38 | 31 | 23 | 0 | 18 |
| AGTGCAGGGA | NA | No reliable match | 0 | 0 | 0 | 2 | 0 | 2 | 15 | 2 | 0 | 0 | 37 | 7 | 38 | 0 | 23 | 1 | 1 | 48 | 20 | 26 | 0 | 7 | 11 |
| ATCAAGAATC | NA | No reliable match | 2 | 0 | 1 | 2 | 3 | 3 | 9 | 8 | 0 | 3 | 9 | 5 | 18 | 13 | 15 | 4 | 16 | 72 | 23 | 22 | 13 | 13 | 16 |
| ATGTGGCACA | NA | No reliable match | 4 | 2 | 3 | 2 | 24 | 0 | 20 | 31 | 1 | 9 | 34 | 15 | 18 | 16 | 12 | 44 | 23 | 8 | 20 | 14 | 15 | 9 | 12 |
| CAAACCTTTA | NA | No reliable match | 0 | 0 | 0 | 11 | 6 | 0 | 16 | 25 | 1 | 5 | 0 | 8 | 16 | 16 | 13 | 23 | 13 | 8 | 15 | 33 | 15 | 34 | 27 |
| CAATGCTGCC | NA | No reliable match | 11 | 12 | 11 | 53 | 12 | 3 | 23 | 33 | 9 | 3 | 64 | 25 | 580 | 145 | 18 | 18 | 26 | 44 | 139 | 588 | 28 | 11 | 209 |
| CAGCTTAATT | NA | No reliable match | 4 | 2 | 3 | 4 | 3 | 0 | 25 | 20 | 0 | 1 | 2 | 7 | 36 | 20 | 0 | 0 | 4 | 4 | 11 | 90 | 6 | 5 | 34 |
| CCGACGGGCG | NA | No reliable match | 4 | 2 | 3 | 67 | 3 | 0 | 3 | 0 | 1 | 4 | 87 | 21 | 7 | 0 | 0 | 0 | 0 | 181 | 31 | 4 | 7 | 0 | 4 |
| CCTTTGAACA | NA | No reliable match | 2 | 0 | 1 | 4 | 6 | 5 | 0 | 10 | 2 | 3 | 14 | 6 | 9 | 13 | 5 | 12 | 6 | 16 | 10 | 2 | 4 | 4 | 3 |
| CCTTTGCCCT | NA | No reliable match | 0 | 0 | 0 | 0 | 9 | 2 | 73 | 16 | 1 | 14 | 5 | 15 | 27 | 26 | 19 | 0 | 9 | 0 | 14 | 28 | 9 | 0 | 12 |
| CGGTTTAATT | NA | No reliable match | 2 | 0 | 1 | 23 | 0 | 0 | 12 | 10 | 1 | 3 | 53 | 13 | 13 | 9 | 26 | 3 | 25 | 16 | 15 | 20 | 0 | 0 | 7 |
| CTTTATTCCA | NA | No reliable match | 0 | 0 | 0 | 19 | 0 | 2 | 48 | 2 | 0 | 0 | 5 | 9 | 25 | 22 | 31 | 4 | 16 | 0 | 16 | 18 | 15 | 3 | 13 |
| GAAGTCGGAA | NA | No reliable match | 4 | 0 | 2 | 48 | 0 | 2 | 3 | 2 | 27 | 3 | 2 | 11 | 20 | 3 | 4 | 12 | 4 | 0 | 7 | 18 | 9 | 7 | 11 |
| GATCTCGCAA | NA | No reliable match | 4 | 7 | 5 | 44 | 21 | 0 | 31 | 25 | 7 | 1 | 0 | 16 | 40 | 13 | 12 | 22 | 16 | 4 | 18 | 47 | 38 | 64 | 50 |
| GCACCTCCTA | NA | No reliable match | 2 | 0 | 1 | 8 | 9 | 2 | 7 | 12 | 4 | 1 | 2 | 6 | 13 | 12 | 6 | 11 | 10 | 0 | 9 | 12 | 6 | 7 | 8 |
| GCCGTGAGCA | NA | No reliable match | 2 | 0 | 1 | 17 | 12 | 0 | 6 | 8 | 2 | 1 | 5 | 6 | 25 | 17 | 1 | 6 | 13 | 0 | 10 | 12 | 31 | 20 | 21 |
| GGAAAGTGAC | NA | No reliable match | 0 | 0 | 0 | 2 | 6 | 2 | 4 | 10 | 0 | 5 | 7 | 5 | 11 | 22 | 12 | 6 | 26 | 0 | 13 | 12 | 23 | 9 | 15 |
| GGACCTTTAT | NA | No reliable match | 2 | 0 | 1 | 23 | 3 | 0 | 1 | 23 | 1 | 0 | 37 | 11 | 2 | 1 | 1 | 0 | 1 | 0 | 1 | 4 | 3 | 0 | 2 |
| GGCAGACAAT | NA | No reliable match | 0 | 0 | 0 | 13 | 0 | 0 | 12 | 14 | 1 | 2 | 7 | 6 | 16 | 5 | 1 | 15 | 7 | 0 | 7 | 18 | 12 | 13 | 14 |
| GGCAGCACAA | NA | No reliable match | 0 | 5 | 2 | 23 | 18 | 0 | 16 | 27 | 20 | 12 | 5 | 15 | 49 | 11 | 5 | 12 | 6 | 4 | 15 | 35 | 25 | 29 | 30 |
| GGTAGCTGCT | NA | No reliable match | 0 | 0 | 0 | 6 | 3 | 0 | 3 | 20 | 0 | 6 | 14 | 7 | 7 | 4 | 4 | 4 | 3 | 0 | 4 | 2 | 1 | 4 | 2 |
| GGTAGTTTTA | NA | No reliable match | 13 | 0 | 6 | 59 | 21 | 3 | 32 | 41 | 2 | 13 | 18 | 24 | 18 | 28 | 39 | 0 | 59 | 16 | 26 | 18 | 79 | 0 | 32 |
| GGTCAGTCGG | NA | No reliable match | 5 | 5 | 5 | 76 | 15 | 2 | 0 | 0 | 39 | 3 | 102 | 30 | 25 | 3 | 1 | 7 | 1 | 80 | 20 | 18 | 13 | 2 | 11 |
| GTAATCCTGC | NA | No reliable match | 4 | 2 | 3 | 34 | 6 | 12 | 0 | 4 | 187 | 28 | 51 | 40 | 22 | 17 | 6 | 25 | 1 | 52 | 21 | 24 | 7 | 7 | 13 |
| GTAGTTACTG | NA | No reliable match | 2 | 2 | 2 | 8 | 120 | 0 | 1 | 25 | 0 | 21 | 4 | 22 | 38 | 33 | 13 | 7 | 19 | 0 | 18 | 8 | 172 | 4 | 61 |
| TCACAGTGCC | NA | No reliable match | 2 | 2 | 2 | 15 | 3 | 2 | 13 | 39 | 1 | 7 | 14 | 12 | 29 | 5 | 42 | 28 | 21 | 8 | 22 | 20 | 6 | 13 | 13 |
| TCTGGTTTGT | NA | No reliable match | 2 | 2 | 2 | 6 | 12 | 3 | 10 | 33 | 5 | 2 | 7 | 10 | 29 | 16 | 4 | 50 | 3 | 12 | 19 | 41 | 6 | 7 | 18 |
| TGAAGCAGTA | NA | No reliable match | 4 | 2 | 3 | 99 | 3 | 2 | 36 | 27 | 9 | 5 | 25 | 16 | 74 | 46 | 122 | 57 | 85 | 12 | 66 | 57 | 40 | 25 | 41 |
| TGTCATAGTT | NA | No reliable match | 0 | 0 | 0 | 0 | 15 | 0 | 9 | 55 | 0 | 3 | 9 | 11 | 34 | 42 | 9 | 4 | 34 | 4 | 21 | 6 | 197 | 0 | 68 |
| TTACGATGAA | NA | No reliable match | 2 | 0 | 1 | 0 | 6 | 0 | 3 | 18 | 1 | 1 | 0 | 4 | 51 | 41 | 4 | 1 | 7 | 0 | 18 | 73 | 9 | 2 | 28 |
| TTCGGTTGGT | NA | No reliable match | 2 | 0 | 1 | 101 | 3 | 0 | 55 | 16 | 0 | 0 | 7 | 23 | 58 | 40 | 40 | 1 | 60 | 4 | 34 | 55 | 12 | 11 | 29 |

*The above sequences are SEQ ID NOs:145-178, respectively
Ave = average number of SAGE tags/histologic stage.

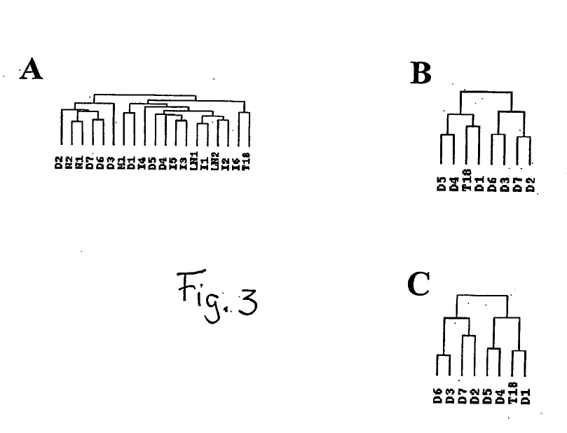

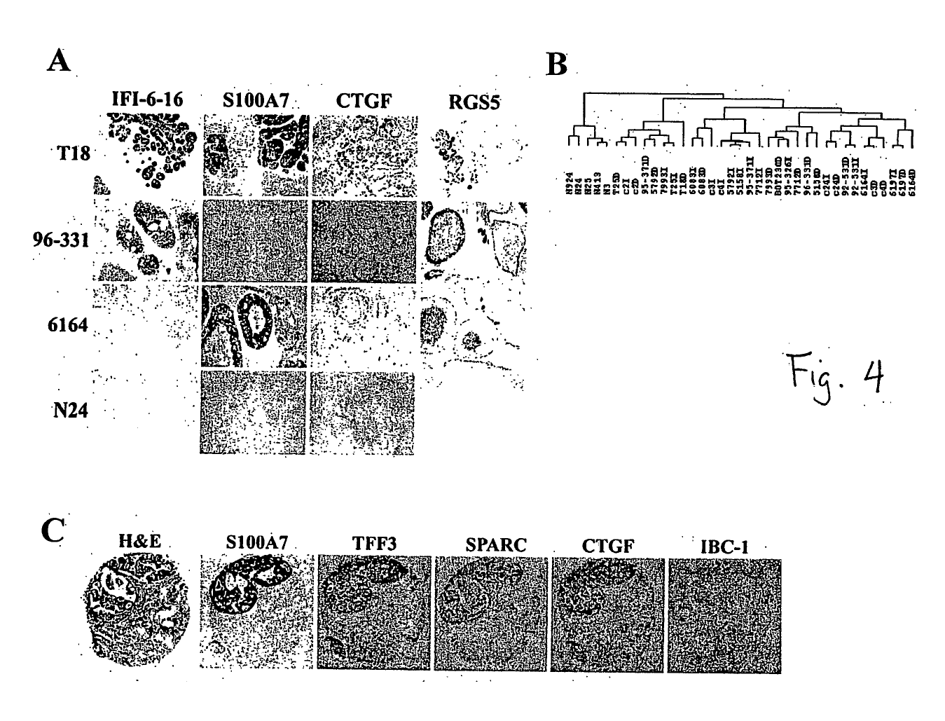

To identify overall similarities and differences among samples, the 19 SAGE libraries were analyzed by hierarchical clustering (FIG. 3A). A dendrogram created using this program revealed that, while the two normal samples (N1 and N2) were more similar to each other than to any other samples, the primary invasive tumor and lymph node metastasis from the first patient (I1 and LN1) were more similar to each other than to any other sample and the primary invasive tumor and lymph node metastasis from the second patient (I2 and LN2) were more similar to each than to any other sample. In situ tumors, invasive tumors, and metastases did not form distinct clusters suggesting that none of these tumor classes is there a pronounced and common “in situ”, “invasive”, or “metastasis” signature. Correlating with this observation, clustering and other statistical analyses failed to identify any gene that was universally and specifically up or down-regulated in DCIS, invasive, or metastatic tumors (FIG. 3A). These findings confirm previous studies performed in invasive breast carcinomas and highlight the fact that DCIS tumors are just as heterogeneous at the molecular level as their invasive counterparts [Perou et al. (2000) Nature 406:747-752].

To analyze the relationships among DCIS tumors in more, detail, hierarchical clustering was performed using the eight DCIS libraries (FIG. 3B). The expression profiles of 582 genes (Table 3) were included in this analysis; while 920 SAGE tags and their corresponding genes are listed in Table 3, many of the genes are represented by more than one tag. The program used for the clustering analysis (see Example 1) filtered for tags at least ten-copies of which were present in at least one library and which were present in at least one library in a number at least ten-fold higher than in a library from another category of breast tissue. Genes expressed by non-epithelial cells apparently play a predominant role in defining the relatedness of samples since the BerEP4 purified (D2, D3, D6, and D7) and unpurified (D1, D4, D5, and T18) tumors formed two distinct clusters. Tumors also appeared to cluster according to their histologic grade with the high-grade tumors (D3, D6, D4, and D5) and the intermediate grade tumors (D2, D7) DCIS showing highest similarity to each other. However, T18, an intermediate grade, non-comedo DCIS, showed highest similarity to D1, a high grade comedo DCIS, suggesting that, despite its histologic features, this DCIS appears to have the molecular profile of a high grade, comedo DCIS.

TABLE 3
Genes employed for the clustering analysis shown in FIG. 3B

| | | | |
|---|---|---|---|
| SEQ | | | |
| ID | | | |
| NO: | Tag | Unigene | Gene name |

| | | | | |
|---|---|---|---|---|
| 179 | AGCGACAAAC | 82109 | syndecan 1 | |
| 180 | AGGAAGGAAC | 323910 | v-erb-b2 erythroblastic leukemia viral oncogene homolog 2, neuro/glioblastoma | |
| | | | derived oncogene homolog (avian) | |
| 181 | CTGTTCCGGC | 286192 | dopamine and cAMP-regulated neuronal phosphoprotein 32 | |
| 182 | ATCGCTTTCT | 177486 | amyloid beta (A4) precursor protein (protease nexin-II, Alzheimer disease) | |
| 183 | GTGGCCACGG | 112405 | S100 calcium binding protein A9 (calgranulin B) | |
| 184 | ATGTGAAGAG | 111779 | secreted protein, acidic, cysteine-rich (osteonectin) | |
| 185 | ATGTGAAGAG | 126515 | EST | |
| 186 | TGAAGCAGTA | 176626 | hemogen | |
| 187 | TGAAGCAGTA | 326248 | programmed cell death 4 (neoplastic transformation inhibitor) | |
| 188 | ACCAAAAACC | 172928 | collagen, type I, alpha 1 | |
| 189 | TTTGCACCTT | 75511 | connective tissue growth factor | |
| 190 | TTTGGTTTTC | 21431 | suppressor of fused homolog (Drosophila) | |
| 191 | TTTGGTTTTC | 179573 | retinoblastoma binding protein 1 | |
| 192 | TGGAAATGAC | 172928 | collagen, type I, alpha 1 | |
| 193 | TGGAAATGAC | 173648 | ESTs, Weakly similar to zinc finger protein ZNF287 [Homo sapiens] [H.sapiens] | |
| 194 | GGGCATCTCT | 76807 | major histocompatibility complex, class II, DR alpha | |
| 195 | TTGCTGACTT | 108885 | collagen, type VI, alpha 1 | |
| 196 | TTGCTGACTT | 238928 | HT002 protein; hypertension-related calcium-regulated gene | |
| 197 | TTTCAGAGAG | 75975 | signal recognition particle 9kD | |
| 198 | TTTCAGAGAG | r 355743 | ESTs, Highly similar to SR09 HUMAN Signal recognition particle 9 kDa protein | |
| | | | (SRP9) [H.sapiens] | |
| 199 | AACTGCTTCA | 11538 | actin related protein 2/3 complex, subunit 1B (41 kD) | |
| 200 | ACTTACCTGC | 12504 | likely ortholog of mouse Arkadia | |
| 201 | ACTTACCTGC | 174031 | cytochrome c oxidase subunit VIb | |
| 202 | TGTGGTGGTG | 83422 | MLN51 protein | |
| 203 | TGTGGTGGTG | 223618 | EST | |
| 204 | TTACTTCCCC | 184641 | fatty acid desaturase 2 | |
| 205 | CATTTCAATA | 75431 | fibrinogen, gamma polypeptide | |
| 206 | CATTTCAATA | 32587 | steroid receptor RNA activator 1 | |
| 207 | GTGCTGATTC | 75584 | polymyositis/scleroderma autoantigen 2 (100kD) | |
| 208 | GTGCTGATTC | 1640 | collagen, type VII, alpha 1 (epidermolysis bullosa, dystrophic, dominant and | |
| | | | recessive) | |
| 209 | CGACCCCACG | 169401 | apolipoprotein E | |
| 210 | TTTTGTAACT | 256549 | nucleotide binding protein 2 (MinD homolog, E. coli) | |
| 211 | TCTAAGTACG | | | |
| 212 | CTTCCTTGCC | 2785 | keratin 17 | |
| 213 | CTTCCTTGCC | 272572 | hemoglobin, alpha 1 | |
| 214 | TTAAGAAGTT | 275360 | ESTs | |
| 215 | GCTCTGCTTG | 112408 | S100 calcium binding protein A7 (psoriasin 1) | |
| 216 | ATTAAGAGGG | | | |
| 217 | GAGCAGCGCC | 112408 | S100 calcium binding protein A7 (psoriasin 1) | |
| 218 | CCTGGGAAGT | 12035 | ESTs, Weakly similar to 2004399A chromosomal protein [Homo sapiens] | |
| | | | [H.sapiens] | |
| 219 | CCTGGGAAGT | 89603 | mucin 1, transmembrane | |
| 220 | CAAACTAACC | 75813 | polycystic kidney disease 1 (autosomal dominant) | |
| 221 | CAAACTAACC | 153261 | immunoglobulin heavy constant mu | |
| 222 | AAACCCCAAT | 8997 | Sad1 unc-84 domain protein 1 | |
| 223 | AAACCCCAAT | 77735 | hypothetical protein FLJ11618 | |
| 224 | GAAATAAAGC | 300697 | immunoglobulin heavy constant gamma 3 (G3m marker) | |
| 225 | GAAATAAAGC | 111334 | ferritin, light polypeptide | |
| 226 | AAGGGAGCAC | 181125 | immunoglobulin lambda locus | |
| 227 | AAGGGAGCAC | 8997 | Sad1 unc-84 domain protein 1 | |
| 228 | GGAGTGTGCT | 9615 | myosin, light polypeptide 9, regulatory | |
| 229 | CATATCATTA | 119206 | insulin-like growth factor binding protein 7 | |
| 230 | TTTTTAATGT | 181307 | H3 histone, family 3A | |
| 231 | TTTTTAATGT | 356202 | ESTs, Highly similar to S06250 histone H3 [similarity] | |
| 232 | CTCCCCCAAG | | | |
| 233 | CTCCCCCAAA | 306886 | Homo sapiens cDNA: FLJ23175 fis, clone LNG10438 | |
| 234 | GTTCACATTA | 51615 | ESTs, Weakly similar to hypothetical protein FLJ20378 [Homo sapiens] | |
| | | | [H.sapiens] | |
| 235 | GTTCACATTA | 84298 | CD74 antigen (invariant polypeptide of major histocompatibility complex, class | |
| | | | II antigen-associated) | |
| 236 | GTACGTATTC | 76325 | immunoglobulin J polypeptide, linker protein for immunoglobulin alpha and mu | |
| | | | polypeptides | |
| 237 | GTACGTATTC | 146657 | ESTs | |
| 238 | TAAAATATTG | 4193 | ortholog of mouse integral membrane glycoprotein LIG-1 | |
| 239 | TAATAAAGGT | 151604 | ribosomal protein S8 | |
| 240 | TAATAAAGGT | 374502 | ESTS, Highly similar to S25022 ribosomal protein S8, cytosolic | |
| 241 | CAATAAATGT | 163109 | ESTs | |
| 242 | CAATAAATGT | 337445 | ribosomal protein L37 | |
| 243 | CTCTCACCCT | 75108 | ribonuclease/angiogenin inhibitor | |
| 244 | CTCTCACCCT | 268189 | hypothetical protein FLJ20436 | |
| 245 | GTGCCTAGGG | 198166 | activating transcription factor 2 | |
| 246 | CCTATTTACT | 347969 | cytochrome c oxidase subunit IV isoform 1 | |
| 247 | CTGTTGATTG | 249495 | heterogeneous nuclear ribonucleoprotein A1 | |
| 248 | CTGTTGATTG | 356723 | ESTs, Highly similar to S04617 heterogeneous ribonuclear particle protein A1 | |
| 249 | GTTGTCTTTG | 258798 | hypothetical protein FLJ20003 | |
| 250 | GTTGTCTTTG | 284394 | complement component 3 | |
| 251 | GCTCACCTGT | 29647 | uncharacterized hematopoietic stem/progenitor cells protein MDS028 | |
| 252 | GCTCACCTGT | 159142 | lunatic fringe homolog (Drosophila) | |
| 253 | GTGTAATAAG | 232400 | heterogeneous nuclear ribonucleoprotein A2/B1 | |
| 254 | CAATGCTGCC | 234518 | ribosomal protein L23 | |
| 255 | GTGATGGTGT | 197345 | thyroid autoantigen 70kD (Ku antigen) | |
| 256 | GTGATGGTGT | 3352 | histone deacetylase 2 | |
| 257 | TGAGGGAATA | 83848 | triosephosphate isomerase 1 | |
| 258 | GGCACAGTAA | 11270 | hypothetical protein MGC2491 | |
| 259 | GGCACAGTAA | 49169 | KIAA1634 protein | |
| 260 | GGCTGTACCC | 108080 | cysteine and glycine-rich protein 1 | |
| 261 | GGCTGTACCC | 96908 | p53-induced protein | |
| 262 | AACACAGCCT | 170250 | complement component 4A | |
| 263 | AACACAGCCT | 278625 | complement component 4B | |
| 264 | CAGTTCTCTG | 279921 | hypothetical protein MGC8721 | |
| 265 | AAGGACCTAG | | | |
| 266 | TAATAAATGC | | | |
| 267 | CCCTATCACA | 150826 | RAB25, member RAS oncogene family | |
| 268 | CGGTTTAATT | | | |
| 269 | TTTCTAGTTT | 111894 | lysosomal-associated protein transmembrane 4 alpha | |
| 270 | CTGGAGGCTG | 98967 | ATPase, H+ transporting, lysosomal V0 subunit a isoform 4 | |
| 271 | CTGGAGGCTG | 149152 | rhophilin 1 | |
| 272 | CCTAGCTGGA | 356332 | ESTs, Moderately similar to S71220 peptidylprolyl isomerase (EC 5.2.1.8) ROC2 | |
| 273 | CCTAGCTGGA | 342389 | peptidylprolyl isomerase A (cyclophilin A) | |
| 274 | TTACCTCCTT | 355815 | Homo sapiens, clone MGC:8772 IMAGE:3862861, mRNA, complete cds | |
| 275 | CAATTAAAAG | 36475 | Homo sapiens cDNA FLJ36837 fis, clone ASTRO2011422 | |
| 276 | CAATTAAAAG | 149923 | X-box binding protein 1 | |
| 277 | CCTTTCACAC | 278589 | general transcription factor II, i | |
| 278 | CCTTTCACAC | 356669 | Homo sapiens cDNA FLJ25021 fis, clone CBL01740 | |
| 279 | TTCGGTTGGT | 24809 | hypothetical protein FLJ10826 | |
| 280 | GGTAGTTTTA | 82302 | Homo sapiens cDNA FLJ32144 fis, clone PLACE5000105, highly similar to Mus | |
| | | | musculus mRNA for heparan sulfate 6-sulfotransferase 2 | |
| 281 | GTAGACACCT | 153 | ribosomal protein L7 | |
| 282 | TTTAATTTGT | 182793 | golgi phosphoprotein 2 | |
| 283 | TTTAATTTGT | 220689 | Ras-GTPase-activating protein SH3-domain-binding protein | |
| 284 | AAGTTGCTAT | 78575 | prosaposin (variant Gaucher disease and variant metachromatic leukodystrophy) | |
| 285 | AAGTTGCTAT | 103382 | phospholipid scramblase 3 | |
| 286 | GGAATGTACG | 429 | ATP synthase, H+ transporting, mitochondrial F0 complex, subunit c (sub- | |
| | | | unit 9) isoform 3 | |
| 287 | CAAGCAGGAC | 179516 | integral type I protein | |
| 288 | TAGGACAACT | 367720 | ESTs, Highly similar to HSHU33 histone H3.3 | |
| 289 | CACCACGGTG | 241471 | RNB6 | |
| 290 | TACAGTATGT | 170171 | glutamate-ammonia ligase (glutamine synthase) | |
| 291 | CTGTTGGTGA | 3463 | ribosomal protein S23 | |
| 292 | CTGTTGGTGA | 356628 | ESTs, Moderately similar to T48317 hypothetical protein F9G14.270 | |
| 293 | TGTATGAATT | 25328 | Homo sapiens, clone IMAGE:4617948, mRNA | |
| 294 | TGTATGAATT | 28777 | H2A histone family, member L | |
| 295 | CTCGCGCTGG | 40369 | Homo sapiens cDNA FLJ33345 fis, clone BRACE2003713 | |
| 296 | CTCGCGCTGG | 25640 | claudin 3 | |
| 297 | GGTGAGACAC | 164280 | solute carrier family 25 (mitochondrial carrier; adenine nucleotide | |
| | | | translocator), member 6 | |
| 298 | GGTGAGACAC | 350927 | Homo sapiens cDNA FLJ30227 fis, clone BRACE2001865 | |
| 299 | GGGGTAAGAA | 80423 | prostatic binding protein | |
| 300 | GCAGCCATCC | 4437 | ribosomal protein L28 | |
| 301 | TGCTGGTGTG | 298573 | KIAA1720 protein | |
| 302 | TGCTGGTGTG | 84883 | KIAA0864 protein | |
| 303 | AGGGCTTCCA | 356767 | ESTs, Weakly similar to 60S ribosomal protein L10, putative [Arabidopsis | |
| | | | thaliana] [A.thaliana] | |
| 304 | AGGGCTTCCA | 29797 | ribosomal protein L10 | |
| 305 | GTAGGGGTAA | | | |
| 306 | CTTGAGCAAT | 848 | FK506 binding protein 4 (59kD) | |
| 307 | GTCTGGGGCT | 75725 | thiopurine S-methyltransferase | |
| 308 | GCCCCCAATA | 227751 | lectin, galactoside-binding, soluble, 1 (galectin 1) | |
| 309 | TGGCTGGGAA | 172684 | vesicle-associated membrane protein 8 (endobrevin) | |
| 310 | GGGCCCAGGA | 25197 | STIP1 homology and U-Box containing protein 1 | |
| 311 | GGGCCCAGGA | 118983 | hypothetical protein FLJ12150 | |
| 312 | CAAGGGCCAA | 170160 | RAB2, member RAS oncogene family-like | |
| 313 | GCAAAAGAAA | 1265 | branched chain keto acid dehydrogenase E1, beta polypeptide (maple syrup urine | |
| | | | disease) | |
| 314 | GCAAAAGAAA | 155543 | proteasome (prosome, macropain) 26S subunit, non-ATPase, 7 (Mov34 homolog) | |
| 315 | CTCCACCCGA | 82961 | Trefoil factor 3 | |
| 316 | AATATGTGGG | 98664 | ESTs, Moderately similar to COXH HUMAN Cytochrome c oxidase polypeptide VIC | |
| | | | precursor [H.sapiens] | |
| 317 | AATATCTGGG | 351875 | cytochrome c oxidase subunit VIc | |
| 318 | GTAGTTACTG | 269021 | ESTs | |
| 319 | TGGCAACCTT | 279952 | glutathione S-transferase subunit 13 homolog | |
| 320 | TGGCAACCTT | 75117 | interleukin enhancer binding factor 2, 45kD | |
| 321 | TGTCATAGTT | | | |
| 322 | GTCCCTGCCT | 279837 | glutathione S-transferase M2 (muscle) | |
| 323 | GTCCCTGCCT | 301961 | glutathione S-transferase M1 | |
| 324 | ATTGTTTATG | 181163 | high-mobility group (nonhistone chromosomal) protein 17 | |
| 325 | ATTGTTTATG | 33317 | KIAA1393 protein | |
| 326 | GCCTGCTGGG | 2706 | glutathione peroxidase 4 (phospholipid hydroperoxidase) | |
| 327 | TGCTGCCTGT | 118110 | bone marrow stromal cell antigen 2 | |
| 328 | TGCTGCCTGT | 145477 | HCGIV-6 protein | |
| 329 | GTGACCTCCT | 180139 | SMT3-suppressor of mif two 3 homolog 2 (yeast) | |
| 330 | CACGCAATGC | 244 | amino-terminal enhancer of split | |
| 331 | CACGCAATGC | 21907 | histone acetyltransferase | |
| 332 | CAAACCATCC | 65114 | keratin 18 | |
| 333 | CAAACCATCC | 348292 | Homo sapiens cDNA: FLJ22448 fis, clone HRC09541 | |
| 334 | ACCGCCTGTG | 79625 | chromosome 20 open reading frame 149 | |
| 335 | CTCAACATCT | 348311 | ribosomal protein, large, P0 pseudogene 2 | |
| 336 | CTCAACATCT | 350108 | ribosomal protein, large. P0 | |
| 337 | TTGTAATCGT | | | |
| 338 | GTGCCATATT | 5337 | isocitrate dehydrogenase 2 (NADP+), mitochondrial | |
| 339 | GTGCCATATT | 254709 | EST | |
| 340 | CATTTGTAAT | 13999 | KIAA0700 protein | |
| 341 | AGTGCCGTGT | 154654 | cytochrome P450, subfamily I (dioxin-inducible), polypeptide 1 (glaucoma 3, | |
| | | | primary infantile) | |
| 342 | AGTGCCGTGT | 76391 | myxovirus (influenza virus) resistance 1, interferon-inducible protein p78 | |
| | | | (mouse) | |
| 343 | ATGGCTGGTA | 182426 | ribosomal protein S2 | |
| 344 | ATGGCTGGTA | 334668 | hypothetical protein FLJ23209 | |
| 345 | GGCTTTACCC | 119140 | eukaryotic translation initiation factor 5A | |
| 346 | CTGGTGAAGG | 75968 | thymosin, beta 4, X chromosome | |
| 347 | TTGGTGAAGG | 356629 | Homo sapiens cDNA FLJ31414 fis, clone NT2NE2000260, weakly similar to THYMOSIN | |
| | | | BETA-4 | |
| 348 | TAGCTCTATG | 76549 | ATPase, Na+/K+ transporting, alpha 1 polypeptide | |
| 349 | AATAAAGAGA | 28149 | hypothetical protein BC010626 | |
| 350 | AATAAAGAGA | 337535 | ESTs | |
| 351 | CAAATAAAAA | 1116 | lymphotoxin beta receptor (TNFR superfamily, member 3) | |
| 352 | CAAATAAAAA | 21198 | translocase of outer mitochondrial membrane 70 homolog A (yeast) | |
| 353 | TACCATCAAT | 79877 | myotubularin related protein 6 | |
| 354 | TACCATCAAT | 169476 | glyceraldehyde-3-phosphate dehydrogenase | |
| 355 | TAAGTAGCAA | 111911 | ESTs, Weakly similar to T06291 extensin homolog T9E8.80 | |
| 356 | TAAGTAGCAA | 239625 | integral membrane protein 2B | |
| 357 | GAAGCAGGAC | 180370 | cofilin 1 (non-muscle) | |
| 358 | TTAGCAATAA | 74346 | hypothetical protein MGC14353 | |
| 359 | TTAGCAATAA | 75798 | chromosome 20 open reading frame 111 | |
| 360 | CAATGTGTTA | 74823 | NADH dehydrogenase (ubiquinone) 1 alpha subcomplex, 1 (7.5kD, MWFE) | |
| 361 | CAATGTGTTA | 181788 | ESTs | |
| 362 | GAGGACCCAA | 77313 | cyclin-dependent kinase (CDC2-like) 10 | |
| 363 | CCGTGCTCAT | 9857 | dicarbonyl/L-xylulose reductase | |
| 364 | GGGTGCTTGG | 6551 | ATPase, H+ transporting, lysosomal interacting protein 1 | |
| 365 | GTGCAGGGAG | 79414 | prostate epithelium-specific Ets transcription factor | |
| 366 | GTGCAGGGAG | 180403 | STRIN protein | |
| 367 | TTACTAAATG | 155560 | calnexin | |
| 368 | TTACTAAATG | 7917 | DKFZPS64K247 protein | |
| 369 | GAAATACAGT | 67201 | 5′,3′-nucleotidase, cytosolic | |
| 370 | GAAATACAGT | 343475 | cathepsin D (lysosomal aspartyl protease) | |
| 371 | CAAATAAAAT | 71465 | squalene epoxidase | |
| 372 | TGCATCTGGT | 75410 | heat shock 70kD proteins 5 (glucose-regulated protein, 78kD) | |
| 373 | TTTCAGGGGA | | | |
| 374 | TTTGGTGTTT | 83190 | fatty acid synthase | |
| 375 | TACCTCTGAT | 2962 | S100 calcium binding protein P | |
| 376 | TACCTCTGAT | 263455 | ESTs, Weakly similar to hypothetical protein FLJ20489 [Homo sapiens] | |
| | | | [H.sapiens] | |
| 377 | GGCCAGCCCT | 155455 | phosphofructokinase, liver | |
| 378 | GGCCAGCCCT | 79 | hypothetical protein MGC15429 | |
| 379 | GCTTTGATGA | 89649 | epoxide hydrolase 1, microsomal (xenobiotic) | |
| 380 | GCTTTGATGA | 279681 | heterogeneous nuclear ribonucleoprotein H3 (2H9) | |
| 381 | AATAAAGGCT | 1815 | myosin, light polypeptide 3, alkali; ventricular, skeletal, slow | |
| 382 | AATAAAGGCT | 179735 | ras homolog gene family, member C | |
| 383 | CCTTTGCCCT | | | |
| 384 | CACTTCAAGG | 77667 | lymphocyte antigen 6 complex, locus E | |
| 385 | TTCATACACC | | | |
| 386 | TCTGTACACC | 182740 | ribosomal protein S11 | |
| 387 | CCATTGCACT | 194382 | ataxia telangiectasia mutated (includes complementation groups A, C and D) | |
| 388 | CCATTGCACT | 244378 | solute carrier family 2 (facilitated glucose transporter), member 6 | |
| 389 | AAATAAAGAA | 14841 | ESTs | |
| 390 | AAATAAAGAA | 355733 | microsomal glutathione S-transferase 1 | |
| 391 | GGGTTGGCTT | 73818 | ubiquinol-cytochrome c reductase hinge protein | |
| 392 | ACTTTTTCAA | 133430 | ESTs | |
| 393 | ACTTTTTCAA | 246501 | EST | |
| 394 | CCCATCGTCC | | | |
| 395 | GCGGCTTTCC | 278431 | SCO cytochrome oxidase deficient homolog 2 (yeast) | |
| 396 | GGGAACCAGA | | | |
| 397 | CTGACCTGTG | 77961 | major histocompatibility complex, class I, B | |
| 398 | CTGACCTGTG | 181244 | major histocompatibility complex, class I, A | |
| 399 | GTAAGTGTAC | | | |
| 400 | TAGTTGGAAA | 1119 | nuclear receptor subfamily 4, group A, member 1 | |
| 401 | ATTTTCTAAA | 91011 | anterior gradient 2 homolog (Xenepus laevis) | |
| 402 | TGCTAAAAAA | 146550 | myosin, heavy polypeptide 9, non-muscle | |
| 403 | TGCTAAAAAA | 313761 | ESTs | |
| 404 | GGAATAAATT | | | |
| 405 | GTGTGTAAAA | 291904 | accessory protein BAP31 | |
| 406 | AGAAAAAAAA | 153834 | pumilio homolog 1 (Drosophila) | |
| 407 | AGAAAAAAAA | 254105 | enolase 1, (alpha) | |
| 408 | TCAAAAAAAA | 10846 | polyamine N-acetyltransferase | |
| 409 | TCAAAAAAAA | 333524 | hypothetical protein MGC13064 | |
| 410 | CTAAAAAAAA | 9873 | likely homolog of rat kinase n-interacting substance of 220 kDa | |
| 411 | CTAAAAAAAA | 54457 | CD81 antigen (target of antiproliferative antibody 1) | |
| 412 | CAAAAAAAAA | 126906 | hypothetical protein FLJ12598 | |
| 413 | CAAAAAAAAA | 234355 | hypothetical protein FLJ22569 | |
| 414 | GACTCACTTT | 699 | peptidylprolyl isomerase B (cyclophilin B) | |
| 415 | AGTTTCCCAA | 312644 | sulfotransferase family, cytosolic, 1C, member 2 | |
| 416 | AGTTTCCCAA | 279929 | gp25L2 protein | |
| 417 | GCAAAAAAAA | 4746 | hypothetical protein FLJ21324 | |
| 418 | GCAAAAAAAA | 91579 | similar to HYPOTHETICAL 34.0 KDA PROTEIN ZK795.3 IN CHROMOSOME IV | |
| 419 | CACTTGCCCT | 14779 | acetyl-Coenzyme A synthetase 2 (ADP forming) | |
| 420 | CACTTGCCCT | 15977 | NADH dehydrogenase (ubiquinone) 1 beta subcomplex, 9 (22kD, B22) | |
| 421 | CTTAATCCTG | 298275 | solute carrier family 38, member 2 | |
| 422 | AAAAAAAAAA | 78713 | solute carrier family 25 (mitochondrial carrier; phosphate carrier), member 3 | |
| 423 | AAAAAAAAAA | 10235 | chromosome 5 open reading frame 4 | |
| 424 | GAAAAAAAAA | 12185 | protein phosphatase 1, regulatory (inhibitor) subunit 16A | |
| 425 | GAAAAAAAAA | 99843 | DKFZP586N0721 protein | |
| 426 | GGGGACTGAA | 438 | mesenchyme homeo box 1 | |
| 427 | GGGGACTGAA | 3709 | low molecular mass ubiquinone-binding protein (9.5kD) | |
| 428 | TTGAATTCCC | 171921 | sema domain, immunoglobulin domain (Ig), short basic domain, secreted, | |
| | | | (semaphorin) 3C | |
| 429 | GCTTTTTAGA | 251064 | high-mobility group (nonhistone chromosomal) protein 14 | |
| 430 | GCTTTTTAGA | 356285 | ESTs, Highly similar to HG14 HUMAN Nonhistone chromosomal protein HMG-14 | |
| | | | [H.sapiens] | |
| 431 | TTTCTGTTAA | 12101 | hypothetical protein LOC51242 | |
| 432 | TGATCTCCAA | 11050 | F-box only protein 9 | |
| 433 | TGATCTCCAA | 83190 | fatty acid synthase | |
| 434 | AAAGTCTAGA | 82932 | cyclin D1 (PRAD1: parathyroid adenomatosis 1) | |
| 435 | CCCTACCCTG | 75736 | apolipoprotein D | |
| 436 | TACATAATTA | 240443 | multiple endocrine neoplasia I | |
| 437 | TTCAATAAAA | 2012 | transcobalamin I (vitamin B12 binding protein, R binder family) | |
| 438 | TTCAATAAAA | 177592 | ribosomal protein, large, P1 | |
| 439 | TAAGGAGCTG | 299465 | ribosomal protein S26 | |
| 440 | TAAGGAGCTG | 355957 | ESTs, Highly similar to RS26 HUMAN 40S ribosomal protein S26 [H.sapiens] | |
| 441 | TAAAAAAAAA | 80612 | ubiquitin-conjugating enzyme E2A (RAD6 homolog) | |
| 442 | TAAAAAAAAA | 244621 | ribosomal protein S14 | |
| 443 | TCTGTTTATC | 180394 | signal recognition particle 14kD (homologous Alu RNA binding protein) | |
| 444 | TCTGTTTATC | 355573 | ESTs, Highly similar to S34196 signal recognition particle 14K chain | |
| 445 | GTAAAAAAAA | 77495 | UBX domain-containing 2 | |
| 446 | GTAAAAAAAA | 279887 | aryl hydrocarbon receptor interacting protein-like 1 | |
| 447 | CCCCAGTTGC | 120811 | ESTs | |
| 448 | CCCCAGTTGC | 74451 | calpain, small subunit 1 | |
| 449 | TGTACCTGTA | 249922 | EST | |
| 450 | TGTACCTGTA | 334842 | tubulin, alpha, ubiquitous | |
| 451 | GAACACATCC | 252723 | ribosomal protein L19 | |
| 452 | AATAGTTGTG | | | |
| 453 | AACTAAAAAA | 3297 | ribosomal protein S27a | |
| 454 | AACTAAAAAA | 55921 | glutamyl-prolyl-tRNA synthetase | |
| 455 | TAGGTTGTCT | 279860 | tumor protein, translationally-controlled 1 | |
| 456 | TAGGTTGTCT | 374596 | ESTs, Highly similar to S06590 IgE-dependent histamine-releasing factor | |
| 457 | TTAAAAAAAA | 19054 | hypothetical protein PRO2521 | |
| 458 | TTAAAAAAAA | 78825 | matrin 3 | |
| 459 | AACTAACAAA | 25996 | ESTs, Moderately similar to UQHUR7 ubiquitin | |
| 460 | AACTAACAAA | 3297 | ribosomal protein S27a | |
| 461 | CAAGGGCTTG | 156764 | RAP1B, member of RAS oncogene family | |
| 462 | AAGGCAATTT | 301626 | Homo sapiens cDNA FLJ11739 fis, clone HEMBAI005497 | |
| 463 | AAGGCAATTT | 164170 | vascular Rab-GAP/TBC-containing | |
| 464 | CTCCTCACCT | 93213 | BCL2-antagonist/killer 1 | |
| 465 | CTCCTCACCT | 119122 | ribosomal protein L13a | |
| 466 | GACTCTGGTG | 334859 | histone methyltransferase DOTIL | |
| 467 | GACTCTGGTG | 356189 | Homo sapiens, ribosomal protein S15a, clone MGC:44895 IMAGE:5580542, mRNA, | |
| | | | complete cds | |
| 468 | ATTCTCCAGT | 234518 | ribosomal protein L23 | |
| 469 | AAAAAACCCA | 111680 | endosulfine alpha | |
| 470 | TGATAATTCA | 171625 | hypothetical protein MGC14697 | |
| 471 | GGGCTGGGGT | 90436 | sperm associated antigen 7 | |
| 472 | GGCTGGGGGT | 350068 | ribosomal protein L29 | |
| 473 | GCTTAACCTG | 77508 | glutamate dehydrogenase 1 | |
| 474 | GGATTTGGCC | 82506 | KIAA1254 protein | |
| 475 | GGATTTGGCC | 343426 | ESTs | |
| 476 | TGCACGTTTT | 169793 | ribosomal protein L32 | |
| 477 | GCATAATAGG | 356482 | ESTs, Weakly similar to putative 60S ribosomal protein L21 [Arabidopsis | |
| | | | thaliana] [A.thaliana] | |
| 478 | GCATAATAGG | 350077 | ribosomal protein L21 | |
| 479 | GCACAAGAAG | 289721 | growth arrest-specific 5 | |
| 480 | TAAACTGTTT | 244621 | ribosomal protein S14 | |
| 481 | TCAGATCTTT | 108124 | ribosomal protein S4, X-linked | |
| 482 | GACAAAAAAA | 343665 | ribosomal protein S15a | |
| 483 | GACAAAAAAA | 356505 | ESTs, Moderately similar to RS1A ARATH 40S ribosomal protein S15A | |
| | | | [A.thaliana] | |
| 484 | GGAACAAACA | 197345 | thyroid autoantigen 70kD (Ku antigen) | |
| 485 | GGAACAAACA | 286124 | CD24 antigen (small cell lung carcinoma cluster 4 antigen) | |
| 486 | CTAACTTCGT | 14838 | likely ortholog of mouse NPC derived proline rich protein 1 | |
| 487 | GCTCAGCTGG | 223241 | eukaryotic translation elongation factor 1 delta (guanine nucleotide exchange | |
| | | | protein) | |
| 488 | CGGCGTGGCC | 8854 | Pvt1 oncogene homolog, MYC activator (mouse) | |
| 489 | AGCCAAAAAA | 235768 | NK inhibitory receptor precursor | |
| 490 | AGCCAAAAAA | 89388 | Homo sapiens cDNA FLJ31372 fis, clone NB9N42000281 | |
| 491 | TGGCGTACGG | | | |
| 492 | GGAGCGTGGG | 286226 | myosin IC | |
| 493 | ACAGCGGCAA | 323462 | DEAD/H (Asp-Glu-Ala-Asp/His) box polypeptide 30 | |
| 494 | ACAGCGGCAA | 349499 | desmoplakin (DPI, DPII) | |
| 495 | TCAAGTTCAC | 351928 | Homo sapiens mRNA full length insert cDNA clone EUROIMAGE 1977059 | |
| 496 | GGAAGCACGG | 355544 | ESTs, Weakly similar to T05691 multiubiquitin chain-binding protein MBP1 | |
| 497 | GGAAGCACGG | 148495 | proteasome (prosome, macropain) 265 subunit, non-ATPase, 4 | |
| 498 | CAGTTACAAA | 7910 | RING1 and YY1 binding protein | |
| 499 | CAGTTACAAA | 312857 | ESTs | |
| 500 | CAGGACAGTT | 78305 | RAB2, member RAS oncogene family | |
| 501 | GGGGAAATCG | 76293 | thymosin, beta 10 | |
| 502 | CAAATCCAAA | 227400 | mitogen-activated protein. kinase kinase kinase kinase 3 | |
| 503 | TCAGAAGTTT | 243901 | Homo sapiens mRNA: cDNA DKFZp564C1563 (from clone DKFZp564C1563) | |
| 504 | AAAGTTCTCA | 284243 | transmembrane 4 superfamily member tetraspan NET-6 | |
| 505 | AAGGATGCCA | 169946 | GATA binding protein 3 | |
| 506 | AAGGATGCCA | 104823 | EST | |
| 507 | GAGGGCCGGT | 36727 | H2A histone family, member J | |
| 508 | CAGCAGAAGC | 323806 | small EDRK-rich factor 2 | |
| 509 | CAGCAGAAGC | 343261 | histocompatibility (minor) 13 | |
| 510 | CCTCCAGCTA | 242463 | keratin 8 | |
| 511 | CCTCCAGCTA | 356123 | ESTs, Moderately similar to I37982 Keratin 8 | |
| 512 | GCCTTCCAAT | 76053 | DEAD/H (Asp-Glu-Ala-Asp/His) box polypeptide 5 (RNA helicase, 68kD) | |
| 513 | GGGAGCCCGG | 183986 | poliovirus receptor-related 2 (herpesvirus entry mediator B) | |
| 514 | GCTCCCAGAC | 5097 | synaptogyrin 2 | |
| 515 | GCAGGGCCTC | 301350 | FXYD domain-containing ion transport regulator 3 | |
| 516 | TTGGAGATCT | 50098 | NADH dehydrogenase (ubiquinone) I alpha subcomplex, 4 (9kD, MLRQ) | |
| 517 | GGAAAAAAAA | 177530 | ATP synthase, H+ transporting, mitochondrial F1 complex, epsilon subunit | |
| 518 | GGAAAAAAAA | 198271 | NADH dehydrogenase (ubiquinone) 1 alpha subcomplex, 10 (42kD) | |
| 519 | AAGAAAACTG | 330208 | crystallin, zeta (quinone reductase)-like 1 | |
| 520 | AAGAAAACTG | 322735 | KIAA1522 protein | |
| 521 | GACATCAAGT | 182265 | Keratin 19 | |
| 522 | GCAGTGGCCT | 184276 | solute carrier family 9 (sodium/hydrogen exchanger), isoform 3 regulatory | |
| | | | factor 1 | |
| 523 | GCAGTGGCCT | 161166 | KIAA1094 protein | |
| 524 | CGCCGACGAT | 265827 | interferon, alpha-inducible protein (clone IFI-6-16) | |
| 525 | ATGTCTTTTC | 1516 | insulin-like growth factor binding protein 4 | |
| 526 | ATGTCTTTTC | 59483 | leucine-rich repeat-containing G protein-coupled receptor 6 | |
| 527 | GCCGTCGGAG | 265827 | interferon, alpha-inducible protein (clone IFI-6-16) | |
| 528 | CGGACTCACT | 84700 | serologically defined colon cancer antigen 28 | |
| 529 | ACGCAGGGAG | 279789 | glucose phosphate isomerase | |
| 530 | CCAGGGGAGA | 254105 | enolase 1, (alpha) | |
| 531 | CCAGGCGAGA | 278613 | interferon, alpha-inducible protein 27 | |
| 532 | AAGAAAACCT | 100686 | anterior gradient protein 3 | |
| 533 | AAGAAAACCT | 274319 | hypothetical protein FLJ10509 | |
| 534 | AGATTCAAAC | 14368 | SH3 domain binding glutamic acid-rich protein like | |
| 535 | TGGGGAGAGG | | | |
| 536 | CCAAACGTGT | 181307 | H3 histone, family 3A | |
| 537 | CCAAACGTGT | 367720 | ESTs, Highly similar to HSHU33 histone H3.3 | |
| 538 | AAGCCTAAAA | 79136 | LIV-1 protein, estrogen regulated | |
| 539 | GTGCTGAATG | 77385 | myosin, light polypeptide 6, alkali, smooth muscle and non-muscle | |
| 540 | GTGCTGAATG | 120260 | immunoglobulin superfamily receptor translocation associated 1 | |
| 541 | AACGCGGCCA | 60300 | hypothetical protein MGC17552 | |
| 542 | AACGCGGCCA | 73798 | macrophage migration inhibitory factor (glycosylation-inhibiting factor) | |
| 543 | GGCAACGTGG | 300954 | Huntingtin interacting protein K | |
| 544 | GGCAACGTGG | 31608 | transient receptor potential cation channel, subfamily M, member 4 | |
| 545 | CGCCGCGGTG | 4835 | eukaryotic translation initiation factor 3, subunit 8 (110kD) | |
| 546 | GTGACCACGG | 299882 | ESTs, Highly similar to N-methyl-D-aspartate receptor 2C subunit precursor | |
| | | | [Homo sapiens] [H.sapiens] | |
| 547 | CCGACGGGCG | | | |
| 548 | GGTGGCACTC | 77273 | ras homolog gene family, member A | |
| 549 | GGTGGCACTC | 77550 | p53-regulated DDA3 | |
| 550 | GGGATCAAGG | 9265 | mitochondrial ribosomal protein L24 | |
| 551 | TGGAGTGGAG | 3764 | guanylate kinase 1 | |
| 552 | TGCCTCTGCG | | | |
| 553 | TCCCTGGCTG | 78575 | prosaposin (variant Gaucher disease and variant metachromatic leukodystrophy) | |
| 554 | TCCCTGGCTG | 166160 | acetyl-Coenzyme A acyltransferase 1 (peroxisomal 3-oxoacyl-Coenzyme A | |
| | | | thiolase) | - |
| 555 | GACGACACGA | 153177 | ribosomal protein S28 | |
| 556 | GACGACACGA | 374547 | ESTs, Moderately similar to RS28 ARATH 40S ribosomal protein S28 [A.thaliana] | |
| 557 | GTGCTGGACC | 20977 | ganglioside-induced differentiation-associated protein 1-like 1 | |
| 558 | GTGCTGGACC | 179774 | proteasome (prosome, macropain) activator subunit 2 (PA28 beta) | |
| 559 | GCAGGCCAAG | 69771 | B-factor, properdin | |
| 560 | GCAGGCCAAG | 159505 | RAB30, member RAS oncogene family | |
| 561 | TGCCTGCACC | 135084 | cystatin C (amyloid angiopathy and cerebral hemorrhage) | |
| 562 | TCAGCCTTCT | 112165 | Homo sapiens cDNA FLJ12198 fis, clone MAMMA1000876 | |
| 563 | TCAGCCTTCT | 179986 | flotillin 1 | |
| 564 | TAGAAAAATA | 79194 | cAMP responsive element binding protein 1 | |
| 565 | TAGAAAAATA | 279789 | glucose phosphate isomerase | |
| 566 | AAGACAGTGG | 3352 | histone deacetylase 2 | |
| 567 | AAGACAGTGG | 296290 | ribosomal protein L37a | |
| 568 | CGTGCTAAAT | 250895 | ribosomal protein L34 | |
| 569 | TGTGCTAAAT | 11387 | KIAA1453 protein | |
| 570 | TCTCCATACC | | | |
| 571 | GGCAAGAAGA | 83321 | neuromedin B | |
| 572 | GGCAAGAAGA | 111611 | ribosomal protein L27 | |
| 573 | GAAAAATTTA | 169248 | cytochrome c | |
| 574 | TTGGTCCTCT | 356796 | Homo sapiens E1BPI pseudogene, mRNA sequence | |
| 575 | TTGGTCCTCT | 356795 | ribosomal protein L41 | |
| 576 | GTGTGGGGGG | 2340 | junction plakoglobin | |
| 577 | GTGTGGGGGG | T117484 | ESTs | |
| 578 | CGTGGGTGGG | 202833 | heme oxygenase (decycling) 1 | |
| 579 | GCGACGAGGC | 2017 | ribosomal protein L38 | |
| 580 | GCCGTTCTTA | | | |
| 581 | ACCCGCCGGG | | | |
| 582 | GGCCTGCTGC | 280792 | hypothetical protein FLJ12387 similar to kinesin light chain | |
| 583 | GGCCTGCTGC | 9634 | hypothetical protein BC009925 | |
| 584 | GGTTTGGCTT | 73818 | ubiquinol-cytochrome c reductase hinge protein | |
| 585 | TCAGTTTGTC | 121397 | ESTs | |
| 586 | TCAGTTTGTC | 15318 | HS1 binding protein | |
| 587 | GGTCAGTCGG | | | |
| 588 | CTAACTAGTT | | | |
| 589 | AAGGTGGAGG | 76171 | CCAAT/enhancer binding protein (C/EBP), alpha | |
| 590 | AAGGTGGAGG | 163593 | ribosomal protein L18a | |
| 591 | AGGCTACGGA | 119122 | ribosomal protein L13a | |
| 592 | AGGCTACGGA | 356678 | ESTs, Weakly similar to T07697 ribosomal protein L13a, cytosolic | |
| 593 | GAAGTTATGA | 4112 | t-complex 1 | |
| 594 | TCACAAGCAA | 32916 | nascent-polypeptlde-associated complex alpha polypeptide | |
| 595 | GCGCTGGAGT | 241432 | ESTs, Highly similar to c380A1.1b [H.sapiens] | |
| 596 | GCGCTGGAGT | 110695 | hypothetical protein MGC3133 | |
| 597 | GGACCACTGA | 119598 | ribosomal protein L3 | |
| 598 | GGACCACTGA | 356258 | ESTs, Weakly similar to ribosomal protein [Arabidopsis thaliana] [A.thaliana] | |
| 599 | GCGGTGAGGT | 203910 | small glutamine-rich tetratricopeptide repeat (TPR)-containing | |
| 600 | CAATAAACTG | 150580 | putative translation initiation factor | |
| 601 | CAATAAACTG | 297112 | ESTs | |
| 602 | AGGAAAGCTG | 227591 | hypothetical protein FLJ11088 | |
| 603 | AGGAAAGCTG | 343443 | ribosomal protein L36 | |
| 604 | CTGGGTTAAT | 356647 | ESTs | |
| 605 | CTGGGTTAAT | 298262 | ribosomal protein S19 | |
| 606 | AAGGAGATGG | 164170 | vascular Rab-GAP/TBC-containing | |
| 607 | AAGGAGATGG | 355990 | ESTs, Highly similar to R5HU31 ribosomal protein L31 | |
| 608 | ACATCATCGA | 182979 | ribosomal protein L12 | |
| 609 | ACATCATCGA | 356318 | ESTs, Weakly similar to T45883 60S RIBOSOMAL PROTEIN L12-like | |
| 610 | ATTATTTTTC | 153 | ribosomal protein L7 | |
| 611 | ATTATTTTTC | 356593 | ribosomal protein L7 | |
| 612 | TAGTTGAAGT | 131255 | ubiquinol-cytochrome c reductase binding protein | |
| 613 | CCAGAACAGA | 79006 | deoxythymidylate kinase (thymidylate kinase) | |
| 614 | CCAGAACAGA | 334807 | ribosomal protein L30 | |
| 615 | GCATTTAAAT | 275959 | eukaryotic translation elongation factor 1 beta 2 | |
| 616 | GCATTTAAAT | 356184 | ESTs, Weakly similar to elongation factor 1-beta, putative [Arabidopsis | |
| | | | thaliana] [A.thaliana] | |
| 617 | GAAAAATGGT | 181357 | laminin receptor 1 (67kD, ribosomal protein SA) | |
| 618 | GAAAAATGGT | 356267 | Homo sapiens laminin receptor-like protein LAMRL5 mRNA, complete cds | |
| 619 | GGTTGGCAGG | 3745 | milk fat globule-EGF factor 8 protein | |
| 620 | GGTTGGCAGG | 17908 | origin recognition complex, subunit 1-like (yeast) | |
| 621 | GTGAAGGCAG | 77039 | ribosomal protein S3A | |
| 622 | GTGAAGGCAG | 356568 | ESTs, Weakly similar to Putative S-phase-specific ribosomal protein | |
| | | | [Arabidopsis thaliana] [A.thaliana] | |
| 623 | TTGCGTTGCG | | | |
| 624 | ATCTCAGCTC | 8036 | RAB3D, member RAS oncogene family | |
| 625 | ATCTCAGCTC | 29736 | TNF receptor-associated factor 5 | |
| 626 | AAAAAATTCA | 254271 | hypothetical protein MGC24009 | |
| 627 | TGGCCCCACC | 146662 | Homo sapiens cDNA FLJ36928 fis,.clone BRACE2005216, weakly similar to Xenopus | |
| | | | laevis bicaudal-C (Bic C) mRNA | |
| 628 | TGGCCCCACC | 198281 | pyruvate kinase, muscle | |
| 629 | TCCATCTGTT | 252189 | syndecan 4 (amphiglycan, ryudocan) | |
| 630 | CAACTGGAGT | 166011 | catenin (cadherin-associated protein), delta 1 | |
| 631 | CAACTGGAGT | 352566 | cytochrome P450 monooxygenase | |
| 632 | GCCCAGCTGG | 12479 | associated molecule with the SH3 domain of STAM | |
| 633 | GCCCAGCTGG | 334798 | hypothetical protein FLJ20897 | |
| 634 | GACGGCGCAG | 73946 | endothelial cell growth factor 1 (platelet-derived) | |
| 635 | ATGAAACCCC | 75470 | chromosome 1 open reading frame 29 | |
| 636 | ATGAAACCCC | 226396 | hypothetical protein FLJ11126 | |
| 637 | AGCCACCGCA | 242 | glucose-6-phosphatase, catalytic (glycogen storage disease type I, von Gierke | |
| | | | disease) | |
| 638 | AGCCACCGCA | 244482 | M-phase phosphoprotein, mpp8 | |
| 639 | CCCAGCTAAT | 73809 | arachidonate 15-lipoxygenase | |
| 640 | CCCAGCTAAT | 200395 | centromere protein H | |
| 641 | GTGAAACCCC | 44396 | coronin, actin binding protein, 2A | |
| 642 | GTGAAACCCC | 323949 | kangai 1 (suppression of tumorigenicity 6, prostate; CD82 antigen (R2 | |
| | | | leukocyte antigen, antigen detected by monoclonal and antibody IA4)) | |
| 643 | GTGAAACCCT | 289053 | CAP-binding protein complex interacting protein 2 | |
| 644 | GTGAAACCCT | 52644 | src family associated phosphoprotein 2 | |
| 645 | GAGAAACCCC | 5719 | chromosome condensation-related SMC-associated protein 1 | |
| 646 | GAGAAACCCC | 114318 | hypothetical protein MGC16385 | |
| 647 | GTGAAACCTT | 365695 | Homo sapiens cDNA FLJ108365, clone PLACE1005232 | |
| 648 | GTGAAACCTT | 264636 | FK506 binding protein 14 (22 kDa) | |
| 649 | GTGAAACTCC | 75410 | heat shock 70kD protein 5 (glucose-regulated protein, 78kD) | |
| 650 | GTGAAACTCC | 256156 | hypothetical protein BC018697 | |
| 651 | GTGAAATCCC | 274448 | hypothetical protein FLJ11029 | |
| 652 | GTGAAATCCC | 287587 | Homo sapiens cDNA FLJ13671 fis, clone PLACE1011729 | |
| 653 | AACCCGGGAG | 118744 | KIAA0408 gene product | |
| 654 | AACCCGGGAG | 173936 | interleukin 10 receptor, beta | |
| 655 | GTGGCGGGCA | 6874 | KIAA0472 protein | |
| 656 | GTGGCCGGCA | 169813 | hypothetical protein FLJ23040 | |
| 657 | TTGCCCAGGC | 9711 | novel protein | |
| 658 | TTGCCCAGGC | 286124 | CD24 antigen (small cell lung carcinoma cluster 4 antigen) | |
| 659 | GTGGTGGGTG | 289020 | Homo sapiens cDNA FLJ11553 fis, clone HEMBA1003034 | |
| 660 | GTGGTGGGTG | 171731 | solute carrier family 14 (urea transporter), member 1 (Kidd blood group) | |
| 661 | CCTGTAATCC | 181874 | interferon-induced protein with tetratricopeptide repeats 4 | |
| 662 | CCTGTAATCC | 292154 | stromal cell protein | |
| 663 | AGCCACTGTG | 147313 | similar to CMRF35 antigen precursor (CMRF-35) | |
| 664 | AGCCACTGTG | 348642 | Homo sapiens FGF2-associated protein GAFA1 (GAFA1) mRNA, complete cds | |
| 665 | GTGGCAGGCA | 13255 | KIAA0930 protein | |
| 666 | GTGGCAGGCA | 47334 | reserved | |
| 667 | GTAAAACCCC | 12106 | hypothetical protein MGC20496 | |
| 668 | GTAAAACCCC | 256278 | tumor necrosis factor receptor superfamily, member 1B | |
| 669 | CCTGGCTAAT | 274170 | Opa-interacting protein 2 | |
| 670 | CCTGGCTAAT | 117062 | apoptosis-inducing factor (AIF)-homologous mitochondrion-associated inducer of | |
| | | | death | |
| 671 | GTGAAATCCT | 301509 | Homo sapiens cDNA FLJ12339 fis, clone MAMMA1002250 | |
| 672 | GTGAAATCCT | 9280 | proteasome (prosome, macropain) subunit, beta type, 9 (large multifunctional | |
| | | | protease 2) | |
| 673 | GTGGCACGTG | 29759 | polymerase land transcript release factor | |
| 674 | GTGGCACGTG | 306850 | Homo sapiens cDNA FLJ22796 fis, clone KAIA2544 | |
| 675 | GTGGCTCACA | 270134 | hypothetical protein FLJ20280 | |
| 676 | GTGGCTCACA | 124813 | hypothetical protein MGC14817 | |
| 677 | TGCCTGTAAT | 349344 | hypothetical protein BC001573 | |
| 678 | TGCCTGTAAT | 342655 | Homo sapiens cDNA FLJ13289 fis, clone OVARC1001170 | |
| 679 | CCACTGCACT | 14992 | hypothetical protein FLJ11151 | |
| 680 | CCACTGCACT | 107003 | enhancer of invasion 10 | |
| 681 | AGAATTGCTT | 78060 | phosphorylase kinase, beta | |
| 682 | AGAATTGCTT | 190311 | nephrosis 1, congenital, Finnish type (nephrin) | |
| 683 | ATCTTGGCTC | 75859 | mitochondrial ribosomal protein L49 | |
| 684 | ATCTTGGCTC | 129228 | galactokinase 2 | |
| 685 | TTGGCCAGGA | 146668 | KIAA1253 protein | |
| 686 | TTGGCCAGGA | 233335 | KIAA1465 protein | |
| 687 | TTGACCAGGC | 193384 | putatative 28 kDa protein | |
| 688 | TTGACCAGGC | 194351 | coagulation factor 11 (thrombin) receptor-like 2 | |
| 689 | ATCCGCCCGC | 352382 | PI-3-kinase-related kinase SMG-1 | |
| 690 | ATCCGCCCGC | 355762 | riomo sapiens cDNA FLJ35653 fis, clone SPLEN2013690 | |
| 691 | AGCCACCACG | 57735 | scavenger receptor expressed by endothelial cells | |
| 692 | AGCCACCACG | 2593 | phosphodiesterase 6B, cGMP-specific, rod, beta (congenital stationary night | |
| | | | blindness 3, autosomal dominant) | |
| 693 | GTGAAACCCG | 278577 | Homo sapiens mRNA cDNA DKFZp564P073 (from clone DKFZp564P073) | |
| 694 | GTGAAACCCG | 302075 | Homo sapiens cDNA FLJ12365 fis, clone MAMMA1002392 | |
| 695 | CCCGGCTAAT | 273759 | Homo sapiens cDNA FLJ11905 fis, clone HEMBB1000050 | |
| 696 | CCCGGCTAAT | 325116 | JM11 protein | |
| 697 | GTGAAACCCA | 17311 | hypothetical protein FLJ20004 | |
| 698 | GTGAAACCCA | 241205 | peroxisomal membrane protein 4 (24kD) | |
| 699 | GTAAAACCCT | 281680 | peroxisomal trans 2-enoyl CoA reductase; putative short chain alcohol | |
| | | | dehydrogenase | |
| 700 | GTAAAACCCT | 282797 | Homo sapiens cDNA FLJ31194 fis, clone KIDNE2000510 | |
| 701 | GTGAAACTCT | 188853 | Homo sapiens cDNA FLJ12246 fis, clone MAMMA1001343 | |
| 702 | GTGAAACTCT | 333449 | Homo sapiens cDNA FLJ12170 fis, clone MAMMA1000664 | |
| 703 | GTGGCGGGTG | 257584 | Homo sapiens cDNA FLJ12138 fis, clone MAMMA1000331 | |
| 704 | GTGGCGGGTG | 296697 | Homo sapiens cDNA FLJ12093 fis, clone HEMBB1002603 | |
| 705 | GTGGCAGGTG | 280380 | aminopeptidase | |
| 706 | GTGGCAGGTG | 333480 | Homo sapiens cDNA FL113757 fis, clone PLACE3000405 | |
| 707 | GCAAAACCCT | 10844 | leucine-rich alpha-2-glycoprotein | |
| 708 | GCAAAACCCT | 121576 | myosin 1B | |
| 709 | GCAAAACCCC | 86412 | chromosome 9 open reading frame 5 | |
| 710 | GCAAAACCCC | 129708 | tumor necrosis factor (ligand) superfamily, member 14 | |
| 711 | AGGTCAGGAG | 209065 | hypothetical protein FLJ14225 | |
| 712 | AGGTCAGGAG | 212414 | sema domain, immunoglobulin domain (Ig), short basic domain, secreted, | |
| | | | (semaphorin) 3E | |
| 713 | AGCCACCGTG | 156051 | KIAA1443 protein | |
| 714 | AGCCACCGTG | 240845 | DKFZP434D146 protein | |
| 715 | GTGGCACACA | 129057 | breast carcinoma amplified sequence 1 | |
| 716 | GTGGCACACA | 207251 | nucleolar autoantigen (55kD) similar to rat synaptonemal complex protein | |
| 717 | ATCTCGGCTC | 156942 | hypothetical protein BC017947 | |
| 718 | ATCTCGGCTC | 271285 | KIAA1510 protein | |
| 719 | TTGGCCAGAC | 91728 | polymyositis/scleroderma autoantigen 1 (75kD) | |
| 720 | TTGGCCAGAC | 374296 | hypothetical protein similar to KIAA0187 gene product | |
| 721 | GTGGCAGGCG | 48604 | DKFZP434B168 protein | |
| 722 | GTGGCAGGCG | 53985 | glycoprotein 2 (zymogen granule membrane) | |
| 723 | CACCTGTAAT | 175613 | claspin | |
| 724 | CACCTGTAAT | 287473 | hypothetical protein FLJ11996 | |
| 725 | TTGGCCAGGG | 321687 | F-box protein FBX30 | |
| 726 | TTGGCCAGGG | 322840 | Homo sapiens, Similar to protein tyrosine phosphatase-like (proline instead of | |
| | | | catalytic arginine), member a, | |
| 727 | GAGAAACCCT | 321149 | hypothetical protein FLJ10257 | |
| 728 | GAGAAACCCT | 274279 | hypothetical protein FLJ10314 | |
| 729 | GCGAAACCCT | 103189 | lipopolysaccharide specific response-68 protein | |
| 730 | GCGAAACCCT | 225084 | hypothetical protein FLJ14280 | |
| 731 | GTGAAACCTC | 168159 | bifunctional apoptosis regulator | |
| 732 | GTGAAACCTC | 334526 | hypothetical protein MGC14126 | |
| 733 | GCGAAACCCC | 30211 | hypothetical protein FLJ22313 | |
| 734 | GCGAAACCCC | 288945 | hypothetical protein FLJ13448 | |
| 735 | AGCCACCGCG | 122660 | RAB, member of RAS oncogene family-like 2A | |
| 736 | AGCCACCGCG | 355874 | RAB, member of RAS oncogene family-like 2B | |
| 737 | CGCCTGTAAT | 154443 | MCM4 minichromosome maintenance deficient 4 (S. cervisiae) | |
| 738 | CGCCTGTAAT | 287594 | hypothetical protein FLJ13769 | |
| 739 | GTGGCGGGCG | 22926 | KIAA0795 protein | |
| 740 | GTGGCGGGCG | 181780 | hypothetical protein FLJ20241 | |
| 741 | AACCTGGGAG | 105658 | DNA fragmentation factor, 45 kD, alpha polypeptide | |
| 742 | AACCTGGGAG | 334638 | hypothetical protein MGC16175 | |
| 743 | GCTTTCTCAC | | | |
| 744 | CTTGTAATCC | 183253 | nucleolar RNA-associated protein | |
| 745 | CTTGTAATCC | 231119 | protocadherin beta 9 | |
| 746 | TCTGTAATCC | 272216 | glycoprotein VI (platelet) | |
| 747 | TCTGTAATCC | 142 | sulfotransferase family, cytosolic, 1A, phenol-preferring, member 1 | |
| 748 | CCTATAATCC | 86228 | TRIAD3 protein | |
| 749 | CCTATAATCC | 189658 | CGI-149 protein | |
| 750 | TAATCCCAGC | 12496 | Homo sapiens cDNA FLJ23834 fis, clone KAIA2087 | |
| 751 | TAATCCCAGC | 278941 | PRO0628 protein | |
| 752 | TGCCTGTAGT | 48469 | LIM domains containing 1 | |
| 753 | TGCCTGTAGT | 274201 | chromosome 1 open reading frame 33 | |
| 754 | AGGGTGTTTT | 75842 | dual-specificity tyrosine-(Y)-phosphorylation regulated kinase 1A | |
| 755 | AGGGTGTTTT | 160416 | ESTs | |
| 756 | CCAGGGCAAC | 240443 | multiple endocrine neoplasia I | |
| 757 | ATTGTGCCAC | 22151 | neurolysin (metallopeptidase M3 family) | |
| 758 | ATTGTGCCAC | 38761 | Homo sapiens cDNA:FLJ21564 fis, clone COL06452 | |
| 759 | CCTGTAATCT | 199067 | v-erb-b2 erythroblastic leukemia viral oncogene homolog 3 (avian) | |
| 760 | CCTGTAATCT | 3530 | FUS interacting protein (serine-arginine rich) 1 | |
| 761 | GTGGTGGGCA | 99975 | cholinergic receptor, nicotinic, delta polypeptide | |
| 762 | GTGGTGGGCA | 374536 | isovaleryl Coenzyme A dehydrogenase | |
| 763 | TACCCTAAAA | 165662 | KIAA0675 gene product | |
| 764 | TACCCTAAAA | 268971 | Homo sapiens clone IMAGE:212461, mRNA sequence | |
| 765 | ATGGTGGGGG | 343586 | zinc finger protein 36, C3H type, homolog (mouse) | |
| 766 | ACCCTTGGCC | | | |
| 767 | GTGAAAACCC | 127305 | agmatine ureohydrolase (agmatinase) | |
| 768 | GTGAAAACCC | 351029 | Homo sapiens cDNA FLJ31803 fis, clone NT2R12009101 | |
| 769 | ATCCACCCGC | 145381 | general transcription factor IIE, polypeptide 1 (alpha subunit, 56kD) | |
| 770 | ATCCACCCGC | 53263 | nucleoporin Nup43 | |
| 771 | TTAGCCAGGA | 196270 | folate transporter/carrier | |
| 772 | TTAGCCAGGA | 350692 | Homo sapiens cDNA FLJ32756 fis, clone TEST12001758 | |
| 773 | ATGAAACCCT | 31330 | Homo sapiens clone HQ0319 | |
| 774 | ATGAAACCCT | 187991 | SOCS box-containing WD protein SWiP-1 | |
| 775 | GTGGCTCACG | 3454 | KIAA1821 protein | |
| 776 | GTGGCTCACG | 127649 | zinc finger protein 297B | |
| 777 | TTGGCCAGGC | 118194 | debranching enzyme homolog 1 (S. cervisiae) | |
| 778 | TTGGCCAGGC | 274382 | protein kinase, interferon-inducible double stranded RNA dependent | |
| 779 | TTGGTCAGGC | 154069 | melan-A | |
| 780 | TTGGTCAGGC | 172012 | hypothetical protein DKFZp434J037 | |
| 781 | TTGTCCAGGC | 99423 | ATP-dependent RNA helicase | |
| 782 | TTGTCCAGGC | 51305 | v-maf musculoaponeurotic fibrosarcoma oncogene homolog F (avian) | |
| 783 | CTTAATCTTG | 75462 | BTG family, member 2 | |
| 784 | CTTAATCTTG | 237356 | stromal cell-derived factor 1 | |
| 785 | TGGGGTTCTT | 62954 | ferritin, heavy polypeptide 1 | |
| 786 | TGGGGTTCTT | 272499 | dehydrogenase/reductase (SDR family) member 2 | |
| 787 | AAGAAGATAG | 350046 | ribosomal protein L23a | |
| 788 | AAGAAGATAG | 356007 | ESTs, Highly similar to RL2B HUMAN 60S ribosomal protein L23a [H.sapiens] | |
| 789 | AGAATCGCTT | 16165 | expressed in activated T/LAK lymphocytes | |
| 790 | AGAATCGCTT | 75887 | coatomer protein complex, subunit alpha | |
| 791 | CCTGTAGTCC | 51305 | v-maf musculoaponeurotic fibrosarcoma oncogene homolog F (avian) | |
| 792 | CCTGTAGTCC | 77510 | hypothetical protein FLJ10520 | |
| 793 | AGCCACCACA | 5999 | hypothetical protein FLJ10298 | |
| 794 | AGCCACCACA | 8768 | hypothetical protein FLJ10849 | |
| 795 | ATTGCACCAC | 210778 | hypothetical protein FLJ10989 | |
| 796 | ATTGCACCAC | 287948 | Homo sapiens cDNA FLJ1405 fis, clone HEMBA1000769 | |
| 797 | CCACTGTACT | 287515 | hypothetical protein FLJ12331 | |
| 798 | CCACTGTACT | 288537 | Homo sapiens cDNA FLJ12199 fis,.clone MAMMA100088O | |
| 799 | CTGTACTTGT | 75678 | FBI murine osteosarcoma viral oncogene homolog B | |
| 800 | CCATTCTCCT | 98711 | hypothetical protein BC006136 | |
| 801 | CCATTCTCCT | 271752 | 3′(2′), 5′-bisphosphate nucleotidase 1 | |
| 802 | GTGGTGGGCG | 73614 | solute carrier family 31 (copper transporters), member 1 | |
| 803 | GTGGTGGGCG | 287522 | Homo sapiens cDNA FLJ12364 fis, clone MAMMA1002384 | |
| 804 | AGCCACTGCG | 193914 | KIAA0575 gene product | |
| 805 | AGCCACTGCG | 356075 | ninjurin 2 | |
| 806 | GCCGGCTCAT | | | |
| 807 | GCTCACTGCA | 93523 | peptidylprolyl isomerase (cyclophilin)-like 2 | |
| 808 | GCTCACTGCA | 117572 | chemokine binding protein 2 | |
| 809 | CCTGTGGTCC | 120769 | Homo sapiens cDNA FLJ20463 fis, clone KAT06143 | |
| 810 | CCTGTGGTCC | 243804 | Homo sapiens cDNA FLJ13800 fis, clone THYRO1000156 | |
| 811 | GGAGGCTGAG | 306189 | DKFZP434F1735 protein | |
| 812 | GGAGGCTGAG | 185973 | degenerative spermatocyte homolog, lipid desaturase (Drosophila) | |
| 813 | AGAATCACTT | 130815 | hypothetical protein FLJ21870 | |
| 814 | AGAATCACTT | 192127 | Homo sapiens, clone MGC:32020 IMAGE:4620233, mRNA, complete cds | |
| 815 | CCTGTAATTC | 129908 | kinesin family member 1B | |
| 816 | CCTGTAATTC | 306678 | hypothetical protein FLJ14326 | |
| 817 | AGCCACTGCA | 4295 | proteasome (prosome, macropain) 26S subunit, non-ATPase, 12 | |
| 818 | AGCCACTGCA | 173508 | P3ECSL | |
| 819 | AACCCACGAG | 262150 | hypothetical protein FLJ22814 | |
| 820 | AACCCAGGAG | 75813 | polycystic kidney disease 1 (autosomal dominant) | |
| 821 | AAGCCAGGAC | 10326 | coatomer protein complex, subunit epsilon | |
| 822 | GACCTCCTGC | 119324 | kinesin-like 4 | |
| 823 | GACCTCCTGC | 89449 | mitogen-activated protein kinase kinase kinase 11 | |
| 824 | CTGCCAAGTT | 75873 | zyxin | |
| 825 | GTTCGTGCCA | 195464 | filamin A, alpha (actin binding protein 280) | |
| 826 | GCGCAGAGGT | 356795 | ribosomal protein L41 | |
| 827 | GCCGTGTCCG | 356666 | ESTs, Highly similar to RS6 HUMAN 40S ribosomal protein S6 (Phosphoprotein | |
| | | | NP33) [H.sapiens] | |
| 828 | GCCGTGTCCG | 350166 | ribosomal protein S6 | |
| 829 | CCCATCCGAA | 91379 | ribosomal protein L26 | |
| 830 | CCCATCCGAA | 356175 | ESTs, Weakly similar to T46057 60S RIBOSOMAL PROTEIN-like | |
| 831 | CCCGAGGCAG | 45057 | Homo sapiens, Similar to doublecortin and CaM kinase-like 1, clone MGC:45428 | |
| | | | IMAGE:5532881, mRNA, complete cds | |
| 832 | CCCGAGGCAG | 155223 | stanniocalcin 2 | |
| 833 | CCTGAAATTT | 7749 | heterogeneous nuclear ribonucleoprotein A0 | |
| 834 | CCTGAAATTT | 12102 | sorting nexin 3 | |
| 835 | CTCACTTTTT | 9585 | Homo sapiens cDNA FLJ30010 fis, clone 3NB692000154 | |
| 836 | CTCACTTTTT | 76722 | CCAAT/enhancer binding protein (C/EBP), delta | |
| 837 | GCTGTTGCGC | 8102 | ribosomal protein S20 | |
| 838 | TCCCCGTACA | | | |
| 839 | CACAAACGGT | 195453 | ribosomal protein S27 (metallopanstimulin 1) | |
| 840 | CACAAACGGT | 356178 | ESTs, Moderately similar to T47903 ribosomal protein S27 | |
| 841 | CCCTGATTTT | 183684 | eukaryotic translation initiation factor 4 gamma, 2 | |
| 842 | CCCTGATTTT | 1799 | CD1D antigen, d polypeptide | |
| 843 | TGGGCAAAGC | 2186 | eukaryotic translation elongation factor 1 gamma | |
| 844 | TAACTTGTGA | 295726 | integrin, alpha V (vitronectin receptor, alpha polypeptide, antigen CD51) | |
| 845 | AGCACCTCCA | 75309 | eukaryotic translation elongation factor 2 | |
| 846 | GAGGGAGTTT | 76064 | ribosomal protein L27a | |
| 847 | GAGGGAGTTT | 356342 | ESTs, Highly similar to 2113200C ribosomal protein L27a [Homo sapiens] | |
| | | | [H.sapiens] | |
| 848 | GCGACAGCTC | 184582 | ribosomal protein L24 | |
| 849 | CGCCGCCGGC | 182825 | ribosomal protein L35 | |
| 850 | GGCAAGCCCC | 334895 | ribosomal protein L10a | |
| 851 | GGCAAGCCCC | 187577 | SRY (sex determining region Y)-box 21 | |
| 852 | AGCTCTCCCT | 82202 | ribosomal protein L17 | |
| 853 | AGCTCTCCCT | 374588 | ESTs, Highly similar to R5HU22 ribosomal protein L17, cytosolic | |
| 854 | CGCTGGTTCC | 179943 | ribosomal protein L11 | |
| 855 | CGCTGGTTCC | 289019 | latent transforming growth factor beta binding protein 3 | |
| 856 | GAAACCGAGG | 268053 | R3H domain (binds single-stranded nucleic acids) containing | |
| 857 | GAAACCGAGG | 279813 | hypothetical protein HSPC014 | |
| 858 | GAGGTCCCTG | 374499 | ESTs, Weakly similar to PS62 ARATH Proteasome subunit alpha type 6-2 (20S | |
| | | | proteasome alpha subunit A2) [A.thaliana] | |
| 859 | GAGGTCCCTG | 74077 | proteasome (prosome, macropain) subunit, alpha type, 6 | |
| 860 | TGAAATAAAA | 9614 | nucleophosmin (nucleolar phosphoprotein B23, numatrin) | |
| 861 | TGAAATAAAA | 48516 | ESTs | |
| 862 | CCCCAGCCAG | 252259 | ribosomal protein S3 | |
| 863 | CCCCAGCCAG | 334861 | hypothetical protein FLJ23059 | |
| 864 | TAAATAATTT | 1197 | heat shock 10kD protein 1 (chaperonin 10) | |
| 865 | ATAATTCTTT | 288806 | Homo sapiens cDNA FLJ11778 fis, clone HEMBA1005911 | |
| 866 | ATAATTCTTT | 539 | ribosomal protein S29 | |
| 867 | TTAAACCTCA | 170311 | heterogeneous nuclear ribonucleoprotein D-like | |
| 868 | TTAAACCTCA | 347810 | ESTs | |
| 869 | GCCGAGGAAG | 339696 | ribosomal protein S12 | |
| 870 | GCCGAGGAAG | 143067 | KIAA1602 protein | |
| 871 | GCCTGTATGA | 180450 | ribosomal protein S24 | |
| 872 | GCCTGTATGA | 356794 | ESTs, Weakly similar to RS24 ARATH 40S ribosomal protein S24 [A.thaliana] | |
| 873 | GTGTTAACCA | 74267 | ribosomal protein L15 | |
| 874 | CTTCGAAACT | 51299 | NADH dehydrogenase (ubiquinone) flavoprotein 2 (24kD) | |
| 875 | AAGGTCGAGC | 184582 | ribosomal protein L24 | |
| 876 | AAGGTCGAGC | 356004 | ESTs, Weakly similar to T47559 60S ribosomal protein-like | |
| 877 | CTTTGGAAAT | 6820 | cyclin fold protein 1 | |
| 878 | CTTTGGAAAT | 184222 | Down syndrome critical region gene 1 | |
| 879 | CCCCCTGOAT | 275243 | S100 calcium binding protein A6 (calcyclin) | |
| 880 | CGCCGGAACA | 356448 | ESTs, Weakly similar to RL4B ARATH 60S ribosomal protein L4-B (L1) | |
| | | | [A.thaliana] | |
| 881 | CGCCGGAACA | 286 | ribosomal protein L4 | |
| 882 | GTGTTGCACA | 301251 | Homo sapiens cDNA FLJ12014 fis, clone HEMBB1001685 | |
| 883 | GTGTTGCACA | 165590 | ribosomal protein S13 | |
| 884 | CAACTTAGTT | 180224 | myosin regulatory light chain | |
| 885 | GGGGCAGGGC | 9383 | cysteine-rich with EGF-like domains 1 | |
| 886 | CCAAGTTTTT | 75914 | coated vesicle membrane protein | |
| 887 | TTGGCAGCCC | 76064 | ribosomal protein L27a | |
| 888 | GTTAACGTCC | 178391 | ribosomal protein L36a | |
| 889 | GTTAACGTCC | 355599 | ESTs, Moderately similar to putative ribosomal protein [Arabidopsis thaliana] | |
| | | | [A.thaliana] | |
| 890 | GGAAGTTTCG | 55847 | mitochondrial ribosomal protein L51 | |
| 891 | CCCGTCCGGA | 180842 | ribosomal protein L13 | |
| 892 | CCCGTCCGGA | 356148 | ESTs, Weakly similar to 60S ribosomal protein L13 [Arabidopsis thaliana] | |
| | | | [A.thaliana] | |
| 893 | GGCCGCGTTC | 5174 | ribosomal protein S17 | |
| 894 | GGCCGCGTTC | 356626 | Homo sapiens cDNA FLJ34449 fis, clone HLUNG2002145 | |
| 895 | AAAAGAAACT | 172182 | poly(A) binding protein, cytoplasmic 1 | |
| 896 | AAAAGAAACT | 354497 | ESTs | |
| 897 | AACTCCCAGT | 110571 | growth arrest and DNA-damage-inducible, beta | |
| 898 | AACTCCCAGT | 118126 | protective protein for beta-galactosidase (galactosialidosis) | |
| 899 | CACTTTTGGG | 321497 | Homo sapiens cDNA FLJ31347 fis, clone MESAN2000023 | |
| 900 | CACTTTTGGG | 334851 | LIM and SH3 protein 1 | |
| 901 | GGGAGGGAAG | 75243 | bromodomain containing 2 | |
| 902 | GGGAGGGAAG | 160953 | p53-regulated apoptosis-inducing protein 1 | |
| 903 | GGGGGAATTT | 129548 | heterogeneous nuclear ribonucleoprotein K | |
| 904 | CATCTAAACT | 180900 | Williarns-Beuren syndrome chromosome region 1 | |
| 905 | TCCCCGTGGC | 75616 | 24-dehydrocholesterol reductase | |
| 906 | TCCCCGTGGC | 356547 | hypothetical protein BC016005 | |
| 907 | GCCTGCAGTC | 31439 | serine protease inhibitor, Kuritz type, 2 | |
| 908 | GCCTGCAGTC | 273385 | GNAS complex locus | |
| 909 | AGAATTTGCA | 250655 | prothymosin, alpha (gene sequence 28) | |
| 910 | AGAATTTGCA | 374658 | ESTs, Highly similar to TNHUA prothymosin alpha | |
| 911 | TCGGAGCTGT | 4055 | Homo sapiens mRNA; cDNA DKFZp564C2063 (from clone DKFZp564C2063) | |
| 912 | CACACAGTTT | 204354 | ras homolog gene family, member B | |
| 913 | GTAATCCTGC | | | |
| 914 | AGAGGTGTAG | | | |
| 915 | TTAGCCAGGC | 71367 | similar to RIKEN cDNA 1110058L19 | |
| 916 | TTAGCCAGGC | 161640 | tyrosine aminotransferase | |
| 917 | TGGAAAGTGA | 25647 | v-fos FBJ murine osteosarcoma viral oncogene homolog | |
| 918 | TGGAAAGTGA | 101047 | transcription factor 3 (E2A immunoglobulin enhancer binding factors E12/E47) | |
| 919 | TCCCTATTAA | | | |
| 920 | AGGAGCGGGG | 252189 | syndecan 4 (amphiglycan, ryudocan) | |
| 921 | GCCCCTCCGG | 83753 | small nuclear ribonucleoprotein polypeptides B and B1 | |
| 922 | GCCCCTCCGG | 180859 | 16.7Kd protein | |
| 923 | GCTGCCCTTG | 348557 | tubulin alpha 6 | |
| 924 | GCTGCCCTTG | 272897 | tubulin, alpha 3 | |
| 925 | CCACCCCGAA | 74637 | testis enhanced gene transcript (BAX inhibitor 1) | |
| 926 | GCTGCGGTCC | 795 | H2A histone family, member O | |
| 927 | GCTGCGGTCC | 106061 | RD RNA-binding protein | |
| 928 | GAGATCCGCA | 75348 | proteasome (prosome, macropain) activator subunit 1 (PA28 alpha) | |
| 929 | CAGAGATGAA | 8997 | Sad1 unc-84 domain protein 1 | |
| 930 | GCAAGCCAAC | | | |
| 931 | TGGCCTGCCC | 181002 | MLL septin-like fusion | |
| 932 | GCGGGGTGGA | 85155 | zinc finger protein 36, C3H type-like 1 | |
| 933 | AGGTGGCAAG | | | |
| 934 | TCGAAGCCCC | 198281 | pyruvate kinase, muscle | |
| 935 | TTTAACGGCC | | | |
| 936 | ACTTTCCAAA | 78921 | A kinase (PRKA) anchor protein 1 | |
| 937 | TGGAAGCACT | 624 | interleukin 8 | |
| 938 | GTCCGAGTGC | 351316 | transmembrane 4 superfamily member 1 | |
| 939 | TAACAGCCAG | 81328 | nuclear factor of kappa light polypeptide gene enhancer in B-cells inhibitor, | |
| | | | alpha | |
| 940 | TAACAGCCAG | 235498 | hypothetical protein FLJ14075 | |
| 941 | GCCTTGGGTG | 2250 | leukemia inhibitory factor (cholinergic differentiation factor) | |
| 942 | TTTGAAATGA | 28491 | spermidine/spermine NI-acetyltransferase | |
| 943 | GGGTAGGGGG | 13323 | hypothetical protein FLJ22059 | |
| 944 | ATCGTGGCGG | 5372 | claudin 4 | |
| 945 | ATCGTGGCGG | 8026 | sestrin 2 | |
| 946 | CCTGGCCTAA | 297285 | ESTs, Weakly similar to ZF37 HUMAN Zinc finger protein ZFP-37 [H.sapiens] | |
| 947 | CCTGGCCTAA | 111676 | protein kinase H11 | |
| 948 | AAGATTGGTG | 1244 | CD9 antigen (p24) | |
| 949 | AATCCTGTGG | 43910 | CD164 antigen, sialomucin | |
| 950 | AATCCTGTGG | 178551 | ribosomal protein L8 | |
| 951 | TGGTGTTGAG | 275865 | ribosomal protein S18 | |
| 952 | TGGTGTTGAG | 374510 | ESTs, Highly similar to S30393 ribosomal protein S18, cytosolic | |
| 953 | CTGGCCCTCG | 350470 | trefoil factor 1 (breast cancer, estrogen-inducible sequence expressed in) | |
| 954 | CTGGCCCTCG | 43654 | ceroid-lipofuscinosis, neuronal 6, late infantile, variant | |
| 955 | GACTCTTCAG | 234726 | serine (or cysteine) proteinase inhibitor, clade A (alpha-1 antiproteinase, | |
| | | | antitrypsin), member 3 | |
| 956 | CTGCCAACTT | 180370 | cofilin 1 (non-muscle) | |
| 957 | GTGCGCTGAG | 181244 | major histocompatibility complex, class I, A | |
| 958 | GTGCGCTGAG | 277477 | major histocompatibility complex, class I, C | |
| 959 | TTGGGGTTTC | 62954 | ferrilin, heavy polypeptide 1 | |
| 960 | TTGGGGTTTC | 374602 | ESTs, Weakly similar to putative ferritin [Arabidopsis thaliana] [A.thaliana] | |
| 961 | GGAGGGGGCT | 77886 | lamin A/C | |
| 962 | GGAGGGGGCT | 110642 | neurotensin receptor 1 (high affinity) | |
| 963 | TTAGTTTTTA | 323949 | kangai 1 (suppression of tumorigenicity 6, prostate; CD82 antigen (R2 | |
| | | | leukocyte antigen, antigen detected by monoclonal and antibody IA4)) | |
| 964 | TTAGTTTTTA | 274404 | plasminogen activator, tissue | |
| 965 | CCCAAGCTAG | 76067 | heat shock 27kD protein 1 | |
| 966 | CCCAAGCTAG | 374617 | ESTs, Highly similar to HHHU27 heat shock protein 27 | |
| 967 | GTGCACTGAG | 181244 | major histocompatibility complex, class I, A | |
| 968 | GTGCACTGAG | 277477 | major histocompatibility complex, class I, C | |
| 969 | CAGACTTTTT | 293884 | helicase/primase complex protein | |
| 970 | CAGACTTTTT | 78683 | ubiquitin specific protease 7 (herpes virus-associated) | |
| 971 | AAAACATTCT | 323562 | hypothetical protein DKFZp564K142 similar to implantation-associated protein | |
| 972 | CACCTAATTG | | | |
| 973 | GGGACGAGTG | | | |
| 974 | CAAGCATCCC | | | |
| 975 | AGCAGATCAG | 119301 | S100 calcium binding protein A10 (annexin II ligand, calpactin I, light poly- | |
| | | | peptide (p11)) | |
| 976 | AGCCCTACAA | 95243 | transcription elongation factor A (SII)-like 1 | |
| 977 | TGAAGTAACA | 150580 | putative translation initiation factor | |
| 978 | GCTAGGTTTA | | | |
| 979 | CAAAATCAGG | 79933 | cyclin I | |
| 980 | GGCTGGGGGC | 75721 | profilin I | |
| 981 | GGCTGGGGGC | 352407 | chromosome 1 amplified sequence 3 | |
| 982 | GGCCCTAGGC | 78909 | zinc finger protein 36, C3H type-like 2 | |
| 983 | GCTGAACGCG | 99029 | CCAAT/enhancer binding protein (C/EBP), beta | |
| 984 | AAGAGCGCCG | 8997 | Sad1 unc-84 domain protein 1 | |
| 985 | AAGAGCGCCG | 274402 | heat shock 70kD protein 1B | |
| 986 | AGGGTGAAAC | 77608 | splicing factor, arginine/serine-rich 9 | |
| 987 | AGGGTGAAAC | 363356 | EST | |
| 988 | GATCCCAACT | 118786 | metallothionein 2A | |
| 989 | GCCTACCCGA | 23582 | tumor-associated calcium signal transducer 2 | |
| 990 | CCAGGAGGAA | 276 | farnesyltransferase, CAAX box, beta | |
| 991 | CCAGGAGGAA | 180414 | heat shock 70kD protein 8 | |
| 992 | CCAGTGGCCC | 180920 | ribosomal protein S9 | |
| 993 | CCAGTGGCCC | 356713 | ESTs, Moderately similar to T49955 40S ribosomal protein-like | |
| 994 | GAAGCTTTGC | 289088 | heat shock 90kD protein 1, alpha | |
| 995 | GAAGCTTTGC | 356532 | ESTs, Moderately similar to 1908431A heat shock protein HSP81-1 [Arabidopsis | |
| | | | thaliana] [A.thaliana] | |
| 996 | TGTGTTGAGA | 181165 | eukaryotic translation elongation factor 1 alpha 1 | |
| 997 | TGTGTTGAGA | 356428 | Homo sapiens mRNA expressed only in placental villi, clone SMAP83 | |
| 998 | GTGACAGAAG | 129673 | eukaryotic translation initiation factor 4A, isoform I | |
| 999 | GTGACAGAAG | 356129 | ESTs, Weakly similar to JC1453 translation initiation factor eIF-4A2 | |
| 1000 | CCTCGGAAAA | 2017 | ribosomal protein L38 | |
| 1001 | CCTCGGAAAA | 343481 | ESTs, Weakly similar to RL38 ARATH 60S ribosomal protein L38 [A.thaliana] | |
| 1002 | CTCATAAGGA | | | |
| 1003 | CTAGCCTCAC | 14376 | actin, gamma 1 | |
| 1004 | GGGCCAACCC | 119475 | cold inducible RNA binding protein | |
| 1005 | GGGCCAACCC | 226795 | glutathione S-transferase pi | |
| 1006 | ACCCCCCCGC | 2780 | jun D proto-oncogene | |
| 1007 | GGTGCCCAGT | 75607 | myristoylated alanine-rich protein kinase C substrate | |
| 1008 | GCTTTATTTG | 288061 | actin, beta | |
| 1009 | GGCTCCCACT | 74335 | heat shock 90kD protein 1, beta | |
| 1010 | CTAAGACTTC | | | |
| 1011 | GGGTAGCTGG | | | |
| 1012 | ACCCACGTCA | 298184 | potassium voltage-gated channel, shaker-related subfamily, beta member 2 | |
| 1013 | ACCCACGTCA | 198951 | jun B proto-oncogene | |
| 1014 | GGGCAGGCGT | 737 | immediate early protein | |
| 1015 | GTTCACTGCA | 77318 | platelet-activating factor acetylhydrolase, isoform Ib, alpha subunit (45kD) | |
| 1016 | GTTCACTGCA | 168383 | intercellular adhesion molecule 1 (CD54), human rhinovirus receptor | |
| 1017 | ACTCAGCCCG | 101382 | tumor necrosis factor, alpha-induced protein 2 | |
| 1018 | ACTCAGCCCG | 4990 | KIAA1089 protein | |
| 1019 | TGATTTCACT | | | |
| 1020 | AGGTTTCCTC | 9736 | proteasome (prosome, macropain) 26S subunit, non-ATPase, 3 | |
| 1021 | ACCATCCTGC | 32963 | cadherin 6, type 2, K-cadherin (fetal kidney) | |
| 1022 | ACCATCCTGC | 76095 | immediate early response 3 | |
| 1023 | GGGAGGTAGC | 171825 | basic helix-loop-helix domain containing, class B, 2 | |
| 1024 | CCGTCCAAGG | 80617 | ribosomal protein S16 | |
| 1025 | CTCACCGCCC | 183650 | cellular retinoic acid binding protein 2 | |
| 1026 | CCCGCCCCCG | 155048 | Lutheran blood group (Auberger b antigen included) | |
| 1027 | ACTAACACCC | | | |
| 1028 | CACTACTCAC | | | |
| 1029 | CAGGAGGAGT | 289101 | glucose regulated protein, 58kD | |
| 1030 | CAGGAGGAGT | 356023 | ESTs, Weakly similar to PDI2 ARATH Probable protein disulfide isomerase 2 | |
| | | | precursor (PDI) [A.thaliana] | |
| 1031 | GCGACCGTCA | 273415 | aldolase A, fructose-bisphosphate | |
| 1032 | AAGGGAGGGT | 182248 | sequestosome 1 | |
| 1033 | GGCAGCCAGA | 75061 | macrophage myristoylated alanine-rich C kinase substrate | |
| 1034 | GGCAGCCAGA | 144501 | ESTs | |
| 1035 | TGTGGGTGCT | 306339 | Homo sapiens mRNA; cDNA DKFZp586N2022 (from clone DKFZp586N2022) | |
| 1036 | CGTGGGTGCT | 194657 | cadherin 1,type 1, E-cadherin (epithelial) | |
| 1037 | ATTTGAGAAG | 178658 | RAD23 homolog B (S. cervisiae) | |
| 1038 | AATGGAAATC | 4943 | melanoma antigen, family D, 2 | |
| 1039 | AATGGAAATC | 58103 | A kinase (PRKA) anchor protein (yotiao) 9 | |
| 1040 | TTTGGGCCTA | 17409 | cystein rich protein (CRPI) | |
| 1041 | CAACTAATTC | 69997 | zinc finger protein 238 | |
| 1042 | CAACTAATTC | 75106 | clusterin (complement lysis inhibitor, SP-40,40, sulfated glycoprotein 2, | |
| | | | testosterone-repressed prostate message 2, apolipoprotein 1) | |
| 1043 | GTTGTGGTTA | 75415 | beta-2-microglobulin | |
| 1044 | GTTGTGGTTA | 99785 | Honio sapiens cDNA: FLJ21245 fis, clone COL01184 | |
| 1045 | TTAAATGGAA | 33944 | ESTs, Weakly similar to hypothetical protein FLJ20489 [Homo sapiens] | |
| | | | [H.sapiens] | |
| 1046 | TTAAATGGAA | 351593 | fibrinogen, A alpha polypeptide | |
| 1047 | CTTAAAAAAA | 306309 | Homo sapiens mRNA; cDNA DKFZp566L0824 (from clone DKFZp566L0824) | |
| 1048 | CTTAAAAAAA | 75063 | human immunodeficiency virus type I enhancer binding protein 2 | |
| 1049 | CTTCTCCAAA | 151242 | serine (or cysteine) proteinase inhibitor, clade G (C1 inhibitor), member 1, | |
| | | | (angioedema, hereditary) | |
| 1050 | CTTCTCCAAA | 6671 | COP9 constitutive photomorphogenic homolog subunit 4 (Arabidopsis) | |
| 1051 | TACCTGCAGA | 100000 | S100 calcium binding protein A8 (calgranulin A) | |
| 1052 | ATAATAAAAG | 89690 | GRO3 oncogene | |
| 1053 | ATAATAAAAG | 250879 | Homo sapiens cDNA FLJ25968 fis, clone CBR01977 | |
| 1054 | AGAAAGATGT | 352541 | hypothetical protein MGC29937 | |
| 1055 | AGAAAGATGT | 78225 | annexin A1 | |
| 1056 | GTGCGGAGGA | 332053 | serum amyloid A1 | |
| 1057 | GTGCGGAGGA | 336462 | serum amyloid A2 | |
| 1058 | GGAAAAGTGG | 265317 | hypothetical protein MGC2562 | |
| 1059 | GGAAAAGTGG | 297681 | serine (or cysteine) proteinase inhibitor, clade A (alpha-I antiproteinase, | |
| | | | antitrypsin), member 1 | |
| 1060 | AATAGGTCCA | 113029 | ribosomal protein S25 | |
| 1061 | AATAGGTCCA | 356801 | ESTs, Weakly similar to T08568 ribosomal protein S25, cytosolic | |
| 1062 | GTTTATGGAT | 365706 | matrix Gla protein | |
| 1063 | CAACAATAAT | 283683 | chromosome 8 open reading frame 4 | |
| 1064 | TTTATTTTAA | 46452 | secretoglobin, family 2A, member 2 | |
| 1065 | CTTCCTGTGA | 348419 | small breast epithelial mucin | |
| 1066 | TAAAAACTTT | 204096 | secretoglobin, family 1D, member 2 | |
| 1067 | TAAAAACTTT | 343411 | Homo sapiens mRNA; cDNA DKFZp586K2322 (from clone DKFZp586K2322) | |
| 1068 | ACACAGCAAG | 27115 | ESTs, Weakly similar to SFRB HUMAN Splicing factor arginine/serine-rich 11 | |
| | | | (Arginine-rich 54 kDa nuclear protein) (P54) [H.sapiens] | |
| 1069 | TGCAGCACGA | 277477 | major histocompatibility complex, class I, C | |
| 1070 | TGCAGCACGA | 110309 | major histocompatibility complex, class I, F | |
| 1071 | ACTCCAAAAA | 356465 | ESTs, Moderately similar to S71259 ribosomal protein S15, cytosolic | |
| 1072 | ACTCCAAAAA | 344078 | Homo sapiens, clone IMAGE:3840457, mRNA | |
| 1073 | GCCTCCTCCC | 283781 | muscle specific gene | |
| 1074 | GCCTCCTCCC | 319084 | EST | |
| 1075 | AAGCTCGCCG | 62492 | secretoglobin, family 3A, member 1, HIN-1 | |
| 1076 | CCTGGTCCCA | 23881 | keratin 7 | |
| 1077 | CCTGGTCCCA | 167679 | SH3-domain binding protein 2 | |
| 1078 | GAATTAACAT | 79474 | tyrosine 3-monooxygenase/tryptophan 5-monooxygenase activation protein, | |
| | | | epsilon polypeptide | |
| 1079 | GAATTAACAT | 90073 | CSE1 chromosome segregation 1-like (yeast) | |
| 1080 | TAATTTGCGT | 79368 | epithelial membrane protein 1 | |
| 1081 | TTGGTTTTTG | 164021 | small inducible cytokine subfamily B (Cys-X-Cys), member 6 (granulocyte | |
| | | | chemotactic protein 2) | |
| 1082 | TTGGTTTTTG | 170088 | SLC2A4 regulator | |
| 1083 | GCTTGCAAAA | 6823 | neuropilin (NRP) and tolloid (TLL)-like 2 | |
| 1084 | GCTTGCAAAA | 372783 | superoxide dismutase 2, mitochondrial | |
| 1085 | GCCGCCCTGC | 76394 | enoyl Coenzyme A hydratase, short chain, 1, mitochondrial | |
| 1086 | GCCGCCCTGC | 82208 | acyl-Coenzyme A dehydrogenase, very long chain | |
| 1087 | CTTCCAGCTA | 217493 | annexin A2 | |
| 1088 | CTTCCAGCTA | 101651 | Homo sapiens mRNA; cDNA DKFZp434C107 (from clone DKFZp434C107) | |
| 1089 | CGAATGTCCT | 335952 | keratin 6B | |
| 1090 | TTGAAACTTT | 789 | GRO1 oncogene (melanoma growth stimulating activity, alpha) | |
| 1091 | TTGAAGCTTT | 302738 | Homo sapiens, cDNA: FLJ21425 fis, clone COL04162 | |
| 1092 | CCCGGGAGCG | 75807 | PDZ and LIM domain 1 (elfin) | |
| 1093 | CCCGGGAGCG | 273186 | chaperone, ABC1 activity of bcI complex like (S. pombe) | |
| 1094 | GGACTCTGGA | 71 | alpha-2-glycoprotein 1, zinc | |
| 1095 | GGACTCTGGA | 56023 | brain-derived neurotrophic factor | |
| 1096 | GTCTTAAAGT | 177781 | Homo sapiens, clone IMAGE:4711494, mRNA | |
| 1097 | CAGCTCACTG | 738 | ribosomal protein L14 | |
| 1098 | CAGCTCACTG | 356012 | ESTs, Weakly similar to T06039 ribosomal protein L14 homolog T24A18.40 | |

### Example 3

### Molecular Markers in DCIS

To determine if there are genes that are statistically significantly more likely to be expressed in DCIS than in invasive tumors (and vice versa), various statistical tests were performed (see Example 1). Based on these analyses, the levels of expression of CD74 and a SAGE tag (CTGGGCGCCC) (SEQ ID NO:1109) with no database match were found to be significantly greater in invasive or metastatic tumors than in DCIS (p=0.02 and p=0.05, respectively, Table 4). The samples studied were the same as those shown in Table 1; the sample designated “M1” in Table 4 was the same as that designated “MET” in Table 1. The expression of MGC2328, IBC-1, and eight other genes was also more likely to occur in invasive/metastatic tumors than in DCIS, but none of these differences in expression reached statistical significance (Table 4). Similarly the expression of S100A7 and keratin 19 (“KRT19”) was more frequent and at higher levels in DCIS than in invasive/metastatic tumors but this difference in expression was only marginally statistically significant.

In a second statistical analysis, ROC (receiver operating characteristic) curve analysis was used to choose the “best cut-off” for values, i.e., the cut-off that results in the most samples being correctly classified as DCIS or invasive, weighing both kinds of misclassification equally (Table 4). Tags that do not include 0.50 in the confidence interval (CI) could be useful for the differential diagnosis of in situ versus invasive carcinomas. Such tags include all those with p≦0.13 using the higher of two normals' cut-off as well as 3 other high in DCIS tags and 3 other high in invasive tags (Table 4). Using the best cut-off values, several of the SAGE tags correctly classified most of the DCIS and invasive SAGE libraries. For example KRT19 expression classified 75% of the DCIS and 0% of the invasive libraries as DCIS, while MGC23280 expression diagnosed 78% of the invasive cancer and 0% of the DCIS libraries as “invasive”. Thus, MGC23280 expression had 78% sensitivity and 100% specificity to correctly categorize breast tumors as DCIS or invasive/metastatic in this data set.

TABLE 4
Genes specific for in situ and invasive or metastatic breast cancer SAGE libraries

| | | | | | | ROC | | | | | | | | | | | | | | | | | | | | | | |
|---|---|---|---|---|---|---|---|---|---|---|---|---|---|---|---|---|---|---|---|---|---|---|---|---|---|---|---|---|
| | | | | | | area | ROC | DCIS | IDC | | | | | | | | | | | | | | | | | | | |
| SEQ | | | | | ROC | x100 | best | % > | % > | | | | | | | | | | | | | | | | | | | |
| ID | Tag | | | | area | 95% | cut- | cut- | cut- | | | | | | | | | | | | | | | | | | | |
| NO: | sequence | Unigene | Gene | P-value | x100 | Cl | off | off | off | N1 | N2 | D1 | D2 | D3 | D4 | D5 | D6 | D7 | T18 | 11 | 12 | 13 | 14 | 15 | 16 | LN1 | LN2 | M1 |

DCIS specific genes

| | | | | | | | | | | | | | | | | | | | | | | | | | | | | |
|---|---|---|---|---|---|---|---|---|---|---|---|---|---|---|---|---|---|---|---|---|---|---|---|---|---|---|---|---|
| 1099 | GAGCAGCGCC | 112408 | S100A7*(psoriasin) | 0.29 | 92 | 71-100 | 2.00 | 88 | 11 | 18 | 0 | 1018 | 3 | 3 | 373 | 16 | 1 | 2 | 890 | 0 | 0 | 0 | 1 | 0 | 20 | 0 | 0 | 0 |
| 1100 | GCTCTGCTTG | 112408 | S100A7*(psoriasin) | 0.08 | 69 | 51-87 | 54.70 | 38 | 0 | 2 | 0 | 76 | 0 | 0 | 20 | 0 | 0 | 0 | 55 | 0 | 0 | 0 | 0 | 0 | 0 | 0 | 0 | 0 |
| 1101 | GGACCTTTAT | 352107 | TFF3*(trefoil factor 3) | 0.33 | 64 | 35-93 | 3.00 | 50 | 11 | 2 | 0 | 23 | 3 | 0 | 1 | 23 | 1 | 0 | 37 | 2 | 1 | 1 | 0 | 1 | 0 | 4 | 3 | 0 |
| 1102 | CTCCACCCGA | 352107 | TFF3*(trefoil factor 3) | 1.00 | 69 | 42-97 | 16.80 | 100 | 56 | 34 | 7 | 511 | 854 | 17 | 26 | 451 | 31 | 38 | 261 | 369 | 124 | 15 | 0 | 94 | 16 | 285 | 244 | 2 |
| 1103 | GTGGCCACGG | 112405 | S100A9 (calgranulin B) | 0.29 | 85 | 63-100 | 4.10 | 88 | 22 | 29 | 30 | 200 | 0 | 9 | 238 | 4 | 20 | 15 | 92 | 0 | 1 | 1 | 3 | 0 | 72 | 0 | 0 | 4 |
| 1104 | GACATCAAGT | 182265 | KRT19 (keratin 19) | 0.06 | 83 | 58-100 | 58.90 | 75 | 0 | 33 | 35 | 59 | 165 | 3 | 118 | 139 | 59 | 153 | 34 | 20 | 40 | 41 | 25 | 31 | 20 | 10 | 34 | 16 |
| 1105 | CCCTACCCTG | 75736 | APOD (apolipoprotein D) | 0.21 | 76 | 52-100 | 7.70 | 100 | 44 | 4 | 58 | 15 | 42 | 8 | 293 | 215 | 9 | 12 | 49 | 2 | 16 | 41 | 3 | 4 | 44 | 0 | 3 | 16 |

Invasive or metastatic breast cancer specific genes

| | | | | | | | | | | | | | | | | | | | | | | | | | | | | |
|---|---|---|---|---|---|---|---|---|---|---|---|---|---|---|---|---|---|---|---|---|---|---|---|---|---|---|---|---|
| 1106 | ACGTTAAAGA | 350570 | IBC-1 (Invasive Breast | 0.13 | 75 | 55-95 | 2.50 | 0 | 56 | 0 | 0 | 0 | 0 | 0 | 1 | 0 | 0 | 0 | 0 | 177 | 101 | 3 | 0 | 0 | 12 | 199 | 0 | 0 |
| | | | Cancer-1) | | | | | | | | | | | | | | | | | | | | | | | | | |
| 1107 | CCAGAGAGTG | 180184 | CPB1 (carboxypeptidase | 0.33 | 67 | 43-91 | 1.30 | 25 | 56 | 0 | 0 | 0 | 9 | 0 | 0 | 0 | 0 | 21 | 0 | 107 | 115 | 0 | 1 | 0 | 0 | 0 | 354 | 2 |
| | | | B1) | | | | | | | | | | | | | | | | | | | | | | | | | |
| 1108 | GGAGTAAGGG | 5163 | MGC23280 (hypothetical | 0.06 | 86 | 68-100 | 1.46 | 0 | 78 | 0 | 0 | 0 | 0 | 0 | 1 | 0 | 0 | 1 | 0 | 22 | 8 | 0 | 3 | 1 | 0 | 22 | 1 | 2 |
| | | | protein) | | | | | | | | | | | | | | | | | | | | | | | | | |
| 1109 | CTGGGCGCCC | NA | No reliable match | 0.05 | 80 | 61-99 | 12.00 | 0 | 56 | 0 | 0 | 0 | 0 | 2 | 0 | 0 | 0 | 0 | 0 | 40 | 25 | 0 | 0 | 0 | 12 | 26 | 1 | 34 |
| 1110 | CCAATAAAGT | 101850 | RBP1 (retinol binding | 0.33 | 78 | 54-100 | 6.40 | 25 | 78 | 2 | 0 | 0 | 3 | 0 | 0 | 2 | 6 | 11 | 7 | 49 | 28 | 6 | 8 | 0 | 0 | 102 | 32 | 21 |
| | | | protein) | | | | | | | | | | | | | | | | | | | | | | | | | |
| 1111 | TTTGTTTTTA | 131740 | FLJ30428 (hypothetical | 1.00 | 84 | 62-100 | 4.01 | 0 | 78 | 0 | 0 | 0 | 3 | 2 | 3 | 2 | 1 | 4 | 2 | 7 | 7 | 27 | 4 | 21 | 4 | 2 | 18 | 0 |
| | | | protein) | | | | | | | | | | | | | | | | | | | | | | | | | |
| 1112 | ATCCGCGAGG | 180142 | CLSP (calmodulin-like | 0.64 | 64 | 38-89 | 19.00 | 25 | 56 | 0 | 0 | 0 | 0 | 3 | 22 | 0 | 20 | 0 | 0 | 47 | 25 | 0 | 52 | 19 | 0 | 20 | 0 | 0 |
| | | | skin protein) | | | | | | | | | | | | | | | | | | | | | | | | | |
| 1113 | GACCACACCG | 367741 | NUDT8 (nudix) | 0.64 | 69 | 43-96 | 8.00 | 0 | 56 | 2 | 2 | 2 | 0 | 0 | 7 | 0 | 7 | 0 | 5 | 27 | 21 | 1 | 0 | 0 | 8 | 33 | 9 | 0 |
| 1114 | CGATATTCCC | 37616 | MGC14480 (hypothetical | 0.33 | 79 | 57-100 | 6.40 | 25 | 78 | 4 | 2 | 4 | 6 | 0 | 3 | 12 | 1 | 6 | 7 | 36 | 26 | 6 | 4 | 9 | 12 | 31 | 13 | 2 |
| | | | protein) | | | | | | | | | | | | | | | | | | | | | | | | | |
| 1115 | AAACCCCAAT | 181125 | IGL (immunoglobulin | 1.00 | 72 | 46-97 | 38.00 | 25 | 67 | 0 | 0 | 15 | 0 | 17 | 102 | 4 | 1 | 1 | 44 | 163 | 87 | 78 | 3 | 0 | 241 | 258 | 10 | 38 |
| | | | lambda) | | | | | | | | | | | | | | | | | | | | | | | | | |
| 1116 | GTTCACATTA | 84298 | CD74 antigen | 0.02 | 93 | 81-100 | 31.70 | 25 | 100 | 7 | 33 | 29 | 6 | 25 | 188 | 70 | 6 | 13 | 28 | 159 | 208 | 226 | 32 | 428 | 474 | 203 | 72 | 72 |

*From two transcripts (S100A7 and TFF3) two independent SAGE tags were derived and both found to be specific for DCIS.
P-value is based on using the SAGE tag number which was highest of two normals as cut-off.
The first ROC column gives the ROC area, the second the approximate 95% Cl, the third column gives the “best” cut-off, while the last two columns show the percent of DCIS specimens with values greater than or equal to the ROC best cut-off and the percent of invasive specimens with values greater than or equal to the ROC best cut-off.

Next, 26 genes that appeared to be the most highly differentially expressed between normal and DCIS samples or between intermediate (D2) and high-grade (D1) DCIS at p≦0.001 using the SAGE 2000 software were selected for further validation studies (Table 5). It was hypothesized that genes most highly differentially expressed between normal and DCIS tissue or two different types of DCIS tumors could be used as molecular markers for defining biologically and potentially clinically meaningful subgroups of DCIS. This concept was supported by the observation that clustering analysis of the eight DCIS libraries using only these 26 genes gave a dendrogram (FIG. 3C) that was almost identical to that obtained using 582 genes (FIG. 3B). In Table 5, the samples shown are the same as those shown in Table 4 and the column labeled “Method” indicates the technique used to validate the conclusions of the relevant SAGE data (ISH, in situ hybridization; IH, immunohistochemistry; ND, not done).

TABLE 5
Genes selected for mRNA in situ hybridization and immunohistochemical analyses

| SEQ | Tag | | | | | | | | | | | | | | | | | | | | | | |
|---|---|---|---|---|---|---|---|---|---|---|---|---|---|---|---|---|---|---|---|---|---|---|---|
| ID | Sequence | Unigene | Gene | N1 | N2 | D1 | D2 | D3 | D4 | D5 | D6 | D7 | T18 | 11 | 12 | 13 | 14 | 15 | 16 | LN1 | LN2 | M1 | Method |

“Normal specific”

| | | | | | | | | | | | | | | | | | | | | | | | |
|---|---|---|---|---|---|---|---|---|---|---|---|---|---|---|---|---|---|---|---|---|---|---|---|
| 1117 | AAGCTCGCCG | 62492 | SCGB3A1 (HIN-1, High in Normal-1) | 125 | 44 | 0 | 0 | 0 | 3 | 0 | 9 | 0 | 0 | 0 | 0 | 0 | 0 | 0 | 0 | 0 | 0 | 4 | ISH |
| 1118 | GTCCGAGTGC | 351316 | TM4SFI (transmembrane 4 superfamily | 134 | 96 | 11 | 33 | 11 | 1 | 2 | 23 | 13 | 4 | 2 | 0 | 0 | 8 | 0 | 8 | 2 | 3 | 5 | ISH |
| | | | member 1) | | | | | | | | | | | | | | | | | | | | |
| 1119 | GACTGCGCGT | 10086 | FN14 (Type I transmembrane protein Fn14) | 40 | 26 | 0 | 36 | 6 | 3 | 4 | 22 | 32 | 4 | 0 | 3 | 0 | 1 | 1 | 8 | 0 | 0 | 0 | ND |
| 1120 | TTGAAGCTTT | 75765 | CXCL2 (GRO2, growth related protein 2) | 122 | 247 | 2 | 3 | 15 | 0 | 0 | 29 | 5 | 0 | 0 | 1 | 4 | 0 | 0 | 0 | 0 | 0 | 0 | IH |
| 1121 | TTGAAACTTT | 789 | CXCL1 (GRO1, growth relaled protein 1) | 394 | 453 | 11 | 12 | 14 | 1 | 0 | 61 | 1 | 4 | 0 | 0 | 1 | 0 | 1 | 0 | 0 | 0 | 2 | IH |
| 1122 | TGGAAGCACT | 624 | IL-8 (interleukin-8) | 368 | 352 | 8 | 39 | 12 | 1 | 0 | 94 | 15 | 0 | 2 | 0 | 1 | 0 | 0 | 0 | 0 | 0 | 0 | IH |
| 1123 | TAACAGCCAG | 81328 | NFKBIA (NFKB inhibitor alpha) | 136 | 152 | 6 | 39 | 23 | 4 | 2 | 28 | 125 | 19 | 4 | 7 | 8 | 7 | 9 | 4 | 2 | 10 | 20 | IH |

“Tumor specific”

| | | | | | | | | | | | | | | | | | | | | | | | |
|---|---|---|---|---|---|---|---|---|---|---|---|---|---|---|---|---|---|---|---|---|---|---|---|
| 1124 | CAATTAAAAG | 149923 | XBP1 (X-box binding protein) | 80 | 58 | 147 | 196 | 29 | 366 | 322 | 27 | 97 | 214 | 244 | 247 | 535 | 18 | 531 | 129 | 199 | 599 | 7 | ISH |
| 1125 | TTTGGTGTTT | 83190 | FASN (fatty acid synthase) | 5 | 0 | 8 | 24 | 2 | 57 | 27 | 5 | 28 | 21 | 36 | 41 | 62 | 14 | 57 | 12 | 28 | 10 | 4 | IH |
| 1126 | TGATCTCCAA | 83190 | FASN (fatty acid synthase) | 16 | 5 | 53 | 63 | 6 | 201 | 182 | 31 | 47 | 5 | 168 | 33 | 105 | 17 | 314 | 4 | 254 | 46 | 21 | IH |
| 1127 | CTCCACCCGA | 82961 | TFF3 (trefoil factor 3) | 34 | 7 | 511 | 854 | 17 | 26 | 451 | 31 | 38 | 261 | 369 | 124 | 15 | 0 | 94 | 16 | 285 | 244 | 2 | ISH + IH |

“Intermediate-grade DCIS specific”

| | | | | | | | | | | | | | | | | | | | | | | | |
|---|---|---|---|---|---|---|---|---|---|---|---|---|---|---|---|---|---|---|---|---|---|---|---|
| 1128 | CGCCGACGAT | 265827 | IFI-6-16 (interferon alpha-uinducible protein) | 4 | 0 | 17 | 644 | 3 | 90 | 418 | 18 | 366 | 4 | 130 | 171 | 5 | 63 | 12 | 161 | 14 | 526 | 181 | ISH |
| 1129 | TTTGGGCCTA | 17409 | CRIP1 (cyteine-rich protein 1) | 33 | 5 | 21 | 66 | 29 | 22 | 33 | 49 | 223 | 4 | 7 | 49 | 37 | 0 | 35 | 4 | 2 | 60 | 7 | ISH |
| 1130 | AATCTGCGCC | 833 | ISG15 (interferon-stimulated protein, 15 kDa) | 0 | 0 | 2 | 48 | 2 | 3 | 20 | 1 | 42 | 2 | 9 | 5 | 1 | 0 | 1 | 28 | 4 | 29 | 16 | ISH |
| 1131 | CCAGGGGAGA | 278613 | IF127 (interferon alpha inducible protein) | 0 | 0 | 4 | 36 | 3 | 4 | 90 | 5 | 176 | 2 | 0 | 21 | 5 | 1 | 3 | 104 | 2 | 31 | 77 | ISH |
| 1132 | GAAAGATGCT | 334370 | BEX1 (brain expressed, X-linked 1) | 2 | 0 | 6 | 48 | 0 | 1 | 0 | 1 | 1 | 0 | 29 | 37 | 1 | 1 | 1 | 0 | 0 | 162 | 2 | ISH |
| 1133 | CAGACTTTTT | 293884 | LOC150678 (helicase/primatase protein) | 7 | 5 | 4 | 54 | 5 | 1 | 4 | 0 | 31 | 5 | 2 | 9 | 4 | 1 | 4 | 0 | 0 | 4 | 4 | ISH |
| 1134 | CTGGCGCCGA | 183180 | ANAPC11 (anaphase promoting complex | 4 | 2 | 11 | 42 | 2 | 7 | 29 | 2 | 2 | 12 | 22 | 17 | 19 | 11 | 15 | 28 | 26 | 28 | 20 | ND |
| | | | subunit 11) | | | | | | | | | | | | | | | | | | | | |
| 1135 | TGAGCTACCC | 72222 | FER1L4 (Fer-1-like 4) | 0 | 0 | 0 | 33 | 0 | 0 | 6 | 0 | 0 | 11 | 2 | 0 | 0 | 1 | 0 | 4 | 0 | 0 | 0 | ND |

“High-grade DCIS specific”

| | | | | | | | | | | | | | | | | | | | | | | | |
|---|---|---|---|---|---|---|---|---|---|---|---|---|---|---|---|---|---|---|---|---|---|---|---|
| 1136 | GAGCAGCGCC | 112408 | S100A7 (psoriasin) | 18 | 0 | 1018 | 3 | 3 | 373 | 16 | 1 | 2 | 890 | 0 | 0 | 0 | 1 | 0 | 20 | 0 | 0 | 0 | ISH + IH |
| 1137 | TTTGCACCTT | 75511 | CTGF (connective tissue growth factor) | 0 | 0 | 141 | 6 | 18 | 63 | 18 | 9 | 6 | 41 | 9 | 42 | 43 | 66 | 19 | 16 | 10 | 7 | 48 | ISH + IH |
| 1138 | TATGAGGGTA | 24950 | RGS5 (regulator of G-protein signaling 5) | 0 | 0 | 40 | 0 | 0 | 1 | 0 | 0 | 6 | 46 | 4 | 0 | 1 | 0 | 0 | 8 | 0 | 1 | 4 | ISH |
| 1139 | GAAGTTATAA | 137476 | PEG10 (paternally expressed 10) | 0 | 7 | 44 | 3 | 0 | 6 | 0 | 33 | 1 | 16 | 0 | 4 | 0 | 4 | 1 | 0 | 8 | 0 | 0 | ISH |
| 1140 | ATGTGAAGAG | 111779 | SPARC (osteonectin) | 4 | 0 | 118 | 3 | 6 | 79 | 39 | 22 | 6 | 12 | 112 | 97 | 185 | 47 | 194 | 96 | 163 | 32 | 129 | IH |
| 1141 | GAGAGAAAAT | 181444 | LOC51235 (hypthetical protein) | 0 | 2 | 40 | 9 | 0 | 10 | 6 | 7 | 7 | 21 | 4 | 8 | 9 | 11 | 18 | 0 | 6 | 10 | 27 | ND |
| 1142 | CTCCCCCAAA | 293441 | SNC73 (immunoglobulin heavy mu chain)* | 2 | 14 | 78 | 0 | 20 | 605 | 37 | 1 | 0 | 11 | 159 | 86 | 186 | 0 | 6 | 12 | 140 | 19 | 109 | ISH |

ISH = in situ hybridization, IH = immunohistochemistry, ND = not determined.
*The expression of SNC73 was found to be localized to leukocytes and was not pursued further.

### Example 4

### Confirmation of SAGE Gene Expression Studies by mRNA In Situ Hybridization

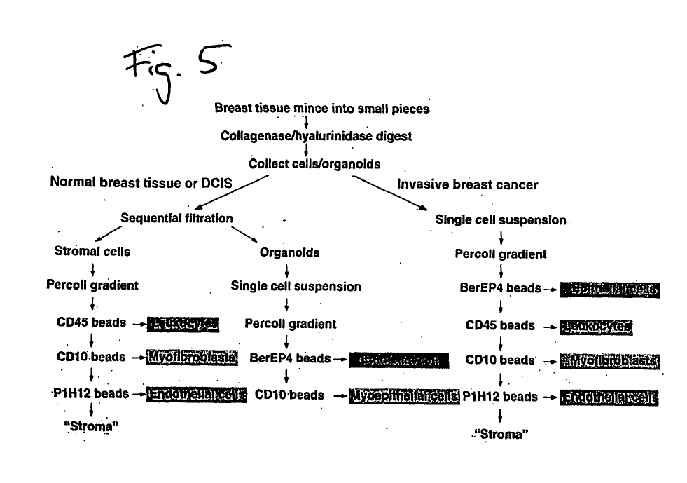

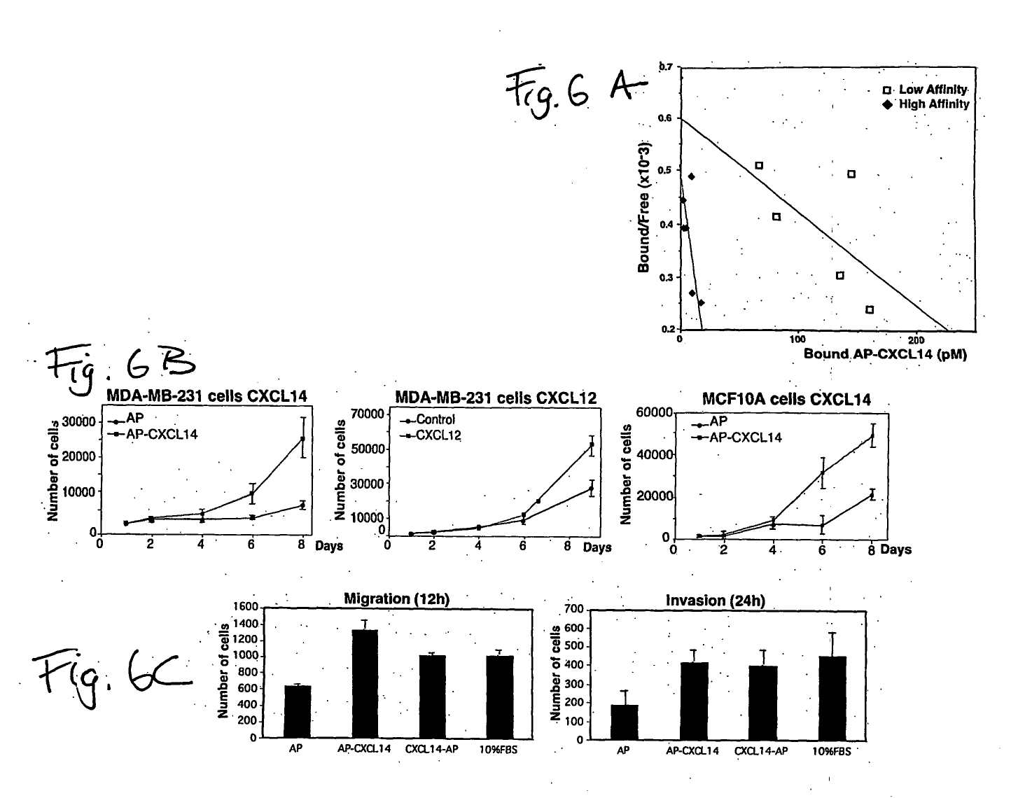

mRNA in situ hybridization determines gene expression at the cellular level and is particularly useful in solid tumors that are heterogeneous in cellular composition. Eighteen frozen DCIS and invasive breast cancer samples were used for such a study. Whenever possible tumors were selected to include normal, DCIS, and invasive components on the same slide in order to obtain expression data in these three stages of breast tumorigenesis. Examples of in situ hybridization results are depicted in FIG. 4A. Interestingly, the upregulation in expression of several genes in DCIS occurred mostly, or exclusively, in non-epithelial cells. Specifically, CTGF (Connective Tissue Growth Factor) and RGS5 (Regulator of G protein Signaling) were highly expressed in DCIS myoepithelial cells and stromal fibroblasts; in certain tumors expression was upregulated in DCIS epithelial cells as well (FIG. 4A). Cumulative scores for in situ hybridization were used for hierarchical clustering analysis and statistical tests. A dendrogram of the 18 different tumors and 5 normal breast tissues showed that, using the expression of 14 genes, it was possible to distinguish between normal and cancer samples and group the tumors into subclasses (FIG. 4B). Although a clustering analysis of gene expression profiles obtained by in situ hybridization in DCIS of different grades contained some inconsistent associations, there was an indication that, as shown by the clustering analysis of DCIS tumors using SAGE data, DCIS tumors of a particular grade were more similar to each other with respect to the expression of the 14 genes than they were to DCIS tumors of a different grade (data not shown). The expression of no single gene was found to distinguish between DCIS and invasive tumors; this finding confirmed the results of the SAGE analysis described above. Surprisingly, in the majority of cases, the in situ and invasive areas within particular tumors did not always show the highest similarity to each other (FIG. 4B). This result is consistent with the idea that gene expression profiles are not the same during tumor progression.

Fisher's exact test revealed significant positive correlation between the expression of TFF3 and IFI-6-16 (p=0.01), LOC51235 and BEX1 (p=0.05), while inverse correlation was found between the expression of S100A7 and RGS5Tu (p=0.04), S100A7 and TFF3 (p=0.04), and CTGF and TM4S5F1 (p=0.01). No statistically significant associations were found between the expression of any of these genes and histo-pathologic features of the tumors.

### Example 5

### Immunohistochemical Analysis of Gene Tissue Microarrays and Clinicopathologic Associations

The expression of 10 genes was analyzed by immunohistochemistry using tissue microarrays composed of tumors of different pathologic stages. In total, 788 tumor samples (675 primary invasive tumors, 33 metastases, 71 pure DCIS, and 9 DCIS with concurrent invasive carcinoma) obtained from eight different cohorts (tissue microarrays) were analyzed. Expression of all 10 genes was not analyzed in all cohorts. An example of immunohistochemical staining of a DCIS with antibodies specific for 5 gene products is depicted in FIG. 4C.

Cumulative scores for immunohistochemical staining were used for statistical analyses to determine associations between the expression of the genes and histo-pathologic features of the tumors or between different genes. In addition, S100A7 expression was analyzed with respect to clinical outcome (overall survival and distant metastasis free survival) in two of the patient cohorts.

As shown by the above-described SAGE analyses, the expression of IBC-1 was almost exclusively limited to a subset of invasive breast carcinomas, with only 2 out of 80 DCIS tumors showing detectable IBC-1 expression (FIG. 4C and data not shown). The expression of CTGF, TFF3, and SPARC in the stroma was statistically significantly related to pathologic stage with TFF3 and SPARC being less likely to be expressed in DCIS than in invasive or metastatic tumors (Table 6). Statistically significant association between S100A7 expression and estrogen receptor (ER) negativity, high histologic grade, and more than 4 positive lymph nodes was demonstrated in logistic-regression models in primary invasive tumors (Table 6). Since all these tumor characteristics are known to correlate with poor prognosis, it is likely that S100A7 expression identifies a clinically meaningful subgroup of tumors. Kaplan-Meier analysis demonstrated decreased overall survival for patients with S1007 A7 positive tumors, but this did not reach statistical significance (p=0.41), possibly due to relatively short patient follow-up data and insufficient sample size (data not shown). The expression of fatty acid synthase (FASN) was higher in ER negative and HER2 positive high-grade tumors, while the expression of SPARC (osteonectin) inversely correlated with high histologic grade and TNM stage 3 (Table 6). The fraction of breast tumors that expressed the cytokines CXCL1 (GRO1), CXCL2 (GRO2), and IL-8 was, as expected, very low, since the genes encoding them were more highly expressed in normal mammary epithelium than in breast cancer assessed by SAGE and immunohistochemistry (data not shown). Finally, using Fisher's exact test the expression of S100A7 was associated with a higher likelihood of expression of FASN (p=9.95×10⁻⁶) and TFF3 (p=0.002), and a lower likelihood of expression of CTGF (p=0.005), while the expression of FASN was associated with that of TFF3 (p=3.5×10⁻⁶) and SPARC in the tumor-cells (p=4×10⁻⁵).

TABLE 6
Relationships between gene expression and histopathologic features of tumors

| | | | DCIS | | | | | Invasive | | | | |
|---|---|---|---|---|---|---|---|---|---|---|---|---|
| | | | | #p- | age | | | Grade | | | | |
| | DCIS | Invasive | Metastasis | value | ≦50 | ER | HER2 | 1 | Grade 3 | Stage 3 | Tumor size | ≧4 pos LN |
| S100A7 | 23 (37.5) | 245 (43.4) | 16 (31.4) | 0.08 | p = | *p = 0.03 | NS | NS | p < | NS | NS | p = 0.0008 |
| | | | | | 0.03 | | | | 0.0001 | | | |
| FASN | 28 (38.9) | 126 (51.0) | 21 (50.0) | 0.2 | NS | p = 0.02 | p = | *p = | NS | NS | NS | NS |
| | | | | | | | 0.002 | 0.03 | | | | |
| TFF3 | 36 (52.2) | 196 (77.2) | 31 (75.6) | 0.0003 | NS | p = 0.02 | NS | NS | NS | NS | NS | NS |
| CTGF | 21 (30.0) | 88 (34.7) | 5 (12.2) | 0.01 | NS | NS | NS | NS | NS | NS | NS | NS |
| SPARC- | 27 (39.1) | 136 (50.4) | 21 (50.0) | 0.25 | NS | NS | NS | NS | *p = | *p = 0.02 | NS | NS |
| Tumor | | | | | | | | | 0.01 | | | |
| SPARC- | 63 (87.5) | 248 (91.2) | 42 (100.0) | 0.04 | NS | NS | NS | NS | NS | *p = 0.002 | p = 0.03 | NS |
| Stroma | | | | | | | | | | | | |
| CXCL1 | ND | 11 (15.9) | ND | NA | NA | NS | NS | NS | NS | NS | NS | NS |
| (GRO1) | | | | | | | | | | | | |
| CXCL2 | ND | 2 (3.1) | ND | NA | NA | NS | NS | NS | NS | NS | NS | NS |
| (GRO2) | | | | | | | | | | | | |
| IL-8 | ND | 5 (7.5) | ND | NA | NA | NS | NS | NS | NS | NS | NS | NS |
| NFKBIA | ND | 46 (93.9) | ND | NA | NA | NS | NS | NS | NS | NS | NS | NS |
| CCND1 | ND | 3 (10.7) | ND | NA | NA | NS | NS | NS | NS | NS | NS | NS |
| CD45 | ND | 28 (96.6) | ND | NA | NA | NS | NS | NS | NS | NS | NS | NS |

Numbers reflect the actual numbers of tumor specimens that were positive for the indicated gene, and the % of positive tumors is indicated in parenthesis.
Only data for which there was at least one statistically significant association is listed in the table.
#p-value is Fisher's exact test p-value for association between gene expression and tumor category (DCIS, Invasive, or Metastasis). All other p-values are likelihood ratio (LR) test p-values.
*denotes p-value for inverse correlation.

### Example 6

### Analysis of SAGE Libraries from Epithelial and Non-Epithelial Cells of Normal Breast and DCIS Tissue

The SAGE analyses described above indicated that, in breast cancer, dramatic changes occur not only in the cancerous epithelial cells, but also in various stromal cells. Surprisingly all these stromal changes were already present in pre-invasive tumors such as DCIS (ductal carcinoma in situ) that have not yet invaded the surrounding tissues. Interestingly, many of the genes up-regulated in tumor epithelial or stromal cells encode secreted proteins (Connective Tissue Growth Factor, Trefoil Factor 3, Osteonectin, IGFBP-7 etc.) implicating autocrine and/or paracrine regulatory loops among epithelial and stromal cells. Based on these results it was concluded that a comprehensive analysis of the gene expression profile of each cell type found in normal breast tissue and DCIS tissue, combined with the analysis of the genetic changes present in these cells would yield important new information on the role of epithelial-stromal interactions in breast tumorigenesis and will help define the cell type of origin of breast carcinomas. In addition, genes and pathways identified by such an approach will likely represent excellent candidate therapeutic targets.

Analysis of SAGE libraries from epithelial and non-epithelial cells from normal breast tissue and DCIS tumors identified 35 tags that are significantly (p≦0.002) differentially expressed between leukocytes (Table 7), 333 tags that are significantly (p≦0.002) differentially expressed between myoepithelial cells (Table 8), 146 tags that are significantly (p≦0.062) differentially expressed between luminal epithelial cells (Table 9), and 175 tags that are significantly (p≦0.002) differentially expressed between endothelial cells (Table 10) isolated from normal and two different DCIS tissue. In Tables 7-10, data obtained with normal breast tissue (NL) and one DCIS sample (Table 10: D6) or two DCIS samples (Tables 7-9: D6 and D7) are shown. The numbers of tags shown are normalized values (see Example 1). The ratio of the number of tags obtained from cells isolated from DCIS tissue to the number obtained with cells from normal breast tissue (d/n, d6/n, or d7/n) for each tag are shown. The tables also include the Unigene numbers and the names of previously identified genes. Where no Unigene number is shown, the relevant gene has not previously been identified.

Analysis of the SAGE data confirmed the findings of the RT-PCR analysis (see Example 1 and FIG. 2) that the cell purification procedure worked well in that certain genes known to be expressed in the cell types of interest were represented in the relevant SAGE libraries. For example, the leukocyte libraries had the highest level of expression of several immunoglobulin and certain interleukins, while the levels of IGFBP-7 and hevin, and selectin E (endothelial cell adhesion molecule) were highest in the endothelial cell SAGE libraries. Interestingly, keratin 7 and 17 were highly abundant in the normal, but significantly decreased in the DCIS myoepithelial libraries suggesting that maintaining the normal differentiation state of myoepithelial cells may require the presence of normal luminal mammary epithelial cells. In many of the genes, there was at least a 10-fold difference in expression between normal and one or both DCIS tissues tested; in Tables 7-10 the relevant genes are indicated by the symbol “d” at the end of the relevant tag sequence. Furthermore, at least among differentially expressed genes that were previously known, 44 in the endothelial, 11 in the leukocyte, 82 in the myoepithelial, and 29 in the luminal epithelial cells encode proteins that are either secreted or expressed on the cell surface and thus likely to be involved in epithelial-stromal cell interactions that regulate (up or down) tumor development and/or progression; Tables 11, 12, 13, and 14 list the relevant genes in leukocytes, myoepithelial cells, luminal epithelial cells, and endothelial cells, respectively.

TABLE 7
Genes differentially expressed in leukocytes from DCIS and normal breast tissue

| | | | | | | | |
|---|---|---|---|---|---|---|---|
| | SEQ | | | | | | |
| | ID | | | | | | |
| Tag_Sequence | NO: | NL | D6 | D7 | d/n | Unigene | Gene |

| | | | | | | | | |
|---|---|---|---|---|---|---|---|---|
| 1 | ACAGCGCTGA d | 1143 | 0 | 192 | 32 | Infinite | 375570 | HLA-DRB1, major histocompatibility complex, class II, |
| | | | | | | | | DR beta 1 |
| 2 | CAATTTGTGT d | 1144 | 0 | 44 | 32 | Infinite | 126256 | interleukin 1, beta |
| 3 | GCCGGGTGGG d | 1145 | 2 | 21 | 32 | 13 | 74631 | basigin (OK blood group), leukocyte activation M6 |
| | | | | | | | | antigen |
| 4 | CGACCCCACG d | 1146 | 14 | 164 | 60 | 8 | 169401 | apolipoprotein E |
| 5 | GCACCAAAGC d | 1147 | 19 | 396 | 192 | 16 | 73817 | small inducible cytokine A3 |
| 6 | GAAATACAGT d | 1148 | 6 | 128 | 69 | 16 | 67201 | NT5C, 5′,3′-nucleotidase, cytosolic |
| 7 | ACCGCCGTGG d | 1149 | 4 | 29 | 50 | 10 | 68877 | cytochrome b-245, alpha polypeptide-neutrophil specific |
| 8 | TCCCTGGCTG d | 1150 | 2 | 31 | 28 | 14 | 78575 | prosaposin, short alt. transcipt, 88% con. Match |
| 9 | GGGCATCTCT d | 1151 | 37 | 810 | 243 | 14 | 76807 | major histocompatibility complex, class II, DR alpha |
| 10 | ATCCGGACCC d | 1152 | 2 | 33 | 32 | 16 | 76556 | protein phosphatase 1, regulatory (inhibitor) subunit |
| | | | | | | | | 15A-induced by dNA damaga, may be involved in apoptosis |
| 11 | TTTGGGCCTA d | 1153 | 2 | 21 | 35 | 13 | 17409 | cysteine-rich protein 1 (intestinal) |
| 12 | GCTTTATTTG d | 1154 | 14 | 51 | 142 | 7 | 288061 | actin, beta |
| 13 | TTCCCTTCTT d | 1155 | 4 | 40 | 35 | 9 | 814 | major histocompatibility complex, class II, DP beta 1 |
| 14 | TCCAAATCGA d | 1156 | 4 | 64 | 38 | 12 | 297753 | vimentin |
| 15 | AACCACATTG d | 1157 | 2 | 22 | 41 | 15 | 179657 | plasminogen activator, urokinase receptor |
| 16 | GCGGTTGTGG d | 1158 | 17 | 181 | 76 | 8 | 79356 | Lysosomal-associated multispanning membrane protein-5, |
| | | | | | | | | haematopoetic cell specific |
| 17 | AAGTTGCTAT | 1159 | 6 | 37 | 54 | 7 | 78575 | prosaposin (variant Gaucher disease and variant meta- |
| | | | | | | | | chromatic leukodystrophy) |
| 18 | ATGTAAAAAA d | 1160 | 2 | 148 | 35 | 44 | 337778 | lysozyme (renal amyloidosis)-leukocyte spec |
| 19 | GTAGGGGTAA d | 1161 | 77 | 7 | 16 | 0 | | no confident match |
| 20 | GGGCCAGGGG d | 1162 | 37 | 7 | 3 | 0 | 111099 | hypothetical protein MGC10974, some homology to |
| | | | | | | | | collagen a |
| 21 | GGGGGACGGC d | 1163 | 41 | 3 | 6 | 0 | 367663 | cDNA FLJ37864 fis, clone BRSSN2015982, 86% conf. match; |
| | | | | | | | | some homology to actinin |
| 22 | CTGTTGGTGA | 1164 | 60 | 11 | 13 | 0 | 3463 | 40S RIBOSOMAL PROTEIN S23 |
| 23 | TAAGGAGCTG d | 1165 | 234 | 17 | 32 | 0 | 299465 | RS26_HUMAN 40S RIBOSOMAL PROTEIN S26 |
| 24 | ACAAAAACTA d | 1166 | 48 | 5 | 6 | 0 | | mitochondrial |
| 25 | TGGCTAAAAA d | 1167 | 35 | 4 | 3 | 0 | T52757 | EST, but only 77% confidence match |
| 26 | ACTTTTTAAA d | 1168 | 66 | 3 | 6 | 0 | BG2161 | ESTs |
| 27 | TACAGAGGGA d | 1169 | 29 | 4 | 0 | 0 | 3776 | zinc finger protein 216 |
| 28 | CTCCACCCGA d | 1170 | 79 | 8 | 0 | 0 | 352107 | trefoil factor 3 (intestinal) |
| 29 | AGCTGTCCCC d | 1171 | 130 | 7 | 3 | 0 | | mitochondrial |
| 30 | TGAAGCAGTA d | 1172 | 27 | 2 | 0 | 0 | AA12959 | EST |
| 31 | TAATAAAGAA d | 1173 | 27 | 1 | 0 | 0 | 17893 | keratin 15, potentail contaminating epithelial cells |
| 32 | GTGCCCGTGC d | 1174 | 27 | 1 | 0 | 0 | 356372 | ESTs, Highly similar to TPIS_HUMAN TRIOSEPHOSPHATE |
| | | | | | | | | ISOMERASE [H.sapiens] |
| 33 | CCCGCCTCTT d | 1175 | 68 | 0 | 3 | 0 | | no confident match, tag highly abundant in some brain |
| | | | | | | | | libs + kidney and norm colon, does not look Ly |
| | | | | | | | | spec |
| 34 | ACACAGCAAG d | 1176 | 358 | 0 | 6 | 0 | AW57269 | ESTs, 77% conf. match, tag high in organoids + norm |
| | | | | | | | | breast epi-probably epi contaminant |
| 35 | GTCCCTGCCT d | 1177 | 33 | 0 | 0 | 0 | 279837 | GSTM2, glutathione S-transferase M2 (muscle) |

TABLE 8
Genes differentially expressed in myoepithelial cells from DCIS and normal breast tissue

| | | | | | | | | |
|---|---|---|---|---|---|---|---|---|
| SEQ | | | | | | | | |
| ID | | | | | | | | |
| NO: | Tag_Sequence | NL | D6 | D7 | 6/n | d7/n | Unigene | Gene |

| | | | | | | | | |
|---|---|---|---|---|---|---|---|---|
| 1178 | ACCAAAAACC d | 2 | 849 | 274 | 553 | 179 | 172928 | collagen, type I, alpha 1, internally primed site |
| 1179 | TGGAAATGAC d | 0 | 228 | 50 | 228 | 50 | 172928 | collagen, type I, alpha 1, shorter alternative |
| | | | | | | | | transcript |
| 1180 | CCACGGGATT d | 0 | 185 | 55 | 185 | 55 | | No match |
| 1181 | GATCAGGCCA d | 0 | 181 | 191 | 181 | 191 | 119571 | Collagen, type III, alpha 1 (Ehlers-Danlos syndrome |
| | | | | | | | | type IV, autosomal dominant, shorter alternative |
| | | | | | | | | transcript |
| 1182 | TTTGGTTTTC d | 0 | 154 | 24 | 154 | 24 | 179573 | retinoblastoma binding protein 1, reliable 3′ end |
| 1183 | AACTCCCAGT d | 3 | 351 | 427 | 114 | 139 | 110571 | growth arrest and DNA damage inducible beta, |
| | | | | | | | | reliable 3′ end |
| 1184 | GACTTTGGAA d | 0 | 110 | 36 | 110 | 36 | 172928 | collagen, type I, alpha 1, internal tag |
| 1185 | CAACCAGTAA d | 0 | 106 | 74 | 106 | 74 | AA723001 | zg89d05.sl Soares_fetal_heart_NbHH19W Homo sapiens |
| | | | | | | | | cDNA clone IMAGE:409737 3′ similar to contains |
| | | | | | | | | LTR2.t3 LTR2 repetitive element;, mRNA sequence, |
| | | | | | | | | internal tag |
| 1186 | CAGATAAGTT d | 0 | 101 | 72 | 101 | 72 | 36131 | collagen, type XIV, alpha 1 (undulin), reliable 3′ |
| | | | | | | | | end |
| 1187 | CATATCATTA d | 0 | 94 | 21 | 94 | 21 | 119206 | insulin-like growth factor binding protein 7, |
| | | | | | | | | reliable 3′ end |
| 1188 | TCACCGGTCA d | 2 | 127 | 224 | 83 | 146 | 290070 | gelsolin (amyloidosis, Finnish type), reliable 3′ |
| | | | | | | | | end |
| 1189 | AGGGAGCAGA d | 0 | 77 | 76 | 77 | 76 | 296049 | microfibrillar-associated protein, undefined 3′ end |
| 1190 | CCCTTGTCCG d | 0 | 75 | 60 | 75 | 60 | 127824 | Homo sapiens cDNA FLJ36047 fis, clone TEST12017951, |
| | | | | | | | | reliable 3′ end |
| 1191 | ATAAAAAGAA d | 0 | 73 | 19 | 73 | 19 | 83942 | cathepsin K (pycnodysostosis), reliable 3′ end |
| 1192 | GTTGTCTTTG d | 0 | 62 | 26 | 62 | 26 | 258798 | Hypothetical protein FLJ20003, reliable 3′ end |
| 1193 | CCGGGGGAGC d | 0 | 61 | 110 | 61 | 110 | 172928 | collagen, type I, alpha 1, internal tag |
| 1194 | TGGCCAGCTC d | 2 | 92 | 64 | 60 | 42 | AW572523 | xw56a11.x2 NC_CGAP_Pan1 Homo sapiens cDNA clone |
| | | | | | | | | IMAGE:2831996 3′, mRNA sequence, reliable 3′ end |
| 1195 | TTCGGTTGGT d | 0 | 59 | 19 | 59 | 19 | BG399135 | cn30g02.x1 Normal Human Trabecular Bone Cells Homo |
| | | | | | | | | sapiens cDNA clone NHTBC_cn30g02 random, mRNA |
| | | | | | | | | sequence, undefined 3′ end |
| 1196 | TCAACTTCTG d | 0 | 58 | 62 | 58 | 62 | N57419 | yw82e04.r1 Soares_placenta_8to9weeks_2NbHP8to9W Homo |
| | | | | | | | | sapiens cDNA clone IMAGE:258750 5′ similar to |
| | | | | | | | | gb:M20681 GLUCOSE TRANSPORTER TYPE 3, BRAIN (HUMAN); |
| | | | | | | | | contains Alu repetitive element;, mRNA sequence, |
| | | | | | | | | undefined 3′ end |
| 1197 | ACCCCCCCGC d | 5 | 253 | 1029 | 55 | 223 | 2780 | jun D proto-oncogene, undefined 3′ end |
| 1198 | GTGCGCTGAG d | 0 | 52 | 33 | 52 | 33 | 277477 | HLA-C Major histocompatibility complex, class 1, C, |
| | | | | | | | | reliable 3′ end |
| 1199 | GACCAGCAGA d | 0 | 48 | 43 | 48 | 43 | 172928 | collagen, type I, alpha 1, internal tag |
| 1200 | GTCAAAATTT d | 0 | 47 | 110 | 47 | 110 | 108623 | thrombospondin 2, reliable 3′ end |
| 1201 | GTGCTAAGCG d | 3 | 141 | 308 | 46 | 100 | 159263 | collagen, type VI, alpha 2, reliable 3′ end |
| 1202 | ATTTCTTCAA d | 0 | 44 | 19 | 44 | 19 | AF311912 | Homo sapiens pancreas tumor-related protein (FKSG12) |
| | | | | | | | | mRNA, complete cds, undefined 3′ end |
| 1203 | ACATTCTTTT d | 0 | 44 | 17 | 44 | 17 | 82226 | GPNMB Glycoprotein (transmembrane) nmb, reliable 3′ |
| | | | | | | | | end |
| 1204 | GGCACCTCAG d | 2 | 65 | 36 | 42 | 23 | 93913 | interleukin 6 (interferon, beta 2), reliable 3′ end |
| 1205 | ACATTCCAAG d | 0 | 42 | 50 | 42 | 50 | 245188 | tissue inhibitor of metalloproteinase 3 (Sorsby |
| | | | | | | | | fundus dystrophy, pseudoinflammatory), shorter |
| | | | | | | | | alternative transcript |
| 1206 | AAAACGTTTT d | 0 | 40 | 117 | 40 | 117 | 25647 | FOS V-fos FBJ murine osteosarcoma viral oncogene |
| | | | | | | | | homolog, internal tag |
| 1207 | TCCAGGAAAC d | 0 | 39 | 72 | 39 | 72 | 11590 | cathepsin F, reliable 3′ end |
| 1208 | CCTCCCAGCT d | 2 | 58 | 74 | 38 | 48 | 98508 | KIAA0150 protein, internal tag (NCB1 only) |
| 1209 | CTTGGGTTTT d | 0 | 37 | 122 | 37 | 122 | 251664 | Homo sapiens cDNA FLJ22066 fis, clone HEP10611, |
| | | | | | | | | reliable 3′ end |
| 1210 | CCAGGGGAGA d | 0 | 37 | 48 | 37 | 48 | 278613 | interferon alpha-inducible protein 27, reliable 3′ |
| | | | | | | | | end |
| 1211 | GGGAGGGGTG d | 3 | 113 | 100 | 37 | 33 | R09745 | yf27d09.s1 Soares fetal liver spleen INFLS Homo |
| | | | | | | | | sapiens cDNA clone IMAGE:128081 3′, mRNA, |
| | | | | | | | | undefined 3′ end |
| 1212 | GCACGGAAAA d | 0 | 36 | 31 | 36 | 31 | BG236552 | nai4Sb05.x1 NCI_CGAP_HN20 Homo sapiens cDNA clone |
| | | | | | | | | IMAGE:4263104 3′, mRNA sequence, undefined 3′ end |
| 1213 | GATGAGGAGA d | 3 | 107 | 74 | 35 | 24 | 179573 | retinoblastoma binding protein 1, internally primed |
| | | | | | | | | site |
| 1214 | TGGAAAGTGA d | 14 | 468 | 654 | 34 | 47 | 25647 | FQS V-fos FBJ murine osteosarcoma viral oncogene |
| | | | | | | | | homolog, reliable 3′ end |
| 1215 | CGCCGACGAT d | 0 | 32 | 100 | 32 | 100 | 265827 | GIP3 interferon alpha-inducible protein, reliable 3′ |
| | | | | | | | | end |
| 1216 | CTGTCAGCGT d | 0 | 32 | 29 | 32 | 29 | 283713 | collagen triple helix repeat containing 1, reliable |
| | | | | | | | | 3′ end |
| 1217 | GTTCCACAGA d | 0 | 32 | 24 | 32 | 24 | 179573 | retinoblastoma binding protein 1, internally primed |
| | | | | | | | | site |
| 1218 | GGAACTTTTA d | 2 | 47 | 33 | 31 | 22 | 43857 | similar to glucosamine-6-sulfatases, reliable 3′ end |
| 1219 | GTATAAACGT d | 0 | 31 | 29 | 31 | 29 | | No match |
| 1220 | GAGGAGGAGA d | 0 | 30 | 26 | 30 | 26 | 78054 | DEAD/H (Asp-Gln-Ala-Asp/His) box polypeptide 38, |
| | | | | | | | | internal tag |
| 1221 | GGGGGGGGGT d | 0 | 29 | 131 | 29 | 131 | 224731 | EST, Weakly similar to 1203377A lamin A [Homo |
| | | | | | | | | sapiens], reliable 3′ end |
| 1222 | TTGGGATGGG d | 0 | 29 | 103 | 29 | 103 | 278568 | H factor (complement)-like 1, reliable 3′ end |
| 1223 | TTCCGGTTCC d | 0 | 29 | 17 | 29 | 17 | 172609 | nucleobindin 1, reliable 3′ end |
| 1224 | GGAAAGTGTT d | 0 | 29 | 17 | 29 | 17 | AW754264 | PM4-CT0331-251199-001-F10 CT0331 Homo sapiens cDNA, |
| | | | | | | | | mRNA sequence, undefined 3′ end |
| 1225 | GCCCAGCTGG d | 0 | 28 | 62 | 28 | 62 | 334798 | hypothetical protein FLJ20897, reliable 3′ end |
| 1226 | TTTCCCTCAA d | 2 | 42 | 21 | 27 | 14 | 75111 | protease, serine, 11 (IGF binding), reliable 3′ end |
| 1227 | GGATGTGAAA d | 0 | 26 | 19 | 26 | 19 | 177543 | MIC2 antigen identified by monoclonal antibodies |
| | | | | | | | | 12E7, F21 and O13, reliable 3′ end |
| 1228 | GCAAAAAAAA d | 5 | 120 | 143 | 26 | 31 | 4746 | Hypothetical protein FLJ21324 reliable 3′ end |
| 1229 | ACCCACGTCA d | 5 | 113 | 317 | 25 | 69 | 198951 | jun B proto-oncogene, reliable 3′ end |
| 1230 | CGGGGTGGCC d | 0 | 24 | 193 | 24 | 193 | 1584 | cartilage oligomeric matrix protein (pseudo- |
| | | | | | | | | achondroplasia, epiphyseal dysplasia 1, multiple), |
| | | | | | | | | reliable 3′ end |
| 1231 | CGCCCCGGCG d | 0 | 24 | 43 | 24 | 43 | BM145074 | TCAAP1D14680 Pediatric acute myelogenous leukemia |
| | | | | | | | | cell (FAB M1) Baylor-HGSC project = TCAA |
| | | | | | | | | Homo sapiens cDNA clone TCAAP1468, mRNA sequence, |
| | | | | | | | | reliable 3′ end |
| 1232 | CAGACTTTTG d | 0 | 24 | 24 | 24 | 24 | 63348 | elastin microfibril interface located protein, |
| | | | | | | | | reliable 3′ end |
| 1233 | TTACTTCTGC d | 0 | 23 | 45 | 23 | 45 | 75736 | apolipoprotein D, internal tag |
| 1234 | CGTCTTTAAA d | 0 | 23 | 26 | 23 | 26 | 21275 | Hypothetical protein FLJ11011, internal tag |
| 1235 | TTGCTGACTT d | 12 | 279 | 122 | 23 | 10 | 108885 | collagen, type VI, alpha 1, reliable 3′ end |
| 1236 | TCGAAGAACC d | 2 | 34 | 60 | 22 | 39 | 76294 | CD63 antigen (melanoma 1 antigen) reliable 3′ end |
| 1237 | GGCCCCTCAC d | 0 | 22 | 74 | 22 | 74 | 274313 | insulin-like growth factor binding protein 6, |
| | | | | | | | | reliable 3′ end |
| 1238 | CAGCTGGCCA d | 0 | 22 | 36 | 22 | 36 | 79732 | fubulin, transcript variant C, reliable 3′ end |
| 1239 | TGTAAACAAT d | 0 | 22 | 19 | 22 | 19 | 170040 | platelet-derived growth factor receptor-like, |
| | | | | | | | | reliable 3′ end |
| 1240 | GAGATCCGCA d | 0 | 21 | 62 | 21 | 62 | 75348 | proteasome (prosome, macropain) activator subunit 1 |
| | | | | | | | | (PA28 alpha), reliable 3′ end |
| 1241 | CCCTGGGTTC d | 6 | 124 | 74 | 20 | 12 | 111334 | FTL Ferritin, light polypeptide, reliabe 3′ end |
| 1242 | TCTAACGGGC d | 0 | 20 | 169 | 20 | 169 | 102171 | immunoglobulin superfamily containing leucine-rich |
| | | | | | | | | repeat, reliable 3′ end |
| 1243 | TGCGCTCTCC d | 0 | 20 | 86 | 20 | 86 | 25391 | Homo-sapiens, clone IMAGE:4691115, mRNA, partial |
| | | | | | | | | cds, reliable 3′ end |
| 1244 | CGCAGTCTGC d | 0 | 20 | 48 | 20 | 48 | 24087 | Arylhydrocarbon receptor repressor, internal tag |
| 1245 | GGAGGAATTC d | 0 | 20 | 21 | 20 | 21 | 78056 | cathepsin L, reliable 3′ end |
| 1246 | AAGAAAGGAG d | 0 | 20 | 21 | 20 | 21 | 202097 | procollagen C-endopeptidase enhancer, reliable 3′ |
| | | | | | | | | end |
| 1247 | ACTTATTATG d | 2 | 30 | 107 | 19 | 70 | 76152 | decorin, reliable 3′ end |
| 1248 | TAGTTGGAAA d | 9 | 173 | 105 | 19 | 11 | 1119 | nuclear receptor subfamily 4, group A, member 1, |
| | | | | | | | | reliable 3′ end |
| 1249 | TCAACAAATT d | 0 | 19 | 48 | 19 | 48 | 9315 | HNOEL-iso protein, reliable 3′ end |
| 1250 | GCGTGAGTGC d | 0 | 19 | 17 | 19 | 17 | AW894414 | CM2-NN0032-050400-142-g12 NN0032 Homo sapiens cDNA, |
| | | | | | | | | mRNA sequence, undefined 3′ end |
| 1251 | CGGCTGAATT d | 0 | 19 | 17 | 19 | 17 | 75888 | phosphogluconate dehydrogenase, reliable 3′ end |
| 1252 | AGCAAACTGA d | 0 | 19 | 17 | 19 | 17 | 182579 | leucine aminopeptidase 3, reliable 3′ end |
| 1253 | GCGCAGAGGT d | 15 | 277 | 148 | 18 | 10 | BQ344433 | MR2-NT0136-161100-003-a05 NT0136 Homo sapiens cDNA, |
| | | | | | | | | mRNA sequence, undefined 3′ end |
| 1254 | TGGGACTCCA d | 2 | 28 | 45 | 18 | 30 | 59384 | hypothetical protein MGC3047, reliable 3′ end |
| 1255 | ACTCAGCCCG d | 2 | 28 | 36 | 18 | 23 | 101382 | tumor necrosis factor, alpha-induced protein 2, |
| | | | | | | | | reliable 3′ end |
| 1256 | CAGCACGGAT d | 2 | 28 | 26 | 18 | 17 | | No match |
| 1257 | GGAAATGTCA d | 18 | 325 | 93 | 18 | 5 | 111301 | Matrix metalloproteinase 2 (gelatinase A, 72kD |
| | | | | | | | | gelatinase, 72kD type IV collagenase, reliable |
| | | | | | | | | 3′ end |
| 1258 | TGCGCTGGCC d | 0 | 18 | 67 | 18 | 67 | 289019 | latent transforming growth factor beta binding |
| | | | | | | | | protein 3, relable 3′ end |
| 1259 | GACGGCTGCA d | 2 | 26 | 74 | 17 | 48 | 258730 | Heme-regulated initiation factor 2-alpha kinase, |
| | | | | | | | | undefined 3′ end |
| 1260 | GGAAGTTTCG d | 2 | 26 | 36 | 17 | 23 | 55847 | mitochondrial ribosomal protein L51, reliable 3′ end |
| 1261 | GGGCCAACCC d | 0 | 17 | 88 | 17 | 88 | 119475 | Cold inducible RNA binding protein, undefined 3′ end |
| 1262 | GACGCGGCGC d | 0 | 17 | 24 | 17 | 24 | 352987 | MGC21945 Binder of Rho GTPase 3-like, reliable 3′ |
| | | | | | | | | end |
| 1263 | TATCCTGAAA d | 0 | 17 | 17 | 17 | 17 | AA778363 | z156g03.s1 Soares_pregnant_uterus_NbHPU Homo sapiens |
| | | | | | | | | cDNA clone IMAGE:505972 3′ similar to contains L1.t3 |
| | | | | | | | | L1 repetitive element;, mRNA sequence, undefined 3′ |
| | | | | | | | | end |
| 1264 | ATGGCAACAG d | 0 | 17 | 17 | 17 | 17 | 149609 | integrin, alpha 5 (fibronectin receptor, alpha poly- |
| | | | | | | | | peptide), reliable 3′ end |
| 1265 | ACGACAAAGC d | 0 | 17 | 17 | 17 | 17 | 83920 | peptidylglycine alpha-amidating monooxygenase, |
| | | | | | | | | reliable 3′ end |
| 1266 | ACTGAAAGAA d | 3 | 50 | 124 | 16 | 40 | 169756 | CIS Complement component 1, s subcomponent, reliable |
| | | | | | | | | 3′ end |
| 1267 | GGCTGCCCTG d | 2 | 24 | 62 | 16 | 40 | 74566 | Dihydropyrimidinase-like-3, reliable 3′ end |
| 1268 | GGCACGCAGC d | 0 | 15 | 79 | 15 | 79 | BF349813 | RCI-HT0217-151099-011-e05 HT0217 Homo sapiens cDNA, |
| | | | | | | | | mRNA sequence, undefined 3′ end |
| 1269 | CAAAAAATTA d | 0 | 15 | 43 | 15 | 43 | H81706 | ys67c09.r1 Soares retina N2b4HR Homo sapiens cDNA |
| | | | | | | | | clone IMAGE:219856 5′, mRNA sequence, undefined |
| | | | | | | | | 3′ end |
| 1270 | GGCCACGTAG d | 0 | 15 | 26 | 15 | 26 | 155597 | DF D component of complement (adipsin), internal tag |
| 1271 | CTAAAAAAAA d | 0 | 15 | 26 | 15 | 26 | 54457 | CD81 antigen (target of antiproliferative antibody |
| | | | | | | | | 1), reliable 3′ end |
| 1272 | CCAAGGTTTT d | 0 | 15 | 19 | 15 | 19 | 99120 | DEAD/H (Asp-Glu-Ala-Asp/His) box polypeptide, Y |
| | | | | | | | | chromosome, internal tag |
| 1273 | GACAAAAAAA d | 6 | 91 | 33 | 15 | 5 | 32366 | DERMOI Likely ortholog of mouse and rat twist- |
| | | | | | | | | related bHLH protein Dermo-1, reliable 3′ end |
| 1274 | CCCTACCCTG d | 11 | 160 | 792 | 15 | 74 | 75736 | apolipoprotein D, reliable 3′ end |
| 1275 | GGAAAAAAAA d | 3 | 45 | 93 | 15 | 30 | 198271 | NADH dehydrogenase (ubiquinone) 1 alpha subcomplex, |
| | | | | | | | | 10 (42kD), reliable 3′ end |
| 1276 | GCGGCGGCTC d | 2 | 2 | 26 | 14 | 17 | BQ339816 | RCS-NN1165-251100-024-F08 NN1165 Homo sapiens cDNA, |
| | | | | | | | | mRNA sequence, undefined 3′ end |
| 1277 | GCGAAACCCA d | 0 | 14 | 67 | 14 | 67 | 359286 | ESTs, Moderately similar to hypothetical protein |
| | | | | | | | | FLJ20378, [Homo sapiens], reliable 3′ end |
| 1278 | CTAATAAACT d | 0 | 14 | 17 | 14 | 17 | 279583 | CGI-81 protein, shorter alternative transcript |
| 1279 | AAGAGCGCCG d | 12 | 172 | 45 | 14 | 4 | 8997 | Sad1 unc-84 domain protein 1, reliable 3′ end |
| 1280 | GCTGAACGCG d | 14 | 193 | 60 | 14 | 4 | 99029 | CCAAT/enhancer binding protein (C/EBP), beta, |
| | | | | | | | | reliable 3′ end |
| 1281 | GCCCCCAATA d | 29 | 400 | 270 | 14 | 9 | 227751 | lectin, galactoside-binding, soluble, 1 (galectin |
| | | | | | | | | 1), reliable 3′ end |
| 1282 | GCGGGGTGGA d | 6 | 83 | 177 | 13 | 29 | 85155 | zinc finger protein 36, C3H type-like 1, internally |
| | | | | | | | | primed site |
| 1283 | TAGTTGGAAC d | 5 | 62 | 41 | 13 | 9 | BG057763 | 7f75e10.x1 Lupski_dorsal_root_ganglion Homo sapiens |
| | | | | | | | | cDNA clone IMAGE:3302875 3′, mRNA, reliable 3′ end |
| 1284 | CAAGTTCTTT d | 3 | 41 | 60 | 13 | 19 | 356629 | Homo sapiens cDNA FLJ31414 fis, clone NT2NE2000260, |
| | | | | | | | | weakly similar to THYMOSIN BETA-4, undefined 3′ end |
| 1285 | CGACCCCACG d | 6 | 81 | 60 | 13 | 10 | 169401 | apolipoprotein E, undefined 3′ end |
| 1286 | GAATTCACAA d | 0 | 13 | 131 | 13 | 131 | 128087 | F2R coagulation factor 11 (thrombin) receptor, |
| | | | | | | | | reliable 3′ end |
| 1287 | GAGTGGGTGC d | 0 | 13 | 69 | 13 | 69 | 12908 | CDC42 binding protein kinase beta (DMPK-like), |
| | | | | | | | | undefined 3′ end |
| 1288 | CAGCGGCGGG d | 0 | 13 | 57 | 13 | 57 | 2420 | superoxide dismutase 3, extracellular, reliable 3′ |
| | | | | | | | | end |
| 1289 | GCCTGTCCCT d | 0 | 13 | 50 | 13 | 50 | 821 | biglycan, reliable 3′ end |
| 1290 | CAGGACAGTT d | 0 | 13 | 48 | 13 | 48 | 78305 | RAB2, member RAS oncogene family, shorter |
| | | | | | | | | alternative transcript |
| 1291 | GCAGAAAATT d | 0 | 13 | 21 | 13 | 21 | 333555 | echinoderm microtubule associated protein like 4, |
| | | | | | | | | reliable 3′ end |
| 1292 | CATAAATGCG d | 0 | 13 | 21 | 13 | 21 | 237356 | stromal cell-derived factor 1, SAGE Genie: no match, |
| | | | | | | | | NCBI: Acc.no.U19495 |
| 1293 | GTGGCAGCGC d | 0 | 13 | 17 | 13 | 17 | 285753 | stathmin-like 3, reliable 3′ end |
| 1294 | CACACAGTTT d | 6 | 80 | 98 | 13 | 16 | 204354 | ras homolog gene family, member B, undefined 3′ end |
| 1295 | GGTGCCCAGT d | 2 | 20 | 76 | 13 | 50 | 75607 | myristoylated alanine-rich protein kinase C sub- |
| | | | | | | | | strate, internally primed site |
| 1296 | TTCTGTGCTG d | 3 | 40 | 105 | 13 | 34 | 1279 | C1R Complement component 1, r subcomponent, reliable |
| | | | | | | | | 3′ end |
| 1297 | CTCTCCAAAC d | 2 | 20 | 26 | 13 | 17 | 151242 | serine (or cysteine) proteinase inhibitor, clade G |
| | | | | | | | | (C1 inhibitor), member 1, (angioedema, heredi- |
| | | | | | | | | tary), reliable 3′ end |
| 1298 | GGCCCTAGGC d | 3 | 39 | 98 | 13 | 32 | 78909 | zinc finger protein 36, C3H type-like 2, reliable 3′ |
| | | | | | | | | end |
| 1299 | CTCAACCCCC d | 2 | 19 | 105 | 12 | 68 | 89137 | Low density lipoprotein-related protein 1 (alpha-2- |
| | | | | | | | | macroglobulin receptor), reliable 3′ end |
| 1300 | AGCCACCGCG d | 2 | 19 | 43 | 12 | 28 | 193716 | Complement component (3b/4b) receptor 1, including |
| | | | | | | | | Knops blood group system, reliable 3′ end |
| 1301 | ACCTTGAAGT d | 2 | 19 | 36 | 12 | 23 | 29352 | tumor necrosis factor, alpha-induced protein 6, |
| | | | | | | | | internally primed site |
| 1302 | TCAGAAGTTT d | 2 | 19 | 29 | 12 | 19 | 243901 | Homo sapiens mRNA; cDNA DKFZp564C1563 (from clone |
| | | | | | | | | DKFZp564C1563), reliable 3′ end |
| 1303 | TGGCAAAATA d | 2 | 19 | 26 | 12 | 17 | BM353720 | ig55c02.y1 HR85 islet Homo sapiens cDNA 5′, mRNA |
| | | | | | | | | sequence, undefined 3′ end |
| 1304 | GGGAGGTAGC d | 2 | 18 | 31 | 11 | 20 | 171825 | Basic helix-loop-helix domain containing, class B, |
| | | | | | | | | 2, reliable 3′ end |
| 1305 | GAAAAATTTA d | 5 | 50 | 86 | 11 | 19 | 169248 | cytochrome c, reliable 3′ end |
| 1306 | GGCAGGCGGG d | 6 | 65 | 55 | 11 | 9 | 333069 | Ets2 repressor factor, reliable 3′ end |
| 1307 | AGATTCAAAC d | 3 | 32 | 41 | 10 | 13 | 14368 | SH3 domain binding glutamic acid-rich protein like, |
| | | | | | | | | reliable 3′ end |
| 1308 | GTAAAAAAAA d | 8 | 78 | 86 | 10 | 11 | 460 | Activating transcription factor 3, reliable 3′ end |
| | | | | | | | | (+at least 10 others) |
| 1309 | AGGCTCCTGG d | 3 | 31 | 217 | 10 | 71 | 24395 | small inducible cytokine subfamily B (Cys-X-Cys), |
| | | | | | | | | member 14 (BRAK), reliable 3′ end |
| 1310 | CGCCGCGGTG d | 3 | 31 | 48 | 10 | 16 | 4835 | eukaryotic translation initiation factor 3, subunit |
| | | | | | | | | 8 (110kD), reliable 3′ end |
| 1311 | TGCCTGCACC d | 5 | 46 | 76 | 10 | 17 | 135084 | cystatin C (amyloid angiopathy and cerebral |
| | | | | | | | | hemorrhage), reliable 3′ end |
| 1312 | GTGACTGCCA d | 5 | 45 | 38 | 10 | 8 | 84183 | Diptheria toxin resistance protein required for |
| | | | | | | | | diphthamide biosynthesis-like 1 (S. cerevisiae), |
| | | | | | | | | reliable 3′ end |
| 1313 | GTTTATGGAT d | 3 | 30 | 26 | 10 | 9 | 365706 | matrix G1a protein, reliable 3′ end |
| 1314 | GCAGCCATCC d | 34 | 321 | 334 | 10 | 10 | 4437 | ribosomal protein L28, reliable 3′ end |
| 1315 | CAGGTTTCAT d | 12 | 117 | 124 | 10 | 10 | 24395 | small inducible cytokine subfamily B (Cys-X-Cys), |
| | | | | | | | | member 14 (BRAK), reliable 3′ end |
| 1316 | GGCCTGCTGC d | 6 | 58 | 45 | 10 | 7 | 9634 | Hypothetical protein BC009925, reliable 3′ end |
| 1317 | CCCCCTGGAT d | 6 | 56 | 119 | 9 | 19 | 275243 | S100 calcium binding protein A6 (calcyclin), |
| | | | | | | | | reliable 3′ end |
| 1318 | GGGGGAATTT d | 3 | 28 | 124 | 9 | 40 | BM805435 | AGENCOURT_6498312 NIH_MGC_124 Homo sapiens cDNA |
| | | | | | | | | clone IMAGE:5728837 5′, mRNA, undefined 3′ end |
| 1319 | AACTTTTGGC d | 3 | 28 | 55 | 9 | 18 | 195471 | 6-phosphofructo-2-kinase/fructose-2,6-biphosphatase |
| | | | | | | | | 3, internally primed site |
| 1320 | AGAATTTGCA | 6 | 53 | 50 | 9 | 8 | 250655 | prothyrnosin, alpha (gene sequence 28), internally |
| | | | | | | | | primed site |
| 1321 | GCCGCCCTGC | 5 | 40 | 33 | 9 | 7 | 82208 | ACADVL Acyl-Coenzyme A dehydrogenase, very long |
| | | | | | | | | chain, reliable 3′ end |
| 1322 | GGGGGTAACT | 5 | 39 | 38 | 8 | 8 | 99969 | fusion, derived from t(12;16) malignant liposarcoma, |
| | | | | | | | | reliable 3′ end |
| 1323 | TGAAAAAAAA | 5 | 35 | 33 | 8 | 7 | 119178 | Cation-chloride cotransporter-interacting protein, |
| | | | | | | | | reliable 3′ end |
| 1324 | GGCCTTTTTT | 5 | 35 | 29 | 8 | 6 | 109804 | HIFX H1 histone family, member X, reliable 3′ end |
| 1325 | GCGACGAGGC | 14 | 95 | 91 | 7 | 7 | 2017 | ribosomal protein L38, internal tag |
| 1326 | GCGCTGGAGT d | 3 | 21 | 33 | 7 | 11 | 110695 | hypothetical protein MGC3133, reliable 3′ end |
| 1327 | GGAGGGGGCT | 9 | 62 | 48 | 7 | 5 | 77886 | Lamin A/C, internally primed site |
| 1328 | GAGGGAGTTT | 152 | 993 | 964 | 7 | 6 | 76064 | ribosomal protein L27a, reliable 3′ end |
| 1329 | CGCTGGTTCC | 37 | 237 | 184 | 6 | 5 | 179943 | ribosomal protein L11, reliable 3′ end |
| 1330 | TCAAGCCATC | 9 | 58 | 45 | 6 | 5 | BG060046 | naf48a07.x1 NCI_CGAP_Brn65 Homo sapiens cDNA clone |
| | | | | | | | | IMAGE:4147116 3′, mRNA sequence, undefined 3′ end |
| 1331 | GCTTTGGAG d | 5 | 29 | 64 | 6 | 14 | 90918 | C11orf10 Chromosome 11 open reading frame 10, |
| | | | | | | | | reliable 3′ end |
| 1332 | CTGCCAAGTT | 14 | 85 | 81 | 6 | 6 | 75873 | Zyxin, reliable 3′ end |
| 1333 | GACTCACTTT | 11 | 65 | 50 | 6 | 5 | 699 | peptidylprolyl isomerase B (cyclophilin B), |
| | | | | | | | | reliable 3′ end |
| 1334 | GGGGAAATCG d | 34 | 195 | 544 | 6 | 16 | 76293 | thymosin, beta 10, internally primed site |
| 1335 | GGCCGCGTTC d | 20 | 115 | 568 | 6 | 28 | 5174 | ribosomal protein S17, reliable 3′ end |
| 1336 | CCGTGACTCT | 12 | 70 | 112 | 6 | 9 | 296267 | follistatin-like 1, reliable 3′ end |
| 1337 | TGCACGTTTT | 117 | 631 | 453 | 5 | 4 | 169793 | ribosomal protein L32, reliable 3′ end |
| 1338 | GTTGTGGTTA | 81 | 429 | 274 | 5 | 3 | 75415 | beta-2-microglobulin, reliable 3′ end |
| 1339 | GTTAACGTCC | 11 | 54 | 100 | 5 | 9 | 178391 | ribosomal protein L36a, reliable 3′ end |
| 1340 | CAGGAGTTCA | 6 | 30 | 50 | 5 | 8 | 83583 | Actin related protein 2/3 complex, subunit 2 (34 |
| | | | | | | | | kD), reliable 3′ end |
| 1341 | CCTCGGAAAA d | 15 | 74 | 224 | 5 | 15 | 2017 | ribosomal protein L38, reliable 3′ end |
| 1342 | CCCGTCCGGA d | 81 | 388 | 1002 | 5 | 12 | 180842 | ribosomal protein L13, reliable 3′ end |
| 1343 | GGAAGCTAAG | 34 | 150 | 181 | 4 | 5 | 136348 | Osteoblast specific factor 2 (fasciclin I-like), |
| | | | | | | | | undefined 3′ end |
| 1344 | CCCATCCGAA | 29 | 129 | 179 | 4 | 6 | 91379 | ribosomal protein L26, reliable 3′ end |
| 1345 | CCCCAGCCAG | 18 | 77 | 98 | 4 | 5 | 252259 | Ribosomal protein S3, reliable 3′ end |
| 1346 | GGTGGCACTC | 11 | 43 | 81 | 4 | 8 | 77273 | ras homolog gene family, member A, reliable 3′ end |
| 1347 | ATGGTGGGGG | 51 | 200 | 17 | 4 | 3 | 343586 | zinc finger protein 36, C3H type, homolog (mouse), |
| | | | | | | | | reliable 3′ end |
| 1348 | CGCCGCCGGC | 68 | 265 | 442 | 4 | 7 | 182825 | ribosomal protein L35, reliable 3′ end |
| 1349 | CAGCAGAAGC | 9 | 35 | 45 | 4 | 5 | 26703 | CCR4-NOT transcription complex, subunit 8, reliable |
| | | | | | | | | 3′ end |
| 1350 | TTGGGGTTTC | 158 | 555 | 515 | 4 | 3 | 62954 | Ferritin, heavy polypeptide 1, reliable 3′ end |
| 1351 | CCAGTGGCCC d | 14 | 47 | 134 | 3 | 10 | 180920 | ribosomal protein S9, reliable 3′ end |
| 1352 | CGCCGGAACA | 29 | 95 | 148 | 3 | 5 | 286 | ribosomal protein L4, reliable 3′ end |
| 1353 | CTGTACTTGT | 18 | 56 | 98 | 3 | 5 | 75678 | FBJ murine osteosarcoma viral oncogene homolog B, |
| | | | | | | | | reliable 3′ end |
| 1354 | ACCATCCTGC | 25 | 68 | 76 | 3 | 3 | 76095 | immediate early response 3, reliable 3′ end |
| 1355 | GTGAAACTCC | 21 | 58 | 93 | 3 | 4 | B1005171 | PM3-HN0076-020401-008-d01 HN0076 Homo sapiens cDNA, |
| | | | | | | | | mRNA sequence, reliable 3′ end |
| 1356 | GCCGTGTCCG | 63 | 151 | 379 | 2 | 6 | 350166 | ribosomal protein S6, reliable 3′ end |
| 1357 | GCGAAACCCC | 48 | 113 | 198 | 2 | 4 | 30211 | hypothetical protein FLJ22313, reliable 3′ end |
| 1358 | GCCGAGGAAG | 55 | 111 | 260 | 2 | 5 | 339696 | ribosomal protein S12, reliable 3′ end |
| 1359 | TTGAATTCCC d | 44 | 15 | 2 | −3 | −19 | 171921 | sema domain, immunoglobulin domain (Ig), short basic |
| | | | | | | | | domain, secreted, (semaphorin) 3C, reliable 3′ end |
| 1360 | GTGCTGAATG | 144 | 50 | 29 | −3 | −5 | 77385 | myosin, light polypeptide 6, alkali, smooth muscle |
| | | | | | | | | and non-muscle, reliable 3′ end |
| 1361 | TTGAAGCTTT d | 451 | 154 | 19 | −3 | −24 | 75765 | GRO2 oncogene, reliable 3′ end |
| 1362 | GCATAATAGG d | 270 | 89 | 14 | −3 | −19 | 350077 | ribosomal protein L21, reliable 3′ end |
| 1363 | AAGACAGTGG | 137 | 44 | 26 | −3 | −5 | 296290 | ribosomal protein L37a, reliable 3′ end |
| 1364 | TGTTCTGGAG | 75 | 24 | 19 | −3 | −4 | 74471 | Gap junction protein, alpha 1, 43kD (connexin 43), |
| | | | | | | | | reliable 3′ end |
| 1365 | ACAGGCTACG | 100 | 31 | 38 | −3 | −3 | 75777 | transgelin, reliable 3′ end |
| 1366 | AAGAAGATAG | 77 | 23 | 12 | −3 | −6 | 182426 | Ribosomal protein S2, reliable 3′ end |
| 1367 | GACTTGTATA | 44 | 13 | 5 | −3 | −9 | 81328 | Nuclear factor of kappa light polypeptide gene |
| | | | | | | | | enhancer in B-cells inhibitor, alpha, internally |
| | | | | | | | | primed site |
| 1368 | ATTCTCCAGT | 121 | 35 | 17 | −3 | −7 | 234518 | ribosomal protein L23, reliable 3′ end |
| 1369 | TTATGGGGAG d | 32 | 9 | 0 | −4 | −32 | 75612 | stress-induced-phosphoprotein 1 (Hsp70/Hsp90- |
| | | | | | | | | organizing protein), reliable 3′ end |
| 1370 | GGCTGTACCC | 118 | 32 | 26 | −4 | −4 | BC007492 | Homo sapiens, cysteine and glycine-rich protein 1, |
| | | | | | | | | clone IMAGE:2966961, mRNA, reliable 3′ end |
| 1371 | ATGGCTGGTA | 156 | 42 | 19 | −4 | −8 | 182426 | ribosomal protein S2, reliable 3′ end |
| 1372 | TGAAGTTATA | 71 | 19 | 24 | −4 | −3 | 287797 | integrin, beta 1 (fibronectin receptor, beta poly- |
| | | | | | | | | peptide, antigen CD29 includes MDF2, MSK12), |
| | | | | | | | | reliable 3′ end |
| 1373 | AGTATGAGGA | 64 | 17 | 7 | −4 | −9 | 211600 | Tumor necrosis factor, alpha-induced protein 3, |
| | | | | | | | | reliable 3′ end |
| 1374 | GCCTACCCGA | 74 | 19 | 12 | −4 | −6 | 23582 | tumor-associated calcium signal transducer 2, |
| | | | | | | | | reliable 3′ end |
| 1375 | CGTGTTAATG d | 26 | 7 | 2 | −4 | −11 | 2110 | zinc finger protein 9 (a cellular retroviral nucleic |
| | | | | | | | | acid binding protein), reliable 3′ end |
| 1376 | TTGTAATCGT d | 57 | 14 | 2 | −4 | −24 | NM_004152 | Homo sapiens ornithine decarboxylase antizyme 1 |
| | | | | | | | | (OAZI), mRNA, reliable 3′ end |
| 1377 | TCTTGTGCAT | 32 | 8 | 5 | −4 | −7 | 2795 | lactate dehydrogenase A, reliable 3′ end |
| 1378 | TTACCATATC d | 74 | 18 | 7 | −4 | −10 | 300141 | ribosomal protein L39, reliable 3′ end |
| 1379 | TGGAAGCACT d | 94 | 22 | 7 | −4 | −13 | 624 | interleukin 8, reliable 3′ end |
| 1380 | CTGCTATACG | 91 | 21 | 21 | −4 | −4 | 180946 | Ribosomal protein L5, reliable 3′ end |
| 1381 | TGCTGTGCAT d | 72 | 17 | 0 | −4 | −72 | 75692 | Asparagine synthetase, reliable 3′ end |
| 1382 | ACTAACACCC | 63 | 14 | 14 | −4 | −4 | BC009321 | Homo sapiens, clone MGC:16650 IMAGE:4123521, mRNA, |
| | | | | | | | | complete cds, reliable 3′ end |
| 1383 | GATCTCTTGG d | 29 | 7 | 0 | −4 | −29 | 38991 | S100 calcium binding protein A2, reliable 3′ end |
| 1384 | TACTCTTGGC d | 25 | 6 | 0 | −4 | −25 | 2730 | heterogeneous nuclear ribonucleoprotein L, reliable |
| | | | | | | | | 3′ end |
| 1385 | CTGTTGATTG | 51 | 11 | 10 | −5 | −5 | 249495 | heterogeneous nuclear ribonucleoprotein A1, shorter |
| | | | | | | | | alternative transcript |
| 1386 | TAATAAAGGT d | 180 | 39 | 7 | −5 | −25 | 151604 | ribosomal protein S8, reliable 3′ end |
| 1387 | CCACTGCACT | 321 | 67 | 67 | −5 | −5 | 68257 | General transcription factor IIF, polypeptide 1 |
| | | | | | | | | (74kD subunit), reliable 3′ end |
| 1388 | AGAAAGATGT d | 229 | 47 | 10 | −5 | −24 | 78225 | annexin A1, reliable 3′ end |
| 1389 | CTGTACAGAC d | 43 | 9 | 5 | −5 | −9 | 251653 | tubulin, beta, 2, reliable 3′ end |
| 1390 | AGAAATGTTG d | 28 | 6 | 0 | −5 | −28 | 146217 | Homo sapiens cDNA FLJ34184 fis, clone FCBBF3017024, |
| | | | | | | | | reliable 3′ end |
| 1391 | GGCTTTACCC d | 74 | 14 | 0 | −5 | −74 | 119140 | eukaryotic translation initiation factor SA, |
| | | | | | | | | reliable 3′ end |
| 1392 | ACAGTGGGGA d | 57 | 11 | 2 | −5 | −24 | 278270 | unactive progesterone receptor, 23 kD, reliable 3′ |
| | | | | | | | | end |
| 1393 | TGTATAAAAA d | 40 | 8 | 2 | −5 | −17 | 82689 | tumor rejection antigen (gp96) 1, reliable 3′ end |
| 1394 | TTATGGGATC | 63 | 12 | 19 | −5 | −3 | 5662 | guanine nucleotide binding protein (G protein), beta |
| | | | | | | | | polypeptide 2-like 1, reliable 3′ end |
| 1395 | TTACTAAATG d | 23 | 4 | 0 | −5 | −23 | 155560 | Calnexin, reliable 3′ end |
| 1396 | GCCTTGGGTG d | 81 | 15 | 0 | −5 | −81 | 2250 | leukemia inhibitory factor (cholinergic differenti- |
| | | | | | | | | ation factor), reliable 3′ end |
| 1397 | ATCAAGGGTG | 92 | 17 | 14 | −6 | −6 | 157850 | ribosomal protein L9, reliable 3′ end |
| 1398 | TAGGTAGCTC d | 25 | 4 | 0 | −6 | −25 | 179999 | Homo sapiens, clone IMAGE:3457003, mRNA, reliable 3′ |
| | | | | | | | | end |
| 1399 | TACCATCAAT d | 198 | 35 | 14 | −6 | −14 | 169476 | glyceraldehyde-3-phosphate debydrogenase, reliable |
| | | | | | | | | 3′ end |
| 1400 | CATTTGTAAT | 32 | 6 | 5 | −6 | −7 | X93334 | mitochondrial |
| 1401 | AAACTGTGGT d | 20 | 3 | 0 | −6 | −20 | W31349 | zb95d06.s1 Soares_parathyroid_tumor_NbHpA Homo |
| | | | | | | | | sapiens cDNA clone IMAGE:320555 3′ similar to |
| | | | | | | | | S W:COX2_GORGO P26456 CYTOCHROME C OXIDASE POLY- |
| | | | | | | | | PEPTIDE II;, mRNA sequence, undefined 3′ end |
| 1402 | AAGCTGTATA d | 34 | 6 | 0 | −6 | −34 | 289114 | hexabrachion (tenascin C, cytotactin), reliable 3′ |
| | | | | | | | | end |
| 1403 | TAAAACAAGA d | 41 | 7 | 2 | −6 | −17 | 1369 | Decay accelerating factor for complement (CD55, |
| | | | | | | | | Cramer blood group system), reliable 3′ end |
| 1404 | TGATATGTCA d | 49 | 8 | 0 | −6 | −49 | A1969049 | wq70c08.x1 NCI_CGAP_GC6 Homo sapiens cDNA clone |
| | | | | | | | | IMAGE:2476622 3′ similar to gb:M36820 MACROPHAGE |
| | | | | | | | | INFLAMMATORY PROTEIN-2-ALPHA PRECURSOR HUMAN);, mRNA |
| | | | | | | | | sequence, undefined 3′ end |
| 1405 | CGAATGTCCT d | 72 | 11 | 0 | −7 | −72 | 335952 | keratin 6B, reliable 3′ end |
| 1406 | GTGCGCCGGA d | 61 | 9 | 0 | −7 | −61 | BQ378038 | QV0-UM0093-250800-360-c02 UM0093 Homo sapiens cDNA, |
| | | | | | | | | mRNA sequence, undefined 3′ end |
| 1407 | GCAACTTAGA d | 80 | 11 | 7 | −7 | −11 | 54451 | Laminin, gamma 2 (nicein (100kD), kalinin (105kD), |
| | | | | | | | | BM600 (100kD), shorter alternative transcript |
| 1408 | TCTCTACTAA d | 49 | 7 | 5 | −7 | −10 | 250641 | Tropomyosin 4, reliable 3′ end |
| 1409 | CCTCAGGATA d | 25 | 3 | 0 | −7 | −25 | BC012090 | Homo sapiens, Similar to heterogeneous nuclear |
| | | | | | | | | ribonucleoprotein A3, clone MGC:20045 IMAGE: |
| | | | | | | | | 4661041, mRNA, complete cds, reliable 3′ end |
| 1410 | TCTGTAATCC d | 34 | 4 | 0 | −8 | −34 | 142 | sulfotransferase family, cytosolic, 1A, phenol- |
| | | | | | | | | preferring, member 1, reliable 3′ end |
| 1411 | TCCTGTAAAG d | 34 | 4 | 0 | −8 | −34 | 74034 | Caveolin 1, caveolae protein, 22kD, reliable 3′ end |
| 1412 | GTGTAATAAG d | 77 | 10 | 2 | −8 | −32 | 232400 | Heterogeneous nuclear ribonucleoprotein A2/B1, |
| | | | | | | | | reliable 3′ end |
| 1413 | TAGCTCTATG d | 43 | 6 | 0 | −8 | −43 | 76549 | ATPase, Na+/K+ transporting, alpha 1 poly- |
| | | | | | | | | peptide, reliable 3′ end |
| 1414 | CTTTCTTTGA d | 35 | 4 | 2 | −8 | −15 | 4909 | Dickkopf homolog 3 (Xenopus laevis), reliable 3′ end |
| 1415 | CTTGAGCAAT d | 63 | 8 | 0 | −8 | −63 | 848 | FK506 binding protein 4 (59kD), reliable 3′ end |
| 1416 | AGGCCTCGGC d | 28 | 3 | 2 | −8 | −12 | 301885 | Homo sapiens cDNA FLJ33794 fis, clone CTONG1000009, |
| | | | | | | | | undefined 3′ end |
| 1417 | TTCTTGTTTT d | 57 | 7 | 5 | −9 | −12 | 74621 | Prion protein (p27-30) (Creutzfeld-Jakob disease, |
| | | | | | | | | Gerstmann-Strausler-Scheinker syndrome, fatal |
| | | | | | | | | familial insomnia) reliable 3′ end |
| 1418 | TGTAGGTCAT d | 29 | 3 | 0 | −9 | −29 | 111554 | ADP-ribosylation factor-like 7, reliable 3′ end |
| 1419 | TTAAGACTTC d | 49 | 6 | 0 | −9 | −49 | 136309 | SH13-domain GRB2-like endophilin B1, internal tag |
| 1420 | GGGTTGGCTT d | 118 | 13 | 19 | −9 | −6 | 348493 | LOC114928 Hypothetical protein BC013576, internal |
| | | | | | | | | tag |
| 1421 | GTACTAGTGT d | 89 | 10 | 5 | −9 | −19 | 303649 | small inducible cytokine A2 (monocyte chemotactic |
| | | | | | | | | protein 1), reliable 3′ end |
| 1422 | GTTTTTGCTT d | 20 | 2 | 0 | −9 | −20 | 7718 | hypothetical protein FLJ22678, reliable 3′ end |
| 1423 | GGGGCACTTG d | 20 | 2 | 0 | −9 | −20 | 54451 | Laminin, gamma 2 (nicein (100kD), kalinin (105kD), |
| | | | | | | | | BM600 (100kD), Herlitz junctional epidermolysis |
| | | | | | | | | bullosa)), reliable 3′ end |
| 1424 | CTCAGTCTTT d | 20 | 2 | 0 | −9 | −20 | AW304910 | xv90h12.x1 NCI_CGAP_Bm53 Homo sapiens cDNA clone |
| | | | | | | | | IMAGE:2825831 3′, mRNA sequence, undefined 3′ end |
| 1425 | AATATTGAGA d | 31 | 3 | 2 | −9 | −13 | 106673 | eukaryotic translation initiation factor 3, subunit |
| | | | | | | | | 6 (48kD), reliable 3′ end |
| 1426 | TTATAAAAGA d | 21 | 2 | 0 | −10 | −21 | BG009283 | RC4-GN0321-011200-011-c02 GN0321 Homo sapiens cDNA, |
| | | | | | | | | mRNA sequence, undefined 3′ end |
| 1427 | TATAAGGTGG d | 21 | 2 | 0 | −10 | −21 | 169531 | DEAD/H (Asp-GLu-Ala-Asp/His) box polypeptide 21, |
| | | | | | | | | reliable 3′ end |
| 1428 | TACTGGAAGT d | 21 | 2 | 0 | −10 | −21 | 9075 | serine/threonine kinase 17a (apoptosis-inducing), |
| | | | | | | | | internally primed site |
| 1429 | CTTTCAGATG d | 21 | 2 | 0 | −10 | −21 | 99910 | phosphofructokinase, platelet, reliable 3′ end |
| 1430 | TCACTGCACT d | 68 | 7 | 0 | −10 | −68 | 287617 | Homo sapiens cDNA FLJ14058 fis, clone HEMEBB1000554, |
| | | | | | | | | undefined 3′ end |
| 1431 | TTAATATATG d | 23 | 2 | 0 | −10 | −23 | 356386 | RAB7, member RAS oncogene family, reliable 3′ end |
| 1432 | TTCATACACC d | 350 | 33 | 19 | −11 | −18 | X93334 | mitochondrial |
| 1433 | TACTAGTCCT d | 48 | 4 | 0 | −11 | −48 | BE969428 | 601649644R2 NH_MGC_74 Homo sapiens cDNA clone IMAGE: |
| | | | | | | | | 3933371 3′, mRNA sequence |
| 1434 | TGGATCAACC d | 25 | 2 | 0 | −11 | −25 | 74034 | caveolin 1, caveolae protein, 22kD, reliable 3′ end |
| 1435 | TCCCTATTAA d | 492 | 43 | 181 | −11 | −3 | | No match |
| 1436 | TACAAACGGT d | 26 | 2 | 2 | −12 | −11 | BG563838 | 602584639F1 NH_MGC_76 Homo sapiens cDNA clone IMAGE: |
| | | | | | | | | 4712624 5′, mRNA sequence, undefined 3′ end |
| 1437 | TCAAATGCAT d | 54 | 4 | 5 | −12 | −11 | 182447 | Heterogeneous nuclear ribonucleoprotein C (C1/C2), |
| | | | | | | | | reliable 3′ end |
| 1438 | AGGTCTTCAA d | 86 | 7 | 17 | −13 | −5 | 87409 | thrombospondin 1, reliable 3′ end |
| 1439 | CCTGGTCCCA d | 43 | 3 | 5 | −13 | −9 | 23881 | keratin 7, reliable 3′ end |
| 1440 | TTTCCTCTCA d | 130 | 10 | 0 | −13 | −130 | 184510 | stratifin, reliable 3′ end |
| 1441 | CTGTTGGCAT d | 31 | 2 | 2 | −14 | −13 | 350077 | Ribosomal protein l21, internally primed site |
| 1442 | TTTGTAGATG d | 31 | 2 | 0 | −14 | −31 | 3069 | heat shock 70kD protein 9B (mortalin-2), reliable 3′ |
| | | | | | | | | end |
| 1443 | TCATCATCTG d | 32 | 2 | 2 | −1 | −13 | 116159 | ESTs, reliable 3′ end |
| 1444 | CCATTGCACT d | 86 | 6 | 0 | −16 | −86 | 211563 | B-cell CLL/lymphoma 7A, reliable 3′ end |
| 1445 | GTCCTTTCTG d | 54 | 3 | 0 | −16 | −54 | 7993 | diphtheria toxin receptor (heparin-binding epidermal |
| | | | | | | | | growth factor-like growth factor), reliable 3′ end |
| 1446 | CTTCCTTGCC d | 1204 | 69 | 17 | −17 | −72 | 2785 | keratin 17, reliable 3′ end |
| 1447 | GTTTCATCTC d | 38 | 2 | 0 | −17 | −38 | 1940 | czystallin, alpha B, reliable 3′ end |
| 1448 | AGTGTCTGTG d | 135 | 8 | 29 | −18 | −5 | 8867 | cysteine-rich, angiogenic inducer, 61, reliable 3′ |
| | | | | | | | | end |
| 1449 | ACCAGTGGTT d | 20 | 1 | 0 | −18 | −20 | A1857657 | wk96a06.x1 NCI_CGAP_Lu19 Homo sapiens cDNA clone |
| | | | | | | | | IMAGE:2423218 3′ similar to gb:M93010 14-3-3 |
| | | | | | | | | PROTEIN HOMOLOG STRATIFIN (HUMAN); contains element |
| | | | | | | | | MSR1 MER22 repetitive element;, mRNA sequence, |
| | | | | | | | | undefined 3′ end |
| 1450 | ACACTTCGAG d | 40 | 2 | 0 | −18 | −40 | BF980200 | 602288029T1 NIH_MGC_97 Homo sapiens cDNA clone |
| | | | | | | | | IMAGE:4373839 3′, mRNA sequence, internal tag |
| 1451 | GCTTAGAAGT d | 41 | 2 | 0 | −19 | −41 | 289088 | heat shock 90kD protein 1, alpha, internally primed |
| | | | | | | | | site |
| 1452 | CAGAAGGCCA d | 21 | 1 | 0 | −20 | −21 | 75668 | Homo sapiens, Similar to RIKEN cDNA 1700018018 gene, |
| | | | | | | | | clone IMAGE:4121436, mRNA, partial cds, reliable 3′ |
| | | | | | | | | end |
| 1453 | TTTACTTTGG d | 20 | 0 | 0 | −20 | −20 | 77889 | Friedreich ataxia region gene X123, reliable 3′ end |
| 1454 | TATCCCAACT d | 20 | 0 | 0 | −20 | −20 | AA729014 | nw25h05.s1 NCI_CGAP_GCB0 Homo sapiens cDNA clone |
| | | | | | | | | IMAGE:1241529 3′, mRNA sequence, reliable 3′ end |
| 1455 | CTGACTTGTG d | 20 | 0 | 0 | −20 | −20 | BF869689 | IL3-ET0116-231000-299-H09 ET0116 Homo sapiens cDNA, |
| | | | | | | | | mRNA sequence, undefined 3′ end |
| 1456 | ACCTTTACTG d | 20 | 0 | 0 | −20 | −20 | 77356 | transferrin receptor (p90, CD71), reliable 3′ end |
| 1457 | AAATACCTAA d | 20 | 0 | 0 | −20 | −20 | AW835549 | QV4-LT0016-271299-068-h02 LT0016 Homo sapiens cDNA, |
| | | | | | | | | mRNA sequence, undefined 3′ end |
| 1458 | CTTAAGGATT d | 46 | 2 | 2 | −21 | −19 | 165998 | PAI-1 mRNA-binding protein, reliable 3′ end |
| 1459 | TTGGGTTAAT d | 23 | 1 | 0 | −21 | −23 | AW834375 | MR2-TT0013-241199-018-d09 TT0013 Homo sapiens cDNA, |
| | | | | | | | | mRNA sequence, undefined 3′ end |
| 1460 | TATTTTTGTT d | 23 | 1 | 0 | −21 | −23 | 9238 | FLJ23516 Hypothetical protein FLJ23516, reliable 3′ |
| | | | | | | | | end |
| 1461 | GTGGATGGAC d | 23 | 1 | 0 | −21 | −23 | 6418 | seven transmembrane domain orphan receptor, reliable |
| | | | | | | | | 3′ end |
| 1462 | ATAGACATAA d | 23 | 1 | 0 | −21 | −23 | 78614 | complement component 1, q subcomponent binding |
| | | | | | | | | protein; reliable 3′ end |
| 1463 | AAGGCTGGAA d | 23 | 1 | 0 | −21 | −23 | 85962 | hyaluronan synthase 3, reliable 3′ end |
| 1464 | TTTGTACACA d | 21 | 0 | 0 | −21 | −21 | BE963003 | 601656371R1 NIH_MGC_66 Homo sapiens cDNA clone |
| | | | | | | | | IMAGE:3856313 3′, mRNA sequence |
| 1465 | TGGGAAGAGG d | 21 | 0 | 0 | −21 | −21 | BG569626 | 602587323F1 NIH_MOC_76 Homo sapiens cDNA clone |
| | | | | | | | | IMAGE:4716100 5′, mRNA sequence, undefined 3′ end |
| 1466 | GTATTTAACA d | 21 | 0 | 0 | −21 | −21 | 9006 | VAMP (vesicle-associated membrane protein)- |
| | | | | | | | | associated protein A (33kD), reliable 3′ end |
| 1467 | GGAAAGATGT d | 21 | 0 | 0 | −21 | −21 | 9398 | FLJ10055 Hypothetical protein FLJ10055, internal tag |
| 1468 | TGGAGAATGT d | 23 | 0 | 0 | −23 | −23 | 287797 | ITGB1 Integrin, beta 1 (fibronectin receptor, beta |
| | | | | | | | | polypeptide, antigen CD29 includes MDF2, MSK12), |
| | | | | | | | | internally primed site |
| 1469 | TATGTATGTT d | 23 | 0 | 0 | −23 | −23 | 283738 | casein kinase 1, alpha 1, reliable 3′ end |
| 1470 | TACCTAATTG d | 23 | 0 | 0 | −23 | −23 | BF896098 | CM2-MT0158-221100-551-c04 MT0158 Homo sapiens cDNA, |
| | | | | | | | | mRNA sequence, undefined 3′ end |
| 1471 | TAATAAAGCA d | 23 | 0 | 0 | −23 | −23 | 4888 | seryl-tRNA synthetase, reliable 3′ end |
| 1472 | GTACTGTATG d | 23 | 0 | 0 | −23 | −23 | 180446 | karyopherin (importin) beta 1, reliable 3′ end |
| 1473 | GCTGTAGCCA d | 23 | 0 | 0 | −23 | −23 | BM145758 | TCAAP1D7727 Pediatric acute myelogenous leukemia |
| | | | | | | | | cell (FAB M1) Baylor-HGSC project = TCAA |
| | | | | | | | | Homo sapiens cDNA clone TCAAP7727, mRNA sequence, |
| | | | | | | | | reliable 3′ end |
| 1474 | TTAGATAAGC d | 26 | 1 | 0 | −24 | −26 | 82916 | chaperonin containing TCP1, subunit 6A (zeta 1), |
| | | | | | | | | reliable 3′ end |
| 1475 | TCATAATAGG d | 25 | 0 | 0 | −25 | −25 | | No match |
| 1476 | TAATTTATAG d | 25 | 0 | 0 | −25 | −25 | | No match |
| 1477 | GGTCACTGAG d | 25 | 0 | 0 | −25 | −25 | 254105 | enolase 1, (alpha), internal tag |
| 1478 | CCTTTTTCAA d | 25 | 0 | 0 | −25 | −25 | A1687998 | wa77h02.x1 Soares_NFL_T_GBC_S1 Homo sapiens cDNA |
| | | | | | | | | clone IMAGE:2302227 3′ similar to S W:COX1_HUMAN |
| | | | | | | | | P00395 CYTOCHROME C OXIDASE POLYPEPTIDE 1;, mRNA |
| | | | | | | | | sequence, undefined 3′ end |
| 1479 | ACTACTAAGG d | 25 | 0 | 0 | −25 | −25 | 2820 | oxytocin receptor, reliable 3′ end |
| 1480 | GATGTGCACG d | 520 | 21 | 12 | −25 | −44 | 117729 | keratin 14 (epidermolysis bullosa simple; Dowling- |
| | | | | | | | | Meara, Koebner), reliable 3′ end |
| 1481 | TTCTTTTCAT d | 26 | 0 | 0 | −26 | −26 | 4310 | eukaryotic translation initiation factor 1A, |
| | | | | | | | | reliable 3′ end |
| 1482 | CGAAAGATGT d | 26 | 0 | 0 | −26 | −26 | | No match |
| 1483 | AAAGTCATTG d | 60 | 2 | 0 | −27 | −60 | 77899 | tropomyosin 1 (alpha), internal tag |
| 1484 | TGTGTTGTCA d | 28 | 0 | 0 | −28 | −28 | 154672 | Methylene tetrahydrofolate dehydrogenase (NAD + |
| | | | | | | | | dependent), methenyltetrahydrofolate cyclohydrolase, |
| | | | | | | | | reliable 3′ end |
| 1485 | TCCATCGTCC d | 28 | 0 | 0 | −28 | −28 | R34920 | yg59g06.r1 Soares infant brain INIB Homo sapiens |
| | | | | | | | | cDNA clone IMAGE:37058 5′ similar to S P:CIKB_DROME |
| | | | | | | | | P17970 POTASSIUM CHANNEL PROTEIN SHAB;, mRNA |
| | | | | | | | | sequence, undefined 3′ end |
| 1486 | GTGCAGAGGA d | 28 | 0 | 0 | −28 | −28 | BE974249 | 601680217R2 NIH_MGC_83 Homo sapiens cDNA clone |
| | | | | | | | | IMAGE:3950476 3′, mRNA sequence, undefined 3′ end |
| 1487 | GATATGTTAT d | 28 | 0 | 0 | −28 | −28 | 117938 | Collagen, type XVII, alpha 1, reliable 3′ end |
| 1488 | ATGGTGTATG d | 31 | 1 | 0 | −28 | −31 | BE619862 | 601473114T1 NIH_MGC_68 Homo sapiens cDNA clone |
| | | | | | | | | IMAGE:3876219 3′, mRNA sequence, undefined 3′ end |
| 1489 | TTACTTATAC d | 63 | 2 | 0 | −29 | −63 | C14491 | C14491 Clontech human aorta polyA + mRNA |
| | | | | | | | | (#6572) Homo sapiens cDNA clone GEN-065B04 5′, |
| | | | | | | | | mRNA, undefined 3′ end |
| 1490 | TTCTATTTCA d | 32 | 1 | 0 | −29 | −32 | 170328 | Moesin, reliable 3′ end |
| 1491 | TGTTCATCAT d | 35 | 1 | 2 | −32 | −15 | 65450 | reticulon 4, reliable 3′ end |
| 1492 | TGTTAATGTT d | 35 | 1 | 2 | −32 | −15 | 261828 | MAP kinase-interacting serine/threonine kinase 2, |
| | | | | | | | | reliable 3′ end |
| 1493 | TTTTGTATTT d | 35 | 1 | 0 | −32 | −35 | DF833948 | RC1-HT0881-041100-019-all HT0881 Homo sapiens cDNA, |
| | | | | | | | | mRNA sequence, undefined 3′ end |
| 1494 | TCAATAAAGG d | 32 | 0 | 0 | −32 | −32 | 118797 | ubiquinn-conjugating enzyme E2D 3 (UBC4/5 homolog, |
| | | | | | | | | yeast), reliable 3′ end |
| 1495 | GTGATGGTGT d | 37 | 1 | 2 | −33 | −15 | 197345 | thyroid autoantigen 70kD (Ku antigen), reliable 3′ |
| | | | | | | | | end |
| 1496 | TCATCATCAG d | 35 | 0 | 0 | −35 | −35 | T94401 | ye35f01.s1 Stratagene lung (#937210) Homo |
| | | | | | | | | sapiens cDNA clone IMAGE:119737 3′ similar to gb:M17886 60S ACIDIC RIBOSOMAL PROTEIN P1 (HUMAN);, mRNA sequence, undefined 3′ end |
| 1497 | GGGAAGGGAC d | 80 | 2 | 0 | −36 | −80 | 189559 | EST, reliable 3′ end |
| 1498 | GTAAATATGG d | 124 | 3 | 0 | −38 | −124 | 198689 | bullous pemphigoid antigen 1 (230/240kD), reliable |
| | | | | | | | | 3′ end |
| 1499 | TACCAGTGTA d | 41 | 1 | 0 | −38 | −41 | 79037 | heat shock 60kD protein 1 (chaperonin), reliable 3′ |
| | | | | | | | | end |
| 1500 | GTATTCTCCA d | 38 | 0 | 0 | −38 | −38 | | No match |
| 1501 | CCCCCGTACA d | 92 | 2 | 19 | −42 | −5 | | No match |
| 1502 | TACATAATTA d | 48 | 1 | 2 | −43 | −20 | 240443 | multiple endocrine neoplasia 1, reliable 3′ end |
| 1503 | TATGTGCACG d | 44 | 0 | 0 | −44 | −44 | A1874331 | tz64c12.x1 NCI_CGAP_Ov35 Homo sapiens cDNA clone |
| | | | | | | | | IMAGE:2293366 3′ similar to TR:Q61402 Q61402 GRANULE |
| | | | | | | | | CELL ANTISERUM POSITIVE 8; contains element LTR4 |
| | | | | | | | | repetitive element;, mRNA undefined 3′ end |
| 1504 | TGATTGGTGG d | 54 | 1 | 2 | −49 | −22 | BQ374288 | MR0-FT0176-040900-202-a01 FT0176 Homo sapiens cDNA, |
| | | | | | | | | mRNA sequence, undefined 3′ end |
| 1505 | TGCTTGTGTA d | 52 | 0 | 0 | −52 | −52 | BQ368670 | PM3-GN0510-260501-010-f03 GN0510 Homo sapiens cDNA, |
| | | | | | | | | mRNA sequence, undefined 3′ end |
| 1506 | CATCTGTCTA d | 60 | 1 | 0 | −54 | −60 | 145279 | SET translocation (mycloid leukemia-associated), |
| | | | | | | | | internally primed site |
| 1507 | ACCTTGGTGC d | 61 | 1 | 0 | −56 | −61 | R72649 | yj95e04.s1 Soares breast 2NbHBst Homo sapiens cDNA |
| | | | | | | | | clone IMAGE:156510 3′ similar to gb:J00124_cds1 |
| | | | | | | | | KERATIN, TYPE 1 CYTOSKELETAL 14 (HUMAN);, mRNA |
| | | | | | | | | sequence, undefined 3′ end |
| 1508 | TTTCCTTGCC d | 63 | 0 | 0 | −63 | −63 | AW070788 | xa30d01.x1 NCI_CGAP_Br18 Homo sapiens cDNA clone |
| | | | | | | | | IMAGE:2568289 3′ similar to gb:Z19574_malKERATIN, |
| | | | | | | | | TYPE 1 CYTOSKELETAL 17 (HUMAN);, mRNA sequence, |
| | | | | | | | | reliable 3′ end |
| 1509 | ACACAGCAAG d | 80 | 0 | 0 | −80 | −80 | AW572695 | xx92h01.x2 NCI_CGAP_Lym12 Homo sapiens cDNA clone |
| | | | | | | | | IMAGE:2851153 3′, mRNA sequence, reliable 3′ end |
| 1510 | TACTTTATAA d | 127 | 1 | 0 | −116 | −127 | 8230 | a disintegrin-like and metalloprotease (reprolysin |
| | | | | | | | | type) with thrombospondin type 1 motif, 1, reliable |
| | | | | | | | | 3′ end |

TABLE 9

Genes differentially_expressed in luminal epithelial
cells from DCIS and normal breast tissue

| | | | | | | | | |
|---|---|---|---|---|---|---|---|---|
| SEQ | | | | | | | | |
| ID | Tag | | | | | | | |
| NO: | Sequence | NL | D6 | D7 | d6/n | d7/n | Unigene | Gene |

| | | | | | | | | |
|---|---|---|---|---|---|---|---|---|
| 1511 | AGGAAGGAAC d | 0 | 110 | 24 | 110 | 24 | 323910 | V-erb-b2 erythroblastic leukemia viral oncogene homolog |
| | | | | | | | | 2, neuro/glioblastoma derived oncogene homolog (avian), |
| | | | | | | | | undefined 3′ end |
| 1512 | GTAATCCTGC d | 4 | 187 | 28 | 52 | 8 | AW450286 | UI-H-B13-akz-e-09-0-ULs1 NCI_CGAP_Sub5 Homo sapiens cDNA |
| | | | | | | | | clone IMAGE:2736089 3′, mRNA, reliable 3′ end |
| 1513 | GCTCAGCTGG d | 0 | 31 | 16 | 31 | 16 | 223241 | eukaryotic translation elongation factor 1 delta (guanine |
| | | | | | | | | nucleotide exchange protein), reliable 3′ end |
| 1514 | CCTGCCCACC d | 0 | 21 | 15 | 21 | 15 | 1892 | phenylethanolamine N-methyltransferase, reliable 3′ end |
| 1515 | CCTGGCTAAT d | 13 | 166 | 49 | 13 | 4 | 274170 | Opa-interacting protein 2, reliable 3′ end |
| 1516 | GCCCACAAGT d | 2 | 22 | 46 | 12 | 25 | 285976 | LAG1 longevity assurance homolog 2 (S. cerevisiae), |
| | | | | | | | | reliable 3′ end |
| 1517 | GGCAGCCAGA d | 9 | 92 | 43 | 10 | 5 | 75061 | Macrophage myristoylated alanine-rich C kinase substrate, |
| | | | | | | | | reliable 3′ end |
| 1518 | ACGCAGGGAG | 11 | 99 | 77 | 9 | 7 | 279789 | glucose phosphate isomerase, internal tag |
| 1519 | TTGGCCAGGA | 11 | 89 | 38 | 8 | 3 | 46798 | Homo sapiens mRNA; cDNA DKFZp434K152 (from clone |
| | | | | | | | | DKFZp434K152), reliable 3′ end |
| 1520 | TACCCTGGCA | 4 | 28 | 23 | 8 | 6 | AY014272 | Homo sapiens FKSG30 (FKSG30) mRNA, shorter alternative |
| | | | | | | | | transcript |
| 1521 | TCCCTATTAA | 76 | 563 | 288 | 7 | 4 | 343430 | ESTs, undefinded 3′ end (NCBI only) |
| 1522 | GCTTATTG | 62 | 365 | 226 | 6 | 4 | 288061 | Actin, beta, reliable 3′ end |
| 1523 | ACCCCCCCGC | 64 | 372 | 364 | 6 | 6 | 2780 | jun D proto-oncogene, undefined 3′ end |
| 1524 | CACACAGTTT | 15 | 70 | 71 | 5 | 5 | 204354 | ras homolog gene family, member B, undefined 3′ end |
| 1525 | AGGTCAGGAG | 73 | 310 | 125 | 4 | 2 | 59498 | Cell division cycle 2-like 5 (cholinesterase-related cell |
| | | | | | | | | division controller), reliable 3′ end |
| 1526 | TGGAAAGTGA | 20 | 76 | 132 | 4 | 7 | 25647 | v-fos FBJ murine osteosarcoma viral oncogene homolog, |
| | | | | | | | | reliable 3′ end |
| 1527 | GTGGCAGGCA | 16 | 60 | 46 | 4 | 3 | 241205 | Peroxisomal membrane protein 4 (24kD), reliable 3′ end |
| 1528 | GCCTGCAGTC | 13 | 45 | 81 | 4 | 6 | 31439 | serine protease inhibitor, Kunitz type, 2, reliable 3′ |
| | | | | | | | | end |
| 1529 | ATGACCCCCG | 13 | 44 | 42 | 3 | 3 | AA918111 | o176d02.s1 NCI_CGAP_Kid3 Homo sapiens cDNA clone IMAGE: |
| | | | | | | | | 1535523 3′, mRNA sequence, undefined 3′ end |
| 1530 | CCTGTAGTCC | 15 | 50 | 50 | 3 | 3 | 306226 | Transmembrane gamma-carboxyglutamic acid protein 4, |
| | | | | | | | | reliable 3′ end |
| 1531 | ATCGTGGCGG d | 42 | 105 | 972 | 3 | 23 | 5372 | claudin 4, reliable 3′ end |
| 1532 | CCTGTAATCC | 152 | 353 | 292 | 2 | 2 | 292154 | stromal cell protein (NCBI), reliable 3′ end |
| 1533 | CCACTGCACT | 125 | 275 | 194 | 2 | 2 | 107003 | enhancer of invasion 10 (NCBI), reliable 3′ end |
| 1534 | TGATTTCACT | 294 | 441 | 865 | 2 | 3 | X93334 | mitochondria |
| 1535 | GTGTGGGGGG | 54 | 18 | 21 | −3 | −3 | 2340 | Junction plakoglobin, reliable 3′ end |
| 1536 | ATTCTCCAGT | 87 | 28 | 22 | −3 | −4 | 234518 | ribosomal protein L23, reliable 3′ end |
| 1537 | GCCGTGTCCG | 258 | 82 | 58 | −3 | −4 | 350166 | ribosomal protein S6, reliable 3′ end |
| 1538 | CAGCTCACTG | 58 | 18 | 17 | −3 | −3 | 738 | ribosomal protein L14, reliable 3′ end |
| 1539 | GCCTGTATGA | 67 | 21 | 20 | −3 | −3 | 180450 | ribosomal protein S24, reliable 3′ end |
| 1540 | CTGCCAACTT | 56 | 17 | 22 | −3 | −3 | 180370 | cofilin 1 (non-muscle), internal tag |
| 1541 | CAAGTTTGCT d | 36 | 11 | 3 | −3 | −12 | 181165 | eukaryotic translation elongation factor 1 alpha 1, |
| | | | | | | | | internal tag |
| 1542 | GGGCTGGGGT | 267 | 78 | 74 | −3 | −4 | 90436 | Sperm associated antigen 7, reliable 3′ end |
| 1543 | CGCCGCCGGC | 281 | 76 | 97 | −4 | −3 | 182825 | ribosomal protein L35, reliable 3′ end |
| 1544 | GTAAAAAAAA | 64 | 17 | 18 | −4 | −4 | 460 | Activating transcription factor 3, reliable 3′ end |
| 1545 | TAGAAAGGCA | 36 | 10 | 6 | −4 | −6 | U07802 | Human Tis11d gene, reliable 3′ end |
| 1546 | TGAAATAAAA | 87 | 23 | 21 | −4 | −4 | 9614 | nucleophosmin (nucleolar phosphoprotein B23, numatrin), |
| | | | | | | | | reliable 3′ end |
| 1547 | TGAAAAAAAA | 33 | 9 | 7 | −4 | −5 | 119178 | Cation-chloride cotransporter-interacting protein, |
| | | | | | | | | reliable 3′ end |
| 1548 | ACTCCAAAAA | 158 | 40 | 48 | −4 | −3 | BC012990 | Homo sapiens clone IMAGE:3840457, mRNA, reliable 3′ end |
| 1549 | TGGAAGCACT d | 368 | 94 | 15 | −4 | −25 | 624 | interleukin 8, reliable 3′ end |
| 1550 | GATGAACTGA | 29 | 7 | 6 | −4 | −5 | 30035 | Splicing factor, arginine/serine-rich 10 (transformer 2 |
| | | | | | | | | homolog, Drosophila), reliable 3′ end |
| 1551 | GCCGCCCTGC | 132 | 33 | 18 | −4 | −7 | 82208 | acyl-Coenzyme A dehydrogenase, very long chain, reliable |
| | | | | | | | | 3′ end |
| 1552 | AGAAAAAAAA | 83 | 21 | 20 | −4 | −4 | 597 | Glutamic-oxaloacetic transaminase 1, soluble (aspastate |
| | | | | | | | | aminotransferase 1), reliable 3′ end |
| 1553 | CCCCAGCCAG | 143 | 35 | 33 | −4 | −4 | 252259 | Ribosomal protein S3, reliable 3′ end |
| 1554 | TTGAAGCTTT d | 122 | 29 | 5 | −4 | −24 | 75765 | GRO2 oncogene, reliable 3′ end |
| 1555 | AGCTCTCCCT | 107 | 26 | 47 | −4 | −2 | 82202 | ribosomal protein L17, reliable 3′ end |
| 1556 | CAAAAAAAAA | 107 | 24 | 22 | −4 | −5 | 1217 | Adenosine deaminase, reliable 3′ end |
| 1557 | CCCATCCGAA | 112 | 26 | 23 | −4 | −5 | 91379 | ribosomal protein L26, reliable 3′ end |
| 1558 | AGGGGCGCAG | 38 | 9 | 11 | −4 | −3 | 97616 | SH3-domain GRB2-like 1, reliable 3′ end |
| 1559 | GTCTGCACCT | 33 | 7 | 8 | −4 | −4 | 376798 | Homo sapiens mRNA; cDNA DKFZp547C162 (from clone |
| | | | | | | | | DKFZp547C162), reliable 3′ end |
| 1560 | CCAGAACAGA | 123 | 27 | 59 | −5 | −2 | 334807 | Ribosomal protein L30, reliable 3′ end |
| 1561 | GTGTTAACCA | 58 | 12 | 20 | −5 | −3 | 74267 | ribosomal protein L15, shorter alternative transcipt |
| 1562 | CTGGGTTAAT | 299 | 62 | 97 | −5 | −3 | 298262 | ribosomal protein S19, reliable 3′ end |
| 1563 | GTCTTAAAGT d | 100 | 21 | 8 | −5 | −12 | 177781 | Homo sapiens, clone IMAGE:4711494, mRNA, reliable 3′ end |
| 1564 | AGAGAAATTT | 54 | 11 | 13 | −5 | −4 | 77028 | SEC61B Protein translocation complex beta, reliable 3′ |
| | | | | | | | | end |
| 1565 | CTTCGAAACT | 67 | 13 | 12 | −5 | −6 | 51299 | NADH dehydrogenase (ubiquinone) flavoprotein2 (24kD), |
| | | | | | | | | reliable 3′ end |
| 1566 | TTGGTCCTCT | 435 | 87 | 185 | −5 | −2 | 356795 | ribosomal protein L41, reliable 3′ end |
| 1567 | TGCACGTTTT | 490 | 97 | 96 | −5 | −5 | 169793 | ribosomal protein L32, reliable 3′ end |
| 1568 | GTGCGCTGAG | 103 | 20 | 56 | −5 | −2 | 277477 | HLA-C Major histocompatibility complex, class I, C, |
| | | | | | | | | reliable 3′ end |
| 1569 | GGGAAGCAGA | 78 | 15 | 158 | −5 | 0 | X93334 | mitochondria |
| 1570 | GCATAATAGG | 82 | 15 | 35 | −6 | −2 | 350077 | ribosomal protein L21, reliable 3′ end |
| 1571 | GAAATAAAGT | 27 | 5 | 4 | −6 | −7 | 26498 | hypothetical protein FLJ21657, short alternative |
| | | | | | | | | transcript |
| 1572 | CAACTAATTC | 116 | 21 | 40 | −6 | −3 | 75106 | clusterin (complement lysis inhibitor, SP-40, 40, |
| | | | | | | | | sulfated glycoprotein 2, testosterone-repressed |
| | | | | | | | | prostate message 2, apolipoprotein J), reliable 3′ end |
| 1573 | GCTGCCCTTG | 103 | 18 | 32 | −6 | −3 | 348557 | tubulin alpha 6, reliable 3′ end |
| 1574 | GTTTATGGAT d | 111 | 20 | 1 | −6 | −111 | 365706 | matrix Gla protein, reliable 3′ end |
| 1575 | AATAGGTCCA | 132 | 23 | 34 | −6 | −4 | 113029 | ribosomal protein S25, reliable 3′ end |
| 1576 | CTTCCTGTGA d | 494 | 82 | 5 | −6 | −99 | 348419 | LOC118430 Small breast epithelial mucin, undefined 3′ end |
| 1577 | AACTAAAAAA | 111 | 18 | 9 | −6 | −12 | 3297 | ribosomal protein S27a, reliable 3′ end |
| 1578 | CCCCCTGGAT | 60 | 10 | 12 | −6 | −5 | 275243 | S100 calcium binding protein A6 (calcyclin), reliable 3′ |
| | | | | | | | | end |
| 1579 | GGCACCTCAG | 31 | 5 | 6 | −6 | −5 | 93913 | interleukin 6 (interferon, beta 2), reliable 3′ end |
| 1580 | TAAGGAGCTG | 125 | 20 | 67 | −6 | −2 | 299465 | ribosomal protein S26, reliable 3′ end |
| 1581 | TTGAAACTTT d | 394 | 61 | 1 | −6 | −394 | 789 | GRO1 oncogene (melanoma growth stimulating activity, |
| | | | | | | | | alpha), reliable 3′ end |
| 1582 | TTGGCCAGGG d | 111 | 17 | 10 | −6 | −11 | 321687 | F-box protein FBX30, reliable 3′ end |
| 1583 | TAAAAAAAAA | 64 | 10 | 14 | −6 | −5 | 77910 | 3-hydroxy-3-methylglutaryl-Coenzyme A synthase 1 |
| | | | | | | | | (soluble) (reliable 3′ end to this and several |
| | | | | | | | | others) |
| 1584 | CAATAAACTG | 103 | 16 | 31 | −7 | −3 | 150580 | putative translation initiation factor, shorter |
| | | | | | | | | alternative transcript |
| 1585 | TTTGAAATGA | 129 | 20 | 55 | −7 | −2 | 28491 | spermidine/spermine NI-acetyltransferase, reliable 3′ end |
| 1586 | CACAAACGGT | 218 | 33 | 109 | −7 | −2 | 195453 | ribosomal protein S27 (metallopanstimulin 1), reliable 3′ |
| | | | | | | | | end |
| 1587 | AAGGAGATGG | 98 | 15 | 31 | −7 | −3 | 164170 | vascular Rab-GAP/TBC-containing, reliable 3′ end |
| 1588 | GTGACCACGG | 132 | 20 | 58 | −7 | −2 | BQ447386 | UI-H-EU1-bae-f-07-0-ULs1 NCI_CGAP_Ct1 Homo sapiens cDNA |
| | | | | | | | | clone UI-H-EU1-bae-f-07-0-UI 3′mRNA, reliable 3′ end |
| 1589 | TAATAAAGGT | 42 | 6 | 11 | −7 | −4 | 151604 | ribosomal protein S8, reliable 3′ end |
| 1590 | CTCACTTTTT | 154 | 22 | 22 | −7 | −7 | 76722 | CCAAT/enhancer binding protein (C/EBP), delta, reliable |
| | | | | | | | | 3′ end |
| 1591 | TTCACTGTGA d | 34 | 5 | 3 | −7 | −11 | 621 | lectin, galactoside-binding, soluble, 3 (galectin 3), |
| | | | | | | | | reliable 3′ end |
| 1592 | CTTCCTTGCC | 27 | 4 | 6 | −7 | −5 | 2785 | keratin 17, reliable 3′ end |
| 1593 | GTGAAAAAAA | 36 | 5 | 4 | −7 | −9 | 352394 | Hypothetical protein BC013113, reliable 3′ end |
| 1594 | TGACTGGCAG | 49 | 6 | 9 | −8 | −5 | 278573 | CD59 antigen p18-20 (antigen identified by monoclonal |
| | | | | | | | | antibodies 16.3A5, EJ16, EJ30, EL32 and G344), reliable |
| | | | | | | | | 3′ end, similarity to urokinase plasminogen activator |
| | | | | | | | | receptor |
| 1595 | AATGAGCAAC | 20 | 2 | 3 | −8 | −7 | 171862 | guanylate binding protein 2, interferon-inducible, |
| | | | | | | | | shorter alternative transcript |
| 1596 | GTGGAGCGGA d | 20 | 2 | 2 | −8 | −10 | 323462 | DEAD/H (Asp-Glu-Ala-Asp/His) box polypeptide 30, reliable |
| | | | | | | | | 3′ end |
| 1597 | CCATTGAAAC d | 20 | 2 | 0 | −8 | −20 | 75517 | laminin, beta 3 (nicein (125kD), kalinin (140kD), BM600 |
| | | | | | | | | (125kD)), reliable 3′ end |
| 1598 | GAAAACAAAG d | 20 | 2 | 1 | −8 | −20 | 99936 | keratin 10 (epidermolytic hyperkeratosis; keratosis |
| | | | | | | | | palmaris et plantaris), reliable 3′ end |
| 1599 | TTGGCTTTTC | 31 | 4 | 4 | −8 | −8 | 41569 | phosphatidic acid phosphatase type 2A, internally primed |
| | | | | | | | | site |
| 1600 | TAAAAACTTT d | 62 | 7 | 4 | −8 | −15 | 204096 | secretoglobin, family ID, member 2, reliable 3′ end |
| 1601 | TCGCCGCGAC | 22 | 2 | 4 | −9 | −5 | 296290 | ribosomal protein L37a, undefined 3′ end |
| 1602 | CAGGCCCCAC d | 47 | 5 | 11 | −10 | −4 | 256290 | S100 calcium binding protein A11 (calgizzarin), reliable |
| | | | | | | | | 3′ end |
| 1603 | AGCAGATCAG d | 189 | 20 | 37 | −10 | −5 | 119301 | S100 calcium binding protein A10 (annexin II ligand, |
| | | | | | | | | calpactin I, light polypeptide (p11)), reliable 3′ end |
| 1604 | ATAATAAAAG d | 24 | 2 | 0 | −10 | −24 | 89690 | GRO3 oncogene, reliable 3′ end |
| 1605 | AGAAAGATGT d | 83 | 9 | 4 | −10 | −21 | 78225 | annexin A1 reliable 3′ end |
| 1606 | GCGACAGCTC d | 36 | 4 | 5 | −10 | −5 | BE719410 | CM2-HT0847-050800-313-c12 HT0847 Homo sapiens cDNA, mRNA |
| | | | | | | | | sequence, undefined 3′ end |
| 1607 | TGCTAATTGT d | 25 | 2 | 6 | −10 | −4 | 71968 | Homo sapiens mRNA cDNA DKFZp564F053 (from clone |
| | | | | | | | | DKFZp564F053), reliable 3′ end |
| 1608 | GCAACTTAGA d | 29 | 2 | 1 | −12 | −29 | 54451 | LAMC2 Laminin, gamma 2 (nicein (100kD), kalinin (105kD), |
| | | | | | | | | BM600 (100kD), Herlitz junctional epidermolysis bullosa)) |
| | | | | | | | | shorter alternative transcript |
| 1609 | TCCCCGTACAd | 439 | 37 | 98 | −12 | −4 | | no match |
| 1610 | CGTGGGTGGG d | 74 | 6 | 0 | −12 | −74 | 202833 | Heme oxygenase (decycling) 1, reliable 3′ end |
| 1611 | TGCAGTGACT d | 13 | 0 | 0 | −13 | −13 | 79691 | LIM domain protein, reliable 3′ end |
| 1612 | TGCAAACAGC d | 13 | 0 | 0 | −13 | −13 | BF675978 | 602083935F1 NIH_MGC_83 Homo sapiens cDNA clone IMAGE: |
| | | | | | | | | 4248177 5′, mRNA sequence, internal tag |
| 1613 | GGGTGGGCAG d | 13 | 0 | 0 | −13 | −13 | 284226 | F-box only protein 6, reliable 3′ end |
| 1614 | CTGAAAATTG d | 13 | 0 | 0 | −13 | −13 | 106880 | bystin-like, reliable 3′ end |
| 1615 | AGGTGTGAGC d | 13 | 0 | 0 | −13 | −13 | 323767 | ESTs, internal tag |
| 1616 | AGCAGTGACG d | 13 | 0 | 0 | −13 | −13 | 116651 | epithelial V-like antigen 1, reiable 3′ end |
| 1617 | AGAATTTAGG d | 13 | 0 | 0 | −13 | −13 | 105094 | ESTs, undefined 3′ end |
| 1618 | TCTGGGGACG d | 16 | 1 | 1 | −13 | −16 | 12163 | eukaryotic translation initiation factor 2, subunit 2 |
| | | | | | | | | (beta, 38kD, internally primed site |
| 1619 | GTACTAGTGT d | 33 | 2 | 1 | −13 | −33 | 303649 | small inducible cytokine A2 (monocyte chemotactic protein |
| | | | | | | | | 1), reliable 3′ end |
| 1620 | CGAATGTCCT d | 53 | 4 | 0 | −14 | −53 | 335952 | keratin 6B, reliable 3′ end |
| 1621 | GCTCAAAAAC d | 15 | 0 | 0 | −15 | −15 | R92600 | yq07f04.s1 Soares fetal liver spleen 1NFLS Homo sapiens |
| | | | | | | | | cDNA clone IMAGE:196255 3′similar to contains Alu |
| | | | | | | | | repetitive element, mRNA sequence, undefined 3′ end |
| 1622 | CCCGCCTCTT d | 15 | 0 | 0 | −15 | −15 | BQ358365 | IL3-HT0617-280800-258-G06 HT0617 Homo sapiens cDNA, mRNA |
| | | | | | | | | sequence, undefined 3′ end |
| 1623 | ACAGGAAACT d | 15 | 0 | 0 | −15 | −15 | 69149 | proline-serine-threonine phosphatase interacting protein |
| | | | | | | | | 2, reliable 3′ end |
| 1624 | TAATTTTGGA d | 15 | 0 | 1 | −15 | −15 | 292457 | Homo sapiens, clone MGC:16362 IMAGE:3927795, mRNA, |
| | | | | | | | | complete cds, reliable 3′ end |
| 1625 | AAGCTCGCCG d | 125 | 9 | 0 | −15 | −125 | 62492 | secretoglobin, family 3A, member 1, reliable 3′ end |
| 1626 | GACTCTTCAG d | 396 | 27 | 119 | −15 | −3 | 234726 | serine (or cysteine) proteinase inhibitor, clade A |
| | | | | | | | | (alpha-1 antiproteinase, antitrypsin), member 3, |
| | | | | | | | | reliable 3′ end |
| 1627 | GAGCAGCGCC d | 18 | 1 | 2 | −15 | −9 | 112408 | S100 calcium binding protein A7 (psoriasin 1), reliable |
| | | | | | | | | 3′ end |
| 1628 | C1TCAAAAAA d | 18 | 1 | 1 | −15 | −18 | 6126 | Mannosidase, beta A, lysosomal-Iike, reliable 3′ end |
| 1629 | CTAAAAAAAA d | 38 | 2 | 8 | −16 | −5 | 54457 | CD81 antigen (target of antiproliferative antibody 1), |
| | | | | | | | | reliable 3′ end |
| 1630 | GGTGAGTTAC d | 16 | 0 | 0 | −16 | −16 | 118183 | hypothetical protein FLJ22833, internally primed site |
| 1631 | GTGGTTAAAA d | 20 | 1 | 0 | −16 | −20 | 99949 | Prolactin-induced protein, internal tag |
| 1632 | CCCGAGGCAG d | 62 | 4 | 4 | −17 | −15 | 155223 | stanniocalcin 2, reliable 3′ end |
| 1633 | GCCTTGGGTG d | 64 | 4 | 10 | −17 | −6 | 2250 | leukemia inhibitory factor (cholinergic differentiation |
| | | | | | | | | factor), internal tag |
| 1634 | GACAAAAAAA d | 44 | 2 | 11 | −18 | −4 | 32366 | DERMO1 Likely ortholog of mouse and rat twist-related |
| | | | | | | | | bHLH protein Dermo-1, reliable 3′ end |
| 1635 | GGGAAGGCAC d | 22 | 1 | 3 | −18 | −7 | 13144 | ORM1-like 2 (S. cerevisiae), reliable 3′ end |
| 1636 | GAGGGTTTAG d | 44 | 2 | 2 | −18 | −22 | 75498 | small inducible cytokine subfamily A (Cys-Cys), member |
| | | | | | | | | 20, reliable 3′ end |
| 1637 | GCGCGATGCA d | 18 | 0 | 2 | −18 | −9 | AI420761 | te91a02.x1 NCI_CGAP_Pr28 Homo sapiens cDNA clone IMAGE: |
| | | | | | | | | 2094026 3′, mRNA sequence, undefined 3′ end |
| 1638 | TTGAATCCCC d | 18 | 0 | 0 | −18 | −18 | 112341 | protease inhibitor 3, skin-derived (SKALP), reliable 3′ |
| | | | | | | | | end |
| 1639 | GACACGAACA d | 45 | 2 | 2 | −19 | −23 | 25829 | RAS, dexamethasone-induced 1, reliable 3′ end |
| 1640 | GCGGCTTTCC d | 51 | 2 | 15 | −21 | −3 | 278431 | SCO cytochrome oxidase deficient homolog 2 (yeast), |
| | | | | | | | | reliable 3′ end |
| 1641 | GCTTGCAAAA d | 210 | 10 | 3 | −22 | −70 | 372783 | superoxide dismutase 2, mitochondrial, reliable 3′ end |
| 1642 | GTGTGGCAGC d | 22 | 0 | 0 | −22 | −22 | 42676 | KIAA0781 protein, undefined 3′ end |
| 1643 | TTTTGTGTGA d | 27 | 1 | 4 | −22 | −7 | 182698 | mitochondrial ribosomal protein L20, undefined 3′ end |
| 1644 | CTGGCCCTCG d | 296 | 12 | 74 | −24 | −4 | 350470 | Trefoil factor 1 (breast cancer, estrogen-inducible |
| | | | | | | | | sequence expressed in), reliable 3′ end |
| 1645 | AGGTCTGCCA d | 27 | 0 | 5 | −27 | −5 | 201967 | aldo-keto reductase family 1, member C2 (dihydrodiol |
| | | | | | | | | dehydrogenase 2; bile acid binding protein; 3-alpha |
| | | | | | | | | hydroxysteroid dehydrogenase, type III), reliable 3′ end |
| 1646 | TCTCCAACAA d | 27 | 0 | 0 | −27 | −27 | T69914 | yc19b07.sl Stratagene lung (#937210) Homo sapiens cDNA |
| | | | | | | | | clone IMAGE:81109 3′ similar to gb:J03600 ARACHIDONATE |
| | | | | | | | | 5-LIPOXYGENASE (HUMAN);, mRNA sequence, undefined 3′ end |
| 1647 | GGTAAAATTA d | 29 | 0 | 2 | −29 | −15 | 340959 | Ts translation elongation factor, mitochondrial, reliable |
| | | | | | | | | 3′ end |
| 1648 | CTTAAAAAAA d | 36 | 1 | 0 | −30 | −36 | 75063 | human immunodeficiency virus type I enhancer binding |
| | | | | | | | | protein 2, reliable 3′ end |
| 1649 | GCAGGCCAAG d | 93 | 2 | 16 | −38 | −6 | 69771 | B-factor, properdin, reliable 3′ end |
| 1650 | GGAAAAGTGG d | 96 | 2 | 2 | −39 | −48 | 297681 | serine (or cysteine) proteinase inhibitor, clade A |
| | | | | | | | | (alpha-1 antiproteinase, antitrypsin), member 1, |
| | | | | | | | | reliable 3′ end |
| 1651 | TTTGCTTTTG d | 40 | 0 | 8 | −40 | −5 | 234642 | aquaporin 3, reliable 3′ end |
| 1652 | CTTCTCCAAA d | 42 | 0 | 0 | −42 | −42 | W03794 | za61g08.r1 Soares fetal liver spleen 1NFLS Homo sapiens |
| | | | | | | | | cDNA clone IMAGE:297086 5′ similar to gb:X54486_mal |
| | | | | | | | | PLASMA PROTEASE C1 INHIBITOR PRECURSOR (HUMAN);, mRNA, |
| | | | | | | | | undefined 3′ end |
| 1653 | TTGGTTTTTG d | 56 | 1 | 0 | −46 | −56 | 164021 | Small inducible cytokine subfamily B (Cys-X-Cys), member |
| | | | | | | | | 6 (granulocyte chemotactic protein 2), reliable 3′ end |
| 1654 | GTGCGGAGGA d | 60 | 0 | 1 | −60 | −60 | 332053 | serum amyloid A1, reliable 3′ end |
| 1655 | TGCAGCACGA d | 67 | 0 | 6 | −67 | −11 | 277477 | HLA-C major histocompatibility complex, class I, C, |
| | | | | | | | | reliable 3′ end |
| 1656 | ACACAGCAAG d | 243 | 0 | 0 | −243 | −243 | AW572695 | xx92h01.x2 NCI_CGAP_Lym12 Homo sapiens cDNA clone |
| | | | | | | | | IMAGE:2851153 3′, mRNA sequence, reliable 3′ end |

TABLE 10

Genes differentially expressed in endothelial
cells from DCIS and normal breast tissue

| | | | | | | |
|---|---|---|---|---|---|---|
| SEQ | | | | | | |
| ID | Tag | | | | | |
| NO: | Sequence | NL | D6 | d6/n | Unigene | Gene |

| | | | | | | |
|---|---|---|---|---|---|---|
| 1657 | CGTGGGTGGG d | 0 | 73 | 73 | 202833 | Homo oxygenase (decycling) 1, reliable 3′ end |
| 1658 | TTTGAGGATT d | 0 | 33 | 33 | 18792 | thioredoxin-like, 32kD, internal tag |
| 1659 | TAAATAATTT d | 0 | 33 | 33 | 1197 | heat shock 10kD protein 1 (chaperonin 10), reliable 3′ end |
| 1660 | GCAGAATAGA d | 0 | 29 | 29 | 236218 | Tripartite motif-containing 32, internal tag |
| 1661 | GATAACTACA d | 0 | 27 | 27 | 119206 | insulin-like growth factor binding protein 7, shorter |
| | | | | | | alternative transcript |
| 1662 | GCTTTCTCAC d | 0 | 26 | 26 | BG223065 | nah42g11.x1 NCI_CGAP_HN21 Homo sapiens cDNA clone IMAGE: |
| | | | | | | 4233812 3′, mRNA sequence, undefined 3′ end |
| 1663 | GAAAAGGTTA d | 0 | 22 | 22 | 16085 | putative G-protein coupled receptor, reliable 3′ end |
| 1664 | AAATTGTTGG d | 0 | 22 | 22 | 120932 | ESTs, reliable 3′ end |
| 1665 | GTAATGACAG d | 0 | 21 | 21 | 25590 | stanniocalcin 1, reliable 3′ end |
| 1666 | TGCCTCTGTC d | 0 | 21 | 21 | AA954388 | oo01c02.s1 Soares_NFL_T_GBC_S1 Homo sapiens cDNA clone IMAGE: |
| | | | | | | 1564898 3′ similar to gb:X00737 PURINE NUCLEOSIDE PHOSPHORY- |
| | | | | | | LASE (HUMAN);, mRNA sequence, reliable 3′ end |
| 1667 | TCTTGATTTA d | 0 | 21 | 21 | 74561 | alpha-2-macroglobulin, reliable 3′ end |
| 1668 | GACGACTGAC d | 0 | 21 | 21 | 155530 | interferon, gamma-inducible protein 16, reliable 3′ end |
| 1669 | CCCCCTGCCC d | 3 | 40 | 15 | 177596 | Hypothetical protein FLJ10350, reliable 3′ end |
| 1670 | CAGTTCTCTG d | 3 | 38 | 15 | 279921 | hypothetical protein MGC8721, reliable 3′ end |
| 1671 | AGACAAGCTG d | 3 | 37 | 14 | 166975 | Splicing factor, arginine/serine-rich 5, reliable 3′ end |
| 1672 | ACAGTGGGGA d | 3 | 37 | 14 | 278270 | Unactive progesterone receptor, 23 kD, reliable 3′ end |
| 1673 | CCTGTGTTGG d | 5 | 71 | 14 | AV728954 | AV728954 HTC Homo sapiens cDNA clone HTCCGG11 5′, mRNA |
| | | | | | | sequence, internal tag |
| 1674 | ATGTCTTTTC d | 3 | 34 | 13 | 1516 | insulin-like growth factor binding protein 4, undefined 3′ end |
| 1675 | CATTTCAGAG d | 3 | 32 | 12 | 15259 | BCL2-associated athanogene 3, reliable 3′ end |
| 1676 | GGATTGTCTG d | 3 | 30 | 12 | 83753 | small nuclear ribonucleoprotein polypeptides B and BI, |
| | | | | | | reliable 3′ end |
| 1677 | TTAGTGTCGT d | 3 | 27 | 11 | AW805523 | QVI-UM0103-250400-173-f02 UMO103 Homo sapiens cDNA, mRNA |
| | | | | | | sequence, undefined 3′ end |
| 1678 | AGGAACTGTA d | 3 | 27 | 11 | 184634 | hypothetical protein FLJ20005, reliable 3′ end |
| 1679 | ACAGCGCTGA d | 3 | 27 | 11 | 352392 | major histocompatibility complex; class II, DR beta 5 |
| 1680 | GGCTGGTCTG d | 10 | 108 | 10 | 337986 | hypothetical protein MGC4677, reliable 3′ end |
| 1681 | GACCGCAGGA d | 16 | 161 | 10 | 119129 | collagen, type IV, alpha 1, reliable 3′ end |
| 1682 | TAATTTGCAT d | 5 | 54 | 10 | 79368 | epithelial membrane protein 1, reliable 3′ end |
| 1683 | AAAACATTCT d | 117 | 1175 | 10 | X93334 | mitochondrial |
| 1684 | TCTCTGAGCA | 5 | 38 | 7 | 211604 | a disintegrin-like and metalloprotease (reprolysin type) with |
| | | | | | | thrombospondin type 1 motif, 4, reliable 3′ end |
| 1685 | TTTAACGGCC | 36 | 268 | 7 | X93334 | mitochondrial |
| 1686 | TGTACCTGTA | 8 | 56 | 7 | 334842 | Tubulin, alpha, ubiquitous, reliable 3′ end |
| 1687 | TCCAGAATCC | 8 | 56 | 7 | 7764 | KIAA0469 gene product, reliable 3′ end |
| 1688 | GGAAGGGGAG | 5 | 37 | 7 | 73090 | Nuclear factor of kappa light polypeptide gene enhancer in B- |
| | | | | | | cells 2 (p49/p100), reliable 3′ end |
| 1689 | AAAACTGCAC | 5 | 37 | 7 | 8084 | hypothetical protein dJ465N24.2.1, reliable 3′ end |
| 1690 | CATATCATTA | 42 | 277 | 7 | 119206 | insulin-like growth factor binding protein 7, reliable 3′ end |
| 1691 | AGACCAAAGT | 13 | 86 | 7 | 82646 | DnaJ (Hsp40) homolog, subfamily B, member 1, reliable 3′ end |
| 1692 | TGTAGTTTGA | 5 | 33 | 6 | 171626 | transcription elongation factor B (SIII), polypeptide 1-like, |
| | | | | | | reliable 3′ end |
| 1693 | TGCTGTGCAT | 10 | 60 | 6 | 75692 | Asparagine synthetase, reliable 3′ end |
| 1694 | TATGAGGGTA | 8 | 45 | 6 | 24950 | regulator of G-protein signalling 5, reliable 3′ end |
| 1695 | GCCATAAAAT | 8 | 45 | 6 | 1908 | proteoglycan 1, secretory granule, reliable 3′ end |
| 1696 | AAGACAGTGG | 21 | 118 | 6 | 296290 | Ribosomal protein L37a, reliable 3′ end |
| 1697 | CCAATTTATC | 8 | 44 | 6 | 94 | DnaJ (Hsp40) homolog, subfamily A, member 1, reliable 3′ end |
| 1698 | AAAGTGAAGA | 8 | 41 | 5 | 334477 | FLJ23277 protein, reliable 3′ end |
| 1699 | CCAGGAGGAA | 18 | 95 | 5 | 180414 | heat shock 70kD protein 8, reliable 3′ end |
| 1700 | GAGAACCGTA | 8 | 40 | 5 | 105547 | neural proliferation, differentiation and control, 1, reliable |
| | | | | | | 3′ end |
| 1701 | TGTTCTGGAG | 10 | 52 | 5 | 74471 | Gap junction protein, alpha 1, 43kD (connexin 43), reliable 3′ |
| | | | | | | end |
| 1702 | AAGGAGATGG | 18 | 91 | 5 | 164170 | vascular Rab-GAP/TBC-containing, reliable 3′ end |
| 1703 | TGTCCTGGTT | 26 | 129 | 5 | 179665 | Cyclin-dependent kinase inhibitor 1A (p21, Cip1), reliable 3′ |
| | | | | | | end |
| 1704 | GGAGAGGAAG | 8 | 38 | 5 | 16313 | Kruppel-like zinc finger protein GLIS2, reliable 3′ end |
| 1705 | CTGACCTGTG | 26 | 126 | 5 | BM151142 | TCBAP1D13652 Pediatric pre-B cell acute lymphoblastic leukemia |
| | | | | | | Baylor-HGSC project = TCBA Homo sapiens cDNA clone TCBAP1365, |
| | | | | | | mRNA sequence, reliable 3′ end |
| 1706 | TGGAAGCACT | 23 | 113 | 5 | 624 | interleukin 8, reliable 3′ end |
| 1707 | CACAAACGGT | 94 | 431 | 5 | 195453 | ribosomal protein S27 (metallopanstimulin 1), reliable 3′ end |
| 1708 | AAGGGAGGGT | 18 | 80 | 4 | 182248 | sequestosome 1, reliable 3′ end |
| 1709 | TAACAGCCAG | 31 | 130 | 4 | 81328 | nuclear factor of kappa light polypeptide gene enhancer in B- |
| | | | | | | cells inhibitor, alpha, reliable 3′ end |
| 1710 | ACATCATCGA | 18 | 76 | 4 | 182979 | ribosomal protein L12, reliable 3′ end |
| 1711 | GTGACCACGG | 10 | 43 | 4 | BQ447386 | UI-H-EU1-bae-f-07-0-ULs1 NCI_CGAP_Ct1 Homo sapiens cDNA clone |
| | | | | | | UI-H-EU1-bae-f-07-0-UI 3′ mRNA, reliable 3′ end |
| 1712 | TGTTGAAAAA | 10 | 43 | 4 | 89546 | selectin E (endothelial adhesion molecule 1), reliable 3′ end |
| 1713 | GTTCACTGCA | 16 | 63 | 4 | 168383 | intercellular adhesion molecule 1 (CD54), human rhinovirus |
| | | | | | | receptor, reliable 3′ end |
| 1714 | CCAGAACAGA | 49 | 198 | 4 | 334807 | ribosomal protein L30, reliable 3′ end |
| 1715 | CTCATAAGGA | 18 | 73 | 4 | X93334 | mitochondrial |
| 1716 | CTTAATCCTG | 16 | 60 | 4 | 298275 | solute carrier family 38, member 2, reliable 3′ end |
| 1717 | TTTGAAATGA | 18 | 70 | 4 | 28491 | spermidine/spermine N1-acetyltransferase, reliable 3′ end |
| 1718 | ATAATTCTTT | 104 | 397 | 4 | 539 | ribosomal protein S29, reliable 3′ end |
| 1719 | AGATTCAAAC | 13 | 49 | 4 | 14368 | SH3 domain binding glutamic acid-rich protein like |
| 1720 | CCGTCCAAGG | 44 | 166 | 4 | 80617 | ribosomal protein S16, reliable 3′ end |
| 1721 | TAATCCTCAA | 18 | 62 | 3 | 78409 | collagen, type XVIII, alpha 1, shorter alternative transcript |
| 1722 | GTGCGCTGAG | 44 | 150 | 3 | 277477 | Major histocompatibility complex, class I, C, reliable 3′ end |
| 1723 | GTTCCCTGGC | 21 | 69 | 3 | 177415 | Finkel-Biskis-Reilly murine sarcoma virus (FBR-MuSV) |
| | | | | | | ubiquitously expressed (fox derived); ribosomal protein S30, |
| | | | | | | reliable 3′ end |
| 1724 | TGAAGTAACA | 18 | 59 | 3 | 150580 | putative translation initiation factor, reliable 3′ end |
| 1725 | CCTAGCTGGA | 36 | 117 | 3 | 342389 | peptidylprolyl isomerase A (cyclophilin A), reliable 3′ end |
| | | | | | | (intracellular receptor) |
| 1726 | TACCATCAAT | 18 | 58 | 3 | 169476 | glyceraldehyde-3-phosphate dehydrogenase, reliable 3′ end |
| 1727 | AATCCTGTGG | 18 | 58 | 3 | 178551 | ribosomal protein L8, reliable 3′ end |
| 1728 | CAGAGATGAA | 57 | 181 | 3 | 8997 | Sad1 unc-84 domain protein 1, reliable 3′ end |
| 1729 | AAGGTGGAGG | 55 | 170 | 3 | 163593 | Ribosomal protein L18a, reliable 3′ end |
| 1730 | TGCACTTCAA | 52 | 155 | 3 | 75445 | SPARC-like 1 (mast9, hevin), reliable 3′ end |
| 1731 | GGCCTGCTGC | 21 | 62 | 3 | 9634 | LOC113246 Hypothetical protein BC009925, reliable 3′ end |
| 1732 | AGGGCTTCCA | 76 | 218 | 3 | 29797 | ribosomal protein L10, shorter alternative transcript |
| 1733 | GTGAAGGCAG | 60 | 173 | 3 | 77039 | ribosomal protein S3A, reliable 3′ end |
| 1734 | CAAGCATCCC | 65 | 187 | 3 | X93334 | mitochondrial |
| 1735 | AGAATCACTT | 26 | 73 | 3 | 130815 | hypothetical protein FLJ21870, reliable 3′ end |
| 1736 | GAAGCAGGAC | 34 | 92 | 3 | 180370 | cofilin 1 (non-muscle), reliable 3′ end |
| 1737 | GCTTTTAAGG | 36 | 99 | 3 | 8102 | Ribosomal protein S20, reliable 3′ end |
| 1738 | GCATAATAGG | 68 | 181 | 3 | 350077 | ribosomal protein L21, reliable 3′ end |
| 1739 | CCCTGGGTTC | 29 | 73 | 3 | 111334 | Ferritin, light polypeptide, reliable 3′ end |
| 1740 | GGGACGAGTG | 68 | 169 | 2 | 351316 | Transmembrane 4 superfamily member 1, reliable 3′ end |
| 1741 | GGCAAGAAGA | 36 | 89 | 2 | 111611 | ribosomal protein L27, reliable 3′ end |
| 1742 | TGTGCTAAAT | 34 | 82 | 2 | 250895 | ribosomal protein L34, shorter alternative transcript |
| 1743 | ATGTGAAGAG | 180 | 432 | 2 | 111779 | secreted protein, acidic, cysteine-rich (osteonectin), |
| | | | | | | reliable 3′ end |
| 1744 | TCAGATCTTT | 109 | 259 | 2 | 108124 | ribosomal protein S4, X-linlced, reliable 3′ end |
| 1745 | CTAAGACTTC | 380 | 885 | 2 | X93334 | mitochondrial |
| 1746 | CAATAAATGT | 60 | 137 | 2 | 337445 | ribosomal protein L37, reliable 3′ end |
| 1747 | GTTGTGGTTA | 219 | 493 | 2 | 75415 | beta-2-microglobulin, reliable 3′ end |
| 1748 | GGATTTGGCC | 182 | 393 | 2 | 351937 | Ribosomal protein, large P2, reliable 3′ end |
| 1749 | GTGCTGAATG | 52 | 111 | 2 | 77385 | Myosin, light polypeptide 6, alkali, smooth muscle and non- |
| | | | | | | muscle, reliable 3′ end |
| 1750 | GGAGTGTGCT | 57 | 114 | 2 | 9615 | myosin, light polypeptide 9, regulatory, reliable 3′ end |
| 1751 | GGCAAGCCCC | 86 | 166 | 2 | 334895 | ribosomal protein L10a, reliable 3′ end |
| 1752 | TAGGTTGTCT | 169 | 327 | 2 | 279860 | Tumor protein, translationally-controlled 1, reliable 3′ end |
| 1753 | TTGGTCCTCT | 180 | 346 | 2 | 356795 | ribosomal protein L41, reliable 3′ end |
| 1754 | TCCAAATCGA | 120 | 218 | 2 | 297753 | vimentin, reliable 3′ end |
| 1755 | CTGGGTTAAT | 177 | 318 | 2 | 298262 | ribosomal protein S19, reliable 3′ end |
| 1756 | TGGAAAGTGA | 175 | 313 | 2 | 25647 | v-fos FBJ murine osteosarcoma viral oncogene homolog, reliable |
| | | | | | | 3′ end |
| 1757 | TGGTGTTGAG | 94 | 165 | 2 | 275865 | ribosomal protein S18, reliable 3′ end |
| 1758 | GCCGAGGAAG | 112 | 196 | 2 | 339696 | ribosomal protein S12, reliable 3′ end |
| 1759 | CACCTAATTG | 175 | 299 | 2 | X93334 | niitochondrial |
| 1760 | GAAAAATGGT | 117 | 191 | 2 | 181357 | laminin receptor 1 (67kD, ribosoinal protein SA), reliable 3′ |
| | | | | | | end |
| 1761 | TGCACGTTTT | 234 | 379 | 2 | 169793 | ribosomal protein L32, reliable 3′ end |
| 1762 | GGGCTGGGGT | 180 | 288 | 2 | 90436 | Sperm associated antigen 7, reliable 3′ end |
| 1763 | AGCACCTCCA | 133 | 211 | 2 | 75309 | eukryotic translation elongation factor 2, reliable 3′ end |
| 1764 | ACCAAAAACC | 201 | 51 | −2 | 172928 | collagen, type I, alpha 1, internally primed site |
| 1765 | CAAATCCAAA | 55 | 14 | −2 | 227400 | mitogen-activated protein kinase kinase kinase kinase 3 |
| 1766 | TTACCATATC | 44 | 11 | −2 | 300141 | ribosomal protein L39 |
| 1767 | GAAATAAAGC | 52 | 12 | −2 | 300697 | immunoglobulin heavy constant gamma 3 (G3m marker), reliable |
| | | | | | | 3′ end |
| 1768 | ACCCCCCCGC | 656 | 147 | −2 | 2780 | jun D proto-oncogen; undefined 3′ end |
| 1769 | CGAGGGGCCA | 39 | 8 | −3 | 182485 | actinin, alpha 4, undefined 3′ end |
| 1770 | GATCAGGCCA | 120 | 25 | −3 | 119571 | Collagen, type III, alpha 1 (Ehlers-Danlos syndrome type IV, |
| | | | | | | autosomal dominant), shorter alternative transcript |
| 1771 | TTTCCCTCAA | 34 | 7 | −3 | 75111 | protease, serine, 11 (IGF binding), similar to IGFBP7, cleaves |
| | | | | | | IGF |
| 1772 | GAGCAGCTGG | 31 | 5 | −3 | 166887 | copine I, reliable 3′ end |
| 1773 | TTTGCACCTT | 120 | 21 | −3 | 75511 | connective tissue growth factor, undefined 3′ end |
| 1774 | AGCCACCGCG | 47 | 7 | −4 | 193716 | Complement component (3b/4b) receptor 1, including Knops blood |
| | | | | | | group system, reliable 3′ end |
| 1775 | GGCCGCGAGG | 47 | 7 | −4 | 78344 | myosin, heavy polypeptide 11, smooth muscle, internally primed |
| | | | | | | site |
| 1776 | GGGGTAAGAA | 29 | 4 | −4 | 80423 | prostatic binding protein, reliable 3′ end |
| 1777 | GGCCCGGCTT | 29 | 4 | −4 | 283639 | chromosome 2 open reading frame 9, reliable 3′ end |
| 1778 | GGGCCAACCC | 65 | 8 | −4 | B1012736 | PM3-ET0153-100101-008-c01 ET0153 Homo sapiens cDNA, mRNA |
| | | | | | | sequence undefined 3′ end |
| 1779 | GACCACCAGA | 34 | 4 | −4 | 172928 | Collagen, type I, alpha 1, internal tag |
| 1780 | CTAAAATAGT | 39 | 4 | −5 | 93557 | proenkephalin (NCBI only) |
| 1781 | GGCAATTCAA | 26 | 3 | −5 | 349150 | Homo sapiens cDNA FLJ33107 fis, clone TRACH2000959, reliable |
| | | | | | | 3′ end |
| 1782 | CCCCGCCAAG | 26 | 3 | −5 | 169718 | Calponin 2, reliable 3′ end |
| 1783 | TCCCTATTAG | 16 | 0 | −6 | | no match |
| 1784 | GCCAAAACCT | 16 | 0 | −6 | 158287 | syndecan 3 (N-syndecan |
| 1785 | CCCCTATTAA | 16 | 0 | −6 | | no match |
| 1786 | GGGGGCTCAG | 31 | 3 | −6 | 276919 | ESTs, reliable 3′ end |
| 1787 | GAGATCCGCA | 31 | 3 | −6 | 75348 | proteasome (prosome, macropain) activator subunit 1 (PA28 |
| | | | | | | alpha), reliable 3′ end |
| 1788 | GCCGGCTCAT | 16 | 0 | −6 | AA213605 | zq93d11.rl Stratagene hNT neuron (#937233) Homo sapiens cDNA |
| | | | | | | clone IMAGE:649557 5′ similar to contains Alu repetitive |
| | | | | | | element;. mRNA sequence, undefined 3′ end |
| 1789 | GATTCTGGGT | 16 | 0 | −6 | 334637 | MGC15619 Hypothetical protein MGC15619, internal tag |
| 1790 | ACACAGCAAG | 125 | 10 | −7 | AW572695 | xx92h01.x2 NCI_CGAP_Lym12 Homo sapiens cDNA clone IMAGE: |
| | | | | | | 2851153 3′, mRNA sequence, reliable 3′ end |
| 1791 | CTCAACCCCC | 36 | 3 | −7 | 89137 | Low density lipoprotein-related protein I (alpha-2-macro- |
| | | | | | | globulin receptor), reliable 3′ end |
| 1792 | CTCTCAATAT | 18 | 0 | −7 | 279518 | amyloid beta (A4) precursor-like protein 2, shorter alterna- |
| | | | | | | tive transcript |
| 1793 | CCCGCCTCTT | 18 | 0 | −7 | BQ358365 | IL3-HT0617-280800-258-G06 HT0617 Homo sapiens cDNA, mRNA |
| | | | | | | sequence, undefined 3′ end |
| 1794 | GGGGTGCTGT | 18 | 0 | −7 | 166161 | dynamin 1, reliable 3′ end |
| 1795 | GCTAGGCCGG | 18 | 0 | −7 | BG876456 | QV0-DT0020-090200-106-b04 DT0020 Homo sapiens cDNA, mRNA |
| | | | | | | sequence, undefined 3′ end |
| 1796 | GAGCCAGGCT | 18 | 0 | −7 | 83326 | matrix metalloproteinase 3 (stromelysin 1, progelatinase), |
| | | | | | | reliable 3′ end |
| 1797 | AGGGTCCCCG | 18 | 0 | −7 | Z00013 | H.sapiens germline gene for the leader peptide and variable |
| | | | | | | region of a kappa immunoglobulin (subgroup V kappa I, |
| | | | | | | undefined 3′ end |
| 1798 | TGGCTGGGAA | 21 | 1 | −8 | 172684 | vesicle-assosiated membrane protein 8 (endobrevin), reliable |
| | | | | | | 3′ end |
| 1799 | GAGAGAAAAT | 21 | 1 | −8 | 181444 | Hypothetical protein LOC51235, reliable 3′ end |
| 1800 | CCTGTGGTCC | 21 | 1 | −8 | 334541 | Similar to Zinc finger protein 20 (Zinc finger protein KOX13), |
| | | | | | | reliable 3′ end |
| 1801 | CCTCCAGCTA | 21 | 1 | −8 | 242463 | keratin 8, reliable 3′ end |
| 1802 | ATCAAATCCA | 21 | 1 | −8 | 288581 | Homo sapiens mRNA for FLJ00239 protein, internal tag |
| 1803 | GTCAAAATTT | 21 | 0 | −8 | 108623 | Thrombospondin 2, reliable 3′ end |
| 1804 | GAAACCCCAG | 21 | 0 | −8 | 84359 | Likely ortholog of Xenopus dullard, reliable 3′ end |
| 1805 | CTCCACCCGA | 21 | 0 | −8 | 311815 | EST, reliable 3′ end |
| 1806 | TTAAATAGCA | 21 | 1 | −8 | 76698 | stress-associated endoplasmic reticulum protein 1; ribosome |
| | | | | | | associated membrane protein 4, internally primed site |
| 1807 | CTAACGGGGC | 21 | 1 | −8 | 102171 | immunoglobulin superfamily containing leucine-rich repeat, |
| | | | | | | reliable 3′ end |
| 1808 | GTGCTAAGCA | 21 | 0 | −8 | AI811424 | tW73h08.x1 NCI_CGAP_U3 Homo sapiens cDNA clone IMAGE:2265375 |
| | | | | | | 3′ similar to SW:CA26_MOUSE Q02788 COLLAGEN ALPHA 2(VI) CHAIN |
| | | | | | | PRECURSOR; contains MER22.t1 MSR1 repetitive element; mRNA |
| | | | | | | sequence, reliable 3′ end |
| 1809 | ATGTTAGTGT | 21 | 0 | −8 | 71573 | Hypothetical protein FLJ10074, internal tag |
| 1810 | GAAATCCAAA | 23 | 1 | −9 | 248396 | EST, Moderately similar to C35863 tryptase (EC 3.4.21.59) III |
| | | | | | | precursor-human, reliable 3′ end |
| 1811 | GGGGGGGGGG | 23 | 0 | −9 | 329973 | EST, Weakly similar to 0903209A peptide PD, basic Pro rich |
| | | | | | | [Homo sapiens], reliable 3′ end |
| 1812 | GACATCAAGT | 23 | 0 | −9 | 182265 | keratin 19, reliable 3′ end |
| 1813 | CTCGCGCTGG | 23 | 0 | −9 | 25640 | claudin 3, reliable 3′ end |
| 1814 | CCTGCCCACC d | 26 | 1 | −10 | 1892 | phenylethanolamine N-methyltransferase, reliable 3′ end |
| 1815 | CTCACCGCCC d | 29 | 1 | −11 | 183650 | cellular retinoic acid binding protein 2, reliable 3′ end |
| 1816 | AGGAGCGGGG d | 29 | 1 | −11 | 252189 | Syndecan 4(amphiglycan, ryudocan), undefined 3′ end |
| 1817 | TCCCTATGAA d | 29 | 0 | −11 | | no match |
| 1818 | GGAACAAACA d | 29 | 0 | −11 | 286124 | CD24 antigen (small cell lung carcinoma cluster 4 antigen), |
| | | | | | | reliable 3′ end |
| 1819 | TCCCTATGAA d | 29 | 0 | −11 | | no match |
| 1820 | TAGGTCCCCT d | 29 | 0 | −11 | 82985 | Collagen, type V, alpha 2, internal tag |
| 1821 | TCCGTATTAA d | 31 | 0 | −12 | | no match |
| 1822 | TCCGTATTAA d | 31 | 0 | −12 | | no match |
| 1823 | GGCTGCCCAG d | 34 | 1 | −13 | 172210 | MUF1 protein, reliable 3′ end |
| 1824 | TTCGGTTGGT d | 34 | 0 | −13 | BG939135 | cn30g02.x1 Normal Human Trabecular Bone Cells Homo sapiens |
| | | | | | | cDNA clone NHTBC_cn30g02 random, mRNA sequence, undefined 3′ |
| | | | | | | end |
| 1825 | TCCCTAGTAA d | 36 | 0 | −14 | | no match |
| 1826 | AGCTGTCCCC d | 39 | 1 | −15 | X93334 | mitochondrial |
| 1827 | ACCTGCACAA d | 39 | 0 | −15 | BM690922 | UI-E-CI1-aaz-e-11-0-ULr1 UI-E-C11 Homo sapiens cDNA clone |
| | | | | | | UI-E-C11-aaz-e-11-0-UI 5′, mRNA, undefined 3′ end |
| 1828 | CCGGGGGAGC d | 44 | 1 | −17 | 172928 | collagen, type I, alpha 1, internal tag |
| 1829 | GCCTACCCGA d | 49 | 1 | −19 | 23582 | tumor-associated calcium signal transducer 2, reliable 3′ end |
| 1830 | TCCCTATTAA d | 2798 | 43 | −35 | | no match |
| 1831 | ATCGTGGCGG d | 177 | 0 | −68 | 5372 | Claudin 4, reliable 3′ end |

TABLE 11
Genes from Table 7 encoding secreted and cell surface proteins

| Unigene | Gene |
|---|---|
| 375570 | HLA-DRB1, major histocompatibility complex, class II, DR |
| | beta 1 |
| 126256 | interleukin 1, beta |
| 76807 | major histocompatibility complex, class II, DR alpha |
| 73817 | small inducible cytokine A3 |
| 169401 | apolipoprotein E |
| 79356 | Lysosomal-associated multispanning membrane protein-5, |
| | haematopoetic cell specific |
| 179657 | plasminogen activator, urokinase receptor |
| 17409 | cysteine-rich protein 1 (intestinal) |
| 74631 | basigin (OK blood group), leukocyte activation M6 antigen |
| 814 | major histocompatibility complex, class II, DP beta 1 |
| 352107 | trefoil factor 3 (intestinal) |

TABLE 12
Genes from Table 8 encoding secreted or cell surface proteins

| Unigene | Gene |
|---|---|
| 119571 | Collagen, type III, alpha 1 (Ehlers-Danlos syndrome type IV, autosomal dominant, shorter alternative |
| | transcript |
| 172928 | collagen, type I, alpha 1, internally primed site |
| 102171 | immunoglobulin superfamily containing leucine-rich repeat, reliable 3′ end |
| 128087 | F2R coagulation factor II (thrombin) receptor, reliable 3′ end |
| 172928 | collagen, type I, alpha 1, internal tag |
| 108623 | thrombospondin 2, reliable 3′ end |
| 278568 | H factor (complement)-like 1, reliable 3′ end |
| 159263 | collagen, type VI, alpha 2, reliable 3′ end |
| 265827 | G1P3 interferon alpha-inducible protein, reliable 3′ end, 97%, IFI-6-16, secreted based on PSORT |
| 296049 | microfibrillar-associated protein, undefined 3′ end |
| 274313 | insulin-like growth factor binding protein 6, reliable 3′ end |
| 75736 | apolipoprotein D, reliable 3′ end |
| 36131 | collagen, type XIV, alpha 1 (undulin), reliable 3′ end |
| 11590 | cathepsin F, reliable 3′ end |
| 24395 | small inducible cytokine subfamily B (Cys-X-Cys), member 14 (BRAK), reliable 3′ end |
| 76152 | decorin, reliable 3′ end |
| 89137 | Low density lipoprotein-related protein 1 (alpha-2-macroglobulin receptor), reliable 3′ end |
| 289019 | latent transforming growth factor beta binding protein 3, relable 3′ end |
| 2420 | superoxide dismutase 3, extracellular, reliable 3′ end |
| 172928 | collagen, type I, alpha 1, shorter alternative transcript |
| 245188 | tissue inhibitor of metalloproteinase 3 (Sorsby fundus dystrophy, pseudoinflammatory), shorter alternative |
| | transcript |
| 821 | biglycan, reliable 3′ end |
| 75736 | apolipoprotein D, internal tag |
| 172928 | collagen, type I, alpha 1, internal tag |
| 76294 | CD63 antigen (melanoma 1 antigen) reliable 3′ end |
| 172928 | collagen, type I, alpha 1, internal tag |
| 79732 | fubulin, transcript variant C, reliable 3′ end |
| 1279 | C1R Complement component 1, r subcomponent, reliable 3′ end |
| 277477 | HLA-C Major histocompatibility complex, class I, C, reliable 3′ end |
| 283713 | collagen triple helix repeat containing 1, reliable 3′ end |
| 193716 | Complement component (3b/4b) receptor 1, including Knops blood group system, reliable 3′ end |
| 155597 | DF D component of complement (adipsin), internal tag |
| 54457 | CD81 antigen (target of antiproliferative antibody 1), reliable 3′ end |
| 93913 | interleukin 6 (interferon, beta 2), reliable 3′ end |
| 101382 | tumor necrosis factor, alpha-induced protein 2, reliable 3′ end |
| 29352 | tumor necrosis factor, alpha-induced protein 6, internally primed site |
| 119206 | insulin-like growth factor binding protein 7, reliable 3′ end |
| 78056 | cathepsin L, reliable 3′ end |
| 202097 | procollagen C-endopeptidase enhancer, reliable 3′ end |
| 237356 | stromal cell-derived factor 1, SAGE Genie: no match, NCBI: Acc.no.U19495 |
| 83942 | cathepsin K (pycnodysostosis), reliable 3′ end |
| 177543 | MIC2 antigen identified by monoclonal antibodies 12E7, F21 and O13, reliable 3′ end, Tcells? |
| 170040 | platelet-derived growth factor receptor-like, reliable 3′ end |
| 151242 | serine (or cysteine) proteinase inhibitor, clade G (C1 inhibitor), member 1, (angioedema, hereditary), reliable |
| | 3′ end |
| 149609 | integrin, alpha 5 (fibronectin receptor, alpha polypeptide), reliable 3′ end |
| 135084 | cystatin C (amyloid angiopathy and cerebral hemorrhage), reliable 3′ end |
| 75111 | protease, serine, 11 (IGF binding), reliable 3′ end |
| 111334 | FTL Ferritin, light polypeptide, reliabe 3′ end |
| 24395 | small inducible cytokine subfamily B (Cys-X-Cys), member 14 (BRAK), reliable 3′ end |
| 108885 | collagen, type VI, alpha 1, reliable 3′ end |
| 169401 | apolipoprotein E, undefined 3′ end |
| 227751 | lectin, gatactoside-binding, soluble, 1 (galectin 1), reliable 3′ end |
| 296267 | follistatin-like 1, reliable 3′ end |
| 119178 | Cation-chloride cotransporter-interacting protein, reliable 3′ end |
| 136348 | Osteoblast specific factor 2 (fasciclin I-like), undefined 3′ end |
| 111301 | Matrix metalloproteinase 2 (gelatinase A, 72 kD gelatinase, 72 kD type IV collagenase, reliable 3′ end |
| 75415 | beta-2-microglobulin, reliable 3′ end |
| 62954 | Ferritin, heavy polypeptide 1, reliable 3′ end |
| 287797 | integrin, beta 1 (fibronectin receptor, beta polypeptide, antigen CD29 includes MDF2, MSK12), reliable 3′ end |
| 74471 | Gap junction protein, alpha 1, 43 kD (connexin 43), reliable 3′ end |
| 8867 | cysteine-rich, angiogenic inducer, 61, reliable 3′ end |
| 87409 | thrombospondin 1, reliable 3′ end |
| 23582 | tumor-associated calcium signal transducer 2, reliable 3′ end |
| 624 | interleukin 8, reliable 3′ end |
| 82689 | tumor rejection antigen (gp96) 1, reliable 3′ end |
| 1369 | Decay accelerating factor for complement (CD55, Cromer blood group system), reliable 3′ end |
| 171921 | sema domain, immunoglobulin domain (Ig), short basic domain, secreted, (semaphorin) 3C, reliable 3′ end |
| 303649 | small inducible cytokine A2 (monocyte chemotactic protein 1), reliable 3′ end |
| 77356 | transferrin receptor (p90, CD71), reliable 3′ end |
| 9006 | VAMP (vesicle-associated membrane protein)-associated protein A (33 kD), reliable 3′ end |
| 6418 | seven transmembrane domain orphan receptor, reliable 3′ end |
| 78614 | complement component 1, q subcomponent binding protein, reliable 3′ end |
| 287797 | ITGB1 Integrin, beta 1 (fibronectin receptor, beta polypeptide, antigen CD29 includes MDF2, MSK12), |
| | internally primed site |
| 75765 | GRO2 oncogene, reliable 3′ end |
| 78225 | annexin A1, reliable 3′ end |
| 2820 | oxytocin receptor, reliable 3′ end |
| 117938 | Collagen, type XVII, alpha 1, reliable 3′ end |
| 289114 | hexabrachion (tenascin C, cytotactin), reliable 3′ end |
| 799 | diphtheria toxin receptor (heparin-binding epidermal growth factor-like growth factor), reliable 3′ end |
| 2250 | leukemia inhibitory factor (cholinergic differentiation factor), reliable 3′ end |
| 198689 | bullous pemphigoid antigen 1 (230/240 kD), reliable 3′ end |
| 8230 | a disintegrin-like and metalloprotease (reprolysin type) with thrombospondin type 1 motif, 1, reliable 3′ end |

TABLE 13
Genes from Table 9 encoding secreted or cell surface proteins

| Unigene | Gene |
|---|---|
| 277477 | HLA-C Major histocompatibility complex, class I, C, reliable 3′ end |
| 332053 | serum amyloid A1, reliable 3′ end |
| 164021 | Small inducible cytokine subfamily B (Cys-X-Cys), member 6 (granulocyte chemotactic protein 2), |
| | reliable 3′ end |
| 297681 | serine (or cysteine) proteinase inhibitor, clade A (alpha-1 antiproteinase, antitrypsin), member 1, reliable |
| | 3′ end |
| 69771 | B-factor, properdin, reliable 3′ end, complement factor |
| 350470 | Trefoil factor 1 (breast cancer, estrogen-inducible sequence expressed in), reliable 3′ end |
| 112341 | protease inhibitor 3, skin-derived (SKALP), reliable 3′ end |
| 75498 | small inducible cytokine subfamily A (Cys-Cys), member 20, reliable 3′ end |
| 2250 | leukemia inhibitory factor (cholinergic differentiation factor), internal tag |
| 155223 | stanniocalcin 2, reliable 3′ end |
| 54457 | CD81 antigen (target of antiproliferative antibody 1), reliable 3′ end |
| 234726 | serine (or cysteine) proteinase inhibitor, clade A (alpha-1 antiproteinase, antitrypsin), member 3, reliable |
| | 3′ end |
| 62492 | HIN-1, secretoglobin, family 3A, member 1, reliable 3′ end |
| 89690 | GRO3 oncogene, reliable 3′ end |
| 204096 | secretoglobin, family 1D, member 2, reliable 3′ end |
| 278573 | CD59 antigen p18-20 (antigen identified by monoclonal antibodies 16.3A5, EJ16, EJ30, EL32 and |
| | G344), reliable 3′end, similarity to urokinase plasminogen activator receptor |
| 621 | lectin, galactoside-binding, soluble, 3 (galectin 3), reliable 3′ end |
| 789 | GRO1 oncogene (melanoma growth stimulating activity, alpha), reliable 3′ end |
| 93913 | interleukin 6 (interferon, beta 2), reliable 3′ end |
| 348419 | LOC118430 Small breast epithelial mucin, undefined 3′ end |
| 75106 | clusterin (complement lysis inhibitor, SP-40, 40, sulfated glycoprotein 2, testosterone-repressed prostate |
| | message 2, apolipoprotein J), reliable 3′ end |
| 277477 | HLA-C Major histocompatibility complex, class I, C, reliable 3′end, 97% |
| 75765 | GRO2 oncogene, reliable 3′ end |
| 624 | interleukin 8, reliable 3′ end |
| 119178 | Cation-chloride cotransporter-interacting protein, reliable 3′ end |
| 5372 | claudin 4, reliable 3′ end |
| 306226 | Transmembrane gamma-carboxyglutamic acid protein 4, reliable 3′ end |
| 31439 | serine protease inhibitor, Kunitz type, 2, reliable 3′ end |
| 323910 | V-erb-b2 erythroblastic leukemia viral oncogene homolog 2, neuro/glioblastoma derived oncogene |
| | homolog (avian), undefined 3′ end |

TABLE 14
Genes from Table 10 encoding secreted or cell surface proteins

| Unigene | Gene |
|---|---|
| 119206 | insulin-like growth factor binding protein 7, shorter alternative transcript |
| 16085 | putative G-protein coupled receptor, reliable 3′ end |
| 25590 | stanniocalcin 1, reliable 3′ end |
| 74561 | alpha-2-macroglobulin, reliable 3′ end |
| 1516 | insulin-like growth factor binding protein 4, undefined 3′ end |
| 352392 | major histocompatibility complex, class II, DR beta 5 |
| 119129 | collagen, type IV, alpha 1, reliable 3′ end |
| 79368 | epithelial membrane protein 1, reliable 3′ end |
| 211604 | a disintegrin-like and metalloprotease (reprolysin type) with thrombospondin type 1 motif 4, reliable 3′ end |
| 119206 | insulin-like growth factor binding protein 7, reliable 3′ end |
| 1908 | proteoglycan 1, secretory granule, reliable 3′ end |
| 74471 | Gap junction protein, alpha 1, 43 kD (connexin 43), reliable 3′ end |
| 624 | interleukin 8, reliable 3′ end |
| 89546 | selectin E (endothelial adhesion molecule 1), reliable 3′ end |
| 168383 | intercellular adhesion molecule 1 (CD54), human rhinovirus receptor, reliable 3′end |
| 298275 | solute carrier family 38, member 2, reliable 3′ end |
| 78409 | collagen, type XVIII, alpha 1, shorter alternative transcript |
| 277477 | Major histocompatibility complex, class I, C, reliable 3′ end |
| 75445 | SPARC-like 1 (mast9, hevin), reliable 3′ end |
| 111334 | Ferritin, light polypeptide, reliable 3′ end |
| 351316 | Transmembrane 4 superfamily member 1, reliable 3′ end |
| 111779 | secreted protein, acidic, cysteine-rich (osteonectin), reliable 3′ end |
| 75415 | beta-2-microglobulin, reliable 3′ end |
| 181357 | laminin receptor 1 (67 kD, ribosomal protein SA), reliable 3′ end |
| 172928 | collagen, type I, alpha 1, internally primed site |
| 300697 | immunoglobulin heavy constant gamma 3 (G3m marker), reliable 3′ end |
| 119571 | Collagen, type III, alpha 1 (Ehlers-Danlos syndrome type IV, autosomal dominant), shorter alternative transcript |
| 75111 | protease, serine, 11 (IGF binding), similar to IGFBP7, cleaves IGF |
| 75511 | connective tissue growth factor, undefined 3′end, 79.6% |
| 193716 | Complement component (3b/4b) receptor 1, including Knops blood group system, reliable 3′ end |
| 172928 | Collagen, type I, alpha 1, internal tag |
| 93557 | proenkephalin (NCBI only) |
| 158287 | syndecan 3 (N-syndecan) |
| 89137 | Low density lipoprotein-related protein 1 (alpha-2-macroglobulin receptor), reliable 3′ end |
| 83326 | matrix metalloproteinase 3 (stromelysin 1, progelatinase), reliable 3′ end |
| 108623 | Thrombospondin 2, reliable 3′ end |
| 102171 | immunoglobulin superfamily containing leucine-rich repeat, reliable 3′ end |
| 25640 | claudin 3, reliable 3′ end |
| 252189 | Syndecan 4 (amphiglycan, ryudocan), undefined 3′ end |
| 286124 | CD24 antigen (small cell lung carcinoma cluster 4 antigen), reliable 3′ end |
| BG939135 | cn30g02.x1 Normal Human Trabecular Bone Cells Homo sapiens cDNA clone NHTBC_cn30g02 random, |
| | mRNA sequence, undefined 3′ end |
| 172928 | collagen, type I, alpha 1, internal tag |
| 23582 | tumor-associated calcium signal transducer 2, reliable 3′ end |
| 5372 | Claudin 4, reliable 3′ end |

### Example 7

### Analysis of SAGE Libraries from Epithelial Cells and Non-Epithelial Cells of Normal Breast Tissue and Breast Tissues from Patients with Various Diseases of the Breast

SAGE analyses were performed on cell types in addition to those described in Example 6 and on breast tissue from patients with a variety of breast conditions. The data described in Example 6 and additional data were analyzed in a manner different to that described in Example 6.

To determine the molecular profile of various cell types that are found in normal and diseased breast tissue (e.g., cancerous epithelial and non-cancerous stromal cells within a breast tumor) and to identify autocrine and paracrine interactions that may play a role in breast tumor progression, a purification procedure (similar to that described in Example 1 for the analysis described in Example 6) was developed that allows the isolation of pure cell populations from normal breast tissue, in situ (DCIS; ductal carcinoma in situ) and invasive breast carcinomas (FIG. 5A). Cell type-specific surface markers and magnetic beads were used for the rapid sequential isolation of the various cell types. The BerEP4 antigen that is restricted to epithelial cells, the CD45 pan-leukocyte marker, and the P1H12 antibody that specifically recognizes endothelial cells were exploited for this purpose. The CD10 antigen is present in myoepithelial cells and myofibroblasts but also in some leukocytes. Thus, to minimize the cross contamination of these different cell types, in the case of normal and DCIS breast tissue, myoepithelial cells were isolated from organoids (breast ducts). On the other hand, in invasive tumors, leukocytes were removed prior to capturing the myofibroblasts using the CD10 beads. There is no antibody is available that specifically recognizes fibroblasts and thereby facilitates their purification. Thus, the unbound fraction, following removal of all other cell types, was used as a fibroblast-enriched “stroma” fraction.

This cell purification protocol includes enzymatic digestion of the tissue and the possibility that the expression of some genes could be altered due to the procedure cannot be, excluded. However, in that it was possible to verify the SAGE data by alternative methods using unprocessed tissue (see below), any such hypothetical changes are likely to be minimal. The success of the purification method and the purity of each cell fraction were confirmed by performing RT-PCR on a small fraction of the isolated cells using cell type-specific genes as was done for the cell fractions described in Example 6 (see Example 1). The remaining portion of the cells (˜110,000-100,000 cells depending on the sample) was used for the generation of micro-SAGE libraries following previously described protocols and for the isolation of genomic DNA to be used for array-Comparative Genomic Hybridization (aCGH) and Single Nucleotide Polymorphism (SNP) array studies [Porter et al. (2003a) Mol. Cancer Res. 1:362-375; Porter et al (2001)].

SAGE libraries were generated using a modified mnicro-SAGE protocol and the I-SAGE or long I-SAGE kits from Invitrogen (Carlsbad, Calif.). Approximately 50,000 tags (mean average tag number 56,647±4,383) were obtained from each library, and the preliminary analysis of the SAGE data was performed essentially as described [Porter et al. (2001)]. Briefly, genes significantly (p≦0.002) differentially expressed between normal and cancerous cells were identified by performing pair-wise comparisons using the SAGE2000 software that includes the software to perform Monte Carlo analysis (obtained from Johns Hopkins University, Baltimore, Md.).

SAGE libraries were generated from epithelial cells, and myoepithelial cells (and myofibroblasts from invasive tumors), infiltrating leukocytes, endothelial cells, and fibroblasts (“stroma”) from one normal breast reduction tissue, two different DCIS, and three invasive breast tumors. Not all libraries were generated from all cases due to the inability to obtain sufficient amounts of purified cells. In addition, a fibroadenoma and a phyllodes tumor were included in the SAGE analysis. Fibroadenomas are the most common benign breast tumors and are not considered to progress to malignancy despite genetic changes detected in the stromal (but not epithelial) cells [Amiel et al. (2003) Cancer Genet. Cytogenet. 142:145-148]. Phyllodes tumors, on the other hand, are rare fibroepithelial tumors that are usually benign but can recur and progress to malignant sarcomas. Phyllodes tumors were initially considered stromal neoplasms but recent molecular studies demonstrating frequently discordant genetic alterations, in both epithelial and stromal cells suggest that phyllodes tumors may represent a true clonal co-evolution of malignant epithelial and stromal cells [Sawyer et al. (2000) Am. J. Pathol. 156:1093-1098; Sawyer et al. (2002) J. Pathol. 196: 437-444]. Analysis of the SAGE data confirmed that the cell purification procedure worked well in that several genes known to be specific for a particular cell type were present in the appropriate SAGE libraries. For example cytokeratins 8 and 19, E-cadherin, HIN-1, CD24 were, highly specific for epithelial cells, myofibroblast and myoepithelial cells demonstrated high levels of smooth muscle actin, various extracellular matrix proteins including collagens, and matrix metalloproteinases, while leukocyte libraries had the highest levels of several chemokines and lysozyme.

Based on statistical methods developed (by bioinformaticians in the Department of Research Computing at the Dana-Farber Cancer Institute and the Department of Biostatistics at the Harvard School of Public Health) for the analysis of SAGE data, genes that are specifically expressed in a particular cell type and tumor progression stage were identified. Genes were defined as specific for a particular cell type if the average tag number in all the SAGE libraries generated from the selected cell type was statistically significantly (P<0.02) different from that of all other cell types. Using these criteria, 357 tags were identified as discriminating epithelial cells from other cell types, 572 tags were identified as discriminating myoepithelial cells and myofibroblasts from all other cell types, 502 tags were identified as discriminating leukocytes from all other cell types, 124 tags were identified as discriminating endothelial cells from all other cell types, and 604 tags were identified as discriminating “stromal” cells depleted of all the above-listed cell types (i.e., mostly fibroblasts) from all other cell types.

To further define SAGE tags specific for each cell type, within each group of tags, those that were not only statistically significantly different, but also more abundant in the specific cell type, were selected. This led to the identification of 70 tags that were most abundant in epithelial cells, 117 tags present at highest levels in myoepithelial cells and myofibroblasts, 70 tags highly expressed in leukocytes, 117 tags in stroma, and 78 endothelium-specific tags. Several of these genes have previously been described as being specific for a particular cell type, e.g., keratins 8 and 19 for epithelial cells, keratins 14 and 17 for myoepithelial cells, and chemokines and chemokine receptors for leukocytes [Page et al. (1999) Proc. Natl. Acad. Sci. USA 96:12589-12594]. However, the cell type-specific expression of the majority of the genes has not been previously documented. The majority of the transcripts corresponding to these cell-type specific SAGE tags encode known genes but a significant fraction either are uncharacterized ESTs or currently have no cDNA match (˜10% of the tags on average belong to each of these latter groups). In stroma 25/117 tags (21%) had no database match suggesting that they correspond to previously unidentified transcripts.

Next, using the 471 SAGE tags most abundantly expressed or 63 of the SAGE tags most highly specifically present in each of the five cell types, a clustering analysis of all 27 SAGE libraries using a new-Poisson model based K-means algorithm (PK algorithm) was performed in order to delineate similarities and differences among the samples. In addition, a clustering analysis of the SAGE libraries using each of the cell type specific genes was performed. The PK clustering method orders the samples according to their relatedness. For example, using the 63 most highly cell type specific SAGE tags, a division of the 27 SAGE libraries according to cell types was obtained and, within each cell type sub-group, the DCIS samples are located between normal breast tissue and invasive breast cancer SAGE libraries. These results confirmed that, not only tumor epithelial cells, but also other cell types in the tumor are different from their corresponding normal counterparts. Since these differences are already pronounced at a pre-invasive (DCIS) tumor stage, they suggest a role for stromal changes not only in tumor invasion and metastasis, but also in the earlier steps of breast tumorigenesis.

The most consistent and dramatic gene expression changes were found to occur in myoepithelial cells. Over 300 genes were differentially expressed at p<0.002 in both DCIS myoepithelial libraries. Interestingly, a significant fraction (89 out of 245 known genes) of these genes encode secreted or cell surface proteins, suggesting extensive abnormal paracrine interactions between myoepithelial and other cell types. Myoepithelial cells are thought to be derived from bi-potential stem cells that also give rise to luminal epithelial cells, although recently another progenitor has also been identified that can differentiate only to myoepithelial cells [Bocker et al. (2002) Lab. Invest. 82:737-746; Dontue et al. (2003) Genes Dev. 17:1253-1270]. The function of myoepithelial cells and their role in breast cancer is not well understood. However, myoepithelial cells have been shown to be able to suppress breast cancer cell growth, invasion, and angiogenesis [Deugnier et al. (2002) Breast Cancer Res. 4:224-230; Sternlicht and Barsky (1997) Clin. Cancer Res. 3:1949-1958]. The main distinguishing feature between in situ and invasive carcinomas, which is also used as a diagnostic criterion, is that: (a) in DCIS the cancer epithelial cells are separated from the stroma by a nearly continuous layer of myoepithelial cells and basement membrane; while (b) in invasive and metastatic tumors cancer cells are admixed with stroma.

In Table 15 are shown the most highly cell type-specific SAGE tags and corresponding genes. Columns 1-27 in Table 15 show data obtained from 27 separate libraries generated from cells from a variety of samples. These samples were:

Columns 1-7 (Myoepithelial Cells and Myofibroblasts)

Column 1: myoepithelial cells isolated from normal breast tissue adjacent to invasive ductal carcinoma (IDC7) tissue.

Column-2: myoepithelial cells isolated from reduction mammoplasty normal breast tissue (RM1).

Column 3: myofibroblasts isolated from an invasive ductal carcinoma (IDC7).

Column 4: myofibroblasts isolated from an invasive ductal carcinoma (IDC8).

Column 5: myofibroblasts isolated from an invasive ductal carcinoma (IDC9).

Column 67 myoepithelial cells isolated from DCIS tissue (D7).

Column 7: myoepithelial cells isolated from DCIS tissue (D6).

Columns 8-10 and 26-(Fibroblast-Enriched Cells):

Column 8: fibroblast-enriched cells from an invasive ductal carcinoma (IDC7).

Column 9: fibroblast-enriched cells from DCIS tissue (D6).

Column 10: fibroblast-enriched cells from reduction mammoplasty normal breast tissue (RM2).

Column 26: fibroblast-enriched cells from a phyllodes tumor.

Columns 11-12 (Endothelial Cells):

Column 11: endothelial cells isolated from reduction mammoplasty normal breast tissue (RM2).

Column 12: endothelial cells isolated from DCIS tissue (D6).

Columns 13-16 (Leukocytes):

Column 13: leukocytes isolated from DCIS tissue (D7).

Column 14: leukocytes isolated from DCIS tissue (D6).

Column 15: leukocytes isolated from an invasive ductal carcinoma (IDC7).

Column 16: leukocytes isolated from reduction mammoplasty normal breast tissue (RM2).

Columns 17-25 (epithelial cells, luminal type):

Column 17: Epithelial Cells Isolated from an Invasive Ductal Carcinoma (IDC7).

Column 18: epithelial cells isolated from an invasive ductal carcinoma (IDC8).

Column 19: epithelial cells isolated from an invasive ductal carcinoma (IDC9).

Column 20: epithelial cells isolated from DCIS tissue (D7).

Column 21: epithelial cells isolated from DCIS tissue (D6).

Column 22: epithelial cells isolated from normal breast tissue adjacent to DCIS (D2) tissue.

Column 23: epithelial cells isolated from reduction mammoplasty normal breast tissue (RM3).

Column 24: epithelial cells isolated from DCIS tissue (D2).

Column 25: epithelial cells isolated from DCIS tissue (D3).

Column 27: (Unseparated Cells of a Juvenile Fibroadenoma)

Rows 1-72 in Table 15 show SAG tags detected in the various libraries depicted in columns 1-27.

Rows 1-27: SAGE tags that were statistically significantly (p<0.02) more abundantly expressed in epithelial cells than in all other cell types.

Rows 28-53: SAGE tags that were statistically significantly (p<0.02) more abundantly expressed in myoepithelial cells than in all other cell types or in myofibroblasts than in all other cell types.

Rows 54-58: SAGE tags that were statistically significantly (p<0.02) more abundantly expressed in leukocytes than in all other cell types.

Rows 59-65: SAGE tags that were statistically significantly (p<0.02) more abundantly expressed in fibroblast-enriched cells than in all other cell types.

Rows 66-72: SAGE tags that were statistically significantly (p<0.02) more abundantly expressed in endothelial cells than in all other cell types.

From Table 15 it can readily be determined, by referring to the intersection of relevant columns and rows, which of the listed genes are differently expressed (more highly or at a lower level) in the various cell types from DCIS and/or invasive breast cancers compared to corresponding cell types from normal tissue. Analogous differences in expression between cells from DCIS and from invasive breast carcinomas can similarly be discerned from the data in Table 15. It is noted that myofibroblasts are cells found only in cancer tissue and thus comparisons of gene expression involving myofibroblasts will be between: (a) myofibroblasts in DCIS and invasive breast carcinomas; or (b) between myofibroblasts in DCIS or invasive breast carcinomas and any other cell type (e.g., myoepithelial cells or fibroblasts) from normal breast tissue.

Follow up studies were focused on myoepithelial cells, with special emphasis on secreted proteins and receptors abnormally expressed in these cells. Several proteases [e.g., cathepsins F, K, and L, MMP2 (matrix metalloproteinase 2), and PRSS11 (protease serine (insulin-like growth factor-binding)], protease inhibitors [thrombospondin 2, SERPING1 (serine (or cysteine) proteinase inhibitor, lade G (C1 inhibitor) member 1), cystatin C, and TIM3 (tissue inhibitor of metalloproteinase 3)], and many different collagens were highly up-regulated in DCIS myoepithelial cells, suggesting a role for these cells in extracellular matrix remodeling (Table 16).

In Table 16, the column labeled “N-MYOEP-1” shows data obtained from a SAGE library generated from myoepithelial cells isolated from reduction mammoplasty normal breast tissue (RM1). The columns labeled “D-MYOEP-7” and “D-MYOEP-6” show data obtained from a SAGE library generated from myoepithelial cells isolated from two DCIS tissue samples (D7 and D6, respectively). The column labeled “Ratio D/N” shows the ratio of the average of the numbers of SAGE tags obtained with the two DCIS tissue samples to the SAGE tag number obtained with normal breast tissue.

Array-Comparative Genomic Hybridization (aCGH) and Single Nucleotide Polymorphism (SNP) array studies indicated that the changes in gene expression in non-cancer cells present in breast tumor tissue detected by the analysis described in Example 6 and this Example were not due to chromosomal gains or losses, e.g., loss of heterozygosity.

TABLE 15
List of most highly cell type-specific SAGE tags and corresponding genes

| | | SEQ: | | | | | | | | | | | | | | | | | | | | | | | | | | | | | |
|---|---|---|---|---|---|---|---|---|---|---|---|---|---|---|---|---|---|---|---|---|---|---|---|---|---|---|---|---|---|---|---|
| | | ID | | | | | | | | | | | | | | | | | | | | | | | | | | | | | |
| | SAGE tag | NO | 1 | 2 | 3 | 4 | 5 | 6 | 7 | 8 | 9 | 10 | 11 | 12 | 13 | 14 | 15 | 16 | 17 | 18 | 19 | 20 | 21 | 22 | 23 | 24 | 25 | 26 | 27 | Unigene | Gene description |
| 1 | CCTCCAGCTA | 1832 | 0 | 5 | 9 | 6 | 0 | 0 | 2 | 28 | 0 | 10 | 8 | 0 | 2 | 4 | 31 | 11 | 118 | 72 | 124 | 159 | 32 | 28 | 62 | 43 | 14 | 3 | 25 | 356123 | KRT8 keratin 8 |
| 2 | GACATCAAGT | 1833 | 0 | 0 | 5 | 0 | 0 | 0 | 0 | 15 | 0 | 4 | 9 | 0 | 5 | 9 | 26 | 11 | 73 | 64 | 59 | 153 | 48 | 15 | 18 | 55 | 2 | 0 | 5 | 309517 | KRT19 keratin 19 |
| 3 | TGTGGGTGCT | 1834 | 0 | 5 | 2 | 0 | 0 | 0 | 3 | 3 | 0 | 2 | 0 | 0 | 0 | 0 | 4 | 0 | 11 | 17 | 25 | 49 | 83 | 14 | 15 | 14 | 5 | 0 | 5 | 194657 | CDN1 cadherin 1, type 1, E- |
| | | | | | | | | | | | | | | | | | | | | | | | | | | | | | | | cadherin |
| 4 | AGGAAGGAAC | 1835 | 0 | 0 | 0 | 2 | 0 | 0 | 0 | 2 | 0 | 0 | 0 | 0 | 0 | 3 | 0 | 0 | 18 | 0 | 2 | 24 | 90 | 0 | 0 | 3 | 7 | 0 | 3 | 446352 | ERBB |
| 5 | CTGGCCCTCG | 1836 | 0 | 0 | 0 | 9 | 0 | 0 | 0 | 2 | 0 | 0 | 4 | 0 | 0 | 2 | 3 | 4 | 33 | 149 | 74 | 74 | 10 | 62 | 163 | 39 | 6 | 0 | 5 | 350470 | TFF1 trefoil factor 1 |
| 6 | CTCCACCCGA | 1837 | 2 | 0 | 3 | 19 | 0 | 0 | 0 | 5 | 0 | 4 | 8 | 0 | 0 | 8 | 12 | 38 | 43 | 297 | 51 | 38 | 25 | 3 | 19 | 284 | 11 | 3 | 50 | 82961 | TFF3 trefoil factor 3 |
| 7 | AAGCTCGCCG | 1838 | 0 | 0 | 2 | 2 | 0 | 0 | 0 | 0 | 0 | 3 | 2 | 0 | 0 | 3 | 0 | 7 | 0 | 24 | 0 | 0 | 7 | 19 | 89 | 0 | 0 | 0 | 0 | 82492 | SCGB3A1 secretoglobin |
| | | | | | | | | | | | | | | | | | | | | | | | | | | | | | | | family 3A, member 1 |
| | | | | | | | | | | | | | | | | | | | | | | | | | | | | | | | (HN-1) |
| 8 | CTTCCTGTGA | 1839 | 0 | 0 | 0 | 0 | 0 | 0 | 0 | 0 | 0 | 5 | 0 | 0 | 0 | 8 | 0 | 0 | 2 | 5 | 0 | 5 | 67 | 98 | 272 | 7 | 10 | 0 | 0 | 348419 | LOC18430 small breast |
| | | | | | | | | | | | | | | | | | | | | | | | | | | | | | | | epithelial mucin |
| 9 | AAGAAAACCT | 1840 | 0 | 0 | 0 | 0 | 0 | 0 | 0 | 0 | 0 | 0 | 0 | 0 | 0 | 0 | 0 | 2 | 22 | 10 | 19 | 22 | 2 | 2 | 8 | 16 | 0 | 0 | 3 | 100685 | BCMP11 breast cancer |
| | | | | | | | | | | | | | | | | | | | | | | | | | | | | | | | membrane protein 11 |
| 10 | ATTTTCTAAA | 1841 | 0 | 0 | 0 | 8 | 0 | 0 | 0 | 2 | 0 | 0 | 0 | 0 | 0 | 0 | 3 | 0 | 8 | 68 | 13 | 5 | 6 | 3 | 2 | 25 | 0 | 0 | 3 | 226391 | AGR2 anterior gradient 2 |
| | | | | | | | | | | | | | | | | | | | | | | | | | | | | | | | homolog (Xenopus laevis) |
| 11 | CGGACTCACT | 1842 | 0 | 2 | 3 | 2 | 2 | 0 | 0 | 0 | 0 | 2 | 4 | 3 | 2 | 0 | 0 | 0 | 9 | 23 | 13 | 89 | 12 | 0 | 3 | 11 | 3 | 0 | 3 | 300446 | STARD10 START domain con- |
| | | | | | | | | | | | | | | | | | | | | | | | | | | | | | | | taining 10 |
| 12 | GGAACAAACA | 1843 | 0 | 0 | 3 | 0 | 0 | 0 | 0 | 11 | 0 | 8 | 11 | 0 | 6 | 6 | 9 | 14 | 62 | 7 | 129 | 94 | 122 | 62 | 30 | 57 | 3 | 0 | 13 | 375108 | CD24 CD24 antigen |
| 13 | AATATGTGGG | 1844 | 13 | 9 | 7 | 17 | 9 | 2 | 0 | 29 | 6 | 6 | 3 | 6 | 0 | 0 | 14 | 0 | 89 | 56 | 80 | 112 | 2 | 2 | 6 | 235 | 4 | 8 | 12 | 89664 | BPA-1 mRNA for brain pep- |
| | | | | | | | | | | | | | | | | | | | | | | | | | | | | | | | tide A1 |
| 14 | GGACTCTGGA | 1845 | 0 | 0 | 4 | 0 | 0 | 0 | 0 | 2 | 0 | 6 | 3 | 0 | 0 | 5 | 7 | 5 | 25 | 39 | 2 | 23 | 31 | 14 | 56 | 11 | 7 | 0 | 0 | 439027 | BDNF brain-derived neuro- |
| | | | | | | | | | | | | | | | | | | | | | | | | | | | | | | | trophic factor |
| 15 | CTGGCCCTCG | 1846 | 0 | 0 | 0 | 9 | 0 | 0 | 0 | 2 | 0 | 0 | 4 | 0 | 0 | 2 | 3 | 4 | 33 | 149 | 74 | 74 | 10 | 62 | 163 | 39 | 6 | 0 | 5 | 43654 | CLN6 ceroid-lipofuscinosis, |
| | | | | | | | | | | | | | | | | | | | | | | | | | | | | | | | neuronal 6, late infanbile, |
| 16 | ATCGTGGCGG | 1847 | 0 | 0 | 60 | 2 | 0 | 7 | 0 | 61 | 0 | 7 | 68 | 0 | 19 | 11 | 69 | 27 | 357 | 36 | 96 | 972 | 86 | 57 | 23 | 36 | 20 | 0 | 0 | 5372 | CLDN4 claudlin 4 |
| 17 | ATCGTGGCGG | 1848 | 0 | 0 | 60 | 2 | 0 | 7 | 0 | 61 | 0 | 7 | 68 | 0 | 19 | 11 | 69 | 27 | 357 | 36 | 96 | 972 | 86 | 57 | 23 | 36 | 20 | 0 | 0 | 8026 | SESN2 sestrin 2 |
| 18 | GCAGGGCCTC | 1849 | 0 | 0 | 9 | 5 | 0 | 0 | 6 | 4 | 0 | 8 | 4 | 0 | 2 | 3 | 0 | 9 | 29 | 39 | 15 | 68 | 16 | 21 | 19 | 44 | 8 | 0 | 15 | 301350 | FXYD3FXYD domain containing |
| | | | | | | | | | | | | | | | | | | | | | | | | | | | | | | | ion transport |
| 19 | TGTGGGTGCT | 1850 | 0 | 5 | 2 | 0 | 0 | 0 | 3 | 3 | 0 | 2 | 0 | 0 | 0 | 0 | 4 | 0 | 11 | 17 | 25 | 49 | 83 | 14 | 15 | 14 | 5 | 0 | 5 | 306339 | SRPUL sushi-repeat protein |
| 20 | GGACTCTGGA | 1851 | 0 | 0 | 4 | 0 | 0 | 0 | 0 | 2 | 0 | 6 | 3 | 0 | 0 | 5 | 7 | 5 | 25 | 39 | 2 | 23 | 31 | 14 | 56 | 11 | 7 | 0 | 0 | 512643 | AZGP1 alpha-2-glycoprotein |
| | | | | | | | | | | | | | | | | | | | | | | | | | | | | | | | 1, zinc |
| 21 | ATGCTCAGCC | 1852 | 0 | 0 | 4 | 2 | 0 | 0 | 0 | 3 | 0 | 0 | 0 | 0 | 0 | 0 | 3 | 0 | 9 | 12 | 86 | 48 | 9 | 2 | 4 | 6 | 0 | 0 | 0 | 96125 | RCP Rab coupling protein |
| 22 | AAATAAAGAA | 1853 | 2 | 2 | 3 | 5 | 0 | 0 | 0 | 10 | 2 | 3 | 2 | 0 | 0 | 2 | 9 | 0 | 33 | 22 | 61 | 28 | 19 | 7 | 5 | 13 | 2 | 0 | 9 | 389700 | MGST1 microsomal gluta- |
| | | | | | | | | | | | | | | | | | | | | | | | | | | | | | | | thlone S-transferase 1 |
| 23 | GCAGTGGCCT | 1854 | 2 | 0 | 0 | 0 | 0 | 0 | 0 | 6 | 0 | 3 | 5 | 0 | 0 | 4 | 0 | 2 | 8 | 26 | 14 | 25 | 11 | 3 | 3 | 32 | 5 | 0 | 11 | 396783 | SLC9A3R1 solute carrier |
| | | | | | | | | | | | | | | | | | | | | | | | | | | | | | | | family 9, isoform 3 |
| | | | | | | | | | | | | | | | | | | | | | | | | | | | | | | | regulatory |
| 24 | TGGGGTTCTT | 1855 | 0 | 0 | 0 | 0 | 0 | 0 | 0 | 0 | 0 | 0 | 4 | 0 | 2 | 0 | 0 | 0 | 8 | 5 | 0 | 84 | 0 | 0 | 0 | 0 | 0 | 0 | 0 | 272499 | DHRS2 dehydrogense/ |
| | | | | | | | | | | | | | | | | | | | | | | | | | | | | | | | reductase (SDR family) |
| 25 | ATGCTCAGCC | 1856 | 0 | 0 | 4 | 2 | 0 | 0 | 0 | 3 | 0 | 0 | 0 | 0 | 0 | 0 | 03 | 0 | 9 | 12 | 86 | 48 | 9 | 2 | 4 | 6 | 0 | 0 | 0 | 98306 | KIAA1862KIAA1862 protein |
| 26 | TTGCGTTGCG | 1857 | 0 | 0 | 0 | 0 | 0 | 0 | 0 | 0 | 0 | 0 | 4 | 0 | 0 | 2 | 0 | 3 | 0 | 0 | 0 | 4 | 16 | 0 | 0 | 0 | 89 | 0 | 0 | no match | |
| 27 | TCTCCATACC | 1858 | 0 | 0 | 0 | 0 | 0 | 0 | 0 | 0 | 0 | 0 | 0 | 0 | 0 | 0 | 0 | 0 | 0 | 0 | 0 | 0 | 0 | 137 | 0 | 183 | 0 | 0 | 0 | no match | |
| 28 | GATGTGCACG | 1859 | 0 | 339 | 41 | 5 | 0 | 5 | 19 | 2 | 0 | 0 | 0 | 0 | 0 | 3 | 0 | 0 | 0 | 0 | 0 | 0 | 0 | 6 | 4 | 0 | 6 | 0 | 2 | 355214 | KRT14 keratin 14 |
| 29 | GACCAGCAGA | 1860 | 0 | 0 | 40 | 15 | 24 | 18 | 44 | 0 | 0 | 3 | 13 | 3 | 4 | 6 | 0 | 0 | 0 | 0 | 0 | 0 | 0 | 0 | 0 | 0 | 0 | 0 | 4 | 137569 | TP73L tumor protein p73- |
| | | | | | | | | | | | | | | | | | | | | | | | | | | | | | | | like |
| 30 | TTAAATAGCA | 1861 | 8 | 0 | 57 | 80 | 181 | 2 | 14 | 11 | 2 | 6 | 8 | 0 | 0 | 0 | 3 | 0 | 2 | 10 | 4 | 0 | 0 | 0 | 0 | 0 | 0 | 18 | 19 | 172928 | COL1A1 collagen, type I, |
| | | | | | | | | | | | | | | | | | | | | | | | | | | | | | | | alpha 1 |
| 31 | CCGGGGGAGC | 1862 | 3 | 0 | 43 | 52 | 104 | 45 | 55 | 8 | 0 | 7 | 17 | 0 | 0 | 4 | 0 | 0 | 0 | 6 | 2 | 2 | 2 | 0 | 0 | 0 | 0 | 18 | 10 | 172928 | COL1A1 collagen, type I, |
| | | | | | | | | | | | | | | | | | | | | | | | | | | | | | | | alpha 1 |
| 32 | GACTTTGGAA | 1863 | 8 | 0 | 18 | 33 | 53 | 15 | 100 | 21 | 6 | 7 | 0 | 2 | 0 | 2 | 0 | 0 | 0 | 2 | 0 | 0 | 2 | 0 | 0 | 0 | 0 | 18 | 27 | 172928 | COL1A1 collagen, type I, |
| | | | | | | | | | | | | | | | | | | | | | | | | | | | | | | | alpha 1 |
| 33 | TGGAAATGAA | 1864 | 4 | 0 | 11 | 16 | 18 | 3 | 24 | 4 | 0 | 6 | 0 | 2 | 0 | 0 | 2 | 0 | 0 | 0 | 0 | 0 | 0 | 0 | 0 | 0 | 0 | 5 | 15 | 172928 | COL1A1 collagen, type I, |
| | | | | | | | | | | | | | | | | | | | | | | | | | | | | | | | alpha 1 |
| 34 | CGGGGTGGCC | 1865 | 8 | 0 | 22 | 11 | 9 | 81 | 22 | 5 | 0 | 2 | 0 | 0 | 4 | 2 | 2 | 0 | 0 | 3 | 0 | 3 | 4 | 0 | 0 | 0 | 0 | 3 | 0 | 1584 | COMP cartilage oligomeric |
| | | | | | | | | | | | | | | | | | | | | | | | | | | | | | | | matrix protein |
| 35 | TGGAAGCAGA | 1866 | 0 | 0 | 42 | 9 | 0 | 28 | 0 | 4 | 0 | 0 | 2 | 0 | 0 | 0 | 0 | 0 | 0 | 0 | 0 | 4 | 0 | 0 | 0 | 0 | 0 | 0 | 0 | 1584 | COMP cartilage oligomeric |
| | | | | | | | | | | | | | | | | | | | | | | | | | | | | | | | matrix protein |
| 36 | CTGTCAGCGT | 1867 | 5 | 0 | 70 | 34 | 107 | 12 | 29 | 5 | 2 | 3 | 0 | 3 | 0 | 3 | 6 | 0 | 0 | 2 | 3 | 0 | 4 | 0 | 0 | 0 | 0 | 9 | 0 | 283713 | CTHRC1 collagen triple |
| | | | | | | | | | | | | | | | | | | | | | | | | | | | | | | | helix repeat containing 1 |
| 37 | CAGGAGACCC | 1868 | 0 | 0 | 33 | 51 | 302 | 0 | 8 | 8 | 2 | 0 | 0 | 0 | 0 | 3 | 2 | 0 | 2 | 3 | 4 | 0 | 0 | 0 | 0 | 0 | 0 | 0 | 0 | 143751 | MMP11 matrix metallo- |
| | | | | | | | | | | | | | | | | | | | | | | | | | | | | | | | proteinase 11 (stromelysin |
| | | | | | | | | | | | | | | | | | | | | | | | | | | | | | | | 3) |
| 38 | TCCCTACCGA | 1869 | 0 | 0 | 10 | 15 | 22 | 0 | 4 | 0 | 2 | 0 | 0 | 3 | 0 | 0 | 0 | 0 | 0 | 0 | 0 | 0 | 0 | 0 | 0 | 0 | 0 | 0 | 0 | 367877 | MMP2 matrix metallo- |
| | | | | | | | | | | | | | | | | | | | | | | | | | | | | | | | proteinase 2 |
| 39 | TGGAAGCAGA | 1870 | 0 | 0 | 42 | 9 | 0 | 28 | 0 | 4 | 0 | 0 | 2 | 0 | 0 | 0 | 0 | 0 | 0 | 0 | 0 | 4 | 0 | 0 | 0 | 0 | 0 | 0 | 0 | 415041 | THBS4 thrombospondin 4 |
| 40 | AGAATGAGAT | 1871 | 8 | 2 | 28 | 17 | 24 | 13 | 12 | 3 | 2 | 8 | 2 | 0 | 0 | 2 | 0 | 0 | 0 | 0 | 2 | 0 | 0 | 0 | 0 | 0 | 0 | 3 | 0 | 156316 | DCN decorin |
| 41 | TATTTTCACA | 1872 | 3 | 0 | 21 | 19 | 31 | 4 | 5 | 2 | 3 | 3 | 2 | 0 | 0 | 0 | 0 | 0 | 0 | 0 | 0 | 0 | 0 | 0 | 0 | 0 | 0 | 0 | 0 | 156316 | DCN decorin |
| 42 | ACATAGACCG | 1873 | 10 | 0 | 27 | 24 | 34 | 5 | 11 | 4 | 9 | 2 | 2 | 4 | 0 | 0 | 2 | 0 | 0 | 0 | 0 | 0 | 0 | 0 | 0 | 0 | 0 | 0 | 0 | 173584 | SERPINF1 |
| 43 | CTATAGGAGA | 1874 | 4 | 2 | 13 | 19 | 61 | 2 | 4 | 9 | 5 | 0 | 0 | 0 | 0 | 0 | 2 | 0 | 0 | 0 | 0 | 0 | 0 | 0 | 0 | 0 | 0 | 13 | 7 | 274520 | ANTXR1 anthrax toxin |
| | | | | | | | | | | | | | | | | | | | | | | | | | | | | | | | receptor 1 |
| 44 | GTAAATATGG | 1875 | 0 | 81 | 12 | 4 | 0 | 0 | 3 | 3 | 0 | 0 | 0 | 0 | 0 | 0 | 0 | 0 | 0 | 0 | 0 | 0 | 0 | 0 | 0 | 0 | 0 | 0 | 0 | 443518 | BPAG1 bullous permphigoid |
| | | | | | | | | | | | | | | | | | | | | | | | | | | | | | | | antigen 1, 23W24DkDa |
| 45 | TTTGTGGGCA | 1876 | 2 | 0 | 8 | 17 | 11 | 11 | 7 | 0 | 0 | 2 | 0 | 3 | 0 | 0 | 0 | 0 | 0 | 0 | 2 | 0 | 0 | 0 | 0 | 0 | 0 | 3 | 4 | 439184 | RCN3 reticulocalbin 3, EF- |
| | | | | | | | | | | | | | | | | | | | | | | | | | | | | | | | hand calcium binding |
| 46 | GGGAAGGGAC | 1877 | 0 | 52 | 6 | 0 | 0 | 0 | 2 | 3 | 0 | 2 | 0 | 0 | 0 | 0 | 0 | 0 | 0 | 0 | 0 | 0 | 0 | 0 | 0 | 0 | 0 | 0 | 0 | 13144 | ORMDL2 CRMM-like 2 |
| 47 | GGGAAGGGAC | 1878 | 0 | 52 | 6 | 0 | 0 | 0 | 2 | 3 | 0 | 2 | 0 | 0 | 0 | 0 | 0 | 0 | 0 | 0 | 0 | 0 | 0 | 0 | 0 | 0 | 0 | 0 | 0 | 431156 | PPP2R1B protein phosphalase |
| | | | | | | | | | | | | | | | | | | | | | | | | | | | | | | | 2, regulatory subunit |
| 48 | CTTCCTTGCC | 1879 | 8 | 785 | 179 | 34 | 4 | 7 | 63 | 19 | 7 | 27 | 0 | 2 | 0 | 3 | 8 | 0 | 6 | 0 | 17 | 6 | 3 | 5 | 15 | 2 | 33 | 0 | 4 | 449630 | HBA2 hemoglobin, alpha 2 |
| 49 | GGGGAAATCG | 1880 | 96 | 22 | 57 | 103 | 112 | 228 | 177 | 19 | 59 | 75 | 71 | 120 | 30 | 330 | 32 | 34 | 59 | 88 | 149 | 188 | 151 | 41 | 90 | 38 | 22 | 45 | 96 | 446574 | TMSB10 thymosin, beta 10 |
| 50 | TATTTTCACA | 1881 | 3 | 0 | 21 | 19 | 31 | 4 | 5 | 2 | 3 | 3 | 2 | 0 | 0 | 0 | 0 | 0 | 0 | 0 | 0 | 0 | 0 | 0 | 0 | 0 | 0 | 0 | 0 | 132131 | Transcribed sequences |
| 51 | CCACGGGATT | 1882 | 20 | 0 | 62 | 164 | 78 | 23 | 168 | 17 | 10 | 0 | 19 | 16 | 2 | 14 | 0 | 4 | 0 | 8 | 5 | 0 | 2 | 0 | 0 | 0 | 2 | 0 | 68 | no match | |
| 52 | GGTCTTCAAG | 1883 | 0 | 0 | 5 | 23 | 27 | 0 | 7 | 2 | 0 | 5 | 0 | 0 | 0 | 0 | 0 | 0 | 0 | 0 | 0 | 0 | 0 | 0 | 0 | 0 | 0 | 0 | 4 | no match | |
| 53 | GTGCGCCGGA | 1884 | 0 | 40 | 7 | 0 | 0 | 0 | 8 | 0 | 0 | 0 | 0 | 0 | 0 | 0 | 0 | 0 | 0 | 0 | 0 | 0 | 0 | 0 | 0 | 0 | 0 | 0 | 0 | no match | |
| 54 | GAGCTGGAAA | 1885 | 0 | 0 | 0 | 0 | 0 | 0 | 0 | 0 | 2 | 0 | 0 | 0 | 0 | 33 | 0 | 0 | 0 | 0 | 0 | 0 | 0 | 0 | 0 | 0 | 0 | 0 | 0 | 73875 | FAH fumarylacetocetate hydro- |
| | | | | | | | | | | | | | | | | | | | | | | | | | | | | | | | lase |
| 55 | GAGCTGGAAA | 1886 | 0 | 0 | 0 | 0 | 0 | 0 | 0 | 0 | 2 | 0 | 0 | 0 | 0 | 33 | 0 | 0 | 0 | 0 | 0 | 0 | 0 | 0 | 0 | 0 | 0 | 0 | 0 | 20950 | LHPP phospholysine phosphohis- |
| | | | | | | | | | | | | | | | | | | | | | | | | | | | | | | | tidine Inorganic |
| 56 | GAGAAATCGT | 1887 | 0 | 0 | 0 | 0 | 0 | 0 | 0 | 0 | 0 | 0 | 0 | 0 | 0 | 33 | 0 | 0 | 0 | 0 | 0 | 0 | 0 | 0 | 0 | 0 | 0 | 0 | 0 | 23734 | LYZ lysozyme |
| 57 | AACGGGGCCC | 1888 | 2 | 0 | 2 | 0 | 0 | 0 | 0 | 0 | 0 | 0 | 0 | 0 | 2 | 17 | 4 | 2 | 0 | 0 | 0 | 0 | 0 | 0 | 0 | 0 | 0 | 0 | 0 | 80420 | CX3CL1 chemoklne |
| 58 | ATTCCTGAGC | 1889 | 2 | 0 | 0 | 0 | 0 | 0 | 0 | 0 | 0 | 0 | 0 | 0 | 2 | 24 | 0 | 2 | 0 | 0 | 0 | 0 | 0 | 0 | 0 | 0 | 0 | 0 | 0 | no match | |
| 59 | ATACAGAATA | 1890 | 2 | 0 | 0 | 0 | 0 | 2 | 0 | 0 | 0 | 64 | 0 | 0 | 0 | 0 | 0 | 0 | 0 | 0 | 0 | 0 | 0 | 0 | 0 | 0 | 0 | 0 | 15 | 169228 | DLK1 delta-like 1 homolog |
| 60 | CAGGAGAAGG | 1891 | 0 | 0 | 0 | 0 | 0 | 0 | 0 | 0 | 0 | 29 | 0 | 0 | 0 | 0 | 0 | 0 | 0 | 0 | 0 | 0 | 0 | 0 | 0 | 0 | 0 | 0 | 0 | 24049 | GOLGA2 golgi autoantigen, |
| | | | | | | | | | | | | | | | | | | | | | | | | | | | | | | | golgin subfamily a, 2 |
| 61 | CAGGAGAAGG | 1892 | 0 | 0 | 0 | 0 | 0 | 0 | 0 | 0 | 0 | 29 | 0 | 0 | 0 | 0 | 0 | 0 | 0 | 0 | 0 | 0 | 0 | 0 | 0 | 0 | 0 | 0 | 0 | 366 | MGC27165 hypothetical protein |
| | | | | | | | | | | | | | | | | | | | | | | | | | | | | | | | MGC27165 |
| 62 | GCGGAGGTGG | 1893 | 2 | 0 | 0 | 0 | 0 | 0 | 2 | 2 | 0 | 283 | 4 | 11 | 10 | 6 | 2 | 4 | 0 | 0 | 0 | 0 | 0 | 0 | 0 | 0 | 0 | 0 | 0 | 366 | MGC27165 hypothetical protein |
| | | | | | | | | | | | | | | | | | | | | | | | | | | | | | | | MGC27165 |
| 63 | GCCGTTCTTA | 1894 | 41 | 0 | 0 | 2 | 0 | 0 | 0 | 42 | 27 | 277 | 0 | 11 | 0 | 0 | 0 | 2 | 0 | 3 | 0 | 0 | 0 | 5 | 5 | 3 | 0 | 32 | 0 | no match | |
| 64 | TGAACAGCAG | 1895 | 2 | 0 | 0 | 0 | 0 | 0 | 0 | 4 | 5 | 18 | 0 | 0 | 0 | 0 | 0 | 0 | 0 | 0 | 0 | 0 | 0 | 0 | 0 | 0 | 0 | 0 | 0 | no match | |
| 65 | GAGTTTATTC | 1896 | 3 | 0 | 0 | 3 | 0 | 0 | 0 | 4 | 2 | 31 | 0 | 0 | 0 | 0 | 0 | 0 | 0 | 0 | 0 | 0 | 0 | 0 | 0 | 0 | 0 | 0 | 5 | no match | |
| 66 | AATGAATTAT | 1897 | 0 | 0 | 0 | 0 | 0 | 0 | 0 | 0 | 0 | 0 | 3 | 9 | 0 | 2 | 0 | 0 | 0 | 0 | 0 | 0 | 0 | 0 | 0 | 0 | 0 | 0 | 0 | 293257 | ECT2 epithelial cell trans- |
| | | | | | | | | | | | | | | | | | | | | | | | | | | | | | | | forming sequence 2 oncogene |
| 67 | TAGGTCAGGA | 1898 | 0 | 0 | 0 | 0 | 0 | 0 | 0 | 0 | 0 | 0 | 7 | 4 | 0 | 2 | 0 | 0 | 0 | 0 | 0 | 0 | 0 | 0 | 0 | 0 | 0 | 0 | 2 | 43666 | PTP4A3 protein tyrosine |
| | | | | | | | | | | | | | | | | | | | | | | | | | | | | | | | phosphatase type IVA, |
| 68 | CGAGAGTGTG | 1899 | 0 | 0 | 0 | 0 | 0 | 0 | 0 | 0 | 0 | 0 | 4 | 15 | 0 | 0 | 0 | 0 | 0 | 0 | 0 | 0 | 0 | 0 | 0 | 0 | 0 | 0 | 0 | 175804 | CDNA FLJ42395 fis, clone |
| | | | | | | | | | | | | | | | | | | | | | | | | | | | | | | | ASTRO2001076 |
| 69 | GCGCCTCCCG | 1900 | 0 | 0 | 0 | 0 | 0 | 0 | 0 | 0 | 0 | 0 | 11 | 5 | 0 | 0 | 0 | 0 | 0 | 0 | 0 | 0 | 0 | 0 | 0 | 0 | 0 | 0 | 0 | 435800 | VIM vimentin |
| 70 | TGTTGAAAAA | 1901 | 104 | 0 | 9 | 0 | 0 | 0 | 3 | 15 | 82 | 0 | 4 | 31 | 2 | 91 | 0 | 2 | 0 | 0 | 0 | 0 | 12 | 3 | 0 | 0 | 0 | 0 | 0 | 89546 | SELE selectin E |
| 71 | AAGTTTGGTG | 1902 | 0 | 0 | 0 | 0 | 0 | 0 | 0 | 0 | 0 | 0 | 3 | 12 | 0 | 0 | 0 | 0 | 0 | 0 | 0 | 0 | 3 | 0 | 0 | 0 | 0 | 0 | 0 | 66727 | KCNJ10 potassium inwardly- |
| | | | | | | | | | | | | | | | | | | | | | | | | | | | | | | | rectifying channel, |
| 72 | GGCCGCGAGG | 1903 | 0 | 0 | 0 | 0 | 0 | 3 | 0 | 2 | 0 | 0 | 18 | 5 | 0 | 0 | 0 | 0 | 0 | 0 | 0 | 0 | 0 | 0 | 0 | 0 | 0 | 0 | 0 | 78344 | MYH11 myosin, heavy poly- |
| | | | | | | | | | | | | | | | | | | | | | | | | | | | | | | | peptide 11, smooth muscle |

TABLE 16

List of genes encoding secreted and cell surface proteins
overexpressed in DCIS myoepithelial cells compared to
normal myoepethelial cells

| | | | | | | | |
|---|---|---|---|---|---|---|---|
| SEQ | | | | | | | |
| ID | | | | | | | |
| NO | SAGE Tag | N-MYOEP-1 | D-MYOEP-7 | D-MYOEP-6 | Ration D/N | Unigene | Gene description |

| | | | | | | | |
|---|---|---|---|---|---|---|---|
| 1904 | ACCAAAAACC | 2 | 274 | 849 | 244 | 172928 | COL1A1 collagen, type I, alpha 1 |
| 1905 | GATCAGGCCA | 0 | 191 | 181 | 124 | 443625 | COL3A1 collagen, type III, alpha 1 |
| 1906 | TGGAAATGAC | 0 | 50 | 228 | 93 | 172928 | COL1A1 collagen, type I, alpha 1 |
| 1907 | CGGGGTGGCC | 0 | 193 | 24 | 73 | 1584 | COMP cartilage oligomeric matrix |
| | | | | | | | protein |
| 1908 | CTAACGGGGC | 0 | 169 | 20 | 63 | 513022 | ISLR immunoglobulin superfamily |
| | | | | | | | containing leucine-rich repeat |
| 1909 | CAGATAAGTT | 0 | 72 | 101 | 58 | 222171 | KIAA0182 KIAA0182 protein |
| 1910 | CCGGGGGAGC | 0 | 110 | 61 | 57 | 172928 | COL1A1 collagen, type I, alpha 1 |
| 1911 | GTCAAAATTT | 0 | 110 | 47 | 52 | 458354 | THBS2 thrombospondin 2 |
| 1912 | GTGCTAAGCG | 3 | 308 | 141 | 49 | 420269 | COL6A2 collagen, type VI, alpha 2 |
| 1913 | GACTTTGGAA | 0 | 36 | 110 | 49 | 172928 | COL1A1 collagen, type I, alpha 1 |
| 1914 | CGCCGACGAT | 0 | 100 | 32 | 44 | 287721 | GIP3 interferon, alpha-inducible |
| | | | | | | | protein (clone IFI-6-16) |
| 1915 | TTGGGATGGG | 0 | 103 | 29 | 44 | 296941 | HFL1 H factor (complement)-like 1 |
| 1916 | CATATCATTA | 0 | 21 | 94 | 38 | 435795 | IGFBP7 insulin-like growth factor |
| | | | | | | | binding protein 7 |
| 1917 | TCCAGGAAAC | 0 | 72 | 39 | 37 | 115900 | CTSF cathepsin F |
| 1918 | GGCCCCTCAC | 0 | 74 | 22 | 32 | 274313 | IGFBP6 insulin-like growth factor |
| | | | | | | | binding protein 6 |
| 1919 | ACATTCCAAG | 0 | 50 | 42 | 31 | 245188 | TIMP3 tissue Inhibitor of metallo- |
| | | | | | | | proteinase 3 |
| 1920 | ATAAAAAGAA | 0 | 19 | 73 | 31 | 83942 | CTSK cathepsin K |
| 1921 | GACCAGCAGA | 0 | 43 | 48 | 30 | 172928 | COL1A1 collagen, type I, alpha 1 |
| 1922 | ACTTATTATG | 2 | 107 | 30 | 30 | 156316 | DCN decorin |
| 1923 | GTGCGCTGAG | 0 | 33 | 52 | 28 | 274485 | HLA-C mdor histocompatibility complex, |
| | | | | | | | class I, C |
| 1924 | TGCGCTGGCC | 0 | 87 | 18 | 28 | 289019 | LTBP3 latent transforming growth factor |
| | | | | | | | beta binding protein 3 |
| 1925 | AGGCTCCTGG | 3 | 217 | 31 | 27 | 24395 | CXCL14 chemokine |
| 1926 | CTCAACCCCC | 2 | 105 | 19 | 27 | 162757 | LRP1 low density lipoprotein-related |
| | | | | | | | protein 1 |
| 1927 | CAGCGGCGGG | 0 | 57 | 13 | 23 | 2420 | SOD3 superoxide dismutase 3, extra- |
| | | | | | | | cellular |
| 1928 | GGCACCTCAG | 2 | 36 | 65 | 22 | 512234 | IL6 interleukin 6 |
| 1929 | GCCTGTCCCT | 0 | 50 | 13 | 21 | 821 | BGN biglycan |
| 1930 | ATTTCTTCAA | 0 | 19 | 44 | 21 | 31386 | SFRP2 secreted frizzled-related |
| | | | | | | | protein 2 |
| 1931 | TCGAAGAACC | 2 | 60 | 34 | 21 | 445570 | CD63 CD63 antigen |
| 1932 | ACATTCTTTT | 0 | 17 | 44 | 20 | 389984 | GPNMB glycoprotein (transmembrane) |
| 1933 | CTGTCAGCGT | 0 | 29 | 32 | 20 | 283713 | CTHRC1 collagen triple helix repeat |
| | | | | | | | containing 1 |
| 1934 | CAGCTGGCCA | 0 | 36 | 22 | 19 | 445240 | FBLN1 fibulin 1 |
| 1935 | ACTGAAAGAA | 3 | 124 | 50 | 19 | 458355 | C1S complement component 1, s sub- |
| | | | | | | | component |
| 1936 | TTCTGTGCTG | 3 | 105 | 40 | 16 | 376414 | C1R complement component 1, r sub- |
| | | | | | | | component |
| 1937 | GGATGTGAAA | 0 | 19 | 26 | 15 | 283477 | CD99 CD99 antigen |
| 1938 | ACTCAGCCCG | 2 | 36 | 28 | 14 | 101382 | TNFAIP2 tumor necrosis factor, alpha- |
| | | | | | | | induced protein 2 |
| 1939 | TTTCCCTCAA | 2 | 21 | 42 | 14 | 75111 | PRSS11 protease, serine, 11 (IGF |
| | | | | | | | binding) |
| 1940 | CTAAAAAAAA | 0 | 26 | 15 | 14 | 54457 | CD81 CD81 antigen (target of antipro- |
| | | | | | | | liferative antibody 1) |
| 1941 | GGCCACGTAG | 0 | 26 | 15 | 14 | 155597 | DF D component of complement |
| 1942 | AAGAAAGGAG | 0 | 21 | 20 | 14 | 202097 | PCOLCE procollagen C-endopeptidase |
| | | | | | | | enhancer |
| 1943 | GGAGGAATTC | 0 | 21 | 20 | 14 | 418123 | CTSL cathepsin L |
| 1944 | AGCCACCGCG | 2 | 43 | 19 | 14 | 355874 | RABL2B RAB, member of RAS oncogene |
| | | | | | | | family-like 2B |
| 1945 | TGTAAACAAT | 0 | 19 | 22 | 14 | 170040 | PDGFRL platelet-derived growth factor |
| | | | | | | | receptor-like |
| 1946 | ACCTTGAAGT | 2 | 36 | 19 | 12 | 407546 | TNFAIP6 tumor necrosis factor, alpha- |
| | | | | | | | induced protein 6 |
| 1947 | CATAAATGCG | 0 | 21 | 13 | 12 | 436042 | CXCL12 chemokine (stromal cell-derived |
| | | | | | | | factor 1) |
| 1948 | TTGCTGACTT | 12 | 122 | 279 | 11 | 415997 | COL6A1 collagen, type VI, alpha 1 |
| 1949 | ATGGCAACAG | 0 | 17 | 17 | 11 | 149609 | ITGA5 integrin, alpha 5 |
| 1950 | CTCTCCAAAC | 2 | 26 | 20 | 10 | 384598 | SERPING1 serine proteinase inhibitor, |
| | | | | | | | dade G, member 1 |
| 1951 | TGCCTGCACC | 5 | 76 | 46 | 9 | 304682 | CST3 cystatin C |
| 1952 | GGAAATGTCA | 18 | 93 | 325 | 8 | 367877 | MMP2 matrix metalloproteinase 2 |
| 1953 | CAGGTTTCAT | 12 | 124 | 117 | 7 | 24395 | CXCL14 chemokine |
| 1954 | CCGTGACTCT | 12 | 112 | 70 | 5 | 433622 | FSTL1 follistatin-like 1 |

### Example 8

### Evaluation of Gene Expression by Immunohistochemistry and mRNA In Situ Hybridization

The generation of the SAGE libraries described in Example 7 involved initial in vitro cell purification steps that could potentially have altered in vivo gene expression patterns, although prior SAGE data from several laboratories suggest that these changes are likely to be minimal [Porter et al. (2003a) Porter et al. (2003b) Proc. Natl. Acad. Sci USA 100:10931-16936; St. Croix et al. (2000) Science 289:1197-1202]. Nevertheless, in order to further investigate the expression of selected genes at the cellular level in vivo, immunohistochemical and mRNA in situ hybridization analyses were performed on a panel of DCIS and invasive breast tumors (different from the tumors used for SAGE). In addition, the cell type, specificity of some genes was verified by RT-PCR in the samples used for SAGE (data not shown).

Immunohistochemical analysis confirmed that two genes, those encoding IL-1β and CCL3 (MIP1α), are highly expressed in leukocytes infiltrating DCIS, but not normal breast tissue, whereas the CD45 (PTPRC) pan-leukocyte marker Was expressed in both cases. Despite the similar number of total leukocytes in invasive tumors the frequency of IL-1β and CCL3 positive leukocytes, although higher than in normal breast tissue, was much lower than in DCIS, suggesting that in situ and invasive breast carcinomas may be immunologically dissimilar.

mRNA in situ hybridization determined that in DCIS tumors: (a) the expression of PDGF (platelet-derived growth factor) receptor β-like (PDGFRBL), cathepsin K (CTSK), and CXCL12 was localized to myofibroblasts as determined by smooth muscle actin (ACTA2) staining; (b) CXCL14 was expressed only in myoepithelial cells; (c) TIMP3, cystatin C(CST3) and collagen triple helix repeat containing 1 (CTHRC1) were expressed in both my epithelial cells and myofibroblasts. In invasive tumors all these genes were expressed in myofibroblasts; there are no myoepithelial cells in invasive breast tumors. No signal was detected in normal breast tissue and with the sense probes (data not shown). Interestingly, although in DCIS tumors CXCL14 expression was detected only in myoepithelial cells, in some invasive breast carcinomas, while present in myofibroblasts, it was much more strongly expressed in tumor epithelial cells (data not shown). Similarly, some breast cancer cell lines expressed high levels of CXCL12 or CXCL14 in vitro suggesting that during tumor progression a paracrine factor may be converted into an autocrine one due to its up-regulation in the tumor epithelial cells. All the CXCL14 positive primary breast tumors and even the CXCL14 expressing breast cancer cell line (UACC812) were obtained from young, pre-menopausal patients (average age of onset 39 years), suggesting a possible association of CXCL14 expression with clinico-pathologic characteristics of the tumors.

### Example 9

### The effect of CXCL12 and CXCL14 Chemokines on Breast Cancer Cells

The high level of expression of two chemokines, CXCL12 and CXCL14, in myoepithelial cells and myofibroblasts, both in DCIS and invasive breast carcinomas, was particularly interesting in view of the known function of chemokines as regulators of cell proliferation, differentiation, migration, and invasion [Gerard et al. (2001) Nat. Immunol. 2:108-115; Muller et al. (2001) Nature 410:50-56; Rossi et al. (2000) Annu. Rev. Immunol. 18:217-2.42]. To determine if CXCL12 and CXCL14 can act as autocrine and/or paracrine factors in breast tumors, an analysis to identify cell types expressing receptors for the two chemokines in primary breast tissue in vivo was cared out.

The signaling receptor for CXCL12 is CXCR4, which is known to be expressed in various lymphoid cells as well as a variety of epithelial cells [Gerard et al. (2001)]. The expression of CXCR4 in lymphoid and breast epithelial cells was confirmed by immunohistochemistry and SAGE data indicated that its expression is increased in invasive tumors compared to DCIS and normal breast tissue (data not shown).

The signaling receptor for CXCL14 is unknown but cell surface ligand binding experiments have suggested the presence of a putative CXCL14 receptor on monocytes and B-cells, suggesting that its receptor is unlikely to be CXCR4 [Kurth et al. (2001) J. Exp. Med. 194:855-861; Sleeman et al. (2000) Int. Immunol. 12:677-689]. To determine if a CXCL14-binding cell surface protein(s) is also present on breast dancer cells, an alkaline phosphatase-CXCL14 (AP-CXCL14) fusion protein to be used as a ligand in receptor binding assays was generated. In this fusion protein the AP was located N-terminal of the CXCL14. Conditioned medium from P-CXCL14- or control AP-expressing cells was used as an affinity reagent to stain normal and cancerous mammary tissue sections. Blue staining indicated the presence of a CXCL14 binding protein in certain leukocytes and breast epithelial cells. These findings suggest the presence of a cell surface CXCL114 binding protein(s) in cancerous and normal mammary epithelial cells and are consistent with a paracrine mechanism of CXCL14 action in the breast. To test further the binding characteristics of AP-CXCL14, in vitro ligand binding assays were carried out using various cell lines. Low level AP-CXCL14 binding was detected in all cell lines tested including MDA-MB-231 and MDA-MB-435 breast cancer and MCF10A immortalized mammary epithelial cells (data not shown). To further characterize the AP-CXCL14-putative CXCL14 receptor interaction, more detailed-binding assays were carried out on MDA-MB-231 breast cancer cells. Scatchard plot analysis showed two binding slopes in MDA-MB-231 cells, thereby indicating the presence of high (Kd=6.1×10⁻⁸ M) and low affinity (Kd=56.7×10⁻⁸ M) binding sites (FIG. 6A).

In previous studies, CXCL12 was demonstrated to enhance breast cancer cell growth, migration and invasion [Hall et al. (2003) Mol. Endocrinol. 17:792-803; Muller et al. (2001)] and it was hypothesized to be involved in metastasis [Kang et al; (2003) Cancer Cell 3:537-549; Muller et al. (2001)]. The present demonstration that it is highly expressed in myofibroblasts from DCIS, a pre-invasive tumor, indicates that it is likely to have additional roles in earlier stages of breast tumorigenesis. In order to determine if CXCL14 has similar effects, the effect of conditioned medium containing AP-CXCL14 on the growth of MDA-MB-231 and MCF10A cells was tested and its effect on cell migration and invasion was investigated using MDA-MB-231 cells. Conditioned media of cells transfected with AP alone and CXCL12 were used as negative and positive controls, respectively. Similar to CXCL12, AP-CXCL14 enhanced the proliferation of MDA-MB-231 and MCF10A cells and the migration and invasion of MDA-MB-231 cells (FIGS. 6B and C and data not shown). In these experiments, the concentration of AP-CXCL14 was 2-30 nM, which is similar to the concentration ranges of several chemokines, including CXCL12, required for biological effects. The same results were obtained in cell migration and invasion assays using CXCL14-AP (C-terminal AP-tag) and CXCL14-HA (C-terminal HA-tag) fusion proteins (FIG. 6C and data not shown). Thus, the observed effects are not likely to be due to the position or identity of the epitope tag. Further suggesting that mammary epithelia cells have a functional CXCL14 receptor, experiments using recombinant CXCL14 protein and CXCL14 expressing adenovirus demonstrated the induction of calcium flux in MDA-MB-231 and activation of Akt kinase in MCF10A cells, respectively (data not shown).

A number of embodiments of the invention have been described. Nevertheless, it will be understood that various modifications may be made without departing from the spirit and scope of the invention. Accordingly, other embodiments are within the scope of the following claims.

1. A method of diagnosis, the method comprising:
(a) providing a test sample of breast tissue;
(b) determining the level of expression in the test sample of a gene selected from those listed in Table 1; and
(c) if the gene is expressed in the test sample at a lower level than in a control normal breast tissue sample, diagnosing the test sample as containing cancer cells.

2. A method of determining the grade of a ductal carcinoma in situ (DCIS), the method comprising:
(a) providing a test sample of DCIS tissue;
(b) deriving a test expression profile for the test sample by determining the level of expression in the test sample of ten or more genes selected from those listed in Tables 2-16;
(c) comparing the test expression profile to control expression profiles of the ten or more genes in control samples of high grade, intermediate grade, and low grade DCIS;
(d) selecting the control expression profile that most closely resembles the test expression profile; and
(e) assigning to the test sample a grade that matches the grade of the control expression profile selected in step (d).

3.-7. (canceled)

8. A method of determining the likelihood of a breast cancer being DCIS or invasive breast cancer, the method comprising:
(a) providing a test sample of breast tissue;
(b) determining the level of expression in the test sample of a gene selected from the group consisting of a gene encoding CD74, a gene encoding MGC2328, a gene encoding S100A7, a gene encoding KRT19, a gene encoding trefoil factor 3 (TFF3), a gene encoding osteonectin, and a gene identified by a SAGE tag consisting of the nucleotide sequence CTGGGCGCCC (SEQ ID NO:1109);
(c) determining whether the level of expression of the selected gene in the test sample more closely resembles the level of expression of the selected gene in control cells of (i) DCIS or (ii) invasive breast cancer; and
(d) classifying the test sample as: (i) likely to be DCIS if the level of expression of the gene in the test sample more closely resembles the level of expression of the gene in DCIS cells; or (ii) likely to be invasive breast cancer if the level of expression of the gene in the test sample more closely resembles the level of expression of the gene in invasive breast cancer cells.

9. A method of predicting the prognosis of a breast cancer patient, the method comprising:
(a) providing a sample of primary invasive breast cancer tissue from a test patient; and
(b) determining the level of expression in the sample of a gene encoding S100A7 or a gene encoding fatty acid synthase (FASN),
wherein a level of expression higher than in a control sample of primary invasive breast carcinoma from a patient with a good prognosis is an indication that the prognosis of the test patient is poor.

10. A method of diagnosis comprising:
(a) providing a test sample of breast tissue comprising a test stromal cell; and
(b) determining the level of expression in the stromal cell of a gene selected from those listed in Tables 7, 8, 10, 15, and 16, wherein the gene is one that is expressed in a cell of the same type as the test stromal cell at a substantially higher level when present in breast cancer tissue than when present in normal breast tissue; and
(c) classifying the test sample as: (i) normal breast tissue if the level of expression of the gene in the test stromal cell is not substantially higher than a control level of expression for a cell of the same type as the test stromal cell in normal breast tissue; (ii) breast cancer tissue if the level of expression of the gene in the test stromal cell is substantially higher than a control level of expression for a cell of the same type as the test stromal cell in normal breast tissue.

11. The method of claim 10, wherein the stromal cells in the test sample and the standard samples are leukocytes and the genes are selected from those listed in Tables 7 and 15.

12. The method of claim 11, wherein the gene encodes interleukin-1β (ILβ) or macrophage inhibitory protein 1α (MIP 1α).

13. The method of claim 10, wherein the stromal cells in the test sample and the standard samples are myoepithelial cells or myofibroblasts and the genes are selected from those listed in Tables 8, 15, and 16.

14. The method of claim 13, wherein the gene encodes a polypeptide selected from the group consisting of cathepsins F, K, and L, MMP2, PRSS11, thrombospondin 2, SERPING1, cystatin C(CST3), TIMP3, platelet-derived growth factor receptor β-like (PDGFRBL), a collagen, collagen triple helix repeat containing 1 (CTHRC1), CXCL12, and CXCL14.

15. The method of claim 10, wherein the stromal cells in the test sample and the standard samples are endothelial cells and the genes are selected from those listed in Tables 10 and 15.

16. The method of claim 10, wherein the stromal cells in the test sample and the standard samples are fibroblasts and the genes are selected from those listed in Table 15.

17. A method of diagnosis comprising:
(a) providing a test sample of breast tissue comprising a test stromal cell; and
(b) determining the level of expression in the stromal cell of a gene selected from those listed in Tables 7, 8, 10, and 15 wherein the gene is one that is expressed in a cell of the same type as the test stromal cell at a substantially higher level when present in normal breast tissue than when present in breast cancer tissue; and
(c) classifying the test sample as: (i) normal breast tissue if the level of expression of the gene in the test stromal cell is not substantially lower than a control level of expression for a cell of the same type as the test stromal cell in normal breast tissue; (ii) breast cancer tissue if the level of expression of the gene in the test stromal cell is substantially lower than a control level of expression for a cell of the same type as the test stromal cell in normal breast tissue.

18. The method of claim 17, wherein the stromal cells in the test sample and the standard samples are leukocytes and the genes are selected from those listed in Tables 7 and 15.

19. The method of claim 17, wherein the stromal cells in the test sample and the standard samples are myoepithelial cells or myofibroblasts and the genes are selected from those listed in Tables 8 and 15.

20. The method of claim 17, wherein the stromal cells in the test sample and the standard samples are endothelial cells and the genes are selected from those listed in Tables 10 and 15.

21. The method of claim 17, wherein the stromal cells in the test sample and the standard samples are fibroblasts and the genes are selected from those listed in Table 15.

22. A method of diagnosis comprising:
(a) providing a test sample of breast tissue comprising a test epithelial cell of the luminal epithelial type;
(b) determining the level of expression in the test epithelial cell of a gene selected from those listed in Tables 9 and 15, wherein the gene is one that is expressed in cancerous epithelial cells of the luminal epithelial cell type at a substantially higher level than those in normal breast tissue; and
(c) classifying the test sample as: (i) normal breast tissue if the level of expression of the gene in the test epithelial cell is not substantially higher than a control level of expression for an epithelial cell of luminal epithelial cell type in normal breast tissue; (ii) breast cancer tissue if the level of expression of the gene in the test epithelial cell is substantially higher than a control level of expression for an epithelial cell of the luminal epithelial type in normal breast tissue.

23. A method of diagnosis comprising:
(a) providing a test sample of breast tissue comprising a test epithelial cell of the luminal epithelial type; and
(b) determining the level of expression in the test epithelial cell of a gene selected from those listed in Tables 9 and 15, wherein the gene is one that is expressed in epithelial cells of the luminal epithelial cell type at a substantially lower level when present in breast cancer tissue than when present in normal breast tissue; and
(c) classifying the test sample as: (i) normal breast tissue if the level of expression of the gene in the test epithelial cell is not substantially lower than a control level of expression for an epithelial cell of luminal epithelial cell type in normal breast tissue; (ii) breast cancer tissue if the level of expression of the gene in the test epithelial cell is substantially lower than a control level of expression for an epithelial cell of the luminal epithelial type in normal breast tissue.

24.-25. (canceled)

26. A method of inhibiting proliferation or survival of a breast cancer cell, the method comprising contacting a breast cancer cell with a polypeptide that is encoded by a gene selected from those listed in Tables 1, 7-10, and 15, wherein the gene is expressed in the cancer cell, or a stromal cell in a tumor comprising the cancer cell, at a level substantially lower than in a normal cell of the same type.

27.-31. (canceled)

32. A method of inhibiting pathogenesis of a breast cancer cell or stromal cell in a tumor of a mammal, the method comprising
(a) identifying a mammal with a breast cancer tumor; and
(b) administering to the mammal an agent that inhibits binding of a polypeptide encoded by a gene selected from those listed in Tables 2-10, 15, and 16 to its receptor or ligand,
wherein the gene is expressed in a breast cancer cell in the tumor, or in a stromal cell in the tumor, at a level substantially higher than in a corresponding cell in a non-cancerous breast, and
wherein the polypeptide is a secreted polypeptide or a cell-surface polypeptide.

33.-39. (canceled)

40. A method of inhibiting expression of a gene in a cell, the method comprising introducing into a target cell selected from the group consisting of (a) a breast cancer cell and (b) stromal cell in a tumor comprising a breast cancer cell, an agent that inhibits expression of a gene selected from those listed in Tables 2-10, 15 and 16, wherein the gene is expressed in the target cell at a level substantially higher than in a corresponding cell in normal breast tissue.

41.-49. (canceled)

50. A single stranded nucleic acid probe comprising:
(a) the nucleotide sequence of a tag selected from those listed in Tables 1-5, 7-10, 15 and 16; or
(b) the complement of the nucleotide sequence.

51. An array comprising a substrate having at least 10 addresses, wherein each address has disposed thereon a capture probe comprising a nucleic acid sequence consisting of a tag nucleotide sequence selected from those listed in Tables 1-5, 7-10, 15, and 16.

52.-57. (canceled)

58. A kit comprising at least 10 probes, each probe comprising a nucleic acid sequence comprising a tag nucleotide sequence selected from those listed in Tables 1-10, 15 and 16.

59.-63. (canceled)

64. A kit comprising at least 10 antibodies each of which is specific for a different protein encoded by a gene identified by a tag selected from the group consisting of the tags listed in Tables 1-5, 7-10, 15 and 16.

65.-70. (canceled)

71. A method of identifying the grade of a DCIS, the method comprising:
(a) providing a test sample of DCIS tissue;
(b) using the array of claim 51 to determine a test expression profile of the sample;
(c) providing a plurality of reference profiles, each derived from a DCIS of a defined grade, wherein the test expression profile and each reference profile has a plurality of values, each value representing the expression level of a gene corresponding to a tag selected from those listed in Tables 1-5, 7-10, 15, and 16; and
(d) selecting the reference profile most similar to the test expression profile, to thereby identify the grade of the test DCIS.

72. A method of determining whether a breast cancer is a DCIS or an invasive breast cancer, the method comprising:
(a) providing a test sample of breast cancer tissue;
(b) determining the level of expression of CXCL14 in myofibroblasts in the test sample;
(c) determining whether the level of expression of CXCL14 in the myofibroblasts in the test sample more closely resembles the level of expression of CXCL14 in control myofibroblasts of (i) DCIS or (ii) invasive breast cancer; and
(d) classifying the test sample as: (i) DCIS if the level of expression of CXCL14 in myofibroblasts in the test sample more closely resembles the level of expression of CXCL14 in control myofibroblasts of DCIS; (ii) invasive breast cancer if the level of expression of CXCL14 in myofibroblasts in the test sample more closely resembles the level of expression of CXCL14 in control myofibroblasts of invasive breast cancer.

73. An isolated DNA comprising:
(a) the nucleotide sequence of a tag selected from those listed in FIG. 7; or
(b) the complement of the nucleotide sequence.

74. A vector comprising the DNA of claim 73.

75.-76. (canceled)

77. An isolated polypeptide encoded by the DNA of claim 73.


SEQUENCE LISTING (1955 sequences)

SEQ ID NO: 1 - DNA, 10 bp, Artificial Sequence
  tag sequence
aaatatccag

SEQ ID NO: 2 - DNA, 10 bp, Artificial Sequence
  tag sequence
tggaagcact

SEQ ID NO: 3 - DNA, 10 bp, Artificial Sequence
  tag sequence
aagctcgccg

SEQ ID NO: 4 - DNA, 10 bp, Artificial Sequence
  tag sequence
ttgaaacttt

SEQ ID NO: 5 - DNA, 10 bp, Artificial Sequence
  tag sequence
ttgcaggctc

SEQ ID NO: 6 - DNA, 10 bp, Artificial Sequence
  tag sequence
ataataaaag

SEQ ID NO: 7 - DNA, 10 bp, Artificial Sequence
  tag sequence
ttggtttttg

SEQ ID NO: 8 - DNA, 10 bp, Artificial Sequence
  tag sequence
gagggtttag

SEQ ID NO: 9 - DNA, 10 bp, Artificial Sequence
  tag sequence
gtactagtgt

SEQ ID NO: 10 - DNA, 10 bp, Artificial Sequence
  tag sequence
gccttaacaa

SEQ ID NO: 11 - DNA, 10 bp, Artificial Sequence
  tag sequence
gccttgggtg

SEQ ID NO: 12 - DNA, 10 bp, Artificial Sequence
  tag sequence
accaaattaa

SEQ ID NO: 13 - DNA, 10 bp, Artificial Sequence
  tag sequence
agaaagatgt

SEQ ID NO: 14 - DNA, 10 bp, Artificial Sequence
  tag sequence
tgactggcag

SEQ ID NO: 15 - DNA, 10 bp, Artificial Sequence
  tag sequence
gtccgagtgc

SEQ ID NO: 16 - DNA, 10 bp, Artificial Sequence
  tag sequence
gcttgcaaaa

SEQ ID NO: 17 - DNA, 10 bp, Artificial Sequence
  tag sequence
accaggccac

SEQ ID NO: 18 - DNA, 10 bp, Artificial Sequence
  tag sequence
tttgaaatga

SEQ ID NO: 19 - DNA, 10 bp, Artificial Sequence
  tag sequence
cttgcaaacc

SEQ ID NO: 20 - DNA, 10 bp, Artificial Sequence
  tag sequence
ccattgaaac

SEQ ID NO: 21 - DNA, 10 bp, Artificial Sequence
  tag sequence
cccgaggcag

SEQ ID NO: 22 - DNA, 10 bp, Artificial Sequence
  tag sequence
ctggccctcg

SEQ ID NO: 23 - DNA, 10 bp, Artificial Sequence
  tag sequence
gacacgaaca

SEQ ID NO: 24 - DNA, 10 bp, Artificial Sequence
  tag sequence
gctgcccttg

SEQ ID NO: 25 - DNA, 10 bp, Artificial Sequence
  tag sequence
cgaatgtcct

SEQ ID NO: 26 - DNA, 10 bp, Artificial Sequence
  tag sequence
ctcacttttt

SEQ ID NO: 27 - DNA, 10 bp, Artificial Sequence
  tag sequence
agaatttagg

SEQ ID NO: 28 - DNA, 10 bp, Artificial Sequence
  tag sequence
agtcaaaaat

SEQ ID NO: 29 - DNA, 10 bp, Artificial Sequence
  tag sequence
attagtgttg

SEQ ID NO: 30 - DNA, 10 bp, Artificial Sequence
  tag sequence
ctttggaaat

SEQ ID NO: 31 - DNA, 10 bp, Artificial Sequence
  tag sequence
gcaacttaga

SEQ ID NO: 32 - DNA, 10 bp, Artificial Sequence
  tag sequence
gggacgagtg

SEQ ID NO: 33 - DNA, 10 bp, Artificial Sequence
  tag sequence
gggtttgttt

SEQ ID NO: 34 - DNA, 10 bp, Artificial Sequence
  tag sequence
gtcttaaagt

SEQ ID NO: 35 - DNA, 10 bp, Artificial Sequence
  tag sequence
atgtcttttc

SEQ ID NO: 36 - DNA, 10 bp, Artificial Sequence
  tag sequence
catatcatta

SEQ ID NO: 37 - DNA, 10 bp, Artificial Sequence
  tag sequence
ctccacccga

SEQ ID NO: 38 - DNA, 10 bp, Artificial Sequence
  tag sequence
acgttaaaga

SEQ ID NO: 39 - DNA, 10 bp, Artificial Sequence
  tag sequence
attttctaaa

SEQ ID NO: 40 - DNA, 10 bp, Artificial Sequence
  tag sequence
agtggtggct

SEQ ID NO: 41 - DNA, 10 bp, Artificial Sequence
  tag sequence
atcttgttac

SEQ ID NO: 42 - DNA, 10 bp, Artificial Sequence
  tag sequence
ttatgtttaa

SEQ ID NO: 43 - DNA, 10 bp, Artificial Sequence
  tag sequence
ctcatctgct

SEQ ID NO: 44 - DNA, 10 bp, Artificial Sequence
  tag sequence
acattccaag

SEQ ID NO: 45 - DNA, 10 bp, Artificial Sequence
  tag sequence
ccagagagtg

SEQ ID NO: 46 - DNA, 10 bp, Artificial Sequence
  tag sequence
tttggttttc

SEQ ID NO: 47 - DNA, 10 bp, Artificial Sequence
  tag sequence
accaaaaacc

SEQ ID NO: 48 - DNA, 10 bp, Artificial Sequence
  tag sequence
tggaaatgac

SEQ ID NO: 49 - DNA, 10 bp, Artificial Sequence
  tag sequence
tttgttttta

SEQ ID NO: 50 - DNA, 10 bp, Artificial Sequence
  tag sequence
tggccccagg

SEQ ID NO: 51 - DNA, 10 bp, Artificial Sequence
  tag sequence
cgaccccacg

SEQ ID NO: 52 - DNA, 10 bp, Artificial Sequence
  tag sequence
aacacagcct

SEQ ID NO: 53 - DNA, 10 bp, Artificial Sequence
  tag sequence
gaatttccca

SEQ ID NO: 54 - DNA, 10 bp, Artificial Sequence
  tag sequence
caaactaacc

SEQ ID NO: 55 - DNA, 10 bp, Artificial Sequence
  tag sequence
gaaataaagc

SEQ ID NO: 56 - DNA, 10 bp, Artificial Sequence
  tag sequence
aaaccccaat

SEQ ID NO: 57 - DNA, 10 bp, Artificial Sequence
  tag sequence
aagcacaaaa

SEQ ID NO: 58 - DNA, 10 bp, Artificial Sequence
  tag sequence
tggtttgcgt

SEQ ID NO: 59 - DNA, 10 bp, Artificial Sequence
  tag sequence
tacaataaac

SEQ ID NO: 60 - DNA, 10 bp, Artificial Sequence
  tag sequence
aggaaggaac

SEQ ID NO: 61 - DNA, 10 bp, Artificial Sequence
  tag sequence
acattctttt

SEQ ID NO: 62 - DNA, 10 bp, Artificial Sequence
  tag sequence
caccctgtac

SEQ ID NO: 63 - DNA, 10 bp, Artificial Sequence
  tag sequence
gttcacatta

SEQ ID NO: 64 - DNA, 10 bp, Artificial Sequence
  tag sequence
caagcaggac

SEQ ID NO: 65 - DNA, 10 bp, Artificial Sequence
  tag sequence
tgctgcctgt

SEQ ID NO: 66 - DNA, 10 bp, Artificial Sequence
  tag sequence
cccatcatcc

SEQ ID NO: 67 - DNA, 10 bp, Artificial Sequence
  tag sequence
gcagtggcct

SEQ ID NO: 68 - DNA, 10 bp, Artificial Sequence
  tag sequence
aaagtctaga

SEQ ID NO: 69 - DNA, 10 bp, Artificial Sequence
  tag sequence
ctggcgccga

SEQ ID NO: 70 - DNA, 10 bp, Artificial Sequence
  tag sequence
tttcagagag

SEQ ID NO: 71 - DNA, 10 bp, Artificial Sequence
  tag sequence
ttcttgctta

SEQ ID NO: 72 - DNA, 10 bp, Artificial Sequence
  tag sequence
gagagtgggg

SEQ ID NO: 73 - DNA, 10 bp, Artificial Sequence
  tag sequence
tgagcaagcc

SEQ ID NO: 74 - DNA, 10 bp, Artificial Sequence
  tag sequence
cctgtacccc

SEQ ID NO: 75 - DNA, 10 bp, Artificial Sequence
  tag sequence
cctttcacac

SEQ ID NO: 76 - DNA, 10 bp, Artificial Sequence
  tag sequence
caccagcatt

SEQ ID NO: 77 - DNA, 10 bp, Artificial Sequence
  tag sequence
ttttgtaatt

SEQ ID NO: 78 - DNA, 10 bp, Artificial Sequence
  tag sequence
gtgcagggag

SEQ ID NO: 79 - DNA, 10 bp, Artificial Sequence
  tag sequence
atgactcaag

SEQ ID NO: 80 - DNA, 10 bp, Artificial Sequence
  tag sequence
attgtttatg

SEQ ID NO: 81 - DNA, 10 bp, Artificial Sequence
  tag sequence
aaggatgcca

SEQ ID NO: 82 - DNA, 10 bp, Artificial Sequence
  tag sequence
cttgtaatcc

SEQ ID NO: 83 - DNA, 10 bp, Artificial Sequence
  tag sequence
tagtttgtgg

SEQ ID NO: 84 - DNA, 10 bp, Artificial Sequence
  tag sequence
cggtcttatg

SEQ ID NO: 85 - DNA, 10 bp, Artificial Sequence
  tag sequence
tgaaaagctt

SEQ ID NO: 86 - DNA, 10 bp, Artificial Sequence
  tag sequence
ttaagaggga

SEQ ID NO: 87 - DNA, 10 bp, Artificial Sequence
  tag sequence
tatttcaccg

SEQ ID NO: 88 - DNA, 10 bp, Artificial Sequence
  tag sequence
gtctttcttg

SEQ ID NO: 89 - DNA, 10 bp, Artificial Sequence
  tag sequence
ccaggggaga

SEQ ID NO: 90 - DNA, 10 bp, Artificial Sequence
  tag sequence
gagcagcgcc

SEQ ID NO: 91 - DNA, 10 bp, Artificial Sequence
  tag sequence
gctctgcttg

SEQ ID NO: 92 - DNA, 10 bp, Artificial Sequence
  tag sequence
cgccgacgat

SEQ ID NO: 93 - DNA, 10 bp, Artificial Sequence
  tag sequence
gtgtgtttgt

SEQ ID NO: 94 - DNA, 10 bp, Artificial Sequence
  tag sequence
ccaataaagt

SEQ ID NO: 95 - DNA, 10 bp, Artificial Sequence
  tag sequence
gtctagaatc

SEQ ID NO: 96 - DNA, 10 bp, Artificial Sequence
  tag sequence
atccgcgagg

SEQ ID NO: 97 - DNA, 10 bp, Artificial Sequence
  tag sequence
gattttgcac

SEQ ID NO: 98 - DNA, 10 bp, Artificial Sequence
  tag sequence
accttgtgcc

SEQ ID NO: 99 - DNA, 10 bp, Artificial Sequence
  tag sequence
tgccgttttg

SEQ ID NO: 100 - DNA, 10 bp, Artificial Sequence
  tag sequence
ccgtgctcat

SEQ ID NO: 101 - DNA, 10 bp, Artificial Sequence
  tag sequence
gtttctatca

SEQ ID NO: 102 - DNA, 10 bp, Artificial Sequence
  tag sequence
caaataaaat

SEQ ID NO: 103 - DNA, 10 bp, Artificial Sequence
  tag sequence
ggaactttta

SEQ ID NO: 104 - DNA, 10 bp, Artificial Sequence
  tag sequence
ttaccttttt

SEQ ID NO: 105 - DNA, 10 bp, Artificial Sequence
  tag sequence
ttggggaaac

SEQ ID NO: 106 - DNA, 10 bp, Artificial Sequence
  tag sequence
tgatctccaa

SEQ ID NO: 107 - DNA, 10 bp, Artificial Sequence
  tag sequence
tttggtgttt

SEQ ID NO: 108 - DNA, 10 bp, Artificial Sequence
  tag sequence
ttaacccctc

SEQ ID NO: 109 - DNA, 10 bp, Artificial Sequence
  tag sequence
gctttgatga

SEQ ID NO: 110 - DNA, 10 bp, Artificial Sequence
  tag sequence
tacagtatgt

SEQ ID NO: 111 - DNA, 10 bp, Artificial Sequence
  tag sequence
tggggttctt

SEQ ID NO: 112 - DNA, 10 bp, Artificial Sequence
  tag sequence
ttacttcccc

SEQ ID NO: 113 - DNA, 10 bp, Artificial Sequence
  tag sequence
aagaatctga

SEQ ID NO: 114 - DNA, 10 bp, Artificial Sequence
  tag sequence
gtccctgcct

SEQ ID NO: 115 - DNA, 10 bp, Artificial Sequence
  tag sequence
aatatgtggg

SEQ ID NO: 116 - DNA, 10 bp, Artificial Sequence
  tag sequence
ggagctctgt

SEQ ID NO: 117 - DNA, 10 bp, Artificial Sequence
  tag sequence
gaaggagata

SEQ ID NO: 118 - DNA, 10 bp, Artificial Sequence
  tag sequence
tcagactttt

SEQ ID NO: 119 - DNA, 10 bp, Artificial Sequence
  tag sequence
tcttgtaact

SEQ ID NO: 120 - DNA, 10 bp, Artificial Sequence
  tag sequence
tgatgagtgt

SEQ ID NO: 121 - DNA, 10 bp, Artificial Sequence
  tag sequence
ctgcaaccta

SEQ ID NO: 122 - DNA, 10 bp, Artificial Sequence
  tag sequence
tgagtggttt

SEQ ID NO: 123 - DNA, 10 bp, Artificial Sequence
  tag sequence
cactgtgttg

SEQ ID NO: 124 - DNA, 10 bp, Artificial Sequence
  tag sequence
ttaagaagtt

SEQ ID NO: 125 - DNA, 10 bp, Artificial Sequence
  tag sequence
gcgacagtaa

SEQ ID NO: 126 - DNA, 10 bp, Artificial Sequence
  tag sequence
tcaacttgaa

SEQ ID NO: 127 - DNA, 10 bp, Artificial Sequence
  tag sequence
tttctggagg

SEQ ID NO: 128 - DNA, 10 bp, Artificial Sequence
  tag sequence
ggggctggag

SEQ ID NO: 129 - DNA, 10 bp, Artificial Sequence
  tag sequence
gtctcatttc

SEQ ID NO: 130 - DNA, 10 bp, Artificial Sequence
  tag sequence
accgcctgtg

SEQ ID NO: 131 - DNA, 10 bp, Artificial Sequence
  tag sequence
gaagaacaga

SEQ ID NO: 132 - DNA, 10 bp, Artificial Sequence
  tag sequence
tcgtaacgag

SEQ ID NO: 133 - DNA, 10 bp, Artificial Sequence
  tag sequence
gtgatggggc

SEQ ID NO: 134 - DNA, 10 bp, Artificial Sequence
  tag sequence
gagagaaaat

SEQ ID NO: 135 - DNA, 10 bp, Artificial Sequence
  tag sequence
gcccacatcc

SEQ ID NO: 136 - DNA, 10 bp, Artificial Sequence
  tag sequence
gtatttaact

SEQ ID NO: 137 - DNA, 10 bp, Artificial Sequence
  tag sequence
ggctggtctc

SEQ ID NO: 138 - DNA, 10 bp, Artificial Sequence
  tag sequence
aacacttctc

SEQ ID NO: 139 - DNA, 10 bp, Artificial Sequence
  tag sequence
aataaagaga

SEQ ID NO: 140 - DNA, 10 bp, Artificial Sequence
  tag sequence
gagaaacatt

SEQ ID NO: 141 - DNA, 10 bp, Artificial Sequence
  tag sequence
tttggtcttt

SEQ ID NO: 142 - DNA, 10 bp, Artificial Sequence
  tag sequence
tgtggtggtg

SEQ ID NO: 143 - DNA, 10 bp, Artificial Sequence
  tag sequence
gaaagatgct

SEQ ID NO: 144 - DNA, 10 bp, Artificial Sequence
  tag sequence
tagcagaccc

SEQ ID NO: 145 - DNA, 10 bp, Artificial Sequence
  tag sequence
aacgctgcga

SEQ ID NO: 146 - DNA, 10 bp, Artificial Sequence
  tag sequence
aatggatgaa

SEQ ID NO: 147 - DNA, 10 bp, Artificial Sequence
  tag sequence
acatcgtagt

SEQ ID NO: 148 - DNA, 10 bp, Artificial Sequence
  tag sequence
acccgccggg

SEQ ID NO: 149 - DNA, 10 bp, Artificial Sequence
  tag sequence
agtgcaggga

SEQ ID NO: 150 - DNA, 10 bp, Artificial Sequence
  tag sequence
atcaagaatc

SEQ ID NO: 151 - DNA, 10 bp, Artificial Sequence
  tag sequence
atgtggcaca

SEQ ID NO: 152 - DNA, 10 bp, Artificial Sequence
  tag sequence
caaaccttta

SEQ ID NO: 153 - DNA, 10 bp, Artificial Sequence
  tag sequence
caatgctgcc

SEQ ID NO: 154 - DNA, 10 bp, Artificial Sequence
  tag sequence
cagcttaatt

SEQ ID NO: 155 - DNA, 10 bp, Artificial Sequence
  tag sequence
ccgacgggcg

SEQ ID NO: 156 - DNA, 10 bp, Artificial Sequence
  tag sequence
cctttgaaca

SEQ ID NO: 157 - DNA, 10 bp, Artificial Sequence
  tag sequence
cctttgccct

SEQ ID NO: 158 - DNA, 10 bp, Artificial Sequence
  tag sequence
cggtttaatt

SEQ ID NO: 159 - DNA, 10 bp, Artificial Sequence
  tag sequence
ctttattcca

SEQ ID NO: 160 - DNA, 10 bp, Artificial Sequence
  tag sequence
gaagtcggaa

SEQ ID NO: 161 - DNA, 10 bp, Artificial Sequence
  tag sequence
gatctcgcaa

SEQ ID NO: 162 - DNA, 10 bp, Artificial Sequence
  tag sequence
gcacctccta

SEQ ID NO: 163 - DNA, 10 bp, Artificial Sequence
  tag sequence
gccgtgagca

SEQ ID NO: 164 - DNA, 10 bp, Artificial Sequence
  tag sequence
ggaaagtgac

SEQ ID NO: 165 - DNA, 10 bp, Artificial Sequence
  tag sequence
ggacctttat

SEQ ID NO: 166 - DNA, 10 bp, Artificial Sequence
  tag sequence
ggcagacaat

SEQ ID NO: 167 - DNA, 10 bp, Artificial Sequence
  tag sequence
ggcagcacaa

SEQ ID NO: 168 - DNA, 10 bp, Artificial Sequence
  tag sequence
ggtagctgct

SEQ ID NO: 169 - DNA, 10 bp, Artificial Sequence
  tag sequence
ggtagtttta

SEQ ID NO: 170 - DNA, 10 bp, Artificial Sequence
  tag sequence
ggtcagtcgg

SEQ ID NO: 171 - DNA, 10 bp, Artificial Sequence
  tag sequence
gtaatcctgc

SEQ ID NO: 172 - DNA, 10 bp, Artificial Sequence
  tag sequence
gtagttactg

SEQ ID NO: 173 - DNA, 10 bp, Artificial Sequence
  tag sequence
tcacagtgcc

SEQ ID NO: 174 - DNA, 10 bp, Artificial Sequence
  tag sequence
tctggtttgt

SEQ ID NO: 175 - DNA, 10 bp, Artificial Sequence
  tag sequence
tgaagcagta

SEQ ID NO: 176 - DNA, 10 bp, Artificial Sequence
  tag sequence
tgtcatagtt

SEQ ID NO: 177 - DNA, 10 bp, Artificial Sequence
  tag sequence
ttacgatgaa

SEQ ID NO: 178 - DNA, 10 bp, Artificial Sequence
  tag sequence
ttcggttggt

SEQ ID NO: 179 - DNA, 10 bp, Artificial Sequence
  tag sequence
agcgacaaac

SEQ ID NO: 180 - DNA, 10 bp, Artificial Sequence
  tag sequence
aggaaggaac

SEQ ID NO: 181 - DNA, 10 bp, Artificial Sequence
  tag sequence
ctgttccggc

SEQ ID NO: 182 - DNA, 10 bp, Artificial Sequence
  tag sequence
atcgctttct

SEQ ID NO: 183 - DNA, 10 bp, Artificial Sequence
  tag sequence
gtggccacgg

SEQ ID NO: 184 - DNA, 10 bp, Artificial Sequence
  tag sequence
atgtgaagag

SEQ ID NO: 185 - DNA, 10 bp, Artificial Sequence
  tag sequence
atgtgaagag

SEQ ID NO: 186 - DNA, 10 bp, Artificial Sequence
  tag sequence
tgaagcagta

SEQ ID NO: 187 - DNA, 10 bp, Artificial Sequence
  tag sequence
tgaagcagta

SEQ ID NO: 188 - DNA, 10 bp, Artificial Sequence
  tag sequence
accaaaaacc

SEQ ID NO: 189 - DNA, 10 bp, Artificial Sequence
  tag sequence
tttgcacctt

SEQ ID NO: 190 - DNA, 10 bp, Artificial Sequence
  tag sequence
tttggttttc

SEQ ID NO: 191 - DNA, 10 bp, Artificial Sequence
  tag sequence
tttggttttc

SEQ ID NO: 192 - DNA, 10 bp, Artificial Sequence
  tag sequence
tggaaatgac

SEQ ID NO: 193 - DNA, 10 bp, Artificial Sequence
  tag sequence
tggaaatgac

SEQ ID NO: 194 - DNA, 10 bp, Artificial Sequence
  tag sequence
gggcatctct

SEQ ID NO: 195 - DNA, 10 bp, Artificial Sequence
  tag sequence
ttgctgactt

SEQ ID NO: 196 - DNA, 10 bp, Artificial Sequence
  tag sequence
ttgctgactt

SEQ ID NO: 197 - DNA, 10 bp, Artificial Sequence
  tag sequence
tttcagagag

SEQ ID NO: 198 - DNA, 10 bp, Artificial Sequence
  tag sequence
tttcagagag

SEQ ID NO: 199 - DNA, 10 bp, Artificial Sequence
  tag sequence
aactgcttca

SEQ ID NO: 200 - DNA, 10 bp, Artificial Sequence
  tag sequence
acttacctgc

SEQ ID NO: 201 - DNA, 10 bp, Artificial Sequence
  tag sequence
acttacctgc

SEQ ID NO: 202 - DNA, 10 bp, Artificial Sequence
  tag sequence
tgtggtggtg

SEQ ID NO: 203 - DNA, 10 bp, Artificial Sequence
  tag sequence
tgtggtggtg

SEQ ID NO: 204 - DNA, 10 bp, Artificial Sequence
  tag sequence
ttacttcccc

SEQ ID NO: 205 - DNA, 10 bp, Artificial Sequence
  tag sequence
catttcaata

SEQ ID NO: 206 - DNA, 10 bp, Artificial Sequence
  tag sequence
catttcaata

SEQ ID NO: 207 - DNA, 10 bp, Artificial Sequence
  tag sequence
gtgctgattc

SEQ ID NO: 208 - DNA, 10 bp, Artificial Sequence
  tag sequence
gtgctgattc

SEQ ID NO: 209 - DNA, 10 bp, Artificial Sequence
  tag sequence
cgaccccacg

SEQ ID NO: 210 - DNA, 10 bp, Artificial Sequence
  tag sequence
ttttgtaact

SEQ ID NO: 211 - DNA, 10 bp, Artificial Sequence
  tag sequence
tctaagtacg

SEQ ID NO: 212 - DNA, 10 bp, Artificial Sequence
  tag sequence
cttccttgcc

SEQ ID NO: 213 - DNA, 10 bp, Artificial Sequence
  tag sequence
cttccttgcc

SEQ ID NO: 214 - DNA, 10 bp, Artificial Sequence
  tag sequence
ttaagaagtt

SEQ ID NO: 215 - DNA, 10 bp, Artificial Sequence
  tag sequence
gctctgcttg

SEQ ID NO: 216 - DNA, 10 bp, Artificial Sequence
  tag sequence
attaagaggg

SEQ ID NO: 217 - DNA, 10 bp, Artificial Sequence
  tag sequence
gagcagcgcc

SEQ ID NO: 218 - DNA, 10 bp, Artificial Sequence
  tag sequence
cctgggaagt

SEQ ID NO: 219 - DNA, 10 bp, Artificial Sequence
  tag sequence
cctgggaagt

SEQ ID NO: 220 - DNA, 10 bp, Artificial Sequence
  tag sequence
caaactaacc

SEQ ID NO: 221 - DNA, 10 bp, Artificial Sequence
  tag sequence
caaactaacc

SEQ ID NO: 222 - DNA, 10 bp, Artificial Sequence
  tag sequence
aaaccccaat

SEQ ID NO: 223 - DNA, 10 bp, Artificial Sequence
  tag sequence
aaaccccaat

SEQ ID NO: 224 - DNA, 10 bp, Artificial Sequence
  tag sequence
gaaataaagc

SEQ ID NO: 225 - DNA, 10 bp, Artificial Sequence
  tag sequence
gaaataaagc

SEQ ID NO: 226 - DNA, 10 bp, Artificial Sequence
  tag sequence
aagggagcac

SEQ ID NO: 227 - DNA, 10 bp, Artificial Sequence
  tag sequence
aagggagcac

SEQ ID NO: 228 - DNA, 10 bp, Artificial Sequence
  tag sequence
ggagtgtgct

SEQ ID NO: 229 - DNA, 10 bp, Artificial Sequence
  tag sequence
catatcatta

SEQ ID NO: 230 - DNA, 10 bp, Artificial Sequence
  tag sequence
tttttaatgt

SEQ ID NO: 231 - DNA, 10 bp, Artificial Sequence
  tag sequence
tttttaatgt

SEQ ID NO: 232 - DNA, 10 bp, Artificial Sequence
  tag sequence
ctcccccaag

SEQ ID NO: 233 - DNA, 10 bp, Artificial Sequence
  tag sequence
ctcccccaaa

SEQ ID NO: 234 - DNA, 10 bp, Artificial Sequence
  tag sequence
gttcacatta

SEQ ID NO: 235 - DNA, 10 bp, Artificial Sequence
  tag sequence
gttcacatta

SEQ ID NO: 236 - DNA, 10 bp, Artificial Sequence
  tag sequence
gtacgtattc

SEQ ID NO: 237 - DNA, 10 bp, Artificial Sequence
  tag sequence
gtacgtattc

SEQ ID NO: 238 - DNA, 10 bp, Artificial Sequence
  tag sequence
taaaatattg

SEQ ID NO: 239 - DNA, 10 bp, Artificial Sequence
  tag sequence
taataaaggt

SEQ ID NO: 240 - DNA, 10 bp, Artificial Sequence
  tag sequence
taataaaggt

SEQ ID NO: 241 - DNA, 10 bp, Artificial Sequence
  tag sequence
caataaatgt

SEQ ID NO: 242 - DNA, 10 bp, Artificial Sequence
  tag sequence
caataaatgt

SEQ ID NO: 243 - DNA, 10 bp, Artificial Sequence
  tag sequence
ctctcaccct

SEQ ID NO: 244 - DNA, 10 bp, Artificial Sequence
  tag sequence
ctctcaccct

SEQ ID NO: 245 - DNA, 10 bp, Artificial Sequence
  tag sequence
gtgcctaggg

SEQ ID NO: 246 - DNA, 10 bp, Artificial Sequence
  tag sequence
cctatttact

SEQ ID NO: 247 - DNA, 10 bp, Artificial Sequence
  tag sequence
ctgttgattg

SEQ ID NO: 248 - DNA, 10 bp, Artificial Sequence
  tag sequence
ctgttgattg

SEQ ID NO: 249 - DNA, 10 bp, Artificial Sequence
  tag sequence
gttgtctttg

SEQ ID NO: 250 - DNA, 10 bp, Artificial Sequence
  tag sequence
gttgtctttg

SEQ ID NO: 251 - DNA, 10 bp, Artificial Sequence
  tag sequence
gctcacctgt

SEQ ID NO: 252 - DNA, 10 bp, Artificial Sequence
  tag sequence
gctcacctgt

SEQ ID NO: 253 - DNA, 10 bp, Artificial Sequence
  tag sequence
gtgtaataag

SEQ ID NO: 254 - DNA, 10 bp, Artificial Sequence
  tag sequence
caatgctgcc

SEQ ID NO: 255 - DNA, 10 bp, Artificial Sequence
  tag sequence
gtgatggtgt

SEQ ID NO: 256 - DNA, 10 bp, Artificial Sequence
  tag sequence
gtgatggtgt

SEQ ID NO: 257 - DNA, 10 bp, Artificial Sequence
  tag sequence
tgagggaata

SEQ ID NO: 258 - DNA, 10 bp, Artificial Sequence
  tag sequence
ggcacagtaa

SEQ ID NO: 259 - DNA, 10 bp, Artificial Sequence
  tag sequence
ggcacagtaa

SEQ ID NO: 260 - DNA, 10 bp, Artificial Sequence
  tag sequence
ggctgtaccc

SEQ ID NO: 261 - DNA, 10 bp, Artificial Sequence
  tag sequence
ggctgtaccc

SEQ ID NO: 262 - DNA, 10 bp, Artificial Sequence
  tag sequence
aacacagcct

SEQ ID NO: 263 - DNA, 10 bp, Artificial Sequence
  tag sequence
aacacagcct

SEQ ID NO: 264 - DNA, 10 bp, Artificial Sequence
  tag sequence
cagttctctg

SEQ ID NO: 265 - DNA, 10 bp, Artificial Sequence
  tag sequence
aaggacctag

SEQ ID NO: 266 - DNA, 10 bp, Artificial Sequence
  tag sequence
taataaatgc

SEQ ID NO: 267 - DNA, 10 bp, Artificial Sequence
  tag sequence
ccctatcaca

SEQ ID NO: 268 - DNA, 10 bp, Artificial Sequence
  tag sequence
cggtttaatt

SEQ ID NO: 269 - DNA, 10 bp, Artificial Sequence
  tag sequence
tttctagttt

SEQ ID NO: 270 - DNA, 10 bp, Artificial Sequence
  tag sequence
ctggaggctg

SEQ ID NO: 271 - DNA, 10 bp, Artificial Sequence
  tag sequence
ctggaggctg

SEQ ID NO: 272 - DNA, 10 bp, Artificial Sequence
  tag sequence
cctagctgga

SEQ ID NO: 273 - DNA, 10 bp, Artificial Sequence
  tag sequence
cctagctgga

SEQ ID NO: 274 - DNA, 10 bp, Artificial Sequence
  tag sequence
ttacctcctt

SEQ ID NO: 275 - DNA, 10 bp, Artificial Sequence
  tag sequence
caattaaaag

SEQ ID NO: 276 - DNA, 10 bp, Artificial Sequence
  tag sequence
caattaaaag

SEQ ID NO: 277 - DNA, 10 bp, Artificial Sequence
  tag sequence
cctttcacac

SEQ ID NO: 278 - DNA, 10 bp, Artificial Sequence
  tag sequence
cctttcacac

SEQ ID NO: 279 - DNA, 10 bp, Artificial Sequence
  tag sequence
ttcggttggt

SEQ ID NO: 280 - DNA, 10 bp, Artificial Sequence
  tag sequence
ggtagtttta

SEQ ID NO: 281 - DNA, 10 bp, Artificial Sequence
  tag sequence
gtagacacct

SEQ ID NO: 282 - DNA, 10 bp, Artificial Sequence
  tag sequence
tttaatttgt

SEQ ID NO: 283 - DNA, 10 bp, Artificial Sequence
  tag sequence
tttaatttgt

SEQ ID NO: 284 - DNA, 10 bp, Artificial Sequence
  tag sequence
aagttgctat

SEQ ID NO: 285 - DNA, 10 bp, Artificial Sequence
  tag sequence
aagttgctat

SEQ ID NO: 286 - DNA, 10 bp, Artificial Sequence
  tag sequence
ggaatgtacg

SEQ ID NO: 287 - DNA, 10 bp, Artificial Sequence
  tag sequence
caagcaggac

SEQ ID NO: 288 - DNA, 10 bp, Artificial Sequence
  tag sequence
taggacaact

SEQ ID NO: 289 - DNA, 10 bp, Artificial Sequence
  tag sequence
caccacggtg

SEQ ID NO: 290 - DNA, 10 bp, Artificial Sequence
  tag sequence
tacagtatgt

SEQ ID NO: 291 - DNA, 10 bp, Artificial Sequence
  tag sequence
ctgttggtga

SEQ ID NO: 292 - DNA, 10 bp, Artificial Sequence
  tag sequence
ctgttggtga

SEQ ID NO: 293 - DNA, 10 bp, Artificial Sequence
  tag sequence
tgtatgaatt

SEQ ID NO: 294 - DNA, 10 bp, Artificial Sequence
  tag sequence
tgtatgaatt

SEQ ID NO: 295 - DNA, 10 bp, Artificial Sequence
  tag sequence
ctcgcgctgg

SEQ ID NO: 296 - DNA, 10 bp, Artificial Sequence
  tag sequence
ctcgcgctgg

SEQ ID NO: 297 - DNA, 10 bp, Artificial Sequence
  tag sequence
ggtgagacac

SEQ ID NO: 298 - DNA, 10 bp, Artificial Sequence
  tag sequence
ggtgagacac

SEQ ID NO: 299 - DNA, 10 bp, Artificial Sequence
  tag sequence
ggggtaagaa

SEQ ID NO: 300 - DNA, 10 bp, Artificial Sequence
  tag sequence
gcagccatcc

SEQ ID NO: 301 - DNA, 10 bp, Artificial Sequence
  tag sequence
tgctggtgtg

SEQ ID NO: 302 - DNA, 10 bp, Artificial Sequence
  tag sequence
tgctggtgtg

SEQ ID NO: 303 - DNA, 10 bp, Artificial Sequence
  tag sequence
agggcttcca

SEQ ID NO: 304 - DNA, 10 bp, Artificial Sequence
  tag sequence
agggcttcca

SEQ ID NO: 305 - DNA, 10 bp, Artificial Sequence
  tag sequence
gtaggggtaa

SEQ ID NO: 306 - DNA, 10 bp, Artificial Sequence
  tag sequence
cttgagcaat

SEQ ID NO: 307 - DNA, 10 bp, Artificial Sequence
  tag sequence
gtctggggct

SEQ ID NO: 308 - DNA, 10 bp, Artificial Sequence
  tag sequence
gcccccaata

SEQ ID NO: 309 - DNA, 10 bp, Artificial Sequence
  tag sequence
tggctgggaa

SEQ ID NO: 310 - DNA, 10 bp, Artificial Sequence
  tag sequence
gggcccagga

SEQ ID NO: 311 - DNA, 10 bp, Artificial Sequence
  tag sequence
gggcccagga

SEQ ID NO: 312 - DNA, 10 bp, Artificial Sequence
  tag sequence
caagggccaa

SEQ ID NO: 313 - DNA, 10 bp, Artificial Sequence
  tag sequence
gcaaaagaaa

SEQ ID NO: 314 - DNA, 10 bp, Artificial Sequence
  tag sequence
gcaaaagaaa

SEQ ID NO: 315 - DNA, 10 bp, Artificial Sequence
  tag sequence
ctccacccga

SEQ ID NO: 316 - DNA, 10 bp, Artificial Sequence
  tag sequence
aatatgtggg

SEQ ID NO: 317 - DNA, 10 bp, Artificial Sequence
  tag sequence
aatatgtggg

SEQ ID NO: 318 - DNA, 10 bp, Artificial Sequence
  tag sequence
gtagttactg

SEQ ID NO: 319 - DNA, 10 bp, Artificial Sequence
  tag sequence
tggcaacctt

SEQ ID NO: 320 - DNA, 10 bp, Artificial Sequence
  tag sequence
tggcaacctt

SEQ ID NO: 321 - DNA, 10 bp, Artificial Sequence
  tag sequence
tgtcatagtt

SEQ ID NO: 322 - DNA, 10 bp, Artificial Sequence
  tag sequence
gtccctgcct

SEQ ID NO: 323 - DNA, 10 bp, Artificial Sequence
  tag sequence
gtccctgcct

SEQ ID NO: 324 - DNA, 10 bp, Artificial Sequence
  tag sequence
attgtttatg

SEQ ID NO: 325 - DNA, 10 bp, Artificial Sequence
  tag sequence
attgtttatg

SEQ ID NO: 326 - DNA, 10 bp, Artificial Sequence
  tag sequence
gcctgctggg

SEQ ID NO: 327 - DNA, 10 bp, Artificial Sequence
  tag sequence
tgctgcctgt

SEQ ID NO: 328 - DNA, 10 bp, Artificial Sequence
  tag sequence
tgctgcctgt

SEQ ID NO: 329 - DNA, 10 bp, Artificial Sequence
  tag sequence
gtgacctcct

SEQ ID NO: 330 - DNA, 10 bp, Artificial Sequence
  tag sequence
cacgcaatgc

SEQ ID NO: 331 - DNA, 10 bp, Artificial Sequence
  tag sequence
cacgcaatgc

SEQ ID NO: 332 - DNA, 10 bp, Artificial Sequence
  tag sequence
caaaccatcc

SEQ ID NO: 333 - DNA, 10 bp, Artificial Sequence
  tag sequence
caaaccatcc

SEQ ID NO: 334 - DNA, 10 bp, Artificial Sequence
  tag sequence
accgcctgtg

SEQ ID NO: 335 - DNA, 10 bp, Artificial Sequence
  tag sequence
ctcaacatct

SEQ ID NO: 336 - DNA, 10 bp, Artificial Sequence
  tag sequence
ctcaacatct

SEQ ID NO: 337 - DNA, 10 bp, Artificial Sequence
  tag sequence
ttgtaatcgt

SEQ ID NO: 338 - DNA, 10 bp, Artificial Sequence
  tag sequence
gtgccatatt

SEQ ID NO: 339 - DNA, 10 bp, Artificial Sequence
  tag sequence
gtgccatatt

SEQ ID NO: 340 - DNA, 10 bp, Artificial Sequence
  tag sequence
catttgtaat

SEQ ID NO: 341 - DNA, 10 bp, Artificial Sequence
  tag sequence
agtgccgtgt

SEQ ID NO: 342 - DNA, 10 bp, Artificial Sequence
  tag sequence
agtgccgtgt

SEQ ID NO: 343 - DNA, 10 bp, Artificial Sequence
  tag sequence
atggctggta

SEQ ID NO: 344 - DNA, 10 bp, Artificial Sequence
  tag sequence
atggctggta

SEQ ID NO: 345 - DNA, 10 bp, Artificial Sequence
  tag sequence
ggctttaccc

SEQ ID NO: 346 - DNA, 10 bp, Artificial Sequence
  tag sequence
ttggtgaagg

SEQ ID NO: 347 - DNA, 10 bp, Artificial Sequence
  tag sequence
ttggtgaagg

SEQ ID NO: 348 - DNA, 10 bp, Artificial Sequence
  tag sequence
tagctctatg

SEQ ID NO: 349 - DNA, 10 bp, Artificial Sequence
  tag sequence
aataaagaga

SEQ ID NO: 350 - DNA, 10 bp, Artificial Sequence
  tag sequence
aataaagaga

SEQ ID NO: 351 - DNA, 10 bp, Artificial Sequence
  tag sequence
caaataaaaa

SEQ ID NO: 352 - DNA, 10 bp, Artificial Sequence
  tag sequence
caaataaaaa

SEQ ID NO: 353 - DNA, 10 bp, Artificial Sequence
  tag sequence
taccatcaat

SEQ ID NO: 354 - DNA, 10 bp, Artificial Sequence
  tag sequence
taccatcaat

SEQ ID NO: 355 - DNA, 10 bp, Artificial Sequence
  tag sequence
taagtagcaa

SEQ ID NO: 356 - DNA, 10 bp, Artificial Sequence
  tag sequence
taagtagcaa

SEQ ID NO: 357 - DNA, 10 bp, Artificial Sequence
  tag sequence
gaagcaggac

SEQ ID NO: 358 - DNA, 10 bp, Artificial Sequence
  tag sequence
ttagcaataa

SEQ ID NO: 359 - DNA, 10 bp, Artificial Sequence
  tag sequence
ttagcaataa

SEQ ID NO: 360 - DNA, 10 bp, Artificial Sequence
  tag sequence
caatgtgtta

SEQ ID NO: 361 - DNA, 10 bp, Artificial Sequence
  tag sequence
caatgtgtta

SEQ ID NO: 362 - DNA, 10 bp, Artificial Sequence
  tag sequence
gaggacccaa

SEQ ID NO: 363 - DNA, 10 bp, Artificial Sequence
  tag sequence
ccgtgctcat

SEQ ID NO: 364 - DNA, 10 bp, Artificial Sequence
  tag sequence
gggtgcttgg

SEQ ID NO: 365 - DNA, 10 bp, Artificial Sequence
  tag sequence
gtgcagggag

SEQ ID NO: 366 - DNA, 10 bp, Artificial Sequence
  tag sequence
gtgcagggag

SEQ ID NO: 367 - DNA, 10 bp, Artificial Sequence
  tag sequence
ttactaaatg

SEQ ID NO: 368 - DNA, 10 bp, Artificial Sequence
  tag sequence
ttactaaatg

SEQ ID NO: 369 - DNA, 10 bp, Artificial Sequence
  tag sequence
gaaatacagt

SEQ ID NO: 370 - DNA, 10 bp, Artificial Sequence
  tag sequence
gaaatacagt

SEQ ID NO: 371 - DNA, 10 bp, Artificial Sequence
  tag sequence
caaataaaat

SEQ ID NO: 372 - DNA, 10 bp, Artificial Sequence
  tag sequence
tgcatctggt

SEQ ID NO: 373 - DNA, 10 bp, Artificial Sequence
  tag sequence
tttcagggga

SEQ ID NO: 374 - DNA, 10 bp, Artificial Sequence
  tag sequence
tttggtgttt

SEQ ID NO: 375 - DNA, 10 bp, Artificial Sequence
  tag sequence
tacctctgat

SEQ ID NO: 376 - DNA, 10 bp, Artificial Sequence
  tag sequence
tacctctgat

SEQ ID NO: 377 - DNA, 10 bp, Artificial Sequence
  tag sequence
ggccagccct

SEQ ID NO: 378 - DNA, 10 bp, Artificial Sequence
  tag sequence
ggccagccct

SEQ ID NO: 379 - DNA, 10 bp, Artificial Sequence
  tag sequence
gctttgatga

SEQ ID NO: 380 - DNA, 10 bp, Artificial Sequence
  tag sequence
gctttgatga

SEQ ID NO: 381 - DNA, 10 bp, Artificial Sequence
  tag sequence
aataaaggct

SEQ ID NO: 382 - DNA, 10 bp, Artificial Sequence
  tag sequence
aataaaggct

SEQ ID NO: 383 - DNA, 10 bp, Artificial Sequence
  tag sequence
cctttgccct

SEQ ID NO: 384 - DNA, 10 bp, Artificial Sequence
  tag sequence
cacttcaagg

SEQ ID NO: 385 - DNA, 10 bp, Artificial Sequence
  tag sequence
ttcatacacc

SEQ ID NO: 386 - DNA, 10 bp, Artificial Sequence
  tag sequence
tctgtacacc

SEQ ID NO: 387 - DNA, 10 bp, Artificial Sequence
  tag sequence
ccattgcact

SEQ ID NO: 388 - DNA, 10 bp, Artificial Sequence
  tag sequence
ccattgcact

SEQ ID NO: 389 - DNA, 10 bp, Artificial Sequence
  tag sequence
aaataaagaa

SEQ ID NO: 390 - DNA, 10 bp, Artificial Sequence
  tag sequence
aaataaagaa

SEQ ID NO: 391 - DNA, 10 bp, Artificial Sequence
  tag sequence
gggttggctt

SEQ ID NO: 392 - DNA, 10 bp, Artificial Sequence
  tag sequence
actttttcaa

SEQ ID NO: 393 - DNA, 10 bp, Artificial Sequence
  tag sequence
actttttcaa

SEQ ID NO: 394 - DNA, 10 bp, Artificial Sequence
  tag sequence
cccatcgtcc

SEQ ID NO: 395 - DNA, 10 bp, Artificial Sequence
  tag sequence
gcggctttcc

SEQ ID NO: 396 - DNA, 10 bp, Artificial Sequence
  tag sequence
gggaagcaga

SEQ ID NO: 397 - DNA, 10 bp, Artificial Sequence
  tag sequence
ctgacctgtg

SEQ ID NO: 398 - DNA, 10 bp, Artificial Sequence
  tag sequence
ctgacctgtg

SEQ ID NO: 399 - DNA, 10 bp, Artificial Sequence
  tag sequence
gtaagtgtac

SEQ ID NO: 400 - DNA, 10 bp, Artificial Sequence
  tag sequence
tagttggaaa

SEQ ID NO: 401 - DNA, 10 bp, Artificial Sequence
  tag sequence
attttctaaa

SEQ ID NO: 402 - DNA, 10 bp, Artificial Sequence
  tag sequence
tgctaaaaaa

SEQ ID NO: 403 - DNA, 10 bp, Artificial Sequence
  tag sequence
tgctaaaaaa

SEQ ID NO: 404 - DNA, 10 bp, Artificial Sequence
  tag sequence
ggaataaatt

SEQ ID NO: 405 - DNA, 10 bp, Artificial Sequence
  tag sequence
gtgtgtaaaa

SEQ ID NO: 406 - DNA, 10 bp, Artificial Sequence
  tag sequence
agaaaaaaaa

SEQ ID NO: 407 - DNA, 10 bp, Artificial Sequence
  tag sequence
agaaaaaaaa

SEQ ID NO: 408 - DNA, 10 bp, Artificial Sequence
  tag sequence
tcaaaaaaaa

SEQ ID NO: 409 - DNA, 10 bp, Artificial Sequence
  tag sequence
tcaaaaaaaa

SEQ ID NO: 410 - DNA, 10 bp, Artificial Sequence
  tag sequence
ctaaaaaaaa

SEQ ID NO: 411 - DNA, 10 bp, Artificial Sequence
  tag sequence
ctaaaaaaaa

SEQ ID NO: 412 - DNA, 10 bp, Artificial Sequence
  tag sequence
caaaaaaaaa

SEQ ID NO: 413 - DNA, 10 bp, Artificial Sequence
  tag sequence
caaaaaaaaa

SEQ ID NO: 414 - DNA, 10 bp, Artificial Sequence
  tag sequence
gactcacttt

SEQ ID NO: 415 - DNA, 10 bp, Artificial Sequence
  tag sequence
agtttcccaa

SEQ ID NO: 416 - DNA, 10 bp, Artificial Sequence
  tag sequence
agtttcccaa

SEQ ID NO: 417 - DNA, 10 bp, Artificial Sequence
  tag sequence
gcaaaaaaaa

SEQ ID NO: 418 - DNA, 10 bp, Artificial Sequence
  tag sequence
gcaaaaaaaa

SEQ ID NO: 419 - DNA, 10 bp, Artificial Sequence
  tag sequence
cacttgccct

SEQ ID NO: 420 - DNA, 10 bp, Artificial Sequence
  tag sequence
cacttgccct

SEQ ID NO: 421 - DNA, 10 bp, Artificial Sequence
  tag sequence
cttaatcctg

SEQ ID NO: 422 - DNA, 10 bp, Artificial Sequence
  tag sequence
aaaaaaaaaa

SEQ ID NO: 423 - DNA, 10 bp, Artificial Sequence
  tag sequence
aaaaaaaaaa

SEQ ID NO: 424 - DNA, 10 bp, Artificial Sequence
  tag sequence
gaaaaaaaaa

SEQ ID NO: 425 - DNA, 10 bp, Artificial Sequence
  tag sequence
gaaaaaaaaa

SEQ ID NO: 426 - DNA, 10 bp, Artificial Sequence
  tag sequence
ggggactgaa

SEQ ID NO: 427 - DNA, 10 bp, Artificial Sequence
  tag sequence
ggggactgaa

SEQ ID NO: 428 - DNA, 10 bp, Artificial Sequence
  tag sequence
ttgaattccc

SEQ ID NO: 429 - DNA, 10 bp, Artificial Sequence
  tag sequence
gctttttaga

SEQ ID NO: 430 - DNA, 10 bp, Artificial Sequence
  tag sequence
gctttttaga

SEQ ID NO: 431 - DNA, 10 bp, Artificial Sequence
  tag sequence
tttctgttaa

SEQ ID NO: 432 - DNA, 10 bp, Artificial Sequence
  tag sequence
tgatctccaa

SEQ ID NO: 433 - DNA, 10 bp, Artificial Sequence
  tag sequence
tgatctccaa

SEQ ID NO: 434 - DNA, 10 bp, Artificial Sequence
  tag sequence
aaagtctaga

SEQ ID NO: 435 - DNA, 10 bp, Artificial Sequence
  tag sequence
ccctaccctg

SEQ ID NO: 436 - DNA, 10 bp, Artificial Sequence
  tag sequence
tacataatta

SEQ ID NO: 437 - DNA, 10 bp, Artificial Sequence
  tag sequence
ttcaataaaa

SEQ ID NO: 438 - DNA, 10 bp, Artificial Sequence
  tag sequence
ttcaataaaa

SEQ ID NO: 439 - DNA, 10 bp, Artificial Sequence
  tag sequence
taaggagctg

SEQ ID NO: 440 - DNA, 10 bp, Artificial Sequence
  tag sequence
taaggagctg

SEQ ID NO: 441 - DNA, 10 bp, Artificial Sequence
  tag sequence
taaaaaaaaa

SEQ ID NO: 442 - DNA, 10 bp, Artificial Sequence
  tag sequence
taaaaaaaaa

SEQ ID NO: 443 - DNA, 10 bp, Artificial Sequence
  tag sequence
tctgtttatc

SEQ ID NO: 444 - DNA, 10 bp, Artificial Sequence
  tag sequence
tctgtttatc

SEQ ID NO: 445 - DNA, 10 bp, Artificial Sequence
  tag sequence
gtaaaaaaaa

SEQ ID NO: 446 - DNA, 10 bp, Artificial Sequence
  tag sequence
gtaaaaaaaa

SEQ ID NO: 447 - DNA, 10 bp, Artificial Sequence
  tag sequence
ccccagttgc

SEQ ID NO: 448 - DNA, 10 bp, Artificial Sequence
  tag sequence
ccccagttgc

SEQ ID NO: 449 - DNA, 10 bp, Artificial Sequence
  tag sequence
tgtacctgta

SEQ ID NO: 450 - DNA, 10 bp, Artificial Sequence
  tag sequence
tgtacctgta

SEQ ID NO: 451 - DNA, 10 bp, Artificial Sequence
  tag sequence
gaacacatcc

SEQ ID NO: 452 - DNA, 10 bp, Artificial Sequence
  tag sequence
aatagttgtg

SEQ ID NO: 453 - DNA, 10 bp, Artificial Sequence
  tag sequence
aactaaaaaa

SEQ ID NO: 454 - DNA, 10 bp, Artificial Sequence
  tag sequence
aactaaaaaa

SEQ ID NO: 455 - DNA, 10 bp, Artificial Sequence
  tag sequence
taggttgtct

SEQ ID NO: 456 - DNA, 10 bp, Artificial Sequence
  tag sequence
taggttgtct

SEQ ID NO: 457 - DNA, 10 bp, Artificial Sequence
  tag sequence
ttaaaaaaaa

SEQ ID NO: 458 - DNA, 10 bp, Artificial Sequence
  tag sequence
ttaaaaaaaa

SEQ ID NO: 459 - DNA, 10 bp, Artificial Sequence
  tag sequence
aactaacaaa

SEQ ID NO: 460 - DNA, 10 bp, Artificial Sequence
  tag sequence
aactaacaaa

SEQ ID NO: 461 - DNA, 10 bp, Artificial Sequence
  tag sequence
caagggcttg

SEQ ID NO: 462 - DNA, 10 bp, Artificial Sequence
  tag sequence
aaggcaattt

SEQ ID NO: 463 - DNA, 10 bp, Artificial Sequence
  tag sequence
aaggcaattt

SEQ ID NO: 464 - DNA, 10 bp, Artificial Sequence
  tag sequence
ctcctcacct

SEQ ID NO: 465 - DNA, 10 bp, Artificial Sequence
  tag sequence
ctcctcacct

SEQ ID NO: 466 - DNA, 10 bp, Artificial Sequence
  tag sequence
gactctggtg

SEQ ID NO: 467 - DNA, 10 bp, Artificial Sequence
  tag sequence
gactctggtg

SEQ ID NO: 468 - DNA, 10 bp, Artificial Sequence
  tag sequence
attctccagt

SEQ ID NO: 469 - DNA, 10 bp, Artificial Sequence
  tag sequence
aaaaaaccca

SEQ ID NO: 470 - DNA, 10 bp, Artificial Sequence
  tag sequence
tgataattca

SEQ ID NO: 471 - DNA, 10 bp, Artificial Sequence
  tag sequence
gggctggggt

SEQ ID NO: 472 - DNA, 10 bp, Artificial Sequence
  tag sequence
gggctggggt

SEQ ID NO: 473 - DNA, 10 bp, Artificial Sequence
  tag sequence
gcttaacctg

SEQ ID NO: 474 - DNA, 10 bp, Artificial Sequence
  tag sequence
ggatttggcc

SEQ ID NO: 475 - DNA, 10 bp, Artificial Sequence
  tag sequence
ggatttggcc

SEQ ID NO: 476 - DNA, 10 bp, Artificial Sequence
  tag sequence
tgcacgtttt

SEQ ID NO: 477 - DNA, 10 bp, Artificial Sequence
  tag sequence
gcataatagg

SEQ ID NO: 478 - DNA, 10 bp, Artificial Sequence
  tag sequence
gcataatagg

SEQ ID NO: 479 - DNA, 10 bp, Artificial Sequence
  tag sequence
gcacaagaag

SEQ ID NO: 480 - DNA, 10 bp, Artificial Sequence
  tag sequence
taaactgttt

SEQ ID NO: 481 - DNA, 10 bp, Artificial Sequence
  tag sequence
tcagatcttt

SEQ ID NO: 482 - DNA, 10 bp, Artificial Sequence
  tag sequence
gacaaaaaaa

SEQ ID NO: 483 - DNA, 10 bp, Artificial Sequence
  tag sequence
gacaaaaaaa

SEQ ID NO: 484 - DNA, 10 bp, Artificial Sequence
  tag sequence
ggaacaaaca

SEQ ID NO: 485 - DNA, 10 bp, Artificial Sequence
  tag sequence
ggaacaaaca

SEQ ID NO: 486 - DNA, 10 bp, Artificial Sequence
  tag sequence
ctaacttcgt

SEQ ID NO: 487 - DNA, 10 bp, Artificial Sequence
  tag sequence
gctcagctgg

SEQ ID NO: 488 - DNA, 10 bp, Artificial Sequence
  tag sequence
tggcgtggcc

SEQ ID NO: 489 - DNA, 10 bp, Artificial Sequence
  tag sequence
agccaaaaaa

SEQ ID NO: 490 - DNA, 10 bp, Artificial Sequence
  tag sequence
agccaaaaaa

SEQ ID NO: 491 - DNA, 10 bp, Artificial Sequence
  tag sequence
tggcgtacgg

SEQ ID NO: 492 - DNA, 10 bp, Artificial Sequence
  tag sequence
ggagcgtggg

SEQ ID NO: 493 - DNA, 10 bp, Artificial Sequence
  tag sequence
acagcggcaa

SEQ ID NO: 494 - DNA, 10 bp, Artificial Sequence
  tag sequence
acagcggcaa

SEQ ID NO: 495 - DNA, 10 bp, Artificial Sequence
  tag sequence
tcaagttcac

SEQ ID NO: 496 - DNA, 10 bp, Artificial Sequence
  tag sequence
ggaagcacgg

SEQ ID NO: 497 - DNA, 10 bp, Artificial Sequence
  tag sequence
ggaagcacgg

SEQ ID NO: 498 - DNA, 10 bp, Artificial Sequence
  tag sequence
cagttacaaa

SEQ ID NO: 499 - DNA, 10 bp, Artificial Sequence
  tag sequence
cagttacaaa

SEQ ID NO: 500 - DNA, 10 bp, Artificial Sequence
  tag sequence
caggacagtt

SEQ ID NO: 501 - DNA, 10 bp, Artificial Sequence
  tag sequence
ggggaaatcg

SEQ ID NO: 502 - DNA, 10 bp, Artificial Sequence
  tag sequence
caaatccaaa

SEQ ID NO: 503 - DNA, 10 bp, Artificial Sequence
  tag sequence
tcagaagttt

SEQ ID NO: 504 - DNA, 10 bp, Artificial Sequence
  tag sequence
aaagttctca

SEQ ID NO: 505 - DNA, 10 bp, Artificial Sequence
  tag sequence
aaggatgcca

SEQ ID NO: 506 - DNA, 10 bp, Artificial Sequence
  tag sequence
aaggatgcca

SEQ ID NO: 507 - DNA, 10 bp, Artificial Sequence
  tag sequence
gagggccggt

SEQ ID NO: 508 - DNA, 10 bp, Artificial Sequence
  tag sequence
cagcagaagc

SEQ ID NO: 509 - DNA, 10 bp, Artificial Sequence
  tag sequence
cagcagaagc

SEQ ID NO: 510 - DNA, 10 bp, Artificial Sequence
  tag sequence
cctccagcta

SEQ ID NO: 511 - DNA, 10 bp, Artificial Sequence
  tag sequence
cctccagcta

SEQ ID NO: 512 - DNA, 10 bp, Artificial Sequence
  tag sequence
gccttccaat

SEQ ID NO: 513 - DNA, 10 bp, Artificial Sequence
  tag sequence
gggagcccgg

SEQ ID NO: 514 - DNA, 10 bp, Artificial Sequence
  tag sequence
gctcccagac

SEQ ID NO: 515 - DNA, 10 bp, Artificial Sequence
  tag sequence
gcagggcctc

SEQ ID NO: 516 - DNA, 10 bp, Artificial Sequence
  tag sequence
ttggagatct

SEQ ID NO: 517 - DNA, 10 bp, Artificial Sequence
  tag sequence
ggaaaaaaaa

SEQ ID NO: 518 - DNA, 10 bp, Artificial Sequence
  tag sequence
ggaaaaaaaa

SEQ ID NO: 519 - DNA, 10 bp, Artificial Sequence
  tag sequence
aagaaaactg

SEQ ID NO: 520 - DNA, 10 bp, Artificial Sequence
  tag sequence
aagaaaactg

SEQ ID NO: 521 - DNA, 10 bp, Artificial Sequence
  tag sequence
gacatcaagt

SEQ ID NO: 522 - DNA, 10 bp, Artificial Sequence
  tag sequence
gcagtggcct

SEQ ID NO: 523 - DNA, 10 bp, Artificial Sequence
  tag sequence
gcagtggcct

SEQ ID NO: 524 - DNA, 10 bp, Artificial Sequence
  tag sequence
cgccgacgat

SEQ ID NO: 525 - DNA, 10 bp, Artificial Sequence
  tag sequence
atgtcttttc

SEQ ID NO: 526 - DNA, 10 bp, Artificial Sequence
  tag sequence
atgtcttttc

SEQ ID NO: 527 - DNA, 10 bp, Artificial Sequence
  tag sequence
gccgtcggag

SEQ ID NO: 528 - DNA, 10 bp, Artificial Sequence
  tag sequence
cggactcact

SEQ ID NO: 529 - DNA, 10 bp, Artificial Sequence
  tag sequence
acgcagggag

SEQ ID NO: 530 - DNA, 10 bp, Artificial Sequence
  tag sequence
ccaggggaga

SEQ ID NO: 531 - DNA, 10 bp, Artificial Sequence
  tag sequence
ccaggggaga

SEQ ID NO: 532 - DNA, 10 bp, Artificial Sequence
  tag sequence
aagaaaacct

SEQ ID NO: 533 - DNA, 10 bp, Artificial Sequence
  tag sequence
aagaaaacct

SEQ ID NO: 534 - DNA, 10 bp, Artificial Sequence
  tag sequence
agattcaaac

SEQ ID NO: 535 - DNA, 10 bp, Artificial Sequence
  tag sequence
tggggagagg

SEQ ID NO: 536 - DNA, 10 bp, Artificial Sequence
  tag sequence
ccaaacgtgt

SEQ ID NO: 537 - DNA, 10 bp, Artificial Sequence
  tag sequence
ccaaacgtgt

SEQ ID NO: 538 - DNA, 10 bp, Artificial Sequence
  tag sequence
aagcctaaaa

SEQ ID NO: 539 - DNA, 10 bp, Artificial Sequence
  tag sequence
gtgctgaatg

SEQ ID NO: 540 - DNA, 10 bp, Artificial Sequence
  tag sequence
gtgctgaatg

SEQ ID NO: 541 - DNA, 10 bp, Artificial Sequence
  tag sequence
aacgcggcca

SEQ ID NO: 542 - DNA, 10 bp, Artificial Sequence
  tag sequence
aacgcggcca

SEQ ID NO: 543 - DNA, 10 bp, Artificial Sequence
  tag sequence
ggcaacgtgg

SEQ ID NO: 544 - DNA, 10 bp, Artificial Sequence
  tag sequence
ggcaacgtgg

SEQ ID NO: 545 - DNA, 10 bp, Artificial Sequence
  tag sequence
cgccgcggtg

SEQ ID NO: 546 - DNA, 10 bp, Artificial Sequence
  tag sequence
gtgaccacgg

SEQ ID NO: 547 - DNA, 10 bp, Artificial Sequence
  tag sequence
ccgacgggcg

SEQ ID NO: 548 - DNA, 10 bp, Artificial Sequence
  tag sequence
ggtggcactc

SEQ ID NO: 549 - DNA, 10 bp, Artificial Sequence
  tag sequence
ggtggcactc

SEQ ID NO: 550 - DNA, 10 bp, Artificial Sequence
  tag sequence
gggatcaagg

SEQ ID NO: 551 - DNA, 10 bp, Artificial Sequence
  tag sequence
tggagtggag

SEQ ID NO: 552 - DNA, 10 bp, Artificial Sequence
  tag sequence
tgcctctgcg

SEQ ID NO: 553 - DNA, 10 bp, Artificial Sequence
  tag sequence
tccctggctg

SEQ ID NO: 554 - DNA, 10 bp, Artificial Sequence
  tag sequence
tccctggctg

SEQ ID NO: 555 - DNA, 10 bp, Artificial Sequence
  tag sequence
gacgacacga

SEQ ID NO: 556 - DNA, 10 bp, Artificial Sequence
  tag sequence
gacgacacga

SEQ ID NO: 557 - DNA, 10 bp, Artificial Sequence
  tag sequence
gtgctggacc

SEQ ID NO: 558 - DNA, 10 bp, Artificial Sequence
  tag sequence
gtgctggacc

SEQ ID NO: 559 - DNA, 10 bp, Artificial Sequence
  tag sequence
gcaggccaag

SEQ ID NO: 560 - DNA, 10 bp, Artificial Sequence
  tag sequence
gcaggccaag

SEQ ID NO: 561 - DNA, 10 bp, Artificial Sequence
  tag sequence
tgcctgcacc

SEQ ID NO: 562 - DNA, 10 bp, Artificial Sequence
  tag sequence
tcagccttct

SEQ ID NO: 563 - DNA, 10 bp, Artificial Sequence
  tag sequence
tcagccttct

SEQ ID NO: 564 - DNA, 10 bp, Artificial Sequence
  tag sequence
tagaaaaata

SEQ ID NO: 565 - DNA, 10 bp, Artificial Sequence
  tag sequence
tagaaaaata

SEQ ID NO: 566 - DNA, 10 bp, Artificial Sequence
  tag sequence
aagacagtgg

SEQ ID NO: 567 - DNA, 10 bp, Artificial Sequence
  tag sequence
aagacagtgg

SEQ ID NO: 568 - DNA, 10 bp, Artificial Sequence
  tag sequence
tgtgctaaat

SEQ ID NO: 569 - DNA, 10 bp, Artificial Sequence
  tag sequence
tgtgctaaat

SEQ ID NO: 570 - DNA, 10 bp, Artificial Sequence
  tag sequence
tctccatacc

SEQ ID NO: 571 - DNA, 10 bp, Artificial Sequence
  tag sequence
ggcaagaaga

SEQ ID NO: 572 - DNA, 10 bp, Artificial Sequence
  tag sequence
ggcaagaaga

SEQ ID NO: 573 - DNA, 10 bp, Artificial Sequence
  tag sequence
gaaaaattta

SEQ ID NO: 574 - DNA, 10 bp, Artificial Sequence
  tag sequence
ttggtcctct

SEQ ID NO: 575 - DNA, 10 bp, Artificial Sequence
  tag sequence
ttggtcctct

SEQ ID NO: 576 - DNA, 10 bp, Artificial Sequence
  tag sequence
gtgtgggggg

SEQ ID NO: 577 - DNA, 10 bp, Artificial Sequence
  tag sequence
gtgtgggggg

SEQ ID NO: 578 - DNA, 10 bp, Artificial Sequence
  tag sequence
cgtgggtggg

SEQ ID NO: 579 - DNA, 10 bp, Artificial Sequence
  tag sequence
gcgacgaggc

SEQ ID NO: 580 - DNA, 10 bp, Artificial Sequence
  tag sequence
gccgttctta

SEQ ID NO: 581 - DNA, 10 bp, Artificial Sequence
  tag sequence
acccgccggg

SEQ ID NO: 582 - DNA, 10 bp, Artificial Sequence
  tag sequence
ggcctgctgc

SEQ ID NO: 583 - DNA, 10 bp, Artificial Sequence
  tag sequence
ggcctgctgc

SEQ ID NO: 584 - DNA, 10 bp, Artificial Sequence
  tag sequence
ggtttggctt

SEQ ID NO: 585 - DNA, 10 bp, Artificial Sequence
  tag sequence
tcagtttgtc

SEQ ID NO: 586 - DNA, 10 bp, Artificial Sequence
  tag sequence
tcagtttgtc

SEQ ID NO: 587 - DNA, 10 bp, Artificial Sequence
  tag sequence
ggtcagtcgg

SEQ ID NO: 588 - DNA, 10 bp, Artificial Sequence
  tag sequence
ctaactagtt

SEQ ID NO: 589 - DNA, 10 bp, Artificial Sequence
  tag sequence
aaggtggagg

SEQ ID NO: 590 - DNA, 10 bp, Artificial Sequence
  tag sequence
aaggtggagg

SEQ ID NO: 591 - DNA, 10 bp, Artificial Sequence
  tag sequence
aggctacgga

SEQ ID NO: 592 - DNA, 10 bp, Artificial Sequence
  tag sequence
aggctacgga

SEQ ID NO: 593 - DNA, 10 bp, Artificial Sequence
  tag sequence
gaagttatga

SEQ ID NO: 594 - DNA, 10 bp, Artificial Sequence
  tag sequence
tcacaagcaa

SEQ ID NO: 595 - DNA, 10 bp, Artificial Sequence
  tag sequence
gcgctggagt

SEQ ID NO: 596 - DNA, 10 bp, Artificial Sequence
  tag sequence
gcgctggagt

SEQ ID NO: 597 - DNA, 10 bp, Artificial Sequence
  tag sequence
ggaccactga

SEQ ID NO: 598 - DNA, 10 bp, Artificial Sequence
  tag sequence
ggaccactga

SEQ ID NO: 599 - DNA, 10 bp, Artificial Sequence
  tag sequence
gcggtgaggt

SEQ ID NO: 600 - DNA, 10 bp, Artificial Sequence
  tag sequence
caataaactg

SEQ ID NO: 601 - DNA, 10 bp, Artificial Sequence
  tag sequence
caataaactg

SEQ ID NO: 602 - DNA, 10 bp, Artificial Sequence
  tag sequence
aggaaagctg

SEQ ID NO: 603 - DNA, 10 bp, Artificial Sequence
  tag sequence
aggaaagctg

SEQ ID NO: 604 - DNA, 10 bp, Artificial Sequence
  tag sequence
ctgggttaat

SEQ ID NO: 605 - DNA, 10 bp, Artificial Sequence
  tag sequence
ctgggttaat

SEQ ID NO: 606 - DNA, 10 bp, Artificial Sequence
  tag sequence
aaggagatgg

SEQ ID NO: 607 - DNA, 10 bp, Artificial Sequence
  tag sequence
aaggagatgg

SEQ ID NO: 608 - DNA, 10 bp, Artificial Sequence
  tag sequence
acatcatcga

SEQ ID NO: 609 - DNA, 10 bp, Artificial Sequence
  tag sequence
acatcatcga

SEQ ID NO: 610 - DNA, 10 bp, Artificial Sequence
  tag sequence
attatttttc

SEQ ID NO: 611 - DNA, 10 bp, Artificial Sequence
  tag sequence
attatttttc

SEQ ID NO: 612 - DNA, 10 bp, Artificial Sequence
  tag sequence
tagttgaagt

SEQ ID NO: 613 - DNA, 10 bp, Artificial Sequence
  tag sequence
ccagaacaga

SEQ ID NO: 614 - DNA, 10 bp, Artificial Sequence
  tag sequence
ccagaacaga

SEQ ID NO: 615 - DNA, 10 bp, Artificial Sequence
  tag sequence
gcatttaaat

SEQ ID NO: 616 - DNA, 10 bp, Artificial Sequence
  tag sequence
gcatttaaat

SEQ ID NO: 617 - DNA, 10 bp, Artificial Sequence
  tag sequence
gaaaaatggt

SEQ ID NO: 618 - DNA, 10 bp, Artificial Sequence
  tag sequence
gaaaaatggt

SEQ ID NO: 619 - DNA, 10 bp, Artificial Sequence
  tag sequence
ggttggcagg

SEQ ID NO: 620 - DNA, 10 bp, Artificial Sequence
  tag sequence
ggttggcagg

SEQ ID NO: 621 - DNA, 10 bp, Artificial Sequence
  tag sequence
gtgaaggcag

SEQ ID NO: 622 - DNA, 10 bp, Artificial Sequence
  tag sequence
gtgaaggcag

SEQ ID NO: 623 - DNA, 10 bp, Artificial Sequence
  tag sequence
ttgcgttgcg

SEQ ID NO: 624 - DNA, 10 bp, Artificial Sequence
  tag sequence
atctcagctc

SEQ ID NO: 625 - DNA, 10 bp, Artificial Sequence
  tag sequence
atctcagctc

SEQ ID NO: 626 - DNA, 10 bp, Artificial Sequence
  tag sequence
aaaaaattca

SEQ ID NO: 627 - DNA, 10 bp, Artificial Sequence
  tag sequence
tggccccacc

SEQ ID NO: 628 - DNA, 10 bp, Artificial Sequence
  tag sequence
tggccccacc

SEQ ID NO: 629 - DNA, 10 bp, Artificial Sequence
  tag sequence
tccatctgtt

SEQ ID NO: 630 - DNA, 10 bp, Artificial Sequence
  tag sequence
caactggagt

SEQ ID NO: 631 - DNA, 10 bp, Artificial Sequence
  tag sequence
caactggagt

SEQ ID NO: 632 - DNA, 10 bp, Artificial Sequence
  tag sequence
gcccagctgg

SEQ ID NO: 633 - DNA, 10 bp, Artificial Sequence
  tag sequence
gcccagctgg

SEQ ID NO: 634 - DNA, 10 bp, Artificial Sequence
  tag sequence
gacggcgcag

SEQ ID NO: 635 - DNA, 10 bp, Artificial Sequence
  tag sequence
atgaaacccc

SEQ ID NO: 636 - DNA, 10 bp, Artificial Sequence
  tag sequence
atgaaacccc

SEQ ID NO: 637 - DNA, 10 bp, Artificial Sequence
  tag sequence
agccaccgca

SEQ ID NO: 638 - DNA, 10 bp, Artificial Sequence
  tag sequence
agccaccgca

SEQ ID NO: 639 - DNA, 10 bp, Artificial Sequence
  tag sequence
cccagctaat

SEQ ID NO: 640 - DNA, 10 bp, Artificial Sequence
  tag sequence
cccagctaat

SEQ ID NO: 641 - DNA, 10 bp, Artificial Sequence
  tag sequence
gtgaaacccc

SEQ ID NO: 642 - DNA, 10 bp, Artificial Sequence
  tag sequence
gtgaaacccc

SEQ ID NO: 643 - DNA, 10 bp, Artificial Sequence
  tag sequence
gtgaaaccct

SEQ ID NO: 644 - DNA, 10 bp, Artificial Sequence
  tag sequence
gtgaaaccct

SEQ ID NO: 645 - DNA, 10 bp, Artificial Sequence
  tag sequence
gagaaacccc

SEQ ID NO: 646 - DNA, 10 bp, Artificial Sequence
  tag sequence
gagaaacccc

SEQ ID NO: 647 - DNA, 10 bp, Artificial Sequence
  tag sequence
gtgaaacctt

SEQ ID NO: 648 - DNA, 10 bp, Artificial Sequence
  tag sequence
gtgaaacctt

SEQ ID NO: 649 - DNA, 10 bp, Artificial Sequence
  tag sequence
gtgaaactcc

SEQ ID NO: 650 - DNA, 10 bp, Artificial Sequence
  tag sequence
gtgaaactcc

SEQ ID NO: 651 - DNA, 10 bp, Artificial Sequence
  tag sequence
gtgaaatccc

SEQ ID NO: 652 - DNA, 10 bp, Artificial Sequence
  tag sequence
gtgaaatccc

SEQ ID NO: 653 - DNA, 10 bp, Artificial Sequence
  tag sequence
aacccgggag

SEQ ID NO: 654 - DNA, 10 bp, Artificial Sequence
  tag sequence
aacccgggag

SEQ ID NO: 655 - DNA, 10 bp, Artificial Sequence
  tag sequence
gtggcgggca

SEQ ID NO: 656 - DNA, 10 bp, Artificial Sequence
  tag sequence
gtggcgggca

SEQ ID NO: 657 - DNA, 10 bp, Artificial Sequence
  tag sequence
ttgcccaggc

SEQ ID NO: 658 - DNA, 10 bp, Artificial Sequence
  tag sequence
ttgcccaggc

SEQ ID NO: 659 - DNA, 10 bp, Artificial Sequence
  tag sequence
gtggtgggtg

SEQ ID NO: 660 - DNA, 10 bp, Artificial Sequence
  tag sequence
gtggtgggtg

SEQ ID NO: 661 - DNA, 10 bp, Artificial Sequence
  tag sequence
cctgtaatcc

SEQ ID NO: 662 - DNA, 10 bp, Artificial Sequence
  tag sequence
cctgtaatcc

SEQ ID NO: 663 - DNA, 10 bp, Artificial Sequence
  tag sequence
agccactgtg

SEQ ID NO: 664 - DNA, 10 bp, Artificial Sequence
  tag sequence
agccactgtg

SEQ ID NO: 665 - DNA, 10 bp, Artificial Sequence
  tag sequence
gtggcaggca

SEQ ID NO: 666 - DNA, 10 bp, Artificial Sequence
  tag sequence
gtggcaggca

SEQ ID NO: 667 - DNA, 10 bp, Artificial Sequence
  tag sequence
gtaaaacccc

SEQ ID NO: 668 - DNA, 10 bp, Artificial Sequence
  tag sequence
gtaaaacccc

SEQ ID NO: 669 - DNA, 10 bp, Artificial Sequence
  tag sequence
cctggctaat

SEQ ID NO: 670 - DNA, 10 bp, Artificial Sequence
  tag sequence
cctggctaat

SEQ ID NO: 671 - DNA, 10 bp, Artificial Sequence
  tag sequence
gtgaaatcct

SEQ ID NO: 672 - DNA, 10 bp, Artificial Sequence
  tag sequence
gtgaaatcct

SEQ ID NO: 673 - DNA, 10 bp, Artificial Sequence
  tag sequence
gtggcacgtg

SEQ ID NO: 674 - DNA, 10 bp, Artificial Sequence
  tag sequence
gtggcacgtg

SEQ ID NO: 675 - DNA, 10 bp, Artificial Sequence
  tag sequence
gtggctcaca

SEQ ID NO: 676 - DNA, 10 bp, Artificial Sequence
  tag sequence
gtggctcaca

SEQ ID NO: 677 - DNA, 10 bp, Artificial Sequence
  tag sequence
tgcctgtaat

SEQ ID NO: 678 - DNA, 10 bp, Artificial Sequence
  tag sequence
tgcctgtaat

SEQ ID NO: 679 - DNA, 10 bp, Artificial Sequence
  tag sequence
ccactgcact

SEQ ID NO: 680 - DNA, 10 bp, Artificial Sequence
  tag sequence
ccactgcact

SEQ ID NO: 681 - DNA, 10 bp, Artificial Sequence
  tag sequence
agaattgctt

SEQ ID NO: 682 - DNA, 10 bp, Artificial Sequence
  tag sequence
agaattgctt

SEQ ID NO: 683 - DNA, 10 bp, Artificial Sequence
  tag sequence
atcttggctc

SEQ ID NO: 684 - DNA, 10 bp, Artificial Sequence
  tag sequence
atcttggctc

SEQ ID NO: 685 - DNA, 10 bp, Artificial Sequence
  tag sequence
ttggccagga

SEQ ID NO: 686 - DNA, 10 bp, Artificial Sequence
  tag sequence
ttggccagga

SEQ ID NO: 687 - DNA, 10 bp, Artificial Sequence
  tag sequence
ttgaccaggc

SEQ ID NO: 688 - DNA, 10 bp, Artificial Sequence
  tag sequence
ttgaccaggc

SEQ ID NO: 689 - DNA, 10 bp, Artificial Sequence
  tag sequence
atccgcccgc

SEQ ID NO: 690 - DNA, 10 bp, Artificial Sequence
  tag sequence
atccgcccgc

SEQ ID NO: 691 - DNA, 10 bp, Artificial Sequence
  tag sequence
agccaccacg

SEQ ID NO: 692 - DNA, 10 bp, Artificial Sequence
  tag sequence
agccaccacg

SEQ ID NO: 693 - DNA, 10 bp, Artificial Sequence
  tag sequence
gtgaaacccg

SEQ ID NO: 694 - DNA, 10 bp, Artificial Sequence
  tag sequence
gtgaaacccg

SEQ ID NO: 695 - DNA, 10 bp, Artificial Sequence
  tag sequence
cccggctaat

SEQ ID NO: 696 - DNA, 10 bp, Artificial Sequence
  tag sequence
cccggctaat

SEQ ID NO: 697 - DNA, 10 bp, Artificial Sequence
  tag sequence
gtgaaaccca

SEQ ID NO: 698 - DNA, 10 bp, Artificial Sequence
  tag sequence
gtgaaaccca

SEQ ID NO: 699 - DNA, 10 bp, Artificial Sequence
  tag sequence
gtaaaaccct

SEQ ID NO: 700 - DNA, 10 bp, Artificial Sequence
  tag sequence
gtaaaaccct

SEQ ID NO: 701 - DNA, 10 bp, Artificial Sequence
  tag sequence
gtgaaactct

SEQ ID NO: 702 - DNA, 10 bp, Artificial Sequence
  tag sequence
gtgaaactct

SEQ ID NO: 703 - DNA, 10 bp, Artificial Sequence
  tag sequence
gtggcgggtg

SEQ ID NO: 704 - DNA, 10 bp, Artificial Sequence
  tag sequence
gtggcgggtg

SEQ ID NO: 705 - DNA, 10 bp, Artificial Sequence
  tag sequence
gtggcaggtg

SEQ ID NO: 706 - DNA, 10 bp, Artificial Sequence
  tag sequence
gtggcaggtg

SEQ ID NO: 707 - DNA, 10 bp, Artificial Sequence
  tag sequence
gcaaaaccct

SEQ ID NO: 708 - DNA, 10 bp, Artificial Sequence
  tag sequence
gcaaaaccct

SEQ ID NO: 709 - DNA, 10 bp, Artificial Sequence
  tag sequence
gcaaaacccc

SEQ ID NO: 710 - DNA, 10 bp, Artificial Sequence
  tag sequence
gcaaaacccc

SEQ ID NO: 711 - DNA, 10 bp, Artificial Sequence
  tag sequence
aggtcaggag

SEQ ID NO: 712 - DNA, 10 bp, Artificial Sequence
  tag sequence
aggtcaggag

SEQ ID NO: 713 - DNA, 10 bp, Artificial Sequence
  tag sequence
agccaccgtg

SEQ ID NO: 714 - DNA, 10 bp, Artificial Sequence
  tag sequence
agccaccgtg

SEQ ID NO: 715 - DNA, 10 bp, Artificial Sequence
  tag sequence
gtggcacaca

SEQ ID NO: 716 - DNA, 10 bp, Artificial Sequence
  tag sequence
gtggcacaca

SEQ ID NO: 717 - DNA, 10 bp, Artificial Sequence
  tag sequence
atctcggctc

SEQ ID NO: 718 - DNA, 10 bp, Artificial Sequence
  tag sequence
atctcggctc

SEQ ID NO: 719 - DNA, 10 bp, Artificial Sequence
  tag sequence
ttggccagac

SEQ ID NO: 720 - DNA, 10 bp, Artificial Sequence
  tag sequence
ttggccagac

SEQ ID NO: 721 - DNA, 10 bp, Artificial Sequence
  tag sequence
gtggcaggcg

SEQ ID NO: 722 - DNA, 10 bp, Artificial Sequence
  tag sequence
gtggcaggcg

SEQ ID NO: 723 - DNA, 10 bp, Artificial Sequence
  tag sequence
cacctgtaat

SEQ ID NO: 724 - DNA, 10 bp, Artificial Sequence
  tag sequence
cacctgtaat

SEQ ID NO: 725 - DNA, 10 bp, Artificial Sequence
  tag sequence
ttggccaggg

SEQ ID NO: 726 - DNA, 10 bp, Artificial Sequence
  tag sequence
ttggccaggg

SEQ ID NO: 727 - DNA, 10 bp, Artificial Sequence
  tag sequence
gagaaaccct

SEQ ID NO: 728 - DNA, 10 bp, Artificial Sequence
  tag sequence
gagaaaccct

SEQ ID NO: 729 - DNA, 10 bp, Artificial Sequence
  tag sequence
gcgaaaccct

SEQ ID NO: 730 - DNA, 10 bp, Artificial Sequence
  tag sequence
gcgaaaccct

SEQ ID NO: 731 - DNA, 10 bp, Artificial Sequence
  tag sequence
gtgaaacctc

SEQ ID NO: 732 - DNA, 10 bp, Artificial Sequence
  tag sequence
gtgaaacctc

SEQ ID NO: 733 - DNA, 10 bp, Artificial Sequence
  tag sequence
gcgaaacccc

SEQ ID NO: 734 - DNA, 10 bp, Artificial Sequence
  tag sequence
gcgaaacccc

SEQ ID NO: 735 - DNA, 10 bp, Artificial Sequence
  tag sequence
agccaccgcg

SEQ ID NO: 736 - DNA, 10 bp, Artificial Sequence
  tag sequence
agccaccgcg

SEQ ID NO: 737 - DNA, 10 bp, Artificial Sequence
  tag sequence
cgcctgtaat

SEQ ID NO: 738 - DNA, 10 bp, Artificial Sequence
  tag sequence
cgcctgtaat

SEQ ID NO: 739 - DNA, 10 bp, Artificial Sequence
  tag sequence
gtggcgggcg

SEQ ID NO: 740 - DNA, 10 bp, Artificial Sequence
  tag sequence
gtggcgggcg

SEQ ID NO: 741 - DNA, 10 bp, Artificial Sequence
  tag sequence
aacctgggag

SEQ ID NO: 742 - DNA, 10 bp, Artificial Sequence
  tag sequence
aacctgggag

SEQ ID NO: 743 - DNA, 10 bp, Artificial Sequence
  tag sequence
gctttctcac

SEQ ID NO: 744 - DNA, 10 bp, Artificial Sequence
  tag sequence
cttgtaatcc

SEQ ID NO: 745 - DNA, 10 bp, Artificial Sequence
  tag sequence
cttgtaatcc

SEQ ID NO: 746 - DNA, 10 bp, Artificial Sequence
  tag sequence
tctgtaatcc

SEQ ID NO: 747 - DNA, 10 bp, Artificial Sequence
  tag sequence
tctgtaatcc

SEQ ID NO: 748 - DNA, 10 bp, Artificial Sequence
  tag sequence
cctataatcc

SEQ ID NO: 749 - DNA, 10 bp, Artificial Sequence
  tag sequence
cctataatcc

SEQ ID NO: 750 - DNA, 10 bp, Artificial Sequence
  tag sequence
taatcccagc

SEQ ID NO: 751 - DNA, 10 bp, Artificial Sequence
  tag sequence
taatcccagc

SEQ ID NO: 752 - DNA, 10 bp, Artificial Sequence
  tag sequence
tgcctgtagt

SEQ ID NO: 753 - DNA, 10 bp, Artificial Sequence
  tag sequence
tgcctgtagt

SEQ ID NO: 754 - DNA, 10 bp, Artificial Sequence
  tag sequence
agggtgtttt

SEQ ID NO: 755 - DNA, 10 bp, Artificial Sequence
  tag sequence
agggtgtttt

SEQ ID NO: 756 - DNA, 10 bp, Artificial Sequence
  tag sequence
ccagggcaac

SEQ ID NO: 757 - DNA, 10 bp, Artificial Sequence
  tag sequence
attgtgccac

SEQ ID NO: 758 - DNA, 10 bp, Artificial Sequence
  tag sequence
attgtgccac

SEQ ID NO: 759 - DNA, 10 bp, Artificial Sequence
  tag sequence
cctgtaatct

SEQ ID NO: 760 - DNA, 10 bp, Artificial Sequence
  tag sequence
cctgtaatct

SEQ ID NO: 761 - DNA, 10 bp, Artificial Sequence
  tag sequence
gtggtgggca

SEQ ID NO: 762 - DNA, 10 bp, Artificial Sequence
  tag sequence
gtggtgggca

SEQ ID NO: 763 - DNA, 10 bp, Artificial Sequence
  tag sequence
taccctaaaa

SEQ ID NO: 764 - DNA, 10 bp, Artificial Sequence
  tag sequence
taccctaaaa

SEQ ID NO: 765 - DNA, 10 bp, Artificial Sequence
  tag sequence
atggtggggg

SEQ ID NO: 766 - DNA, 10 bp, Artificial Sequence
  tag sequence
acccttggcc

SEQ ID NO: 767 - DNA, 10 bp, Artificial Sequence
  tag sequence
gtgaaaaccc

SEQ ID NO: 768 - DNA, 10 bp, Artificial Sequence
  tag sequence
gtgaaaaccc

SEQ ID NO: 769 - DNA, 10 bp, Artificial Sequence
  tag sequence
atccacccgc

SEQ ID NO: 770 - DNA, 10 bp, Artificial Sequence
  tag sequence
atccacccgc

SEQ ID NO: 771 - DNA, 10 bp, Artificial Sequence
  tag sequence
ttagccagga

SEQ ID NO: 772 - DNA, 10 bp, Artificial Sequence
  tag sequence
ttagccagga

SEQ ID NO: 773 - DNA, 10 bp, Artificial Sequence
  tag sequence
atgaaaccct

SEQ ID NO: 774 - DNA, 10 bp, Artificial Sequence
  tag sequence
atgaaaccct

SEQ ID NO: 775 - DNA, 10 bp, Artificial Sequence
  tag sequence
gtggctcacg

SEQ ID NO: 776 - DNA, 10 bp, Artificial Sequence
  tag sequence
gtggctcacg

SEQ ID NO: 777 - DNA, 10 bp, Artificial Sequence
  tag sequence
ttggccaggc

SEQ ID NO: 778 - DNA, 10 bp, Artificial Sequence
  tag sequence
ttggccaggc

SEQ ID NO: 779 - DNA, 10 bp, Artificial Sequence
  tag sequence
ttggtcaggc

SEQ ID NO: 780 - DNA, 10 bp, Artificial Sequence
  tag sequence
ttggtcaggc

SEQ ID NO: 781 - DNA, 10 bp, Artificial Sequence
  tag sequence
ttgtccaggc

SEQ ID NO: 782 - DNA, 10 bp, Artificial Sequence
  tag sequence
ttgtccaggc

SEQ ID NO: 783 - DNA, 10 bp, Artificial Sequence
  tag sequence
cttaatcttg

SEQ ID NO: 784 - DNA, 10 bp, Artificial Sequence
  tag sequence
cttaatcttg

SEQ ID NO: 785 - DNA, 10 bp, Artificial Sequence
  tag sequence
tggggttctt

SEQ ID NO: 786 - DNA, 10 bp, Artificial Sequence
  tag sequence
tggggttctt

SEQ ID NO: 787 - DNA, 10 bp, Artificial Sequence
  tag sequence
aagaagatag

SEQ ID NO: 788 - DNA, 10 bp, Artificial Sequence
  tag sequence
aagaagatag

SEQ ID NO: 789 - DNA, 10 bp, Artificial Sequence
  tag sequence
agaatcgctt

SEQ ID NO: 790 - DNA, 10 bp, Artificial Sequence
  tag sequence
agaatcgctt

SEQ ID NO: 791 - DNA, 10 bp, Artificial Sequence
  tag sequence
cctgtagtcc

SEQ ID NO: 792 - DNA, 10 bp, Artificial Sequence
  tag sequence
cctgtagtcc

SEQ ID NO: 793 - DNA, 10 bp, Artificial Sequence
  tag sequence
agccaccaca

SEQ ID NO: 794 - DNA, 10 bp, Artificial Sequence
  tag sequence
agccaccaca

SEQ ID NO: 795 - DNA, 10 bp, Artificial Sequence
  tag sequence
attgcaccac

SEQ ID NO: 796 - DNA, 10 bp, Artificial Sequence
  tag sequence
attgcaccac

SEQ ID NO: 797 - DNA, 10 bp, Artificial Sequence
  tag sequence
ccactgtact

SEQ ID NO: 798 - DNA, 10 bp, Artificial Sequence
  tag sequence
ccactgtact

SEQ ID NO: 799 - DNA, 10 bp, Artificial Sequence
  tag sequence
ctgtacttgt

SEQ ID NO: 800 - DNA, 10 bp, Artificial Sequence
  tag sequence
ccattctcct

SEQ ID NO: 801 - DNA, 10 bp, Artificial Sequence
  tag sequence
ccattctcct

SEQ ID NO: 802 - DNA, 10 bp, Artificial Sequence
  tag sequence
gtggtgggcg

SEQ ID NO: 803 - DNA, 10 bp, Artificial Sequence
  tag sequence
gtggtgggcg

SEQ ID NO: 804 - DNA, 10 bp, Artificial Sequence
  tag sequence
agccactgcg

SEQ ID NO: 805 - DNA, 10 bp, Artificial Sequence
  tag sequence
agccactgcg

SEQ ID NO: 806 - DNA, 10 bp, Artificial Sequence
  tag sequence
gccggctcat

SEQ ID NO: 807 - DNA, 10 bp, Artificial Sequence
  tag sequence
gctcactgca

SEQ ID NO: 808 - DNA, 10 bp, Artificial Sequence
  tag sequence
gctcactgca

SEQ ID NO: 809 - DNA, 10 bp, Artificial Sequence
  tag sequence
cctgtggtcc

SEQ ID NO: 810 - DNA, 10 bp, Artificial Sequence
  tag sequence
cctgtggtcc

SEQ ID NO: 811 - DNA, 10 bp, Artificial Sequence
  tag sequence
ggaggctgag

SEQ ID NO: 812 - DNA, 10 bp, Artificial Sequence
  tag sequence
ggaggctgag

SEQ ID NO: 813 - DNA, 10 bp, Artificial Sequence
  tag sequence
agaatcactt

SEQ ID NO: 814 - DNA, 10 bp, Artificial Sequence
  tag sequence
agaatcactt

SEQ ID NO: 815 - DNA, 10 bp, Artificial Sequence
  tag sequence
cctgtaattc

SEQ ID NO: 816 - DNA, 10 bp, Artificial Sequence
  tag sequence
cctgtaattc

SEQ ID NO: 817 - DNA, 10 bp, Artificial Sequence
  tag sequence
agccactgca

SEQ ID NO: 818 - DNA, 10 bp, Artificial Sequence
  tag sequence
agccactgca

SEQ ID NO: 819 - DNA, 10 bp, Artificial Sequence
  tag sequence
aacccaggag

SEQ ID NO: 820 - DNA, 10 bp, Artificial Sequence
  tag sequence
aacccaggag

SEQ ID NO: 821 - DNA, 10 bp, Artificial Sequence
  tag sequence
aagccaggac

SEQ ID NO: 822 - DNA, 10 bp, Artificial Sequence
  tag sequence
gacctcctgc

SEQ ID NO: 823 - DNA, 10 bp, Artificial Sequence
  tag sequence
gacctcctgc

SEQ ID NO: 824 - DNA, 10 bp, Artificial Sequence
  tag sequence
ctgccaagtt

SEQ ID NO: 825 - DNA, 10 bp, Artificial Sequence
  tag sequence
gttcgtgcca

SEQ ID NO: 826 - DNA, 10 bp, Artificial Sequence
  tag sequence
gcgcagaggt

SEQ ID NO: 827 - DNA, 10 bp, Artificial Sequence
  tag sequence
gccgtgtccg

SEQ ID NO: 828 - DNA, 10 bp, Artificial Sequence
  tag sequence
gccgtgtccg

SEQ ID NO: 829 - DNA, 10 bp, Artificial Sequence
  tag sequence
cccatccgaa

SEQ ID NO: 830 - DNA, 10 bp, Artificial Sequence
  tag sequence
cccatccgaa

SEQ ID NO: 831 - DNA, 10 bp, Artificial Sequence
  tag sequence
cccgaggcag

SEQ ID NO: 832 - DNA, 10 bp, Artificial Sequence
  tag sequence
cccgaggcag

SEQ ID NO: 833 - DNA, 10 bp, Artificial Sequence
  tag sequence
cctgaaattt

SEQ ID NO: 834 - DNA, 10 bp, Artificial Sequence
  tag sequence
cctgaaattt

SEQ ID NO: 835 - DNA, 10 bp, Artificial Sequence
  tag sequence
ctcacttttt

SEQ ID NO: 836 - DNA, 10 bp, Artificial Sequence
  tag sequence
ctcacttttt

SEQ ID NO: 837 - DNA, 10 bp, Artificial Sequence
  tag sequence
gctgttgcgc

SEQ ID NO: 838 - DNA, 10 bp, Artificial Sequence
  tag sequence
tccccgtaca

SEQ ID NO: 839 - DNA, 10 bp, Artificial Sequence
  tag sequence
cacaaacggt

SEQ ID NO: 840 - DNA, 10 bp, Artificial Sequence
  tag sequence
cacaaacggt

SEQ ID NO: 841 - DNA, 10 bp, Artificial Sequence
  tag sequence
ccctgatttt

SEQ ID NO: 842 - DNA, 10 bp, Artificial Sequence
  tag sequence
ccctgatttt

SEQ ID NO: 843 - DNA, 10 bp, Artificial Sequence
  tag sequence
tgggcaaagc

SEQ ID NO: 844 - DNA, 10 bp, Artificial Sequence
  tag sequence
taacttgtga

SEQ ID NO: 845 - DNA, 10 bp, Artificial Sequence
  tag sequence
agcacctcca

SEQ ID NO: 846 - DNA, 10 bp, Artificial Sequence
  tag sequence
gagggagttt

SEQ ID NO: 847 - DNA, 10 bp, Artificial Sequence
  tag sequence
gagggagttt

SEQ ID NO: 848 - DNA, 10 bp, Artificial Sequence
  tag sequence
gcgacagctc

SEQ ID NO: 849 - DNA, 10 bp, Artificial Sequence
  tag sequence
cgccgccggc

SEQ ID NO: 850 - DNA, 10 bp, Artificial Sequence
  tag sequence
ggcaagcccc

SEQ ID NO: 851 - DNA, 10 bp, Artificial Sequence
  tag sequence
ggcaagcccc

SEQ ID NO: 852 - DNA, 10 bp, Artificial Sequence
  tag sequence
agctctccct

SEQ ID NO: 853 - DNA, 10 bp, Artificial Sequence
  tag sequence
agctctccct

SEQ ID NO: 854 - DNA, 10 bp, Artificial Sequence
  tag sequence
cgctggttcc

SEQ ID NO: 855 - DNA, 10 bp, Artificial Sequence
  tag sequence
cgctggttcc

SEQ ID NO: 856 - DNA, 10 bp, Artificial Sequence
  tag sequence
gaaaccgagg

SEQ ID NO: 857 - DNA, 10 bp, Artificial Sequence
  tag sequence
gaaaccgagg

SEQ ID NO: 858 - DNA, 10 bp, Artificial Sequence
  tag sequence
gaggtccctg

SEQ ID NO: 859 - DNA, 10 bp, Artificial Sequence
  tag sequence
gaggtccctg

SEQ ID NO: 860 - DNA, 10 bp, Artificial Sequence
  tag sequence
tgaaataaaa

SEQ ID NO: 861 - DNA, 10 bp, Artificial Sequence
  tag sequence
tgaaataaaa

SEQ ID NO: 862 - DNA, 10 bp, Artificial Sequence
  tag sequence
ccccagccag

SEQ ID NO: 863 - DNA, 10 bp, Artificial Sequence
  tag sequence
ccccagccag

SEQ ID NO: 864 - DNA, 10 bp, Artificial Sequence
  tag sequence
taaataattt

SEQ ID NO: 865 - DNA, 10 bp, Artificial Sequence
  tag sequence
ataattcttt

SEQ ID NO: 866 - DNA, 10 bp, Artificial Sequence
  tag sequence
ataattcttt

SEQ ID NO: 867 - DNA, 10 bp, Artificial Sequence
  tag sequence
ttaaacctca

SEQ ID NO: 868 - DNA, 10 bp, Artificial Sequence
  tag sequence
ttaaacctca

SEQ ID NO: 869 - DNA, 10 bp, Artificial Sequence
  tag sequence
gccgaggaag

SEQ ID NO: 870 - DNA, 10 bp, Artificial Sequence
  tag sequence
gccgaggaag

SEQ ID NO: 871 - DNA, 10 bp, Artificial Sequence
  tag sequence
gcctgtatga

SEQ ID NO: 872 - DNA, 10 bp, Artificial Sequence
  tag sequence
gcctgtatga

SEQ ID NO: 873 - DNA, 10 bp, Artificial Sequence
  tag sequence
gtgttaacca

SEQ ID NO: 874 - DNA, 10 bp, Artificial Sequence
  tag sequence
cttcgaaact

SEQ ID NO: 875 - DNA, 10 bp, Artificial Sequence
  tag sequence
aaggtcgagc

SEQ ID NO: 876 - DNA, 10 bp, Artificial Sequence
  tag sequence
aaggtcgagc

SEQ ID NO: 877 - DNA, 10 bp, Artificial Sequence
  tag sequence
ctttggaaat

SEQ ID NO: 878 - DNA, 10 bp, Artificial Sequence
  tag sequence
ctttggaaat

SEQ ID NO: 879 - DNA, 10 bp, Artificial Sequence
  tag sequence
ccccctggat

SEQ ID NO: 880 - DNA, 10 bp, Artificial Sequence
  tag sequence
cgccggaaca

SEQ ID NO: 881 - DNA, 10 bp, Artificial Sequence
  tag sequence
cgccggaaca

SEQ ID NO: 882 - DNA, 10 bp, Artificial Sequence
  tag sequence
gtgttgcaca

SEQ ID NO: 883 - DNA, 10 bp, Artificial Sequence
  tag sequence
gtgttgcaca

SEQ ID NO: 884 - DNA, 10 bp, Artificial Sequence
  tag sequence
caacttagtt

SEQ ID NO: 885 - DNA, 10 bp, Artificial Sequence
  tag sequence
ggggcagggc

SEQ ID NO: 886 - DNA, 10 bp, Artificial Sequence
  tag sequence
ccaagttttt

SEQ ID NO: 887 - DNA, 10 bp, Artificial Sequence
  tag sequence
ttggcagccc

SEQ ID NO: 888 - DNA, 10 bp, Artificial Sequence
  tag sequence
gttaacgtcc

SEQ ID NO: 889 - DNA, 10 bp, Artificial Sequence
  tag sequence
gttaacgtcc

SEQ ID NO: 890 - DNA, 10 bp, Artificial Sequence
  tag sequence
ggaagtttcg

SEQ ID NO: 891 - DNA, 10 bp, Artificial Sequence
  tag sequence
cccgtccgga

SEQ ID NO: 892 - DNA, 10 bp, Artificial Sequence
  tag sequence
cccgtccgga

SEQ ID NO: 893 - DNA, 10 bp, Artificial Sequence
  tag sequence
ggccgcgttc

SEQ ID NO: 894 - DNA, 10 bp, Artificial Sequence
  tag sequence
ggccgcgttc

SEQ ID NO: 895 - DNA, 10 bp, Artificial Sequence
  tag sequence
aaaagaaact

SEQ ID NO: 896 - DNA, 10 bp, Artificial Sequence
  tag sequence
aaaagaaact

SEQ ID NO: 897 - DNA, 10 bp, Artificial Sequence
  tag sequence
aactcccagt

SEQ ID NO: 898 - DNA, 10 bp, Artificial Sequence
  tag sequence
aactcccagt

SEQ ID NO: 899 - DNA, 10 bp, Artificial Sequence
  tag sequence
cacttttggg

SEQ ID NO: 900 - DNA, 10 bp, Artificial Sequence
  tag sequence
cacttttggg

SEQ ID NO: 901 - DNA, 10 bp, Artificial Sequence
  tag sequence
gggagggaag

SEQ ID NO: 902 - DNA, 10 bp, Artificial Sequence
  tag sequence
gggagggaag

SEQ ID NO: 903 - DNA, 10 bp, Artificial Sequence
  tag sequence
gggggaattt

SEQ ID NO: 904 - DNA, 10 bp, Artificial Sequence
  tag sequence
catctaaact

SEQ ID NO: 905 - DNA, 10 bp, Artificial Sequence
  tag sequence
tccccgtggc

SEQ ID NO: 906 - DNA, 10 bp, Artificial Sequence
  tag sequence
tccccgtggc

SEQ ID NO: 907 - DNA, 10 bp, Artificial Sequence
  tag sequence
gcctgcagtc

SEQ ID NO: 908 - DNA, 10 bp, Artificial Sequence
  tag sequence
gcctgcagtc

SEQ ID NO: 909 - DNA, 10 bp, Artificial Sequence
  tag sequence
agaatttgca

SEQ ID NO: 910 - DNA, 10 bp, Artificial Sequence
  tag sequence
agaatttgca

SEQ ID NO: 911 - DNA, 10 bp, Artificial Sequence
  tag sequence
tcggagctgt

SEQ ID NO: 912 - DNA, 10 bp, Artificial Sequence
  tag sequence
cacacagttt

SEQ ID NO: 913 - DNA, 10 bp, Artificial Sequence
  tag sequence
gtaatcctgc

SEQ ID NO: 914 - DNA, 10 bp, Artificial Sequence
  tag sequence
agaggtgtag

SEQ ID NO: 915 - DNA, 10 bp, Artificial Sequence
  tag sequence
ttagccaggc

SEQ ID NO: 916 - DNA, 10 bp, Artificial Sequence
  tag sequence
ttagccaggc

SEQ ID NO: 917 - DNA, 10 bp, Artificial Sequence
  tag sequence
tggaaagtga

SEQ ID NO: 918 - DNA, 10 bp, Artificial Sequence
  tag sequence
tggaaagtga

SEQ ID NO: 919 - DNA, 10 bp, Artificial Sequence
  tag sequence
tccctattaa

SEQ ID NO: 920 - DNA, 10 bp, Artificial Sequence
  tag sequence
aggagcgggg

SEQ ID NO: 921 - DNA, 10 bp, Artificial Sequence
  tag sequence
gcccctccgg

SEQ ID NO: 922 - DNA, 10 bp, Artificial Sequence
  tag sequence
gcccctccgg

SEQ ID NO: 923 - DNA, 10 bp, Artificial Sequence
  tag sequence
gctgcccttg

SEQ ID NO: 924 - DNA, 10 bp, Artificial Sequence
  tag sequence
gctgcccttg

SEQ ID NO: 925 - DNA, 10 bp, Artificial Sequence
  tag sequence
ccaccccgaa

SEQ ID NO: 926 - DNA, 10 bp, Artificial Sequence
  tag sequence
gctgcggtcc

SEQ ID NO: 927 - DNA, 10 bp, Artificial Sequence
  tag sequence
gctgcggtcc

SEQ ID NO: 928 - DNA, 10 bp, Artificial Sequence
  tag sequence
gagatccgca

SEQ ID NO: 929 - DNA, 10 bp, Artificial Sequence
  tag sequence
cagagatgaa

SEQ ID NO: 930 - DNA, 10 bp, Artificial Sequence
  tag sequence
gcaagccaac

SEQ ID NO: 931 - DNA, 10 bp, Artificial Sequence
  tag sequence
tggcctgccc

SEQ ID NO: 932 - DNA, 10 bp, Artificial Sequence
  tag sequence
gcggggtgga

SEQ ID NO: 933 - DNA, 10 bp, Artificial Sequence
  tag sequence
aggtggcaag

SEQ ID NO: 934 - DNA, 10 bp, Artificial Sequence
  tag sequence
tcgaagcccc

SEQ ID NO: 935 - DNA, 10 bp, Artificial Sequence
  tag sequence
tttaacggcc

SEQ ID NO: 936 - DNA, 10 bp, Artificial Sequence
  tag sequence
actttccaaa

SEQ ID NO: 937 - DNA, 10 bp, Artificial Sequence
  tag sequence
tggaagcact

SEQ ID NO: 938 - DNA, 10 bp, Artificial Sequence
  tag sequence
gtccgagtgc

SEQ ID NO: 939 - DNA, 10 bp, Artificial Sequence
  tag sequence
taacagccag

SEQ ID NO: 940 - DNA, 10 bp, Artificial Sequence
  tag sequence
taacagccag

SEQ ID NO: 941 - DNA, 10 bp, Artificial Sequence
  tag sequence
gccttgggtg

SEQ ID NO: 942 - DNA, 10 bp, Artificial Sequence
  tag sequence
tttgaaatga

SEQ ID NO: 943 - DNA, 10 bp, Artificial Sequence
  tag sequence
gggtaggggg

SEQ ID NO: 944 - DNA, 10 bp, Artificial Sequence
  tag sequence
atcgtggcgg

SEQ ID NO: 945 - DNA, 10 bp, Artificial Sequence
  tag sequence
atcgtggcgg

SEQ ID NO: 946 - DNA, 10 bp, Artificial Sequence
  tag sequence
cctggcctaa

SEQ ID NO: 947 - DNA, 10 bp, Artificial Sequence
  tag sequence
cctggcctaa

SEQ ID NO: 948 - DNA, 10 bp, Artificial Sequence
  tag sequence
aagattggtg

SEQ ID NO: 949 - DNA, 10 bp, Artificial Sequence
  tag sequence
aatcctgtgg

SEQ ID NO: 950 - DNA, 10 bp, Artificial Sequence
  tag sequence
aatcctgtgg

SEQ ID NO: 951 - DNA, 10 bp, Artificial Sequence
  tag sequence
tggtgttgag

SEQ ID NO: 952 - DNA, 10 bp, Artificial Sequence
  tag sequence
tggtgttgag

SEQ ID NO: 953 - DNA, 10 bp, Artificial Sequence
  tag sequence
ctggccctcg

SEQ ID NO: 954 - DNA, 10 bp, Artificial Sequence
  tag sequence
ctggccctcg

SEQ ID NO: 955 - DNA, 10 bp, Artificial Sequence
  tag sequence
gactcttcag

SEQ ID NO: 956 - DNA, 10 bp, Artificial Sequence
  tag sequence
ctgccaactt

SEQ ID NO: 957 - DNA, 10 bp, Artificial Sequence
  tag sequence
gtgcgctgag

SEQ ID NO: 958 - DNA, 10 bp, Artificial Sequence
  tag sequence
gtgcgctgag

SEQ ID NO: 959 - DNA, 10 bp, Artificial Sequence
  tag sequence
ttggggtttc

SEQ ID NO: 960 - DNA, 10 bp, Artificial Sequence
  tag sequence
ttggggtttc

SEQ ID NO: 961 - DNA, 10 bp, Artificial Sequence
  tag sequence
ggagggggct

SEQ ID NO: 962 - DNA, 10 bp, Artificial Sequence
  tag sequence
ggagggggct

SEQ ID NO: 963 - DNA, 10 bp, Artificial Sequence
  tag sequence
ttagttttta

SEQ ID NO: 964 - DNA, 10 bp, Artificial Sequence
  tag sequence
ttagttttta

SEQ ID NO: 965 - DNA, 10 bp, Artificial Sequence
  tag sequence
cccaagctag

SEQ ID NO: 966 - DNA, 10 bp, Artificial Sequence
  tag sequence
cccaagctag

SEQ ID NO: 967 - DNA, 10 bp, Artificial Sequence
  tag sequence
gtgcactgag

SEQ ID NO: 968 - DNA, 10 bp, Artificial Sequence
  tag sequence
gtgcactgag

SEQ ID NO: 969 - DNA, 10 bp, Artificial Sequence
  tag sequence
cagacttttt

SEQ ID NO: 970 - DNA, 10 bp, Artificial Sequence
  tag sequence
cagacttttt

SEQ ID NO: 971 - DNA, 10 bp, Artificial Sequence
  tag sequence
aaaacattct

SEQ ID NO: 972 - DNA, 10 bp, Artificial Sequence
  tag sequence
cacctaattg

SEQ ID NO: 973 - DNA, 10 bp, Artificial Sequence
  tag sequence
gggacgagtg

SEQ ID NO: 974 - DNA, 10 bp, Artificial Sequence
  tag sequence
caagcatccc

SEQ ID NO: 975 - DNA, 10 bp, Artificial Sequence
  tag sequence
agcagatcag

SEQ ID NO: 976 - DNA, 10 bp, Artificial Sequence
  tag sequence
agccctacaa

SEQ ID NO: 977 - DNA, 10 bp, Artificial Sequence
  tag sequence
tgaagtaaca

SEQ ID NO: 978 - DNA, 10 bp, Artificial Sequence
  tag sequence
gctaggttta

SEQ ID NO: 979 - DNA, 10 bp, Artificial Sequence
  tag sequence
caaaatcagg

SEQ ID NO: 980 - DNA, 10 bp, Artificial Sequence
  tag sequence
ggctgggggc

SEQ ID NO: 981 - DNA, 10 bp, Artificial Sequence
  tag sequence
ggctgggggc

SEQ ID NO: 982 - DNA, 10 bp, Artificial Sequence
  tag sequence
ggccctaggc

SEQ ID NO: 983 - DNA, 10 bp, Artificial Sequence
  tag sequence
gctgaacgcg

SEQ ID NO: 984 - DNA, 10 bp, Artificial Sequence
  tag sequence
aagagcgccg

SEQ ID NO: 985 - DNA, 10 bp, Artificial Sequence
  tag sequence
aagagcgccg

SEQ ID NO: 986 - DNA, 10 bp, Artificial Sequence
  tag sequence
agggtgaaac

SEQ ID NO: 987 - DNA, 10 bp, Artificial Sequence
  tag sequence
agggtgaaac

SEQ ID NO: 988 - DNA, 10 bp, Artificial Sequence
  tag sequence
gatcccaact

SEQ ID NO: 989 - DNA, 10 bp, Artificial Sequence
  tag sequence
gcctacccga

SEQ ID NO: 990 - DNA, 10 bp, Artificial Sequence
  tag sequence
ccaggaggaa

SEQ ID NO: 991 - DNA, 10 bp, Artificial Sequence
  tag sequence
ccaggaggaa

SEQ ID NO: 992 - DNA, 10 bp, Artificial Sequence
  tag sequence
ccagtggccc

SEQ ID NO: 993 - DNA, 10 bp, Artificial Sequence
  tag sequence
ccagtggccc

SEQ ID NO: 994 - DNA, 10 bp, Artificial Sequence
  tag sequence
gaagctttgc

SEQ ID NO: 995 - DNA, 10 bp, Artificial Sequence
  tag sequence
gaagctttgc

SEQ ID NO: 996 - DNA, 10 bp, Artificial Sequence
  tag sequence
tgtgttgaga

SEQ ID NO: 997 - DNA, 10 bp, Artificial Sequence
  tag sequence
tgtgttgaga

SEQ ID NO: 998 - DNA, 10 bp, Artificial Sequence
  tag sequence
gtgacagaag

SEQ ID NO: 999 - DNA, 10 bp, Artificial Sequence
  tag sequence
gtgacagaag

SEQ ID NO: 1000 - DNA, 10 bp, Artificial Sequence
  tag sequence
cctcggaaaa

SEQ ID NO: 1001 - DNA, 10 bp, Artificial Sequence
  tag sequence
cctcggaaaa

SEQ ID NO: 1002 - DNA, 10 bp, Artificial Sequence
  tag sequence
ctcataagga

SEQ ID NO: 1003 - DNA, 10 bp, Artificial Sequence
  tag sequence
ctagcctcac

SEQ ID NO: 1004 - DNA, 10 bp, Artificial Sequence
  tag sequence
gggccaaccc

SEQ ID NO: 1005 - DNA, 10 bp, Artificial Sequence
  tag sequence
gggccaaccc

SEQ ID NO: 1006 - DNA, 10 bp, Artificial Sequence
  tag sequence
acccccccgc

SEQ ID NO: 1007 - DNA, 10 bp, Artificial Sequence
  tag sequence
ggtgcccagt

SEQ ID NO: 1008 - DNA, 10 bp, Artificial Sequence
  tag sequence
gctttatttg

SEQ ID NO: 1009 - DNA, 10 bp, Artificial Sequence
  tag sequence
ggctcccact

SEQ ID NO: 1010 - DNA, 10 bp, Artificial Sequence
  tag sequence
ctaagacttc

SEQ ID NO: 1011 - DNA, 10 bp, Artificial Sequence
  tag sequence
gggtagctgg

SEQ ID NO: 1012 - DNA, 10 bp, Artificial Sequence
  tag sequence
acccacgtca

SEQ ID NO: 1013 - DNA, 10 bp, Artificial Sequence
  tag sequence
acccacgtca

SEQ ID NO: 1014 - DNA, 10 bp, Artificial Sequence
  tag sequence
gggcaggcgt

SEQ ID NO: 1015 - DNA, 10 bp, Artificial Sequence
  tag sequence
gttcactgca

SEQ ID NO: 1016 - DNA, 10 bp, Artificial Sequence
  tag sequence
gttcactgca

SEQ ID NO: 1017 - DNA, 10 bp, Artificial Sequence
  tag sequence
actcagcccg

SEQ ID NO: 1018 - DNA, 10 bp, Artificial Sequence
  tag sequence
actcagcccg

SEQ ID NO: 1019 - DNA, 10 bp, Artificial Sequence
  tag sequence
tgatttcact

SEQ ID NO: 1020 - DNA, 10 bp, Artificial Sequence
  tag sequence
aggtttcctc

SEQ ID NO: 1021 - DNA, 10 bp, Artificial Sequence
  tag sequence
accatcctgc

SEQ ID NO: 1022 - DNA, 10 bp, Artificial Sequence
  tag sequence
accatcctgc

SEQ ID NO: 1023 - DNA, 10 bp, Artificial Sequence
  tag sequence
gggaggtagc

SEQ ID NO: 1024 - DNA, 10 bp, Artificial Sequence
  tag sequence
ccgtccaagg

SEQ ID NO: 1025 - DNA, 10 bp, Artificial Sequence
  tag sequence
ctcaccgccc

SEQ ID NO: 1026 - DNA, 10 bp, Artificial Sequence
  tag sequence
cccgcccccg

SEQ ID NO: 1027 - DNA, 10 bp, Artificial Sequence
  tag sequence
actaacaccc

SEQ ID NO: 1028 - DNA, 10 bp, Artificial Sequence
  tag sequence
cactactcac

SEQ ID NO: 1029 - DNA, 10 bp, Artificial Sequence
  tag sequence
caggaggagt

SEQ ID NO: 1030 - DNA, 10 bp, Artificial Sequence
  tag sequence
caggaggagt

SEQ ID NO: 1031 - DNA, 10 bp, Artificial Sequence
  tag sequence
gcgaccgtca

SEQ ID NO: 1032 - DNA, 10 bp, Artificial Sequence
  tag sequence
aagggagggt

SEQ ID NO: 1033 - DNA, 10 bp, Artificial Sequence
  tag sequence
ggcagccaga

SEQ ID NO: 1034 - DNA, 10 bp, Artificial Sequence
  tag sequence
ggcagccaga

SEQ ID NO: 1035 - DNA, 10 bp, Artificial Sequence
  tag sequence
tgtgggtgct

SEQ ID NO: 1036 - DNA, 10 bp, Artificial Sequence
  tag sequence
tgtgggtgct

SEQ ID NO: 1037 - DNA, 10 bp, Artificial Sequence
  tag sequence
atttgagaag

SEQ ID NO: 1038 - DNA, 10 bp, Artificial Sequence
  tag sequence
aatggaaatc

SEQ ID NO: 1039 - DNA, 10 bp, Artificial Sequence
  tag sequence
aatggaaatc

SEQ ID NO: 1040 - DNA, 10 bp, Artificial Sequence
  tag sequence
tttgggccta

SEQ ID NO: 1041 - DNA, 10 bp, Artificial Sequence
  tag sequence
caactaattc

SEQ ID NO: 1042 - DNA, 10 bp, Artificial Sequence
  tag sequence
caactaattc

SEQ ID NO: 1043 - DNA, 10 bp, Artificial Sequence
  tag sequence
gttgtggtta

SEQ ID NO: 1044 - DNA, 10 bp, Artificial Sequence
  tag sequence
gttgtggtta

SEQ ID NO: 1045 - DNA, 10 bp, Artificial Sequence
  tag sequence
ttaaatggaa

SEQ ID NO: 1046 - DNA, 10 bp, Artificial Sequence
  tag sequence
ttaaatggaa

SEQ ID NO: 1047 - DNA, 10 bp, Artificial Sequence
  tag sequence
cttaaaaaaa

SEQ ID NO: 1048 - DNA, 10 bp, Artificial Sequence
  tag sequence
cttaaaaaaa

SEQ ID NO: 1049 - DNA, 10 bp, Artificial Sequence
  tag sequence
cttctccaaa

SEQ ID NO: 1050 - DNA, 10 bp, Artificial Sequence
  tag sequence
cttctccaaa

SEQ ID NO: 1051 - DNA, 10 bp, Artificial Sequence
  tag sequence
tacctgcaga

SEQ ID NO: 1052 - DNA, 10 bp, Artificial Sequence
  tag sequence
ataataaaag

SEQ ID NO: 1053 - DNA, 10 bp, Artificial Sequence
  tag sequence
ataataaaag

SEQ ID NO: 1054 - DNA, 10 bp, Artificial Sequence
  tag sequence
agaaagatgt

SEQ ID NO: 1055 - DNA, 10 bp, Artificial Sequence
  tag sequence
agaaagatgt

SEQ ID NO: 1056 - DNA, 10 bp, Artificial Sequence
  tag sequence
gtgcggagga

SEQ ID NO: 1057 - DNA, 10 bp, Artificial Sequence
  tag sequence
gtgcggagga

SEQ ID NO: 1058 - DNA, 10 bp, Artificial Sequence
  tag sequence
ggaaaagtgg

SEQ ID NO: 1059 - DNA, 10 bp, Artificial Sequence
  tag sequence
ggaaaagtgg

SEQ ID NO: 1060 - DNA, 10 bp, Artificial Sequence
  tag sequence
aataggtcca

SEQ ID NO: 1061 - DNA, 10 bp, Artificial Sequence
  tag sequence
aataggtcca

SEQ ID NO: 1062 - DNA, 10 bp, Artificial Sequence
  tag sequence
gtttatggat

SEQ ID NO: 1063 - DNA, 10 bp, Artificial Sequence
  tag sequence
caacaataat

SEQ ID NO: 1064 - DNA, 10 bp, Artificial Sequence
  tag sequence
tttattttaa

SEQ ID NO: 1065 - DNA, 10 bp, Artificial Sequence
  tag sequence
cttcctgtga

SEQ ID NO: 1066 - DNA, 10 bp, Artificial Sequence
  tag sequence
taaaaacttt

SEQ ID NO: 1067 - DNA, 10 bp, Artificial Sequence
  tag sequence
taaaaacttt

SEQ ID NO: 1068 - DNA, 10 bp, Artificial Sequence
  tag sequence
acacagcaag

SEQ ID NO: 1069 - DNA, 10 bp, Artificial Sequence
  tag sequence
tgcagcacga

SEQ ID NO: 1070 - DNA, 10 bp, Artificial Sequence
  tag sequence
tgcagcacga

SEQ ID NO: 1071 - DNA, 10 bp, Artificial Sequence
  tag sequence
actccaaaaa

SEQ ID NO: 1072 - DNA, 10 bp, Artificial Sequence
  tag sequence
actccaaaaa

SEQ ID NO: 1073 - DNA, 10 bp, Artificial Sequence
  tag sequence
gcctcctccc

SEQ ID NO: 1074 - DNA, 10 bp, Artificial Sequence
  tag sequence
gcctcctccc

SEQ ID NO: 1075 - DNA, 10 bp, Artificial Sequence
  tag sequence
aagctcgccg

SEQ ID NO: 1076 - DNA, 10 bp, Artificial Sequence
  tag sequence
cctggtccca

SEQ ID NO: 1077 - DNA, 10 bp, Artificial Sequence
  tag sequence
cctggtccca

SEQ ID NO: 1078 - DNA, 10 bp, Artificial Sequence
  tag sequence
gaattaacat

SEQ ID NO: 1079 - DNA, 10 bp, Artificial Sequence
  tag sequence
gaattaacat

SEQ ID NO: 1080 - DNA, 10 bp, Artificial Sequence
  tag sequence
taatttgcgt

SEQ ID NO: 1081 - DNA, 10 bp, Artificial Sequence
  tag sequence
ttggtttttg

SEQ ID NO: 1082 - DNA, 10 bp, Artificial Sequence
  tag sequence
ttggtttttg

SEQ ID NO: 1083 - DNA, 10 bp, Artificial Sequence
  tag sequence
gcttgcaaaa

SEQ ID NO: 1084 - DNA, 10 bp, Artificial Sequence
  tag sequence
gcttgcaaaa

SEQ ID NO: 1085 - DNA, 10 bp, Artificial Sequence
  tag sequence
gccgccctgc

SEQ ID NO: 1086 - DNA, 10 bp, Artificial Sequence
  tag sequence
gccgccctgc

SEQ ID NO: 1087 - DNA, 10 bp, Artificial Sequence
  tag sequence
cttccagcta

SEQ ID NO: 1088 - DNA, 10 bp, Artificial Sequence
  tag sequence
cttccagcta

SEQ ID NO: 1089 - DNA, 10 bp, Artificial Sequence
  tag sequence
cgaatgtcct

SEQ ID NO: 1090 - DNA, 10 bp, Artificial Sequence
  tag sequence
ttgaaacttt

SEQ ID NO: 1091 - DNA, 10 bp, Artificial Sequence
  tag sequence
ttgaagcttt

SEQ ID NO: 1092 - DNA, 10 bp, Artificial Sequence
  tag sequence
cccgggagcg

SEQ ID NO: 1093 - DNA, 10 bp, Artificial Sequence
  tag sequence
cccgggagcg

SEQ ID NO: 1094 - DNA, 10 bp, Artificial Sequence
  tag sequence
ggactctgga

SEQ ID NO: 1095 - DNA, 10 bp, Artificial Sequence
  tag sequence
ggactctgga

SEQ ID NO: 1096 - DNA, 10 bp, Artificial Sequence
  tag sequence
gtcttaaagt

SEQ ID NO: 1097 - DNA, 10 bp, Artificial Sequence
  tag sequence
cagctcactg

SEQ ID NO: 1098 - DNA, 10 bp, Artificial Sequence
  tag sequence
cagctcactg

SEQ ID NO: 1099 - DNA, 10 bp, Artificial Sequence
  tag sequence
gagcagcgcc

SEQ ID NO: 1100 - DNA, 10 bp, Artificial Sequence
  tag sequence
gctctgcttg

SEQ ID NO: 1101 - DNA, 10 bp, Artificial Sequence
  tag sequence
ggacctttat

SEQ ID NO: 1102 - DNA, 10 bp, Artificial Sequence
  tag sequence
ctccacccga

SEQ ID NO: 1103 - DNA, 10 bp, Artificial Sequence
  tag sequence
gtggccacgg

SEQ ID NO: 1104 - DNA, 10 bp, Artificial Sequence
  tag sequence
gacatcaagt

SEQ ID NO: 1105 - DNA, 10 bp, Artificial Sequence
  tag sequence
ccctaccctg

SEQ ID NO: 1106 - DNA, 10 bp, Artificial Sequence
  tag sequence
acgttaaaga

SEQ ID NO: 1107 - DNA, 10 bp, Artificial Sequence
  tag sequence
ccagagagtg

SEQ ID NO: 1108 - DNA, 10 bp, Artificial Sequence
  tag sequence
ggagtaaggg

SEQ ID NO: 1109 - DNA, 10 bp, Artificial Sequence
  tag sequence
ctgggcgccc

SEQ ID NO: 1110 - DNA, 10 bp, Artificial Sequence
  tag sequence
ccaataaagt

SEQ ID NO: 1111 - DNA, 10 bp, Artificial Sequence
  tag sequence
tttgttttta

SEQ ID NO: 1112 - DNA, 10 bp, Artificial Sequence
  tag sequence
atccgcgagg

SEQ ID NO: 1113 - DNA, 10 bp, Artificial Sequence
  tag sequence
gaccacaccg

SEQ ID NO: 1114 - DNA, 10 bp, Artificial Sequence
  tag sequence
cgatattccc

SEQ ID NO: 1115 - DNA, 10 bp, Artificial Sequence
  tag sequence
aaaccccaat

SEQ ID NO: 1116 - DNA, 10 bp, Artificial Sequence
  tag sequence
gttcacatta

SEQ ID NO: 1117 - DNA, 10 bp, Artificial Sequence
  tag sequence
aagctcgccg

SEQ ID NO: 1118 - DNA, 10 bp, Artificial Sequence
  tag sequence
gtccgagtgc

SEQ ID NO: 1119 - DNA, 10 bp, Artificial Sequence
  tag sequence
gactgcgcgt

SEQ ID NO: 1120 - DNA, 10 bp, Artificial Sequence
  tag sequence
ttgaagcttt

SEQ ID NO: 1121 - DNA, 10 bp, Artificial Sequence
  tag sequence
ttgaaacttt

SEQ ID NO: 1122 - DNA, 10 bp, Artificial Sequence
  tag sequence
tggaagcact

SEQ ID NO: 1123 - DNA, 10 bp, Artificial Sequence
  tag sequence
taacagccag

SEQ ID NO: 1124 - DNA, 10 bp, Artificial Sequence
  tag sequence
caattaaaag

SEQ ID NO: 1125 - DNA, 10 bp, Artificial Sequence
  tag sequence
tttggtgttt

SEQ ID NO: 1126 - DNA, 10 bp, Artificial Sequence
  tag sequence
tgatctccaa

SEQ ID NO: 1127 - DNA, 10 bp, Artificial Sequence
  tag sequence
ctccacccga

SEQ ID NO: 1128 - DNA, 10 bp, Artificial Sequence
  tag sequence
cgccgacgat

SEQ ID NO: 1129 - DNA, 10 bp, Artificial Sequence
  tag sequence
tttgggccta

SEQ ID NO: 1130 - DNA, 10 bp, Artificial Sequence
  tag sequence
aatctgcgcc

SEQ ID NO: 1131 - DNA, 10 bp, Artificial Sequence
  tag sequence
ccaggggaga

SEQ ID NO: 1132 - DNA, 10 bp, Artificial Sequence
  tag sequence
gaaagatgct

SEQ ID NO: 1133 - DNA, 10 bp, Artificial Sequence
  tag sequence
cagacttttt

SEQ ID NO: 1134 - DNA, 10 bp, Artificial Sequence
  tag sequence
ctggcgccga

SEQ ID NO: 1135 - DNA, 10 bp, Artificial Sequence
  tag sequence
tgagctaccc

SEQ ID NO: 1136 - DNA, 10 bp, Artificial Sequence
  tag sequence
gagcagcgcc

SEQ ID NO: 1137 - DNA, 10 bp, Artificial Sequence
  tag sequence
tttgcacctt

SEQ ID NO: 1138 - DNA, 10 bp, Artificial Sequence
  tag sequence
tatgagggta

SEQ ID NO: 1139 - DNA, 10 bp, Artificial Sequence
  tag sequence
gaagttataa

SEQ ID NO: 1140 - DNA, 10 bp, Artificial Sequence
  tag sequence
atgtgaagag

SEQ ID NO: 1141 - DNA, 10 bp, Artificial Sequence
  tag sequence
gagagaaaat

SEQ ID NO: 1142 - DNA, 10 bp, Artificial Sequence
  tag sequence
ctcccccaaa

SEQ ID NO: 1143 - DNA, 10 bp, Artificial Sequence
  tag sequence
acagcgctga

SEQ ID NO: 1144 - DNA, 10 bp, Artificial Sequence
  tag sequence
caatttgtgt

SEQ ID NO: 1145 - DNA, 10 bp, Artificial Sequence
  tag sequence
gccgggtggg

SEQ ID NO: 1146 - DNA, 10 bp, Artificial Sequence
  tag sequence
cgaccccacg

SEQ ID NO: 1147 - DNA, 10 bp, Artificial Sequence
  tag sequence
gcaccaaagc

SEQ ID NO: 1148 - DNA, 10 bp, Artificial Sequence
  tag sequence
gaaatacagt

SEQ ID NO: 1149 - DNA, 10 bp, Artificial Sequence
  tag sequence
accgccgtgg

SEQ ID NO: 1150 - DNA, 10 bp, Artificial Sequence
  tag sequence
tccctggctg

SEQ ID NO: 1151 - DNA, 10 bp, Artificial Sequence
  tag sequence
gggcatctct

SEQ ID NO: 1152 - DNA, 10 bp, Artificial Sequence
  tag sequence
atccggaccc

SEQ ID NO: 1153 - DNA, 10 bp, Artificial Sequence
  tag sequence
tttgggccta

SEQ ID NO: 1154 - DNA, 10 bp, Artificial Sequence
  tag sequence
gctttatttg

SEQ ID NO: 1155 - DNA, 10 bp, Artificial Sequence
  tag sequence
ttcccttctt

SEQ ID NO: 1156 - DNA, 10 bp, Artificial Sequence
  tag sequence
tccaaatcga

SEQ ID NO: 1157 - DNA, 10 bp, Artificial Sequence
  tag sequence
aaccacattg

SEQ ID NO: 1158 - DNA, 10 bp, Artificial Sequence
  tag sequence
gcggttgtgg

SEQ ID NO: 1159 - DNA, 10 bp, Artificial Sequence
  tag sequence
aagttgctat

SEQ ID NO: 1160 - DNA, 10 bp, Artificial Sequence
  tag sequence
atgtaaaaaa

SEQ ID NO: 1161 - DNA, 10 bp, Artificial Sequence
  tag sequence
gtaggggtaa

SEQ ID NO: 1162 - DNA, 10 bp, Artificial Sequence
  tag sequence
gggccagggg

SEQ ID NO: 1163 - DNA, 10 bp, Artificial Sequence
  tag sequence
gggggacggc

SEQ ID NO: 1164 - DNA, 10 bp, Artificial Sequence
  tag sequence
ctgttggtga

SEQ ID NO: 1165 - DNA, 10 bp, Artificial Sequence
  tag sequence
taaggagctg

SEQ ID NO: 1166 - DNA, 10 bp, Artificial Sequence
  tag sequence
acaaaaacta

SEQ ID NO: 1167 - DNA, 10 bp, Artificial Sequence
  tag sequence
tggctaaaaa

SEQ ID NO: 1168 - DNA, 10 bp, Artificial Sequence
  tag sequence
actttttaaa

SEQ ID NO: 1169 - DNA, 10 bp, Artificial Sequence
  tag sequence
tacagaggga

SEQ ID NO: 1170 - DNA, 10 bp, Artificial Sequence
  tag sequence
ctccacccga

SEQ ID NO: 1171 - DNA, 10 bp, Artificial Sequence
  tag sequence
agctgtcccc

SEQ ID NO: 1172 - DNA, 10 bp, Artificial Sequence
  tag sequence
tgaagcagta

SEQ ID NO: 1173 - DNA, 10 bp, Artificial Sequence
  tag sequence
taataaagaa

SEQ ID NO: 1174 - DNA, 10 bp, Artificial Sequence
  tag sequence
gtgcccgtgc

SEQ ID NO: 1175 - DNA, 10 bp, Artificial Sequence
  tag sequence
cccgcctctt

SEQ ID NO: 1176 - DNA, 10 bp, Artificial Sequence
  tag sequence
acacagcaag

SEQ ID NO: 1177 - DNA, 10 bp, Artificial Sequence
  tag sequence
gtccctgcct

SEQ ID NO: 1178 - DNA, 10 bp, Artificial Sequence
  tag sequence
accaaaaacc

SEQ ID NO: 1179 - DNA, 10 bp, Artificial Sequence
  tag sequence
tggaaatgac

SEQ ID NO: 1180 - DNA, 10 bp, Artificial Sequence
  tag sequence
ccacgggatt

SEQ ID NO: 1181 - DNA, 10 bp, Artificial Sequence
  tag sequence
gatcaggcca

SEQ ID NO: 1182 - DNA, 10 bp, Artificial Sequence
  tag sequence
tttggttttc

SEQ ID NO: 1183 - DNA, 10 bp, Artificial Sequence
  tag sequence
aactcccagt

SEQ ID NO: 1184 - DNA, 10 bp, Artificial Sequence
  tag sequence
gactttggaa

SEQ ID NO: 1185 - DNA, 10 bp, Artificial Sequence
  tag sequence
caaccagtaa

SEQ ID NO: 1186 - DNA, 10 bp, Artificial Sequence
  tag sequence
cagataagtt

SEQ ID NO: 1187 - DNA, 10 bp, Artificial Sequence
  tag sequence
catatcatta

SEQ ID NO: 1188 - DNA, 10 bp, Artificial Sequence
  tag sequence
tcaccggtca

SEQ ID NO: 1189 - DNA, 10 bp, Artificial Sequence
  tag sequence
agggagcaga

SEQ ID NO: 1190 - DNA, 10 bp, Artificial Sequence
  tag sequence
cccttgtccg

SEQ ID NO: 1191 - DNA, 10 bp, Artificial Sequence
  tag sequence
ataaaaagaa

SEQ ID NO: 1192 - DNA, 10 bp, Artificial Sequence
  tag sequence
gttgtctttg

SEQ ID NO: 1193 - DNA, 10 bp, Artificial Sequence
  tag sequence
ccgggggagc

SEQ ID NO: 1194 - DNA, 10 bp, Artificial Sequence
  tag sequence
tggccagctc

SEQ ID NO: 1195 - DNA, 10 bp, Artificial Sequence
  tag sequence
ttcggttggt

SEQ ID NO: 1196 - DNA, 10 bp, Artificial Sequence
  tag sequence
tcaacttctg

SEQ ID NO: 1197 - DNA, 10 bp, Artificial Sequence
  tag sequence
acccccccgc

SEQ ID NO: 1198 - DNA, 10 bp, Artificial Sequence
  tag sequence
gtgcgctgag

SEQ ID NO: 1199 - DNA, 10 bp, Artificial Sequence
  tag sequence
gaccagcaga

SEQ ID NO: 1200 - DNA, 10 bp, Artificial Sequence
  tag sequence
gtcaaaattt

SEQ ID NO: 1201 - DNA, 10 bp, Artificial Sequence
  tag sequence
gtgctaagcg

SEQ ID NO: 1202 - DNA, 10 bp, Artificial Sequence
  tag sequence
atttcttcaa

SEQ ID NO: 1203 - DNA, 10 bp, Artificial Sequence
  tag sequence
acattctttt

SEQ ID NO: 1204 - DNA, 10 bp, Artificial Sequence
  tag sequence
ggcacctcag

SEQ ID NO: 1205 - DNA, 10 bp, Artificial Sequence
  tag sequence
acattccaag

SEQ ID NO: 1206 - DNA, 10 bp, Artificial Sequence
  tag sequence
aaaacgtttt

SEQ ID NO: 1207 - DNA, 10 bp, Artificial Sequence
  tag sequence
tccaggaaac

SEQ ID NO: 1208 - DNA, 10 bp, Artificial Sequence
  tag sequence
cctcccagct

SEQ ID NO: 1209 - DNA, 10 bp, Artificial Sequence
  tag sequence
cttgggtttt

SEQ ID NO: 1210 - DNA, 10 bp, Artificial Sequence
  tag sequence
ccaggggaga

SEQ ID NO: 1211 - DNA, 10 bp, Artificial Sequence
  tag sequence
gggaggggtg

SEQ ID NO: 1212 - DNA, 10 bp, Artificial Sequence
  tag sequence
gcacggaaaa

SEQ ID NO: 1213 - DNA, 10 bp, Artificial Sequence
  tag sequence
gatgaggaga

SEQ ID NO: 1214 - DNA, 10 bp, Artificial Sequence
  tag sequence
tggaaagtga

SEQ ID NO: 1215 - DNA, 10 bp, Artificial Sequence
  tag sequence
cgccgacgat

SEQ ID NO: 1216 - DNA, 10 bp, Artificial Sequence
  tag sequence
ctgtcagcgt

SEQ ID NO: 1217 - DNA, 10 bp, Artificial Sequence
  tag sequence
gttccacaga

SEQ ID NO: 1218 - DNA, 10 bp, Artificial Sequence
  tag sequence
ggaactttta

SEQ ID NO: 1219 - DNA, 10 bp, Artificial Sequence
  tag sequence
gtataaacgt

SEQ ID NO: 1220 - DNA, 10 bp, Artificial Sequence
  tag sequence
gaggaggaga

SEQ ID NO: 1221 - DNA, 10 bp, Artificial Sequence
  tag sequence
gggggggggt

SEQ ID NO: 1222 - DNA, 10 bp, Artificial Sequence
  tag sequence
ttgggatggg

SEQ ID NO: 1223 - DNA, 10 bp, Artificial Sequence
  tag sequence
ttccggttcc

SEQ ID NO: 1224 - DNA, 10 bp, Artificial Sequence
  tag sequence
ggaaagtgtt

SEQ ID NO: 1225 - DNA, 10 bp, Artificial Sequence
  tag sequence
gcccagctgg

SEQ ID NO: 1226 - DNA, 10 bp, Artificial Sequence
  tag sequence
tttccctcaa

SEQ ID NO: 1227 - DNA, 10 bp, Artificial Sequence
  tag sequence
ggatgtgaaa

SEQ ID NO: 1228 - DNA, 10 bp, Artificial Sequence
  tag sequence
gcaaaaaaaa

SEQ ID NO: 1229 - DNA, 10 bp, Artificial Sequence
  tag sequence
acccacgtca

SEQ ID NO: 1230 - DNA, 10 bp, Artificial Sequence
  tag sequence
cggggtggcc

SEQ ID NO: 1231 - DNA, 10 bp, Artificial Sequence
  tag sequence
cgccccggcg

SEQ ID NO: 1232 - DNA, 10 bp, Artificial Sequence
  tag sequence
cagacttttg

SEQ ID NO: 1233 - DNA, 10 bp, Artificial Sequence
  tag sequence
ttacttctgc

SEQ ID NO: 1234 - DNA, 10 bp, Artificial Sequence
  tag sequence
cgtctttaaa

SEQ ID NO: 1235 - DNA, 10 bp, Artificial Sequence
  tag sequence
ttgctgactt

SEQ ID NO: 1236 - DNA, 10 bp, Artificial Sequence
  tag sequence
tcgaagaacc

SEQ ID NO: 1237 - DNA, 10 bp, Artificial Sequence
  tag sequence
ggcccctcac

SEQ ID NO: 1238 - DNA, 10 bp, Artificial Sequence
  tag sequence
cagctggcca

SEQ ID NO: 1239 - DNA, 10 bp, Artificial Sequence
  tag sequence
tgtaaacaat

SEQ ID NO: 1240 - DNA, 10 bp, Artificial Sequence
  tag sequence
gagatccgca

SEQ ID NO: 1241 - DNA, 10 bp, Artificial Sequence
  tag sequence
ccctgggttc

SEQ ID NO: 1242 - DNA, 10 bp, Artificial Sequence
  tag sequence
ctaacggggc

SEQ ID NO: 1243 - DNA, 10 bp, Artificial Sequence
  tag sequence
tgcgctctcc

SEQ ID NO: 1244 - DNA, 10 bp, Artificial Sequence
  tag sequence
cgcagtctgc

SEQ ID NO: 1245 - DNA, 10 bp, Artificial Sequence
  tag sequence
ggaggaattc

SEQ ID NO: 1246 - DNA, 10 bp, Artificial Sequence
  tag sequence
aagaaaggag

SEQ ID NO: 1247 - DNA, 10 bp, Artificial Sequence
  tag sequence
acttattatg

SEQ ID NO: 1248 - DNA, 10 bp, Artificial Sequence
  tag sequence
tagttggaaa

SEQ ID NO: 1249 - DNA, 10 bp, Artificial Sequence
  tag sequence
tcaacaaatt

SEQ ID NO: 1250 - DNA, 10 bp, Artificial Sequence
  tag sequence
gcgtgagtgc

SEQ ID NO: 1251 - DNA, 10 bp, Artificial Sequence
  tag sequence
cggctgaatt

SEQ ID NO: 1252 - DNA, 10 bp, Artificial Sequence
  tag sequence
agcaaactga

SEQ ID NO: 1253 - DNA, 10 bp, Artificial Sequence
  tag sequence
gcgcagaggt

SEQ ID NO: 1254 - DNA, 10 bp, Artificial Sequence
  tag sequence
tgggactcca

SEQ ID NO: 1255 - DNA, 10 bp, Artificial Sequence
  tag sequence
actcagcccg

SEQ ID NO: 1256 - DNA, 10 bp, Artificial Sequence
  tag sequence
cagcacggat

SEQ ID NO: 1257 - DNA, 10 bp, Artificial Sequence
  tag sequence
ggaaatgtca

SEQ ID NO: 1258 - DNA, 10 bp, Artificial Sequence
  tag sequence
tgcgctggcc

SEQ ID NO: 1259 - DNA, 10 bp, Artificial Sequence
  tag sequence
gacggctgca

SEQ ID NO: 1260 - DNA, 10 bp, Artificial Sequence
  tag sequence
ggaagtttcg

SEQ ID NO: 1261 - DNA, 10 bp, Artificial Sequence
  tag sequence
gggccaaccc

SEQ ID NO: 1262 - DNA, 10 bp, Artificial Sequence
  tag sequence
gacgcggcgc

SEQ ID NO: 1263 - DNA, 10 bp, Artificial Sequence
  tag sequence
tatcctgaaa

SEQ ID NO: 1264 - DNA, 10 bp, Artificial Sequence
  tag sequence
atggcaacag

SEQ ID NO: 1265 - DNA, 10 bp, Artificial Sequence
  tag sequence
acgacaaagc

SEQ ID NO: 1266 - DNA, 10 bp, Artificial Sequence
  tag sequence
actgaaagaa

SEQ ID NO: 1267 - DNA, 10 bp, Artificial Sequence
  tag sequence
ggctgccctg

SEQ ID NO: 1268 - DNA, 10 bp, Artificial Sequence
  tag sequence
ggcacgcagc

SEQ ID NO: 1269 - DNA, 10 bp, Artificial Sequence
  tag sequence
caaaaaatta

SEQ ID NO: 1270 - DNA, 10 bp, Artificial Sequence
  tag sequence
ggccacgtag

SEQ ID NO: 1271 - DNA, 10 bp, Artificial Sequence
  tag sequence
ctaaaaaaaa

SEQ ID NO: 1272 - DNA, 10 bp, Artificial Sequence
  tag sequence
ccaaggtttt

SEQ ID NO: 1273 - DNA, 10 bp, Artificial Sequence
  tag sequence
gacaaaaaaa

SEQ ID NO: 1274 - DNA, 10 bp, Artificial Sequence
  tag sequence
ccctaccctg

SEQ ID NO: 1275 - DNA, 10 bp, Artificial Sequence
  tag sequence
ggaaaaaaaa

SEQ ID NO: 1276 - DNA, 10 bp, Artificial Sequence
  tag sequence
gcggcggctc

SEQ ID NO: 1277 - DNA, 10 bp, Artificial Sequence
  tag sequence
gcgaaaccca

SEQ ID NO: 1278 - DNA, 10 bp, Artificial Sequence
  tag sequence
ctaataaact

SEQ ID NO: 1279 - DNA, 10 bp, Artificial Sequence
  tag sequence
aagagcgccg

SEQ ID NO: 1280 - DNA, 10 bp, Artificial Sequence
  tag sequence
gctgaacgcg

SEQ ID NO: 1281 - DNA, 10 bp, Artificial Sequence
  tag sequence
gcccccaata

SEQ ID NO: 1282 - DNA, 10 bp, Artificial Sequence
  tag sequence
gcggggtgga

SEQ ID NO: 1283 - DNA, 10 bp, Artificial Sequence
  tag sequence
tagttggaac

SEQ ID NO: 1284 - DNA, 10 bp, Artificial Sequence
  tag sequence
caagttcttt

SEQ ID NO: 1285 - DNA, 10 bp, Artificial Sequence
  tag sequence
cgaccccacg

SEQ ID NO: 1286 - DNA, 10 bp, Artificial Sequence
  tag sequence
gaattcacaa

SEQ ID NO: 1287 - DNA, 10 bp, Artificial Sequence
  tag sequence
gagtgggtgc

SEQ ID NO: 1288 - DNA, 10 bp, Artificial Sequence
  tag sequence
cagcggcggg

SEQ ID NO: 1289 - DNA, 10 bp, Artificial Sequence
  tag sequence
gcctgtccct

SEQ ID NO: 1290 - DNA, 10 bp, Artificial Sequence
  tag sequence
caggacagtt

SEQ ID NO: 1291 - DNA, 10 bp, Artificial Sequence
  tag sequence
gcagaaaatt

SEQ ID NO: 1292 - DNA, 10 bp, Artificial Sequence
  tag sequence
cataaatgcg

SEQ ID NO: 1293 - DNA, 10 bp, Artificial Sequence
  tag sequence
gtggcagcgc

SEQ ID NO: 1294 - DNA, 10 bp, Artificial Sequence
  tag sequence
cacacagttt

SEQ ID NO: 1295 - DNA, 10 bp, Artificial Sequence
  tag sequence
ggtgcccagt

SEQ ID NO: 1296 - DNA, 10 bp, Artificial Sequence
  tag sequence
ttctgtgctg

SEQ ID NO: 1297 - DNA, 10 bp, Artificial Sequence
  tag sequence
ctctccaaac

SEQ ID NO: 1298 - DNA, 10 bp, Artificial Sequence
  tag sequence
ggccctaggc

SEQ ID NO: 1299 - DNA, 10 bp, Artificial Sequence
  tag sequence
ctcaaccccc

SEQ ID NO: 1300 - DNA, 10 bp, Artificial Sequence
  tag sequence
agccaccgcg

SEQ ID NO: 1301 - DNA, 10 bp, Artificial Sequence
  tag sequence
accttgaagt

SEQ ID NO: 1302 - DNA, 10 bp, Artificial Sequence
  tag sequence
tcagaagttt

SEQ ID NO: 1303 - DNA, 10 bp, Artificial Sequence
  tag sequence
tggcaaaata

SEQ ID NO: 1304 - DNA, 10 bp, Artificial Sequence
  tag sequence
gggaggtagc

SEQ ID NO: 1305 - DNA, 10 bp, Artificial Sequence
  tag sequence
gaaaaattta

SEQ ID NO: 1306 - DNA, 10 bp, Artificial Sequence
  tag sequence
ggcaggcggg

SEQ ID NO: 1307 - DNA, 10 bp, Artificial Sequence
  tag sequence
agattcaaac

SEQ ID NO: 1308 - DNA, 10 bp, Artificial Sequence
  tag sequence
gtaaaaaaaa

SEQ ID NO: 1309 - DNA, 10 bp, Artificial Sequence
  tag sequence
aggctcctgg

SEQ ID NO: 1310 - DNA, 10 bp, Artificial Sequence
  tag sequence
cgccgcggtg

SEQ ID NO: 1311 - DNA, 10 bp, Artificial Sequence
  tag sequence
tgcctgcacc

SEQ ID NO: 1312 - DNA, 10 bp, Artificial Sequence
  tag sequence
gtgactgcca

SEQ ID NO: 1313 - DNA, 10 bp, Artificial Sequence
  tag sequence
gtttatggat

SEQ ID NO: 1314 - DNA, 10 bp, Artificial Sequence
  tag sequence
gcagccatcc

SEQ ID NO: 1315 - DNA, 10 bp, Artificial Sequence
  tag sequence
caggtttcat

SEQ ID NO: 1316 - DNA, 10 bp, Artificial Sequence
  tag sequence
ggcctgctgc

SEQ ID NO: 1317 - DNA, 10 bp, Artificial Sequence
  tag sequence
ccccctggat

SEQ ID NO: 1318 - DNA, 10 bp, Artificial Sequence
  tag sequence
gggggaattt

SEQ ID NO: 1319 - DNA, 10 bp, Artificial Sequence
  tag sequence
aacttttggc

SEQ ID NO: 1320 - DNA, 10 bp, Artificial Sequence
  tag sequence
agaatttgca

SEQ ID NO: 1321 - DNA, 10 bp, Artificial Sequence
  tag sequence
gccgccctgc

SEQ ID NO: 1322 - DNA, 10 bp, Artificial Sequence
  tag sequence
gggggtaact

SEQ ID NO: 1323 - DNA, 10 bp, Artificial Sequence
  tag sequence
tgaaaaaaaa

SEQ ID NO: 1324 - DNA, 10 bp, Artificial Sequence
  tag sequence
ggcctttttt

SEQ ID NO: 1325 - DNA, 10 bp, Artificial Sequence
  tag sequence
gcgacgaggc

SEQ ID NO: 1326 - DNA, 10 bp, Artificial Sequence
  tag sequence
gcgctggagt

SEQ ID NO: 1327 - DNA, 10 bp, Artificial Sequence
  tag sequence
ggagggggct

SEQ ID NO: 1328 - DNA, 10 bp, Artificial Sequence
  tag sequence
gagggagttt

SEQ ID NO: 1329 - DNA, 10 bp, Artificial Sequence
  tag sequence
cgctggttcc

SEQ ID NO: 1330 - DNA, 10 bp, Artificial Sequence
  tag sequence
tcaagccatc

SEQ ID NO: 1331 - DNA, 10 bp, Artificial Sequence
  tag sequence
ggctttggag

SEQ ID NO: 1332 - DNA, 10 bp, Artificial Sequence
  tag sequence
ctgccaagtt

SEQ ID NO: 1333 - DNA, 10 bp, Artificial Sequence
  tag sequence
gactcacttt

SEQ ID NO: 1334 - DNA, 10 bp, Artificial Sequence
  tag sequence
ggggaaatcg

SEQ ID NO: 1335 - DNA, 10 bp, Artificial Sequence
  tag sequence
ggccgcgttc

SEQ ID NO: 1336 - DNA, 10 bp, Artificial Sequence
  tag sequence
ccgtgactct

SEQ ID NO: 1337 - DNA, 10 bp, Artificial Sequence
  tag sequence
tgcacgtttt

SEQ ID NO: 1338 - DNA, 10 bp, Artificial Sequence
  tag sequence
gttgtggtta

SEQ ID NO: 1339 - DNA, 10 bp, Artificial Sequence
  tag sequence
gttaacgtcc

SEQ ID NO: 1340 - DNA, 10 bp, Artificial Sequence
  tag sequence
caggagttca

SEQ ID NO: 1341 - DNA, 10 bp, Artificial Sequence
  tag sequence
cctcggaaaa

SEQ ID NO: 1342 - DNA, 10 bp, Artificial Sequence
  tag sequence
cccgtccgga

SEQ ID NO: 1343 - DNA, 10 bp, Artificial Sequence
  tag sequence
ggaagctaag

SEQ ID NO: 1344 - DNA, 10 bp, Artificial Sequence
  tag sequence
cccatccgaa

SEQ ID NO: 1345 - DNA, 10 bp, Artificial Sequence
  tag sequence
ccccagccag

SEQ ID NO: 1346 - DNA, 10 bp, Artificial Sequence
  tag sequence
ggtggcactc

SEQ ID NO: 1347 - DNA, 10 bp, Artificial Sequence
  tag sequence
atggtggggg

SEQ ID NO: 1348 - DNA, 10 bp, Artificial Sequence
  tag sequence
cgccgccggc

SEQ ID NO: 1349 - DNA, 10 bp, Artificial Sequence
  tag sequence
cagcagaagc

SEQ ID NO: 1350 - DNA, 10 bp, Artificial Sequence
  tag sequence
ttggggtttc

SEQ ID NO: 1351 - DNA, 10 bp, Artificial Sequence
  tag sequence
ccagtggccc

SEQ ID NO: 1352 - DNA, 10 bp, Artificial Sequence
  tag sequence
cgccggaaca

SEQ ID NO: 1353 - DNA, 10 bp, Artificial Sequence
  tag sequence
ctgtacttgt

SEQ ID NO: 1354 - DNA, 10 bp, Artificial Sequence
  tag sequence
accatcctgc

SEQ ID NO: 1355 - DNA, 10 bp, Artificial Sequence
  tag sequence
gtgaaactcc

SEQ ID NO: 1356 - DNA, 10 bp, Artificial Sequence
  tag sequence
gccgtgtccg

SEQ ID NO: 1357 - DNA, 10 bp, Artificial Sequence
  tag sequence
gcgaaacccc

SEQ ID NO: 1358 - DNA, 10 bp, Artificial Sequence
  tag sequence
gccgaggaag

SEQ ID NO: 1359 - DNA, 10 bp, Artificial Sequence
  tag sequence
ttgaattccc

SEQ ID NO: 1360 - DNA, 10 bp, Artificial Sequence
  tag sequence
gtgctgaatg

SEQ ID NO: 1361 - DNA, 10 bp, Artificial Sequence
  tag sequence
ttgaagcttt

SEQ ID NO: 1362 - DNA, 10 bp, Artificial Sequence
  tag sequence
gcataatagg

SEQ ID NO: 1363 - DNA, 10 bp, Artificial Sequence
  tag sequence
aagacagtgg

SEQ ID NO: 1364 - DNA, 10 bp, Artificial Sequence
  tag sequence
tgttctggag

SEQ ID NO: 1365 - DNA, 10 bp, Artificial Sequence
  tag sequence
acaggctacg

SEQ ID NO: 1366 - DNA, 10 bp, Artificial Sequence
  tag sequence
aagaagatag

SEQ ID NO: 1367 - DNA, 10 bp, Artificial Sequence
  tag sequence
gacttgtata

SEQ ID NO: 1368 - DNA, 10 bp, Artificial Sequence
  tag sequence
attctccagt

SEQ ID NO: 1369 - DNA, 10 bp, Artificial Sequence
  tag sequence
ttatggggag

SEQ ID NO: 1370 - DNA, 10 bp, Artificial Sequence
  tag sequence
ggctgtaccc

SEQ ID NO: 1371 - DNA, 10 bp, Artificial Sequence
  tag sequence
atggctggta

SEQ ID NO: 1372 - DNA, 10 bp, Artificial Sequence
  tag sequence
tgaagttata

SEQ ID NO: 1373 - DNA, 10 bp, Artificial Sequence
  tag sequence
agtatgagga

SEQ ID NO: 1374 - DNA, 10 bp, Artificial Sequence
  tag sequence
gcctacccga

SEQ ID NO: 1375 - DNA, 10 bp, Artificial Sequence
  tag sequence
cgtgttaatg

SEQ ID NO: 1376 - DNA, 10 bp, Artificial Sequence
  tag sequence
ttgtaatcgt

SEQ ID NO: 1377 - DNA, 10 bp, Artificial Sequence
  tag sequence
tcttgtgcat

SEQ ID NO: 1378 - DNA, 10 bp, Artificial Sequence
  tag sequence
ttaccatatc

SEQ ID NO: 1379 - DNA, 10 bp, Artificial Sequence
  tag sequence
tggaagcact

SEQ ID NO: 1380 - DNA, 10 bp, Artificial Sequence
  tag sequence
ctgctatacg

SEQ ID NO: 1381 - DNA, 10 bp, Artificial Sequence
  tag sequence
tgctgtgcat

SEQ ID NO: 1382 - DNA, 10 bp, Artificial Sequence
  tag sequence
actaacaccc

SEQ ID NO: 1383 - DNA, 10 bp, Artificial Sequence
  tag sequence
gatctcttgg

SEQ ID NO: 1384 - DNA, 10 bp, Artificial Sequence
  tag sequence
tactcttggc

SEQ ID NO: 1385 - DNA, 10 bp, Artificial Sequence
  tag sequence
ctgttgattg

SEQ ID NO: 1386 - DNA, 10 bp, Artificial Sequence
  tag sequence
taataaaggt

SEQ ID NO: 1387 - DNA, 10 bp, Artificial Sequence
  tag sequence
ccactgcact

SEQ ID NO: 1388 - DNA, 10 bp, Artificial Sequence
  tag sequence
agaaagatgt

SEQ ID NO: 1389 - DNA, 10 bp, Artificial Sequence
  tag sequence
ctgtacagac

SEQ ID NO: 1390 - DNA, 10 bp, Artificial Sequence
  tag sequence
agaaatgttg

SEQ ID NO: 1391 - DNA, 10 bp, Artificial Sequence
  tag sequence
ggctttaccc

SEQ ID NO: 1392 - DNA, 10 bp, Artificial Sequence
  tag sequence
acagtgggga

SEQ ID NO: 1393 - DNA, 10 bp, Artificial Sequence
  tag sequence
tgtataaaaa

SEQ ID NO: 1394 - DNA, 10 bp, Artificial Sequence
  tag sequence
ttatgggatc

SEQ ID NO: 1395 - DNA, 10 bp, Artificial Sequence
  tag sequence
ttactaaatg

SEQ ID NO: 1396 - DNA, 10 bp, Artificial Sequence
  tag sequence
gccttgggtg

SEQ ID NO: 1397 - DNA, 10 bp, Artificial Sequence
  tag sequence
atcaagggtg

SEQ ID NO: 1398 - DNA, 10 bp, Artificial Sequence
  tag sequence
taggtagctc

SEQ ID NO: 1399 - DNA, 10 bp, Artificial Sequence
  tag sequence
taccatcaat

SEQ ID NO: 1400 - DNA, 10 bp, Artificial Sequence
  tag sequence
catttgtaat

SEQ ID NO: 1401 - DNA, 10 bp, Artificial Sequence
  tag sequence
aaactgtggt

SEQ ID NO: 1402 - DNA, 10 bp, Artificial Sequence
  tag sequence
aagctgtata

SEQ ID NO: 1403 - DNA, 10 bp, Artificial Sequence
  tag sequence
taaaacaaga

SEQ ID NO: 1404 - DNA, 10 bp, Artificial Sequence
  tag sequence
tgatatgtca

SEQ ID NO: 1405 - DNA, 10 bp, Artificial Sequence
  tag sequence
cgaatgtcct

SEQ ID NO: 1406 - DNA, 10 bp, Artificial Sequence
  tag sequence
gtgcgccgga

SEQ ID NO: 1407 - DNA, 10 bp, Artificial Sequence
  tag sequence
gcaacttaga

SEQ ID NO: 1408 - DNA, 10 bp, Artificial Sequence
  tag sequence
tctctactaa

SEQ ID NO: 1409 - DNA, 10 bp, Artificial Sequence
  tag sequence
cctcaggata

SEQ ID NO: 1410 - DNA, 10 bp, Artificial Sequence
  tag sequence
tctgtaatcc

SEQ ID NO: 1411 - DNA, 10 bp, Artificial Sequence
  tag sequence
tcctgtaaag

SEQ ID NO: 1412 - DNA, 10 bp, Artificial Sequence
  tag sequence
gtgtaataag

SEQ ID NO: 1413 - DNA, 10 bp, Artificial Sequence
  tag sequence
tagctctatg

SEQ ID NO: 1414 - DNA, 10 bp, Artificial Sequence
  tag sequence
ctttctttga

SEQ ID NO: 1415 - DNA, 10 bp, Artificial Sequence
  tag sequence
cttgagcaat

SEQ ID NO: 1416 - DNA, 10 bp, Artificial Sequence
  tag sequence
aggcctcggc

SEQ ID NO: 1417 - DNA, 10 bp, Artificial Sequence
  tag sequence
ttcttgtttt

SEQ ID NO: 1418 - DNA, 10 bp, Artificial Sequence
  tag sequence
tgtaggtcat

SEQ ID NO: 1419 - DNA, 10 bp, Artificial Sequence
  tag sequence
ttaagacttc

SEQ ID NO: 1420 - DNA, 10 bp, Artificial Sequence
  tag sequence
gggttggctt

SEQ ID NO: 1421 - DNA, 10 bp, Artificial Sequence
  tag sequence
gtactagtgt

SEQ ID NO: 1422 - DNA, 10 bp, Artificial Sequence
  tag sequence
gtttttgctt

SEQ ID NO: 1423 - DNA, 10 bp, Artificial Sequence
  tag sequence
ggggcacttg

SEQ ID NO: 1424 - DNA, 10 bp, Artificial Sequence
  tag sequence
ctcagtcttt

SEQ ID NO: 1425 - DNA, 10 bp, Artificial Sequence
  tag sequence
aatattgaga

SEQ ID NO: 1426 - DNA, 10 bp, Artificial Sequence
  tag sequence
ttataaaaga

SEQ ID NO: 1427 - DNA, 10 bp, Artificial Sequence
  tag sequence
tataaggtgg

SEQ ID NO: 1428 - DNA, 10 bp, Artificial Sequence
  tag sequence
tactggaagt

SEQ ID NO: 1429 - DNA, 10 bp, Artificial Sequence
  tag sequence
ctttcagatg

SEQ ID NO: 1430 - DNA, 10 bp, Artificial Sequence
  tag sequence
tcactgcact

SEQ ID NO: 1431 - DNA, 10 bp, Artificial Sequence
  tag sequence
ttaatatatg

SEQ ID NO: 1432 - DNA, 10 bp, Artificial Sequence
  tag sequence
ttcatacacc

SEQ ID NO: 1433 - DNA, 10 bp, Artificial Sequence
  tag sequence
tactagtcct

SEQ ID NO: 1434 - DNA, 10 bp, Artificial Sequence
  tag sequence
tggatcaacc

SEQ ID NO: 1435 - DNA, 10 bp, Artificial Sequence
  tag sequence
tccctattaa

SEQ ID NO: 1436 - DNA, 10 bp, Artificial Sequence
  tag sequence
tacaaacggt

SEQ ID NO: 1437 - DNA, 10 bp, Artificial Sequence
  tag sequence
tcaaatgcat

SEQ ID NO: 1438 - DNA, 10 bp, Artificial Sequence
  tag sequence
aggtcttcaa

SEQ ID NO: 1439 - DNA, 10 bp, Artificial Sequence
  tag sequence
cctggtccca

SEQ ID NO: 1440 - DNA, 10 bp, Artificial Sequence
  tag sequence
tttcctctca

SEQ ID NO: 1441 - DNA, 10 bp, Artificial Sequence
  tag sequence
ctgttggcat

SEQ ID NO: 1442 - DNA, 10 bp, Artificial Sequence
  tag sequence
tttgtagatg

SEQ ID NO: 1443 - DNA, 10 bp, Artificial Sequence
  tag sequence
tcatcatctg

SEQ ID NO: 1444 - DNA, 10 bp, Artificial Sequence
  tag sequence
ccattgcact

SEQ ID NO: 1445 - DNA, 10 bp, Artificial Sequence
  tag sequence
gtcctttctg

SEQ ID NO: 1446 - DNA, 10 bp, Artificial Sequence
  tag sequence
cttccttgcc

SEQ ID NO: 1447 - DNA, 10 bp, Artificial Sequence
  tag sequence
gtttcatctc

SEQ ID NO: 1448 - DNA, 10 bp, Artificial Sequence
  tag sequence
agtgtctgtg

SEQ ID NO: 1449 - DNA, 10 bp, Artificial Sequence
  tag sequence
accagtggtt

SEQ ID NO: 1450 - DNA, 10 bp, Artificial Sequence
  tag sequence
acacttggag

SEQ ID NO: 1451 - DNA, 10 bp, Artificial Sequence
  tag sequence
gcttagaagt

SEQ ID NO: 1452 - DNA, 10 bp, Artificial Sequence
  tag sequence
cagaaggcca

SEQ ID NO: 1453 - DNA, 10 bp, Artificial Sequence
  tag sequence
tttactttgg

SEQ ID NO: 1454 - DNA, 10 bp, Artificial Sequence
  tag sequence
tatcccaact

SEQ ID NO: 1455 - DNA, 10 bp, Artificial Sequence
  tag sequence
ctgacttgtg

SEQ ID NO: 1456 - DNA, 10 bp, Artificial Sequence
  tag sequence
acctttactg

SEQ ID NO: 1457 - DNA, 10 bp, Artificial Sequence
  tag sequence
aaatacctaa

SEQ ID NO: 1458 - DNA, 10 bp, Artificial Sequence
  tag sequence
cttaaggatt

SEQ ID NO: 1459 - DNA, 10 bp, Artificial Sequence
  tag sequence
ttgggttaat

SEQ ID NO: 1460 - DNA, 10 bp, Artificial Sequence
  tag sequence
tatttttgtt

SEQ ID NO: 1461 - DNA, 10 bp, Artificial Sequence
  tag sequence
gtggatggac

SEQ ID NO: 1462 - DNA, 10 bp, Artificial Sequence
  tag sequence
atagacataa

SEQ ID NO: 1463 - DNA, 10 bp, Artificial Sequence
  tag sequence
aaggctggaa

SEQ ID NO: 1464 - DNA, 10 bp, Artificial Sequence
  tag sequence
tttgtacaca

SEQ ID NO: 1465 - DNA, 10 bp, Artificial Sequence
  tag sequence
tgggaagagg

SEQ ID NO: 1466 - DNA, 10 bp, Artificial Sequence
  tag sequence
gtatttaaca

SEQ ID NO: 1467 - DNA, 10 bp, Artificial Sequence
  tag sequence
ggaaagatgt

SEQ ID NO: 1468 - DNA, 10 bp, Artificial Sequence
  tag sequence
tggagaatgt

SEQ ID NO: 1469 - DNA, 10 bp, Artificial Sequence
  tag sequence
tatgtatgtt

SEQ ID NO: 1470 - DNA, 10 bp, Artificial Sequence
  tag sequence
tacctaattg

SEQ ID NO: 1471 - DNA, 10 bp, Artificial Sequence
  tag sequence
taataaagca

SEQ ID NO: 1472 - DNA, 10 bp, Artificial Sequence
  tag sequence
gtactgtatg

SEQ ID NO: 1473 - DNA, 10 bp, Artificial Sequence
  tag sequence
gctgtagcca

SEQ ID NO: 1474 - DNA, 10 bp, Artificial Sequence
  tag sequence
ttagataagc

SEQ ID NO: 1475 - DNA, 10 bp, Artificial Sequence
  tag sequence
tcataatagg

SEQ ID NO: 1476 - DNA, 10 bp, Artificial Sequence
  tag sequence
taatttatag

SEQ ID NO: 1477 - DNA, 10 bp, Artificial Sequence
  tag sequence
ggtcactgag

SEQ ID NO: 1478 - DNA, 10 bp, Artificial Sequence
  tag sequence
cctttttcaa

SEQ ID NO: 1479 - DNA, 10 bp, Artificial Sequence
  tag sequence
actactaagg

SEQ ID NO: 1480 - DNA, 10 bp, Artificial Sequence
  tag sequence
gatgtgcacg

SEQ ID NO: 1481 - DNA, 10 bp, Artificial Sequence
  tag sequence
ttcttttcat

SEQ ID NO: 1482 - DNA, 10 bp, Artificial Sequence
  tag sequence
cgaaagatgt

SEQ ID NO: 1483 - DNA, 10 bp, Artificial Sequence
  tag sequence
aaagtcattg

SEQ ID NO: 1484 - DNA, 10 bp, Artificial Sequence
  tag sequence
tgtgttgtca

SEQ ID NO: 1485 - DNA, 10 bp, Artificial Sequence
  tag sequence
tccatcgtcc

SEQ ID NO: 1486 - DNA, 10 bp, Artificial Sequence
  tag sequence
gtgcagagga

SEQ ID NO: 1487 - DNA, 10 bp, Artificial Sequence
  tag sequence
gatatgttat

SEQ ID NO: 1488 - DNA, 10 bp, Artificial Sequence
  tag sequence
atggtgtatg

SEQ ID NO: 1489 - DNA, 10 bp, Artificial Sequence
  tag sequence
ttacttatac

SEQ ID NO: 1490 - DNA, 10 bp, Artificial Sequence
  tag sequence
ttctatttca

SEQ ID NO: 1491 - DNA, 10 bp, Artificial Sequence
  tag sequence
tgttcatcat

SEQ ID NO: 1492 - DNA, 10 bp, Artificial Sequence
  tag sequence
tgttaatgtt

SEQ ID NO: 1493 - DNA, 10 bp, Artificial Sequence
  tag sequence
ttttgtattt

SEQ ID NO: 1494 - DNA, 10 bp, Artificial Sequence
  tag sequence
tcaataaagg

SEQ ID NO: 1495 - DNA, 10 bp, Artificial Sequence
  tag sequence
gtgatggtgt

SEQ ID NO: 1496 - DNA, 10 bp, Artificial Sequence
  tag sequence
tcatcatcag

SEQ ID NO: 1497 - DNA, 10 bp, Artificial Sequence
  tag sequence
gggaagggac

SEQ ID NO: 1498 - DNA, 10 bp, Artificial Sequence
  tag sequence
gtaaatatgg

SEQ ID NO: 1499 - DNA, 10 bp, Artificial Sequence
  tag sequence
taccagtgta

SEQ ID NO: 1500 - DNA, 10 bp, Artificial Sequence
  tag sequence
gtattctcca

SEQ ID NO: 1501 - DNA, 10 bp, Artificial Sequence
  tag sequence
tccccgtaca

SEQ ID NO: 1502 - DNA, 10 bp, Artificial Sequence
  tag sequence
tacataatta

SEQ ID NO: 1503 - DNA, 10 bp, Artificial Sequence
  tag sequence
tatgtgcacg

SEQ ID NO: 1504 - DNA, 10 bp, Artificial Sequence
  tag sequence
tgattggtgg

SEQ ID NO: 1505 - DNA, 10 bp, Artificial Sequence
  tag sequence
tgcttgtgta

SEQ ID NO: 1506 - DNA, 10 bp, Artificial Sequence
  tag sequence
tatctgtcta

SEQ ID NO: 1507 - DNA, 10 bp, Artificial Sequence
  tag sequence
accttggtgc

SEQ ID NO: 1508 - DNA, 10 bp, Artificial Sequence
  tag sequence
tttccttgcc

SEQ ID NO: 1509 - DNA, 10 bp, Artificial Sequence
  tag sequence
acacagcaag

SEQ ID NO: 1510 - DNA, 10 bp, Artificial Sequence
  tag sequence
tactttataa

SEQ ID NO: 1511 - DNA, 10 bp, Artificial Sequence
  tag sequence
aggaaggaac

SEQ ID NO: 1512 - DNA, 10 bp, Artificial Sequence
  tag sequence
gtaatcctgc

SEQ ID NO: 1513 - DNA, 10 bp, Artificial Sequence
  tag sequence
gctcagctgg

SEQ ID NO: 1514 - DNA, 10 bp, Artificial Sequence
  tag sequence
cctgcccacc

SEQ ID NO: 1515 - DNA, 10 bp, Artificial Sequence
  tag sequence
cctggctaat

SEQ ID NO: 1516 - DNA, 10 bp, Artificial Sequence
  tag sequence
gcccacaagt

SEQ ID NO: 1517 - DNA, 10 bp, Artificial Sequence
  tag sequence
ggcagccaga

SEQ ID NO: 1518 - DNA, 10 bp, Artificial Sequence
  tag sequence
acgcagggag

SEQ ID NO: 1519 - DNA, 10 bp, Artificial Sequence
  tag sequence
ttggccagga

SEQ ID NO: 1520 - DNA, 10 bp, Artificial Sequence
  tag sequence
taccctggca

SEQ ID NO: 1521 - DNA, 10 bp, Artificial Sequence
  tag sequence
tccctattaa

SEQ ID NO: 1522 - DNA, 10 bp, Artificial Sequence
  tag sequence
gctttatttg

SEQ ID NO: 1523 - DNA, 10 bp, Artificial Sequence
  tag sequence
acccccccgc

SEQ ID NO: 1524 - DNA, 10 bp, Artificial Sequence
  tag sequence
cacacagttt

SEQ ID NO: 1525 - DNA, 10 bp, Artificial Sequence
  tag sequence
aggtcaggag

SEQ ID NO: 1526 - DNA, 10 bp, Artificial Sequence
  tag sequence
tggaaagtga

SEQ ID NO: 1527 - DNA, 10 bp, Artificial Sequence
  tag sequence
gtggcaggca

SEQ ID NO: 1528 - DNA, 10 bp, Artificial Sequence
  tag sequence
gcctgcagtc

SEQ ID NO: 1529 - DNA, 10 bp, Artificial Sequence
  tag sequence
atgacccccg

SEQ ID NO: 1530 - DNA, 10 bp, Artificial Sequence
  tag sequence
cctgtagtcc

SEQ ID NO: 1531 - DNA, 10 bp, Artificial Sequence
  tag sequence
atcgtggcgg

SEQ ID NO: 1532 - DNA, 10 bp, Artificial Sequence
  tag sequence
cctgtaatcc

SEQ ID NO: 1533 - DNA, 10 bp, Artificial Sequence
  tag sequence
ccactgcact

SEQ ID NO: 1534 - DNA, 10 bp, Artificial Sequence
  tag sequence
tgatttcact

SEQ ID NO: 1535 - DNA, 10 bp, Artificial Sequence
  tag sequence
gtgtgggggg

SEQ ID NO: 1536 - DNA, 10 bp, Artificial Sequence
  tag sequence
attctccagt

SEQ ID NO: 1537 - DNA, 10 bp, Artificial Sequence
  tag sequence
gccgtgtccg

SEQ ID NO: 1538 - DNA, 10 bp, Artificial Sequence
  tag sequence
cagctcactg

SEQ ID NO: 1539 - DNA, 10 bp, Artificial Sequence
  tag sequence
gcctgtatga

SEQ ID NO: 1540 - DNA, 10 bp, Artificial Sequence
  tag sequence
ctgccaactt

SEQ ID NO: 1541 - DNA, 10 bp, Artificial Sequence
  tag sequence
caagtttgct

SEQ ID NO: 1542 - DNA, 10 bp, Artificial Sequence
  tag sequence
gggctggggt

SEQ ID NO: 1543 - DNA, 10 bp, Artificial Sequence
  tag sequence
cgccgccggc

SEQ ID NO: 1544 - DNA, 10 bp, Artificial Sequence
  tag sequence
gtaaaaaaaa

SEQ ID NO: 1545 - DNA, 10 bp, Artificial Sequence
  tag sequence
tagaaaggca

SEQ ID NO: 1546 - DNA, 10 bp, Artificial Sequence
  tag sequence
tgaaataaaa

SEQ ID NO: 1547 - DNA, 10 bp, Artificial Sequence
  tag sequence
tgaaaaaaaa

SEQ ID NO: 1548 - DNA, 10 bp, Artificial Sequence
  tag sequence
actccaaaaa

SEQ ID NO: 1549 - DNA, 10 bp, Artificial Sequence
  tag sequence
tggaagcact

SEQ ID NO: 1550 - DNA, 10 bp, Artificial Sequence
  tag sequence
gatgaactga

SEQ ID NO: 1551 - DNA, 10 bp, Artificial Sequence
  tag sequence
gccgccctgc

SEQ ID NO: 1552 - DNA, 10 bp, Artificial Sequence
  tag sequence
agaaaaaaaa

SEQ ID NO: 1553 - DNA, 10 bp, Artificial Sequence
  tag sequence
ccccagccag

SEQ ID NO: 1554 - DNA, 10 bp, Artificial Sequence
  tag sequence
ttgaagcttt

SEQ ID NO: 1555 - DNA, 10 bp, Artificial Sequence
  tag sequence
agctctccct

SEQ ID NO: 1556 - DNA, 10 bp, Artificial Sequence
  tag sequence
caaaaaaaaa

SEQ ID NO: 1557 - DNA, 10 bp, Artificial Sequence
  tag sequence
cccatccgaa

SEQ ID NO: 1558 - DNA, 10 bp, Artificial Sequence
  tag sequence
aggggcgcag

SEQ ID NO: 1559 - DNA, 10 bp, Artificial Sequence
  tag sequence
gtctgcacct

SEQ ID NO: 1560 - DNA, 10 bp, Artificial Sequence
  tag sequence
ccagaacaga

SEQ ID NO: 1561 - DNA, 10 bp, Artificial Sequence
  tag sequence
gtgttaacca

SEQ ID NO: 1562 - DNA, 10 bp, Artificial Sequence
  tag sequence
ctgggttaat

SEQ ID NO: 1563 - DNA, 10 bp, Artificial Sequence
  tag sequence
gtcttaaagt

SEQ ID NO: 1564 - DNA, 10 bp, Artificial Sequence
  tag sequence
agagaaattt

SEQ ID NO: 1565 - DNA, 10 bp, Artificial Sequence
  tag sequence
cttcgaaact

SEQ ID NO: 1566 - DNA, 10 bp, Artificial Sequence
  tag sequence
ttggtcctct

SEQ ID NO: 1567 - DNA, 10 bp, Artificial Sequence
  tag sequence
tgcacgtttt

SEQ ID NO: 1568 - DNA, 10 bp, Artificial Sequence
  tag sequence
gtgcgctgag

SEQ ID NO: 1569 - DNA, 10 bp, Artificial Sequence
  tag sequence
gggaagcaga

SEQ ID NO: 1570 - DNA, 10 bp, Artificial Sequence
  tag sequence
gcataatagg

SEQ ID NO: 1571 - DNA, 10 bp, Artificial Sequence
  tag sequence
gaaataaagt

SEQ ID NO: 1572 - DNA, 10 bp, Artificial Sequence
  tag sequence
caactaattc

SEQ ID NO: 1573 - DNA, 10 bp, Artificial Sequence
  tag sequence
gctgcccttg

SEQ ID NO: 1574 - DNA, 10 bp, Artificial Sequence
  tag sequence
gtttatggat

SEQ ID NO: 1575 - DNA, 10 bp, Artificial Sequence
  tag sequence
aataggtcca

SEQ ID NO: 1576 - DNA, 10 bp, Artificial Sequence
  tag sequence
cttcctgtga

SEQ ID NO: 1577 - DNA, 10 bp, Artificial Sequence
  tag sequence
aactaaaaaa

SEQ ID NO: 1578 - DNA, 10 bp, Artificial Sequence
  tag sequence
ccccctggat

SEQ ID NO: 1579 - DNA, 10 bp, Artificial Sequence
  tag sequence
ggcacctcag

SEQ ID NO: 1580 - DNA, 10 bp, Artificial Sequence
  tag sequence
taaggagctg

SEQ ID NO: 1581 - DNA, 10 bp, Artificial Sequence
  tag sequence
ttgaaacttt

SEQ ID NO: 1582 - DNA, 10 bp, Artificial Sequence
  tag sequence
ttggccaggg

SEQ ID NO: 1583 - DNA, 10 bp, Artificial Sequence
  tag sequence
taaaaaaaaa

SEQ ID NO: 1584 - DNA, 10 bp, Artificial Sequence
  tag sequence
caataaactg

SEQ ID NO: 1585 - DNA, 10 bp, Artificial Sequence
  tag sequence
tttgaaatga

SEQ ID NO: 1586 - DNA, 10 bp, Artificial Sequence
  tag sequence
cacaaacggt

SEQ ID NO: 1587 - DNA, 10 bp, Artificial Sequence
  tag sequence
aaggagatgg

SEQ ID NO: 1588 - DNA, 10 bp, Artificial Sequence
  tag sequence
gtgaccacgg

SEQ ID NO: 1589 - DNA, 10 bp, Artificial Sequence
  tag sequence
taataaaggt

SEQ ID NO: 1590 - DNA, 10 bp, Artificial Sequence
  tag sequence
ctcacttttt

SEQ ID NO: 1591 - DNA, 10 bp, Artificial Sequence
  tag sequence
ttcactgtga

SEQ ID NO: 1592 - DNA, 10 bp, Artificial Sequence
  tag sequence
cttccttgcc

SEQ ID NO: 1593 - DNA, 10 bp, Artificial Sequence
  tag sequence
gtgaaaaaaa

SEQ ID NO: 1594 - DNA, 10 bp, Artificial Sequence
  tag sequence
tgactggcag

SEQ ID NO: 1595 - DNA, 10 bp, Artificial Sequence
  tag sequence
aatgagcaac

SEQ ID NO: 1596 - DNA, 10 bp, Artificial Sequence
  tag sequence
gtggagcgga

SEQ ID NO: 1597 - DNA, 10 bp, Artificial Sequence
  tag sequence
ccattgaaac

SEQ ID NO: 1598 - DNA, 10 bp, Artificial Sequence
  tag sequence
gaaaacaaag

SEQ ID NO: 1599 - DNA, 10 bp, Artificial Sequence
  tag sequence
ttggcttttc

SEQ ID NO: 1600 - DNA, 10 bp, Artificial Sequence
  tag sequence
taaaaacttt

SEQ ID NO: 1601 - DNA, 10 bp, Artificial Sequence
  tag sequence
tcgccgcgac

SEQ ID NO: 1602 - DNA, 10 bp, Artificial Sequence
  tag sequence
caggccccac

SEQ ID NO: 1603 - DNA, 10 bp, Artificial Sequence
  tag sequence
agcagatcag

SEQ ID NO: 1604 - DNA, 10 bp, Artificial Sequence
  tag sequence
ataataaaag

SEQ ID NO: 1605 - DNA, 10 bp, Artificial Sequence
  tag sequence
agaaagatgt

SEQ ID NO: 1606 - DNA, 10 bp, Artificial Sequence
  tag sequence
gcgacagctc

SEQ ID NO: 1607 - DNA, 10 bp, Artificial Sequence
  tag sequence
tgctaattgt

SEQ ID NO: 1608 - DNA, 10 bp, Artificial Sequence
  tag sequence
gcaacttaga

SEQ ID NO: 1609 - DNA, 10 bp, Artificial Sequence
  tag sequence
tccccgtaca

SEQ ID NO: 1610 - DNA, 10 bp, Artificial Sequence
  tag sequence
cgtgggtggg

SEQ ID NO: 1611 - DNA, 10 bp, Artificial Sequence
  tag sequence
tgcagtgact

SEQ ID NO: 1612 - DNA, 10 bp, Artificial Sequence
  tag sequence
tgcaaacagc

SEQ ID NO: 1613 - DNA, 10 bp, Artificial Sequence
  tag sequence
gggtgggcag

SEQ ID NO: 1614 - DNA, 10 bp, Artificial Sequence
  tag sequence
ctgaaaattg

SEQ ID NO: 1615 - DNA, 10 bp, Artificial Sequence
  tag sequence
aggtgtgagc

SEQ ID NO: 1616 - DNA, 10 bp, Artificial Sequence
  tag sequence
agcagtgacg

SEQ ID NO: 1617 - DNA, 10 bp, Artificial Sequence
  tag sequence
agaatttagg

SEQ ID NO: 1618 - DNA, 10 bp, Artificial Sequence
  tag sequence
tctggggacg

SEQ ID NO: 1619 - DNA, 10 bp, Artificial Sequence
  tag sequence
gtactagtgt

SEQ ID NO: 1620 - DNA, 10 bp, Artificial Sequence
  tag sequence
cgaatgtcct

SEQ ID NO: 1621 - DNA, 10 bp, Artificial Sequence
  tag sequence
gctcaaaaac

SEQ ID NO: 1622 - DNA, 10 bp, Artificial Sequence
  tag sequence
cccgcctctt

SEQ ID NO: 1623 - DNA, 10 bp, Artificial Sequence
  tag sequence
acaggaaact

SEQ ID NO: 1624 - DNA, 10 bp, Artificial Sequence
  tag sequence
taattttgga

SEQ ID NO: 1625 - DNA, 10 bp, Artificial Sequence
  tag sequence
aagctcgccg

SEQ ID NO: 1626 - DNA, 10 bp, Artificial Sequence
  tag sequence
gactcttcag

SEQ ID NO: 1627 - DNA, 10 bp, Artificial Sequence
  tag sequence
gagcagcgcc

SEQ ID NO: 1628 - DNA, 10 bp, Artificial Sequence
  tag sequence
cttcaaaaaa

SEQ ID NO: 1629 - DNA, 10 bp, Artificial Sequence
  tag sequence
ctaaaaaaaa

SEQ ID NO: 1630 - DNA, 10 bp, Artificial Sequence
  tag sequence
ggtgagttac

SEQ ID NO: 1631 - DNA, 10 bp, Artificial Sequence
  tag sequence
gtggttaaaa

SEQ ID NO: 1632 - DNA, 10 bp, Artificial Sequence
  tag sequence
cccgaggcag

SEQ ID NO: 1633 - DNA, 10 bp, Artificial Sequence
  tag sequence
gccttgggtg

SEQ ID NO: 1634 - DNA, 10 bp, Artificial Sequence
  tag sequence
gacaaaaaaa

SEQ ID NO: 1635 - DNA, 10 bp, Artificial Sequence
  tag sequence
gggaaggcac

SEQ ID NO: 1636 - DNA, 10 bp, Artificial Sequence
  tag sequence
gagggtttag

SEQ ID NO: 1637 - DNA, 10 bp, Artificial Sequence
  tag sequence
gcgcgatgca

SEQ ID NO: 1638 - DNA, 10 bp, Artificial Sequence
  tag sequence
ttgaatcccc

SEQ ID NO: 1639 - DNA, 10 bp, Artificial Sequence
  tag sequence
gacacgaaca

SEQ ID NO: 1640 - DNA, 10 bp, Artificial Sequence
  tag sequence
gcggctttcc

SEQ ID NO: 1641 - DNA, 10 bp, Artificial Sequence
  tag sequence
gcttgcaaaa

SEQ ID NO: 1642 - DNA, 10 bp, Artificial Sequence
  tag sequence
gtgtggcagc

SEQ ID NO: 1643 - DNA, 10 bp, Artificial Sequence
  tag sequence
ttttgtgtga

SEQ ID NO: 1644 - DNA, 10 bp, Artificial Sequence
  tag sequence
ctggccctcg

SEQ ID NO: 1645 - DNA, 10 bp, Artificial Sequence
  tag sequence
aggtctgcca

SEQ ID NO: 1646 - DNA, 10 bp, Artificial Sequence
  tag sequence
tctccaacaa

SEQ ID NO: 1647 - DNA, 10 bp, Artificial Sequence
  tag sequence
ggtaaaatta

SEQ ID NO: 1648 - DNA, 10 bp, Artificial Sequence
  tag sequence
cttaaaaaaa

SEQ ID NO: 1649 - DNA, 10 bp, Artificial Sequence
  tag sequence
gcaggccaag

SEQ ID NO: 1650 - DNA, 10 bp, Artificial Sequence
  tag sequence
ggaaaagtgg

SEQ ID NO: 1651 - DNA, 10 bp, Artificial Sequence
  tag sequence
tttgcttttg

SEQ ID NO: 1652 - DNA, 10 bp, Artificial Sequence
  tag sequence
cttctccaaa

SEQ ID NO: 1653 - DNA, 10 bp, Artificial Sequence
  tag sequence
ttggtttttg

SEQ ID NO: 1654 - DNA, 10 bp, Artificial Sequence
  tag sequence
gtgcggagga

SEQ ID NO: 1655 - DNA, 10 bp, Artificial Sequence
  tag sequence
tgcagcacga

SEQ ID NO: 1656 - DNA, 10 bp, Artificial Sequence
  tag sequence
acacagcaag

SEQ ID NO: 1657 - DNA, 10 bp, Artificial Sequence
  tag sequence
cgtgggtggg

SEQ ID NO: 1658 - DNA, 10 bp, Artificial Sequence
  tag sequence
tttgaggatt

SEQ ID NO: 1659 - DNA, 10 bp, Artificial Sequence
  tag sequence
taaataattt

SEQ ID NO: 1660 - DNA, 10 bp, Artificial Sequence
  tag sequence
gcagaataga

SEQ ID NO: 1661 - DNA, 10 bp, Artificial Sequence
  tag sequence
gataactaca

SEQ ID NO: 1662 - DNA, 10 bp, Artificial Sequence
  tag sequence
gctttctcac

SEQ ID NO: 1663 - DNA, 10 bp, Artificial Sequence
  tag sequence
gaaaaggtta

SEQ ID NO: 1664 - DNA, 10 bp, Artificial Sequence
  tag sequence
aaattgttgg

SEQ ID NO: 1665 - DNA, 10 bp, Artificial Sequence
  tag sequence
gtaatgacag

SEQ ID NO: 1666 - DNA, 10 bp, Artificial Sequence
  tag sequence
tgcctctgtc

SEQ ID NO: 1667 - DNA, 10 bp, Artificial Sequence
  tag sequence
tcttgattta

SEQ ID NO: 1668 - DNA, 10 bp, Artificial Sequence
  tag sequence
gacgactgac

SEQ ID NO: 1669 - DNA, 10 bp, Artificial Sequence
  tag sequence
ccccctgccc

SEQ ID NO: 1670 - DNA, 10 bp, Artificial Sequence
  tag sequence
cagttctctg

SEQ ID NO: 1671 - DNA, 10 bp, Artificial Sequence
  tag sequence
agacaagctg

SEQ ID NO: 1672 - DNA, 10 bp, Artificial Sequence
  tag sequence
acagtgggga

SEQ ID NO: 1673 - DNA, 10 bp, Artificial Sequence
  tag sequence
cctgtgttgg

SEQ ID NO: 1674 - DNA, 10 bp, Artificial Sequence
  tag sequence
atgtcttttc

SEQ ID NO: 1675 - DNA, 10 bp, Artificial Sequence
  tag sequence
catttcagag

SEQ ID NO: 1676 - DNA, 10 bp, Artificial Sequence
  tag sequence
ggattgtctg

SEQ ID NO: 1677 - DNA, 10 bp, Artificial Sequence
  tag sequence
ttagtgtcgt

SEQ ID NO: 1678 - DNA, 10 bp, Artificial Sequence
  tag sequence
aggaactgta

SEQ ID NO: 1679 - DNA, 10 bp, Artificial Sequence
  tag sequence
acagcgctga

SEQ ID NO: 1680 - DNA, 10 bp, Artificial Sequence
  tag sequence
ggctggtctg

SEQ ID NO: 1681 - DNA, 10 bp, Artificial Sequence
  tag sequence
gaccgcagga

SEQ ID NO: 1682 - DNA, 10 bp, Artificial Sequence
  tag sequence
taatttgcat

SEQ ID NO: 1683 - DNA, 10 bp, Artificial Sequence
  tag sequence
aaaacattct

SEQ ID NO: 1684 - DNA, 10 bp, Artificial Sequence
  tag sequence
tctctgagca

SEQ ID NO: 1685 - DNA, 10 bp, Artificial Sequence
  tag sequence
tttaacggcc

SEQ ID NO: 1686 - DNA, 10 bp, Artificial Sequence
  tag sequence
tgtacctgta

SEQ ID NO: 1687 - DNA, 10 bp, Artificial Sequence
  tag sequence
tccagaatcc

SEQ ID NO: 1688 - DNA, 10 bp, Artificial Sequence
  tag sequence
ggaaggggag

SEQ ID NO: 1689 - DNA, 10 bp, Artificial Sequence
  tag sequence
aaaactgcac

SEQ ID NO: 1690 - DNA, 10 bp, Artificial Sequence
  tag sequence
catatcatta

SEQ ID NO: 1691 - DNA, 10 bp, Artificial Sequence
  tag sequence
agaccaaagt

SEQ ID NO: 1692 - DNA, 10 bp, Artificial Sequence
  tag sequence
tgtagtttga

SEQ ID NO: 1693 - DNA, 10 bp, Artificial Sequence
  tag sequence
tgctgtgcat

SEQ ID NO: 1694 - DNA, 10 bp, Artificial Sequence
  tag sequence
tatgagggta

SEQ ID NO: 1695 - DNA, 10 bp, Artificial Sequence
  tag sequence
gccataaaat

SEQ ID NO: 1696 - DNA, 10 bp, Artificial Sequence
  tag sequence
aagacagtgg

SEQ ID NO: 1697 - DNA, 10 bp, Artificial Sequence
  tag sequence
ccaatttatc

SEQ ID NO: 1698 - DNA, 10 bp, Artificial Sequence
  tag sequence
aaagtgaaga

SEQ ID NO: 1699 - DNA, 10 bp, Artificial Sequence
  tag sequence
ccaggaggaa

SEQ ID NO: 1700 - DNA, 10 bp, Artificial Sequence
  tag sequence
gagaaccgta

SEQ ID NO: 1701 - DNA, 10 bp, Artificial Sequence
  tag sequence
tgttctggag

SEQ ID NO: 1702 - DNA, 10 bp, Artificial Sequence
  tag sequence
aaggagatgg

SEQ ID NO: 1703 - DNA, 10 bp, Artificial Sequence
  tag sequence
tgtcctggtt

SEQ ID NO: 1704 - DNA, 10 bp, Artificial Sequence
  tag sequence
ggagaggaag

SEQ ID NO: 1705 - DNA, 10 bp, Artificial Sequence
  tag sequence
ctgacctgtg

SEQ ID NO: 1706 - DNA, 10 bp, Artificial Sequence
  tag sequence
tggaagcact

SEQ ID NO: 1707 - DNA, 10 bp, Artificial Sequence
  tag sequence
cacaaacggt

SEQ ID NO: 1708 - DNA, 10 bp, Artificial Sequence
  tag sequence
aagggagggt

SEQ ID NO: 1709 - DNA, 10 bp, Artificial Sequence
  tag sequence
taacagccag

SEQ ID NO: 1710 - DNA, 10 bp, Artificial Sequence
  tag sequence
acatcatcga

SEQ ID NO: 1711 - DNA, 10 bp, Artificial Sequence
  tag sequence
gtgaccacgg

SEQ ID NO: 1712 - DNA, 10 bp, Artificial Sequence
  tag sequence
tgttgaaaaa

SEQ ID NO: 1713 - DNA, 10 bp, Artificial Sequence
  tag sequence
gttcactgca

SEQ ID NO: 1714 - DNA, 10 bp, Artificial Sequence
  tag sequence
ccagaacaga

SEQ ID NO: 1715 - DNA, 10 bp, Artificial Sequence
  tag sequence
ctcataagga

SEQ ID NO: 1716 - DNA, 10 bp, Artificial Sequence
  tag sequence
cttaatcctg

SEQ ID NO: 1717 - DNA, 10 bp, Artificial Sequence
  tag sequence
tttgaaatga

SEQ ID NO: 1718 - DNA, 10 bp, Artificial Sequence
  tag sequence
ataattcttt

SEQ ID NO: 1719 - DNA, 10 bp, Artificial Sequence
  tag sequence
agattcaaac

SEQ ID NO: 1720 - DNA, 10 bp, Artificial Sequence
  tag sequence
ccgtccaagg

SEQ ID NO: 1721 - DNA, 10 bp, Artificial Sequence
  tag sequence
taatcctcaa

SEQ ID NO: 1722 - DNA, 10 bp, Artificial Sequence
  tag sequence
gtgcgctgag

SEQ ID NO: 1723 - DNA, 10 bp, Artificial Sequence
  tag sequence
gttccctggc

SEQ ID NO: 1724 - DNA, 10 bp, Artificial Sequence
  tag sequence
tgaagtaaca

SEQ ID NO: 1725 - DNA, 10 bp, Artificial Sequence
  tag sequence
cctagctgga

SEQ ID NO: 1726 - DNA, 10 bp, Artificial Sequence
  tag sequence
taccatcaat

SEQ ID NO: 1727 - DNA, 10 bp, Artificial Sequence
  tag sequence
aatcctgtgg

SEQ ID NO: 1728 - DNA, 10 bp, Artificial Sequence
  tag sequence
cagagatgaa

SEQ ID NO: 1729 - DNA, 10 bp, Artificial Sequence
  tag sequence
aaggtggagg

SEQ ID NO: 1730 - DNA, 10 bp, Artificial Sequence
  tag sequence
tgcacttcaa

SEQ ID NO: 1731 - DNA, 10 bp, Artificial Sequence
  tag sequence
ggcctgctgc

SEQ ID NO: 1732 - DNA, 10 bp, Artificial Sequence
  tag sequence
agggcttcca

SEQ ID NO: 1733 - DNA, 10 bp, Artificial Sequence
  tag sequence
gtgaaggcag

SEQ ID NO: 1734 - DNA, 10 bp, Artificial Sequence
  tag sequence
caagcatccc

SEQ ID NO: 1735 - DNA, 10 bp, Artificial Sequence
  tag sequence
agaatcactt

SEQ ID NO: 1736 - DNA, 10 bp, Artificial Sequence
  tag sequence
gaagcaggac

SEQ ID NO: 1737 - DNA, 10 bp, Artificial Sequence
  tag sequence
gcttttaagg

SEQ ID NO: 1738 - DNA, 10 bp, Artificial Sequence
  tag sequence
gcataatagg

SEQ ID NO: 1739 - DNA, 10 bp, Artificial Sequence
  tag sequence
ccctgggttc

SEQ ID NO: 1740 - DNA, 10 bp, Artificial Sequence
  tag sequence
gggacgagtg

SEQ ID NO: 1741 - DNA, 10 bp, Artificial Sequence
  tag sequence
ggcaagaaga

SEQ ID NO: 1742 - DNA, 10 bp, Artificial Sequence
  tag sequence
tgtgctaaat

SEQ ID NO: 1743 - DNA, 10 bp, Artificial Sequence
  tag sequence
atgtgaagag

SEQ ID NO: 1744 - DNA, 10 bp, Artificial Sequence
  tag sequence
tcagatcttt

SEQ ID NO: 1745 - DNA, 10 bp, Artificial Sequence
  tag sequence
ctaagacttc

SEQ ID NO: 1746 - DNA, 10 bp, Artificial Sequence
  tag sequence
caataaatgt

SEQ ID NO: 1747 - DNA, 10 bp, Artificial Sequence
  tag sequence
gttgtggtta

SEQ ID NO: 1748 - DNA, 10 bp, Artificial Sequence
  tag sequence
ggatttggcc

SEQ ID NO: 1749 - DNA, 10 bp, Artificial Sequence
  tag sequence
gtgctgaatg

SEQ ID NO: 1750 - DNA, 10 bp, Artificial Sequence
  tag sequence
ggagtgtgct

SEQ ID NO: 1751 - DNA, 10 bp, Artificial Sequence
  tag sequence
ggcaagcccc

SEQ ID NO: 1752 - DNA, 10 bp, Artificial Sequence
  tag sequence
taggttgtct

SEQ ID NO: 1753 - DNA, 10 bp, Artificial Sequence
  tag sequence
ttggtcctct

SEQ ID NO: 1754 - DNA, 10 bp, Artificial Sequence
  tag sequence
tccaaatcga

SEQ ID NO: 1755 - DNA, 10 bp, Artificial Sequence
  tag sequence
ctgggttaat

SEQ ID NO: 1756 - DNA, 10 bp, Artificial Sequence
  tag sequence
tggaaagtga

SEQ ID NO: 1757 - DNA, 10 bp, Artificial Sequence
  tag sequence
tggtgttgag

SEQ ID NO: 1758 - DNA, 10 bp, Artificial Sequence
  tag sequence
gccgaggaag

SEQ ID NO: 1759 - DNA, 10 bp, Artificial Sequence
  tag sequence
cacctaattg

SEQ ID NO: 1760 - DNA, 10 bp, Artificial Sequence
  tag sequence
gaaaaatggt

SEQ ID NO: 1761 - DNA, 10 bp, Artificial Sequence
  tag sequence
tgcacgtttt

SEQ ID NO: 1762 - DNA, 10 bp, Artificial Sequence
  tag sequence
gggctggggt

SEQ ID NO: 1763 - DNA, 10 bp, Artificial Sequence
  tag sequence
agcacctcca

SEQ ID NO: 1764 - DNA, 10 bp, Artificial Sequence
  tag sequence
accaaaaacc

SEQ ID NO: 1765 - DNA, 10 bp, Artificial Sequence
  tag sequence
caaatccaaa

SEQ ID NO: 1766 - DNA, 10 bp, Artificial Sequence
  tag sequence
ttaccatatc

SEQ ID NO: 1767 - DNA, 10 bp, Artificial Sequence
  tag sequence
gaaataaagc

SEQ ID NO: 1768 - DNA, 10 bp, Artificial Sequence
  tag sequence
acccccccgc

SEQ ID NO: 1769 - DNA, 10 bp, Artificial Sequence
  tag sequence
cgaggggcca

SEQ ID NO: 1770 - DNA, 10 bp, Artificial Sequence
  tag sequence
gatcaggcca

SEQ ID NO: 1771 - DNA, 10 bp, Artificial Sequence
  tag sequence
tttccctcaa

SEQ ID NO: 1772 - DNA, 10 bp, Artificial Sequence
  tag sequence
gagcagctgg

SEQ ID NO: 1773 - DNA, 10 bp, Artificial Sequence
  tag sequence
tttgcacctt

SEQ ID NO: 1774 - DNA, 10 bp, Artificial Sequence
  tag sequence
agccaccgcg

SEQ ID NO: 1775 - DNA, 10 bp, Artificial Sequence
  tag sequence
ggccgcgagg

SEQ ID NO: 1776 - DNA, 10 bp, Artificial Sequence
  tag sequence
ggggtaagaa

SEQ ID NO: 1777 - DNA, 10 bp, Artificial Sequence
  tag sequence
ggcccggctt

SEQ ID NO: 1778 - DNA, 10 bp, Artificial Sequence
  tag sequence
gggccaaccc

SEQ ID NO: 1779 - DNA, 10 bp, Artificial Sequence
  tag sequence
gaccagcaga

SEQ ID NO: 1780 - DNA, 10 bp, Artificial Sequence
  tag sequence
ctaaaatagt

SEQ ID NO: 1781 - DNA, 10 bp, Artificial Sequence
  tag sequence
ggcaattcaa

SEQ ID NO: 1782 - DNA, 10 bp, Artificial Sequence
  tag sequence
ccccgccaag

SEQ ID NO: 1783 - DNA, 10 bp, Artificial Sequence
  tag sequence
tccctattag

SEQ ID NO: 1784 - DNA, 10 bp, Artificial Sequence
  tag sequence
gccaaaacct

SEQ ID NO: 1785 - DNA, 10 bp, Artificial Sequence
  tag sequence
cccctattaa

SEQ ID NO: 1786 - DNA, 10 bp, Artificial Sequence
  tag sequence
gggggctcag

SEQ ID NO: 1787 - DNA, 10 bp, Artificial Sequence
  tag sequence
gagatccgca

SEQ ID NO: 1788 - DNA, 10 bp, Artificial Sequence
  tag sequence
gccggctcat

SEQ ID NO: 1789 - DNA, 10 bp, Artificial Sequence
  tag sequence
gattctgggt

SEQ ID NO: 1790 - DNA, 10 bp, Artificial Sequence
  tag sequence
acacagcaag

SEQ ID NO: 1791 - DNA, 10 bp, Artificial Sequence
  tag sequence
ctcaaccccc

SEQ ID NO: 1792 - DNA, 10 bp, Artificial Sequence
  tag sequence
ctctcaatat

SEQ ID NO: 1793 - DNA, 10 bp, Artificial Sequence
  tag sequence
cccgcctctt

SEQ ID NO: 1794 - DNA, 10 bp, Artificial Sequence
  tag sequence
ggggtgctgt

SEQ ID NO: 1795 - DNA, 10 bp, Artificial Sequence
  tag sequence
gctaggccgg

SEQ ID NO: 1796 - DNA, 10 bp, Artificial Sequence
  tag sequence
gagccaggct

SEQ ID NO: 1797 - DNA, 10 bp, Artificial Sequence
  tag sequence
agggtccccg

SEQ ID NO: 1798 - DNA, 10 bp, Artificial Sequence
  tag sequence
tggctgggaa

SEQ ID NO: 1799 - DNA, 10 bp, Artificial Sequence
  tag sequence
gagagaaaat

SEQ ID NO: 1800 - DNA, 10 bp, Artificial Sequence
  tag sequence
cctgtggtcc

SEQ ID NO: 1801 - DNA, 10 bp, Artificial Sequence
  tag sequence
cctccagcta

SEQ ID NO: 1802 - DNA, 10 bp, Artificial Sequence
  tag sequence
atcaaatcca

SEQ ID NO: 1803 - DNA, 10 bp, Artificial Sequence
  tag sequence
gtcaaaattt

SEQ ID NO: 1804 - DNA, 10 bp, Artificial Sequence
  tag sequence
gaaaccccag

SEQ ID NO: 1805 - DNA, 10 bp, Artificial Sequence
  tag sequence
ctccacccga

SEQ ID NO: 1806 - DNA, 10 bp, Artificial Sequence
  tag sequence
ttaaatagca

SEQ ID NO: 1807 - DNA, 10 bp, Artificial Sequence
  tag sequence
ctaacggggc

SEQ ID NO: 1808 - DNA, 10 bp, Artificial Sequence
  tag sequence
gtgctaagca

SEQ ID NO: 1809 - DNA, 10 bp, Artificial Sequence
  tag sequence
atgttagtgt

SEQ ID NO: 1810 - DNA, 10 bp, Artificial Sequence
  tag sequence
gaaatccaaa

SEQ ID NO: 1811 - DNA, 10 bp, Artificial Sequence
  tag sequence
gggggggggg

SEQ ID NO: 1812 - DNA, 10 bp, Artificial Sequence
  tag sequence
gacatcaagt

SEQ ID NO: 1813 - DNA, 10 bp, Artificial Sequence
  tag sequence
ctcgcgctgg

SEQ ID NO: 1814 - DNA, 10 bp, Artificial Sequence
  tag sequence
cctgcccacc

SEQ ID NO: 1815 - DNA, 10 bp, Artificial Sequence
  tag sequence
ctcaccgccc

SEQ ID NO: 1816 - DNA, 10 bp, Artificial Sequence
  tag sequence
aggagcgggg

SEQ ID NO: 1817 - DNA, 10 bp, Artificial Sequence
  tag sequence
tccctatgaa

SEQ ID NO: 1818 - DNA, 10 bp, Artificial Sequence
  tag sequence
ggaacaaaca

SEQ ID NO: 1819 - DNA, 10 bp, Artificial Sequence
  tag sequence
tccctatgaa

SEQ ID NO: 1820 - DNA, 10 bp, Artificial Sequence
  tag sequence
taggtcccct

SEQ ID NO: 1821 - DNA, 10 bp, Artificial Sequence
  tag sequence
tccgtattaa

SEQ ID NO: 1822 - DNA, 10 bp, Artificial Sequence
  tag sequence
tccgtattaa

SEQ ID NO: 1823 - DNA, 10 bp, Artificial Sequence
  tag sequence
ggctgcccag

SEQ ID NO: 1824 - DNA, 10 bp, Artificial Sequence
  tag sequence
ttcggttggt

SEQ ID NO: 1825 - DNA, 10 bp, Artificial Sequence
  tag sequence
tccctagtaa

SEQ ID NO: 1826 - DNA, 10 bp, Artificial Sequence
  tag sequence
agctgtcccc

SEQ ID NO: 1827 - DNA, 10 bp, Artificial Sequence
  tag sequence
acctgcacaa

SEQ ID NO: 1828 - DNA, 10 bp, Artificial Sequence
  tag sequence
ccgggggagc

SEQ ID NO: 1829 - DNA, 10 bp, Artificial Sequence
  tag sequence
gcctacccga

SEQ ID NO: 1830 - DNA, 10 bp, Artificial Sequence
  tag sequence
tccctattaa

SEQ ID NO: 1831 - DNA, 10 bp, Artificial Sequence
  tag sequence
atcgtggcgg

SEQ ID NO: 1832 - DNA, 10 bp, Artificial Sequence
  tag sequence
cctccagcta

SEQ ID NO: 1833 - DNA, 10 bp, Artificial Sequence
  tag sequence
gacatcaagt

SEQ ID NO: 1834 - DNA, 10 bp, Artificial Sequence
  tag sequence
tgtgggtgct

SEQ ID NO: 1835 - DNA, 10 bp, Artificial Sequence
  tag sequence
aggaaggaac

SEQ ID NO: 1836 - DNA, 10 bp, Artificial Sequence
  tag sequence
ctggccctcg

SEQ ID NO: 1837 - DNA, 10 bp, Artificial Sequence
  tag sequence
ctccacccga

SEQ ID NO: 1838 - DNA, 10 bp, Artificial Sequence
  tag sequence
aagctcgccg

SEQ ID NO: 1839 - DNA, 10 bp, Artificial Sequence
  tag sequence
cttcctgtga

SEQ ID NO: 1840 - DNA, 10 bp, Artificial Sequence
  tag sequence
aagaaaacct

SEQ ID NO: 1841 - DNA, 10 bp, Artificial Sequence
  tag sequence
attttctaaa

SEQ ID NO: 1842 - DNA, 10 bp, Artificial Sequence
  tag sequence
cggactcact

SEQ ID NO: 1843 - DNA, 10 bp, Artificial Sequence
  tag sequence
ggaacaaaca

SEQ ID NO: 1844 - DNA, 10 bp, Artificial Sequence
  tag sequence
aatatgtggg

SEQ ID NO: 1845 - DNA, 10 bp, Artificial Sequence
  tag sequence
ggactctgga

SEQ ID NO: 1846 - DNA, 10 bp, Artificial Sequence
  tag sequence
ctggccctcg

SEQ ID NO: 1847 - DNA, 10 bp, Artificial Sequence
  tag sequence
atcgtggcgg

SEQ ID NO: 1848 - DNA, 10 bp, Artificial Sequence
  tag sequence
atcgtggcgg

SEQ ID NO: 1849 - DNA, 10 bp, Artificial Sequence
  tag sequence
gcagggcctc

SEQ ID NO: 1850 - DNA, 10 bp, Artificial Sequence
  tag sequence
tgtgggtgct

SEQ ID NO: 1851 - DNA, 10 bp, Artificial Sequence
  tag sequence
ggactctgga

SEQ ID NO: 1852 - DNA, 10 bp, Artificial Sequence
  tag sequence
atgctcagcc

SEQ ID NO: 1853 - DNA, 10 bp, Artificial Sequence
  tag sequence
aaataaagaa

SEQ ID NO: 1854 - DNA, 10 bp, Artificial Sequence
  tag sequence
gcagtggcct

SEQ ID NO: 1855 - DNA, 10 bp, Artificial Sequence
  tag sequence
tggggttctt

SEQ ID NO: 1856 - DNA, 10 bp, Artificial Sequence
  tag sequence
atgctcagcc

SEQ ID NO: 1857 - DNA, 10 bp, Artificial Sequence
  tag sequence
ttgcgttgcg

SEQ ID NO: 1858 - DNA, 10 bp, Artificial Sequence
  tag sequence
tctccatacc

SEQ ID NO: 1859 - DNA, 10 bp, Artificial Sequence
  tag sequence
gatgtgcacg

SEQ ID NO: 1860 - DNA, 10 bp, Artificial Sequence
  tag sequence
gaccagcaga

SEQ ID NO: 1861 - DNA, 10 bp, Artificial Sequence
  tag sequence
ttaaatagca

SEQ ID NO: 1862 - DNA, 10 bp, Artificial Sequence
  tag sequence
ccgggggagc

SEQ ID NO: 1863 - DNA, 10 bp, Artificial Sequence
  tag sequence
gactttggaa

SEQ ID NO: 1864 - DNA, 10 bp, Artificial Sequence
  tag sequence
tggaaatgaa

SEQ ID NO: 1865 - DNA, 10 bp, Artificial Sequence
  tag sequence
cggggtggcc

SEQ ID NO: 1866 - DNA, 10 bp, Artificial Sequence
  tag sequence
tggaagcaga

SEQ ID NO: 1867 - DNA, 10 bp, Artificial Sequence
  tag sequence
ctgtcagcgt

SEQ ID NO: 1868 - DNA, 10 bp, Artificial Sequence
  tag sequence
caggagaccc

SEQ ID NO: 1869 - DNA, 10 bp, Artificial Sequence
  tag sequence
tccctaccga

SEQ ID NO: 1870 - DNA, 10 bp, Artificial Sequence
  tag sequence
tggaagcaga

SEQ ID NO: 1871 - DNA, 10 bp, Artificial Sequence
  tag sequence
agaatgagat

SEQ ID NO: 1872 - DNA, 10 bp, Artificial Sequence
  tag sequence
tattttcaca

SEQ ID NO: 1873 - DNA, 10 bp, Artificial Sequence
  tag sequence
acatagaccg

SEQ ID NO: 1874 - DNA, 10 bp, Artificial Sequence
  tag sequence
ctataggaga

SEQ ID NO: 1875 - DNA, 10 bp, Artificial Sequence
  tag sequence
gtaaatatgg

SEQ ID NO: 1876 - DNA, 10 bp, Artificial Sequence
  tag sequence
tttgtgggca

SEQ ID NO: 1877 - DNA, 10 bp, Artificial Sequence
  tag sequence
gggaagggac

SEQ ID NO: 1878 - DNA, 10 bp, Artificial Sequence
  tag sequence
gggaagggac

SEQ ID NO: 1879 - DNA, 10 bp, Artificial Sequence
  tag sequence
cttccttgcc

SEQ ID NO: 1880 - DNA, 10 bp, Artificial Sequence
  tag sequence
ggggaaatcg

SEQ ID NO: 1881 - DNA, 10 bp, Artificial Sequence
  tag sequence
tattttcaca

SEQ ID NO: 1882 - DNA, 10 bp, Artificial Sequence
  tag sequence
ccacgggatt

SEQ ID NO: 1883 - DNA, 10 bp, Artificial Sequence
  tag sequence
ggtcttcaag

SEQ ID NO: 1884 - DNA, 10 bp, Artificial Sequence
  tag sequence
gtgcgccgga

SEQ ID NO: 1885 - DNA, 10 bp, Artificial Sequence
  tag sequence
gagctggaaa

SEQ ID NO: 1886 - DNA, 10 bp, Artificial Sequence
  tag sequence
gagctggaaa

SEQ ID NO: 1887 - DNA, 10 bp, Artificial Sequence
  tag sequence
gagaaatcgt

SEQ ID NO: 1888 - DNA, 10 bp, Artificial Sequence
  tag sequence
aacggggccc

SEQ ID NO: 1889 - DNA, 10 bp, Artificial Sequence
  tag sequence
attcctgagc

SEQ ID NO: 1890 - DNA, 10 bp, Artificial Sequence
  tag sequence
atacagaata

SEQ ID NO: 1891 - DNA, 10 bp, Artificial Sequence
  tag sequence
caggagaagg

SEQ ID NO: 1892 - DNA, 10 bp, Artificial Sequence
  tag sequence
caggagaagg

SEQ ID NO: 1893 - DNA, 10 bp, Artificial Sequence
  tag sequence
gcggaggtgg

SEQ ID NO: 1894 - DNA, 10 bp, Artificial Sequence
  tag sequence
gccgttctta

SEQ ID NO: 1895 - DNA, 10 bp, Artificial Sequence
  tag sequence
tgaacagcag

SEQ ID NO: 1896 - DNA, 10 bp, Artificial Sequence
  tag sequence
gagtttattc

SEQ ID NO: 1897 - DNA, 10 bp, Artificial Sequence
  tag sequence
aatgaattat

SEQ ID NO: 1898 - DNA, 10 bp, Artificial Sequence
  tag sequence
taggtcagga

SEQ ID NO: 1899 - DNA, 10 bp, Artificial Sequence
  tag sequence
cgagagtgtg

SEQ ID NO: 1900 - DNA, 10 bp, Artificial Sequence
  tag sequence
gcgcctcccg

SEQ ID NO: 1901 - DNA, 10 bp, Artificial Sequence
  tag sequence
tgttgaaaaa

SEQ ID NO: 1902 - DNA, 10 bp, Artificial Sequence
  tag sequence
aagtttggtg

SEQ ID NO: 1903 - DNA, 10 bp, Artificial Sequence
  tag sequence
ggccgcgagg

SEQ ID NO: 1904 - DNA, 10 bp, Artificial Sequence
  tag sequence
accaaaaacc

SEQ ID NO: 1905 - DNA, 10 bp, Artificial Sequence
  tag sequence
gatcaggcca

SEQ ID NO: 1906 - DNA, 10 bp, Artificial Sequence
  tag sequence
tggaaatgac

SEQ ID NO: 1907 - DNA, 10 bp, Artificial Sequence
  tag sequence
cggggtggcc

SEQ ID NO: 1908 - DNA, 10 bp, Artificial Sequence
  tag sequence
ctaacggggc

SEQ ID NO: 1909 - DNA, 10 bp, Artificial Sequence
  tag sequence
cagataagtt

SEQ ID NO: 1910 - DNA, 10 bp, Artificial Sequence
  tag sequence
ccgggggagc

SEQ ID NO: 1911 - DNA, 10 bp, Artificial Sequence
  tag sequence
gtcaaaattt

SEQ ID NO: 1912 - DNA, 10 bp, Artificial Sequence
  tag sequence
gtgctaagcg

SEQ ID NO: 1913 - DNA, 10 bp, Artificial Sequence
  tag sequence
gactttggaa

SEQ ID NO: 1914 - DNA, 10 bp, Artificial Sequence
  tag sequence
cgccgacgat

SEQ ID NO: 1915 - DNA, 10 bp, Artificial Sequence
  tag sequence
ttgggatggg

SEQ ID NO: 1916 - DNA, 10 bp, Artificial Sequence
  tag sequence
catatcatta

SEQ ID NO: 1917 - DNA, 10 bp, Artificial Sequence
  tag sequence
tccaggaaac

SEQ ID NO: 1918 - DNA, 10 bp, Artificial Sequence
  tag sequence
ggcccctcac

SEQ ID NO: 1919 - DNA, 10 bp, Artificial Sequence
  tag sequence
acattccaag

SEQ ID NO: 1920 - DNA, 10 bp, Artificial Sequence
  tag sequence
ataaaaagaa

SEQ ID NO: 1921 - DNA, 10 bp, Artificial Sequence
  tag sequence
gaccagcaga

SEQ ID NO: 1922 - DNA, 10 bp, Artificial Sequence
  tag sequence
acttattatg

SEQ ID NO: 1923 - DNA, 10 bp, Artificial Sequence
  tag sequence
gtgcgctgag

SEQ ID NO: 1924 - DNA, 10 bp, Artificial Sequence
  tag sequence
tgcgctggcc

SEQ ID NO: 1925 - DNA, 10 bp, Artificial Sequence
  tag sequence
aggctcctgg

SEQ ID NO: 1926 - DNA, 10 bp, Artificial Sequence
  tag sequence
ctcaaccccc

SEQ ID NO: 1927 - DNA, 10 bp, Artificial Sequence
  tag sequence
cagcggcggg

SEQ ID NO: 1928 - DNA, 10 bp, Artificial Sequence
  tag sequence
ggcacctcag

SEQ ID NO: 1929 - DNA, 10 bp, Artificial Sequence
  tag sequence
gcctgtccct

SEQ ID NO: 1930 - DNA, 10 bp, Artificial Sequence
  tag sequence
atttcttcaa

SEQ ID NO: 1931 - DNA, 10 bp, Artificial Sequence
  tag sequence
tcgaagaacc

SEQ ID NO: 1932 - DNA, 10 bp, Artificial Sequence
  tag sequence
acattctttt

SEQ ID NO: 1933 - DNA, 10 bp, Artificial Sequence
  tag sequence
ctgtcagcgt

SEQ ID NO: 1934 - DNA, 10 bp, Artificial Sequence
  tag sequence
cagctggcca

SEQ ID NO: 1935 - DNA, 10 bp, Artificial Sequence
  tag sequence
actgaaagaa

SEQ ID NO: 1936 - DNA, 10 bp, Artificial Sequence
  tag sequence
ttctgtgctg

SEQ ID NO: 1937 - DNA, 10 bp, Artificial Sequence
  tag sequence
ggatgtgaaa

SEQ ID NO: 1938 - DNA, 10 bp, Artificial Sequence
  tag sequence
actcagcccg

SEQ ID NO: 1939 - DNA, 10 bp, Artificial Sequence
  tag sequence
tttccctcaa

SEQ ID NO: 1940 - DNA, 10 bp, Artificial Sequence
  tag sequence
ctaaaaaaaa

SEQ ID NO: 1941 - DNA, 10 bp, Artificial Sequence
  tag sequence
ggccacgtag

SEQ ID NO: 1942 - DNA, 10 bp, Artificial Sequence
  tag sequence
aagaaaggag

SEQ ID NO: 1943 - DNA, 10 bp, Artificial Sequence
  tag sequence
ggaggaattc

SEQ ID NO: 1944 - DNA, 10 bp, Artificial Sequence
  tag sequence
agccaccgcg

SEQ ID NO: 1945 - DNA, 10 bp, Artificial Sequence
  tag sequence
tgtaaacaat

SEQ ID NO: 1946 - DNA, 10 bp, Artificial Sequence
  tag sequence
accttgaagt

SEQ ID NO: 1947 - DNA, 10 bp, Artificial Sequence
  tag sequence
cataaatgcg

SEQ ID NO: 1948 - DNA, 10 bp, Artificial Sequence
  tag sequence
ttgctgactt

SEQ ID NO: 1949 - DNA, 10 bp, Artificial Sequence
  tag sequence
atggcaacag

SEQ ID NO: 1950 - DNA, 10 bp, Artificial Sequence
  tag sequence
ctctccaaac

SEQ ID NO: 1951 - DNA, 10 bp, Artificial Sequence
  tag sequence
tgcctgcacc

SEQ ID NO: 1952 - DNA, 10 bp, Artificial Sequence
  tag sequence
ggaaatgtca

SEQ ID NO: 1953 - DNA, 10 bp, Artificial Sequence
  tag sequence
caggtttcat

SEQ ID NO: 1954 - DNA, 10 bp, Artificial Sequence
  tag sequence
ccgtgactct

SEQ ID NO: 1955 - protein, 4 aa, Homo sapiens
KDEL